(19) United States
(12) Patent Application Publication
(10) Pub. No.: US 2024/0376073 A1
(43) Pub. Date: Nov. 14, 2024

(54) QUATERNARY AMMONIUM CATION SUBSTITUTED COMPOUNDS FOR THE TREATMENT OF BACTERIAL INFECTIONS

(71) Applicant: Hoffmann-La Roche Inc., Little Falls, NJ (US)

(72) Inventors: Sandra Marie Joseph GRALL-ULSEMER, Rosenau (FR); Xingchun HAN, Shanghai (CN); Joel Lukas KNECHT, Muensingen (CH); Christian LERNER, Bottmingen (CH); Mingming LI, Shanghai (CN); Yongqiang LIU, Shanghai (CN); Patrizio MATTEI, Riehen (CH); Matthias NETTEKOVEN, Grenzach-Wyhlen (DE); Philippe PFLIEGER, Schwoben (FR); Theodor STOLL, Binningen (CH); Jianhua WANG, Shanghai (CN); Min WANG, Shanghai (CN); Yongguang WANG, Shanghai (CN); Song YANG, Shanghai (CN); Chengang ZHOU, Shanghai (CN)

(73) Assignee: Hoffmann-La Roche Inc., Little Falls, NJ (US)

(21) Appl. No.: 18/404,194

(22) Filed: Jan. 4, 2024

Related U.S. Application Data
(63) Continuation of application No. PCT/EP2022/068362, filed on Jul. 4, 2022.

(30) Foreign Application Priority Data
Jul. 6, 2021 (WO) PCT/CN2021/104682

(51) Int. Cl. C07D 401/12; A61K 31/4164; A61K 31/4178; A61K 31/427; A61K 31/454; A61K 31/4545; A61K 31/496; A61K 31/5355; A61P 31/04; C07C 53/06; C07C 53/10; C07D 233/90; C07D 401/14; C07D 403/12; C07D 403/14; C07D 413/12; C07D 417/14; C07D 487/04

(52) U.S. Cl. CPC C07D 401/12; A61K 31/4164; A61K 31/4178; A61K 31/427; A61K 31/454; A61K 31/4545; A61K 31/496; A61K 31/5355; A61P 31/04; C07C 53/06; C07C 53/10; C07D 233/90; C07D 401/14; C07D 403/12; C07D 403/14; C07D 413/12; C07D 417/14; C07D 487/04

(57) ABSTRACT

The invention provides novel heterocyclic compounds having the general formula (I), and pharmaceutically acceptable salts thereof, wherein R¹ to R⁶ are as described herein:

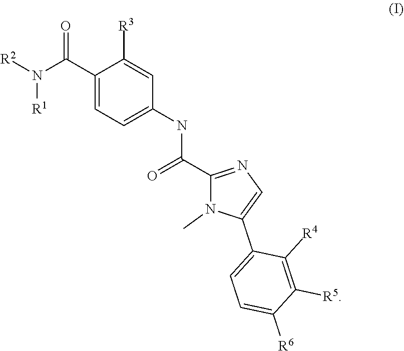

F [symbol] rther provided are pharmaceutical compositions including the compounds, processes of manufacturing the compounds and methods of using the compounds as medicaments, in particular methods of using the compounds as antibiotics for the treatment or prevention of bacterial infections and resulting diseases.

### CROSS-REFERENCE TO RELATED APPLICATIONS

This application is a Continuation of International Application No. PCT/EP2022/068362, filed on Jul. 4, 2022, which claims benefit of priority to International Application No. PCT/CN2021/104682, filed on Jul. 6, 2021, each of which is incorporated herein by reference in its entirety.

### FIELD OF THE INVENTION

The present invention relates to novel quaternary ammonium cation (QAC) substituted heterocyclic compounds which exhibit antibacterial properties. The invention also relates to methods of using the compounds for the treatment or prevention of bacterial infections and resulting diseases, in particular for the treatment or prevention of infections with Acinetobacter baumannii and resulting diseases.

### BACKGROUND

Acinetobacter baumannii is a Gram-negative, aerobic, nonfermenting bacterium recognized over the last decades as an emergining pathogen with very limited treatment options. A. baumannii is considered to be a serious threat by the US Centers for Disease Control and Prevention and belongs to the so called ‘ESKAPE’ pathogens (Enterococcus faecium, Staphylococcus aureus, Klebsiella pneumoniae, Acinetobacter baumannii, Pseudomonas aeruginosa and Enterobacter species & E. coli) that currently cause the majority of nosocomial infections and effectively “escape” the activity of antimicrobial agents. A. baumannii is most often encountered in intensive care units and surgical wards, where extensive antibiotic use has enabled selection for resistance against all known antimicrobials and where it causes infections that include bacteremia, pneumonia, meningitis, urinary tract infection, and wound infection.

A. baumannii has an exceptional ability to upregulate and acquire resistance determinants and shows an environmental persistence that allows its survival and spread in the nosocomial setting, making this organism a frequent cause of outbreaks of infection and an endemic, health care-associated pathogen.

Due to increasing antibiotic resistance to most if not all available therapeutic options, Muti-Drug Resistant (MDR) A. baumanniii infections, especially those caused by Carbapenem resistant A. baumannii, are extremely difficult or even impossible to treat with high mortality rate as well as increased morbidity and length of stay in intensive care unit.

Acinetobacter baumannii has been defined and still remains “a prime example of a mismatch between unmet medical needs and the current antimicrobial research and development pipeline” according to the Antimicrobial Availability Task Force (AATF) of the Infectious Diseases Society of America (IDSA). Thus, there is a high demand and need to identify compounds suitable for the treatment of diseases and infections caused by Acinetobacter baumannii.

The present invention provides novel compounds which exhibit activity against drug-susceptible as well as drug-resistant strains of Acinetobacter baumannii.

### SUMMARY OF THE DISCLOSURE

In a first aspect, the present invention provides a compound of formula (I):

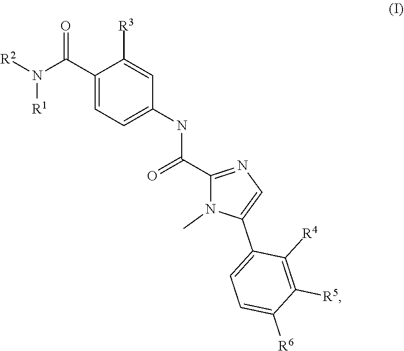

or a pharmaceutically acceptable salt thereof, wherein R¹ to R⁶ are as defined herein.

In one aspect, the present invention provides a process of manufacturing the compounds of formula (I) described herein, wherein said process is as described in any one of Schemes 1 to 4 herein.

In a further aspect, the present invention provides a compound of formula (I) as described herein, when manufactured according to the processes described herein.

In a further aspect, the present invention provides a compound of formula (I) as described herein, or a pharmaceutically acceptable salt thereof, for use as therapeutically active substance.

In a further aspect, the present invention provides a pharmaceutical composition comprising a compound of formula (I) as described herein, or a pharmaceutically acceptable salt thereof, and a therapeutically inert carrier.

In a further aspect, the present invention provides a compound of formula (I) as described herein, or a pharmaceutically acceptable salt thereof, for use as antibiotic.

In a further aspect, the present invention provides a compound of formula (I) as described herein, or a pharmaceutically acceptable salt thereof, for use in the treatment or prevention of nosocomial infections and resulting diseases.

In a further aspect, the present invention provides a compound of formula (I) as described herein, or a pharmaceutically acceptable salt thereof, for use in the treatment or prevention of infections and resulting diseases caused by Gram-negative bacteria.

In a further aspect, the present invention provides a compound of formula (I) as described herein, or a pharmaceutically acceptable salt thereof, for use in the treatment or prevention of infections and resulting diseases caused by Enterococcus faecium, Staphylococcus aureus, Klebsiella pneumoniae, Acinetobacter baumannii, Pseudomonas aeruginosa, Enterobacter species or E. coli, or a combination thereof.

### DETAILED DESCRIPTION OF THE DISCLOSURE

### Definitions

Features, integers, characteristics, compounds, chemical moieties or groups described in conjunction with a particular aspect, embodiment or example of the invention are to be understood to be applicable to any other aspect, embodiment or example described herein, unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The following definitions are provided to facilitate understanding of certain terms used frequently herein and are not meant to limit the scope of the present disclosure. All references referred to herein are incorporated by reference in their entirety.

The term “alkyl” refers to a mono- or multivalent, e.g., a mono- or bivalent, linear or branched saturated hydrocarbon group of 1 to 6 carbon atoms (“C₁-C₆-alkyl”), e.g., 1, 2, 3, 4, 5, or 6 carbon atoms. In some embodiments, the alkyl group contains 1 to 3 carbon atoms, e.g., 1, 2 or 3 carbon atoms. Some non-limiting examples of alkyl include methyl, ethyl, propyl, 2-propyl (isopropyl), n-butyl, iso-butyl, sec-butyl, tert-butyl, and 2,2-dimethylpropyl. Particularly preferred, yet non-limiting examples of alkyl include methyl and ethyl.

The term “alkoxy” refers to an alkyl group, as previously defined, attached to the parent molecular moiety via an oxygen atom. Unless otherwise specified, the alkoxy group contains 1 to 6 carbon atoms (“C₁-C₆-alkoxy”). In some preferred embodiments, the alkoxy group contains contains 1 to 4 carbon atoms. In still other embodiments, the alkoxy group contains 1 to 3 carbon atoms. Some non-limiting examples of alkoxy groups include methoxy, ethoxy, n-propoxy, isopropoxy, n-butoxy, isobutoxy and tert-butoxy. A particularly preferred, yet non-limiting example of alkoxy is methoxy.

The term “halogen” or “halo” refers to fluoro (F), chloro (Cl), bromo (Br), or iodo (I). Preferably, the term “halogen” or “halo” refers to fluoro (F), chloro (Cl) or bromo (Br). Particularly preferred, yet non-limiting examples of “halogen” or “halo” are fluoro (F) and chloro (Cl).

The term “heterocyclyl” refers to a saturated or partly unsaturated mono- or bicyclic, preferably monocyclic ring system of 3 to 14 ring atoms, preferably 3 to 10 ring atoms, more preferably 3 to 8 ring atoms wherein 1, 2, or 3 of said ring atoms are heteroatoms selected from N, O and S, the remaining ring atoms being carbon. Preferably, 1 to 2 of said ring atoms are selected from N and O, the remaining ring atoms being carbon. “Bicyclic heterocyclyl” refers to heterocyclic moieties consisting of two cycles having two ring atoms in common, i.e., the bridge separating the two rings is either a single bond or a chain of one or two ring atoms, and to spirocyclic moieties, i.e., the two rings are connected via one common ring atom. Some non-limiting examples of heterocyclyl groups include azetidin-3-yl; azetidin-2-yl; oxetan-3-yl; oxetan-2-yl; piperidyl; piperazinyl; pyrrolidinyl; 2-oxopyrrolidin-1-yl; 2-oxopyrrolidin-3-yl; 5-oxopyrrolidin-2-yl; 5-oxopyrrolidin-3-yl; 2-oxo-1-piperidyl; 2-oxo-3-piperidyl; 2-oxo-4-piperidyl; 6-oxo-2-piperidyl; 6-oxo-3-piperidyl; 1-piperidinyl; 2-piperidinyl; 3-piperidinyl; 4-piperidinyl; morpholino; morpholin-2-yl; morpholin-3-yl; pyrrolidinyl (e.g., pyrrolidin-3-yl); 3-azabicyclo[3.1.0]hexan-6-yl; 2,5-diazabicyclo[2.2.1]heptan-2-yl; 2-azaspiro[3.3]heptan-2-yl; 2,6-diazaspiro[3.3]heptan-2-yl; and 2,3,3a,4,6,6a-hexahydro-1H-pyrrolo[3,4-c]pyrrol-5-yl.

The term “heterocyclylalkyl” refers to a heterocyclyl moiety that is bound to the parent molecule via an alkyldiyl group. A non-limiting example of heterocyclylalkyl is piperidylmethyl.

The term “alkyldiyl” refers to a saturated linear or branched-chain divalent hydrocarbon radical of about one to six carbon atoms (C₁-C₆). Examples of alkyldiyl groups include, but are not limited to, methylene (—CH₂—), ethylene (—CH₂CH₂—), propylene (—CH₂CH₂CH₂—), and the like. An alkyldiyl group may also be referred to as an “alkylene” group.

The term “heteroaryl” refers to a mono- or multivalent, monocyclic or bicyclic, preferably bicyclic ring system having a total of 5 to 14 ring members, preferably, 5 to 12 ring members, and more preferably 5 to 10 ring members, wherein at least one ring in the system is aromatic, and at least one ring in the system contains one or more heteroatoms. Preferably, “heteroaryl” refers to a 5-10 membered heteroaryl comprising 1, 2, 3 or 4 heteroatoms independently selected from O, S and N. Most preferably, “heteroaryl” refers to a 5-10 membered heteroaryl comprising 1 to 2 heteroatoms independently selected from O and N. Some non-limiting examples of heteroaryl include 2-pyridyl, 3-pyridyl, 4-pyridyl, pyrimidin-2-yl, pyrimidin-4-yl, pyrimidin-5-yl, pyrimidin-6-yl, indol-1-yl, 1H-indol-2-yl, 1H-indol-3-yl, 1H-indol-4-yl, 1H-indol-5-yl, 1H-indol-6-yl, 1H-indol-7-yl, 1,2-benzoxazol-3-yl, 1,2-benzoxazol-4-yl, 1,2-benzoxazol-5-yl, 1,2-benzoxazol-6-yl, 1,2-benzoxazol-7-yl, 1H-indazol-3-yl, 1H-indazol-4-yl, 1H-indazol-5-yl, 1H-indazol-6-yl, 1H-indazol-7-yl, pyrazol-1-yl, 1H-pyrazol-3-yl, 1H-pyrazol-4-yl, 1H-pyrazol-5-yl, imidazol-1-yl, 1H-imidazol-2-yl, 1H-imidazol-4-yl, 1H-imidazol-5-yl, oxazol-2-yl, oxazol-4-yl, oxazol-5-yl, thiazol-4-yl, and 1,2,4-oxadiazol-3-yl.

The term “hydroxy” refers to an —OH group.

The term “amino” refers to an —NH₂ group.

The term “cyano” refers to a —CN (nitrile) group.

The term “carbamoyl” refers to a —C(O)NH₂ group.

The term “carboxy” refers to a —C(O)OH group.

The term “carbonyl” refers to a carbon radical having two of the four covalent bonds shared with an oxygen atom (C=O).

The term “haloalkyl” refers to an alkyl group, wherein at least one of the hydrogen atoms of the alkyl group has been replaced by a halogen atom, preferably fluoro. Preferably, “haloalkyl” refers to an alkyl group wherein 1, 2 or 3 hydrogen atoms of the alkyl group have been replaced by a halogen atom, most preferably fluoro. Non-limiting examples of haloalkyl are fluoromethyl, difluoromethyl, trifluoromethyl, trifluoroethyl, 2-fluoroethyl, and 2,2-difluoroethyl. A particularly preferred, yet non-limiting example of haloalkyl is trifluoromethyl.

The term “cyanoalkyl” refers to an alkyl group, wherein at least one of the hydrogen atoms of the alkyl group has been replaced by cyano group. Preferably, “cyanoalkyl” refers to an alkyl group wherein 1, 2 or 3 hydrogen atoms of the alkyl group have been replaced by a cyano group. Most preferably, “cyanoalkyl” refers to an alkyl group wherein 1 hydrogen atom of the alkyl group has been replaced by a cyano group. A preferred, yet non-limiting example of cyanoalkyl is cyanomethyl.

The term “haloalkoxy” refers to an alkoxy group, wherein at least one of the hydrogen atoms of the alkoxy group has been replaced by a halogen atom, preferably fluoro. Preferably, “haloalkoxy” refers to an alkoxy group wherein 1, 2 or 3 hydrogen atoms of the alkoxy group have been replaced by a halogen atom, most preferably fluoro. Particularly preferred, yet non-limiting examples of haloalkoxy are fluoromethoxy (FCH₂O—), difluoromethoxy (F₂CHO—), and trifluoromethoxy (F₃CO—).

The term “cyanoalkoxy” refers to an alkoxy group, wherein at least one of the hydrogen atoms of the alkoxy group has been replaced by cyano group. Preferably, “cyanoalkoxy” refers to an alkoxy group wherein 1, 2 or 3 hydrogen atoms of the alkoxy group have been replaced by a cyano group. Most preferably, “cyanoalkoxy” refers to an alkoxy group wherein 1 hydrogen atom of the alkoxy group has been replaced by a cyano group. A preferred, yet non-limiting example of cyanoalkoxy is cyanomethoxy.

The term “carbamoylalkyl” refers to an alkyl group, wherein at least one of the hydrogen atoms of the alkyl group has been replaced by a carbamoyl group. Preferably, “carbamoylalkyl” refers to an alkyl group wherein 1, 2 or 3 hydrogen atoms of the alkyl group have been replaced by a carbamoyl group. Most preferably, “carbamoylalkyl” refers to an alkyl group wherein 1 hydrogen atom of the alkoxy group has been replaced by a carbamoyl group. A preferred, yet non-limiting example of carbamoylalkyl is 2-amino-2-oxo-ethyl.

The term “hydroxyalkyl” refers to an alkyl group, wherein at least one of the hydrogen atoms of the alkyl group has been replaced by a hydroxy group. Preferably, “hydroxyalkyl” refers to an alkyl group wherein 1, 2 or 3 hydrogen atoms, most preferably 1 hydrogen atom of the alkyl group have been replaced by a hydroxy group. Preferred, yet non-limiting examples of hydroxyalkyl are hydroxymethyl, hydroxyethyl (e.g. 2-hydroxyethyl), and 3-hydroxy-3-methyl-butyl.

The term “carboxyalkyl” refers to an alkyl group, wherein at least one of the hydrogen atoms of the alkyl group has been replaced by a carboxy group. Preferably, “carboxyalkyl” refers to an alkyl group wherein 1, 2 or 3 hydrogen atoms, most preferably 1 hydrogen atom of the alkyl group have been replaced by a carboxy group. A preferred, yet non-limiting example of carboxyalkyl is carboxymethyl.

The term “alkoxyalkyl” refers to an alkyl group, wherein at least one of the hydrogen atoms of the alkyl group has been replaced by an alkoxy group. Preferably, “alkoxyalkyl” refers to an alkyl group wherein 1, 2 or 3 hydrogen atoms, most preferably 1 hydrogen atom of the alkyl group have been replaced by an alkoxy group. Preferred, yet non-limiting examples of alkoxyalkyl are methoxymethyl and 2-ethoxyethyl.

The term “aminoalkyl” refers to an alkyl group, wherein at least one of the hydrogen atoms of the alkyl group has been replaced by an amino group. Preferably, “aminoalkyl” refers to an alkyl group wherein 1, 2 or 3 hydrogen atoms, most preferably 1 hydrogen atom of the alkyl group have been replaced by an amino group. Preferred, yet non-limiting examples of aminoalkyl are aminomethyl, aminoethyl (e.g. 2-aminoethyl), 3-amino-3-methyl-butyl, aminopentyl (e.g., 5-aminopentyl), and aminohexyl (e.g., 6-aminohexyl).

The term “pharmaceutically acceptable salt” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid and the like, in particular hydrochloric acid, and organic acids such as acetic acid, trifluoroacetic acid, propionic acid, glycolic acid, pyruvic acid, oxalic acid, maleic acid, malonic acid, succinic acid, fumaric acid, tartaric acid, lactic acid, citric acid, benzoic acid, cinnamic acid, mandelic acid, methanesulfonic acid, ethanesulfonic acid, p-toluenesulfonic acid, salicylic acid, N-acetylcystein and the like. In addition these salts may be prepared by addition of an inorganic base or an organic base to the free acid. Salts derived from an inorganic base include, but are not limited to, the sodium, potassium, lithium, ammonium, calcium, magnesium salts and the like. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyimine resins and the like. Particular pharmaceutically acceptable salts of compounds of formula (I) are hydrochlorides, fumarates, lactates (in particular derived from L-(+)-lactic acid), tartrates (in particular derived from L-(+)-tartaric acid) and trifluoroacetates.

The compounds of formula (I) can contain several asymmetric centers and can be present in the form of optically pure enantiomers, mixtures of enantiomers such as, for example, racemates, optically pure diastereioisomers, mixtures of diastereoisomers, diastereoisomeric racemates or mixtures of diastereoisomeric racemates.

According to the Cahn-Ingold-Prelog Convention, the asymmetric carbon atom can be of the “R” or “S” configuration.

The term “treatment” as used herein includes: (1) inhibiting the state, disorder or condition (e.g. arresting, reducing or delaying the development of the disease, or a relapse thereof in case of maintenance treatment, of at least one clinical or subclinical symptom thereof); and/or (2) relieving the condition (i.e., causing regression of the state, disorder or condition or at least one of its clinical or subclinical symptoms). The benefit to a patient to be treated is either statistically significant or at least perceptible to the patient or to the physician. However, it will be appreciated that when a medicament is administered to a patient to treat a disease, the outcome may not always be effective treatment.

The term “mammal” as used herein includes both humans and non-humans and includes but is not limited to humans, non-human primates, canines, felines, murines, bovines, equines, and porcines. In a particularly preferred embodiment, the term “mammal” refers to humans.

The term “nosocomial infection” refers to a hospital-acquired infection (HAI), which is an infection that is acquired in a hospital or other health care facility. To emphasize both hospital and nonhospital settings, it is sometimes instead called a health care-associated infection (HAI or HCAI). Such an infection can be acquired in hospitals, nursing homes, rehabilitation facilities, outpatient clinics, or other clinical settings.

### Compounds of the Invention

In a first aspect, the present invention provides a compound of formula (I):

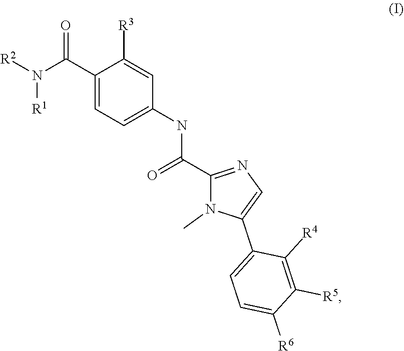

or a pharmaceutically acceptable salt thereof, wherein:
R¹ is selected from (C₁-C₆-alkyl)₃N⁺—C₁-C₆-alkyl-O—C₁-C₆-alkyl-, (C₁-C₆-alkyl)₃N⁺—C₁-C₆-alkyl-, (C₁-C₆-alkyl)₃N⁺—C₁-C₆-alkyl-C(O)—NH—C₁-C₆-alkyl-, carbamoyl-C₁-C₆-alkyl-(C₁-C₆-alkyl)₂N⁺—C₁-C₆-alkyl-O—C₁-C₆-alkyl-, carbamoyl-C₁-C₆-alkyl-(C₁-C₆-alkyl)₂N⁺—C₁-C₆-alkyl-, a group

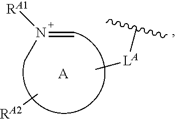

and a group

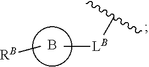

and R² is hydrogen; or
R¹ and R², taken together with the nitrogen atom to which they are attached, form a group

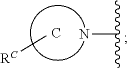

R³ is halogen or C₁-C₆-alkyl;
R⁴, R⁵ and R⁶ are each independently selected from hydrogen, halogen, cyano, halo-C₁-C₆-alkyl, C₁-C₆-alkoxy, halo-C₁-C₆-alkoxy, and cyano-C₁-C₆-alkoxy;
R^{A1} is C₁-C₆-alkyl or cyano-C₁-C₆-alkyl;
R^{A2} is selected from C₁-C₆-alkyl, hydroxy-C₁-C₆-alkyl, hydroxy-C₁-C₆-alkyl-O—C₁-C₆-alkyl-, carbamoyl-C₁-C₆-alkyl, C₁-C₆-alkoxy-C₁-C₆-alkyl-, C₁-C₆-alkyl-S(O)₂—C₁-C₆-alkyl-, and (C₁-C₆-alkyl)₃N⁺—C₁-C₆-alkyl-C(O)—NH—;
R^{B} is selected from (C₁-C₆-alkyl)₃N⁺—C₁-C₆-alkyl-NH—, (C₁-C₆-alkyl)₃N⁺—C₁-C₆-alkyl-C(O)—NH—, carbamoyl-C₁-C₆-alkyl-(C₁-C₆-alkyl)₂N⁺—C₁-C₆-alkyl-C(O)—, and a group

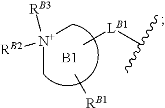

R^{B1} is hydrogen or hydroxy;
R^{B2} and R^{B3} are each independently C₁-C₆-alkyl or carbamoyl-C₁-C₆-alkyl;
R^{C} is selected from (R^{C8})₃N⁺—C₁-C₆-alkyl-L^{C2}- a group

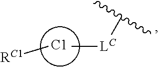

a group

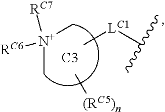

and a group

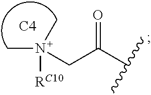

R^{C1} is (C₁-C₆-alkyl)₃N⁺—, (R^{C9})₃N⁺—C₁-C₆-alkyl-C(O)—, or a group

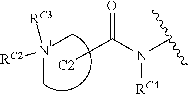

R^{C2}, R^{C3}, R^{C6}, R^{C7}, and R^{C10} are independently selected from C₁-C₆-alkyl, carboxy-C₁-C₆-alkyl-, amino-C₁-C₆-alkyl, carbamoyl-C₁-C₆-alkyl, (C₁-C₆-alkyl)₂N—C₁-C₆-alkyl-, (3- to 14-membered heterocyclyl)-C₁-C₆-alkyl-;
R^{C4} is hydrogen or C₁-C₆-alkyl;
each R^{C5} is independently selected from hydrogen, hydroxy, carbamoyl, C₁-C₆-alkyl, hydroxy-C₁-C₆-alkyl, and (3- to 14-membered heterocyclyl)-C₁-C₆-alkyl-; each R^{C8} and each R^{C9} is independently selected from amino-C₁-C₆-alkyl-, carboxy-C₁-C₆-alkyl-, carbamoyl-C₁-C₆-alkyl-, and C₁-C₆-alkyl-;
A is 3- to 14-membered heteroaryl;
B is selected from 3- to 14-membered heteroaryl, 3- to 14-membered heterocyclyl, and C₃-C₁₀-cycloalkyl;
B1, C, C2, C3, and C4 are all 3- to 14-membered heterocyclyl;
C1 is 3- to 14-membered heterocyclyl or 3- to 14-membered heteroaryl;
L^{A} is C₁-C₆-alkyldiyl;
L^{B} is a covalent bond or C₁-C₆-alkyldiyl;
L^{B1} is selected from —C₁-C₆-alkyldiyl-, —C(O)—C₁-C₆-alkyldiyl-, carbonyl, —NH—C(O)—, and —C(O)—NH—;
L^{C} is carbonyl or —C₁-C₆-alkyldiyl-NH—C(O)—;
L^{C1} is selected from carbonyl, —CH₂C(O)—, —NH—C(O)—, and —C(O)—NH—;
L^{C2} is selected from carbonyl, a covalent bond, —O—C₁-C₆-alkyl-NH—C(O)—, —C(O)—NH—, and —NH—C(O)—; and
n is 1 or 2.

In one embodiment, there is provided a compound of formula (I) as described herein, or a pharmaceutically acceptable salt thereof, wherein
R₁ is selected from (C₁-C₆-alkyl)₃N⁺—C₁-C₆-alkyl-O—C₁-C₆-alkyl-, (C₁-C₆-alkyl)₃N⁺—C₁-C₆-alkyl-, (C₁-C₆-alkyl)₃N⁺—C₁-C₆-alkyl-C(O)—NH—C₁-C₆-alkyl-, carbamoyl-C₁-C₆-alkyl-(C₁-C₆-alkyl)₂N⁺—C₁-C₆-alkyl-O—C₁-C₆-alkyl-, carbamoyl-C₁-C₆-alkyl-(C₁-C₆-alkyl)₂N⁺-C1-C₆-alkyl-, a group

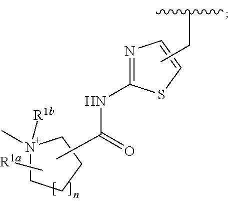

a group

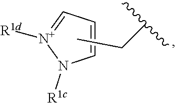

a group

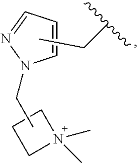

a group

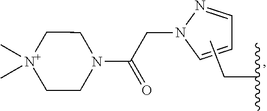

a group

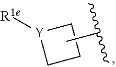

a group

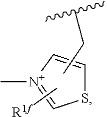

and a group

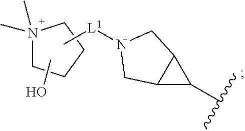

and R² is hydrogen; or
R¹ and R², taken together with the nitrogen atom to which they are attached, form a group

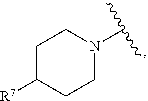

a group

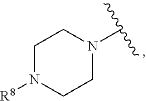

a group

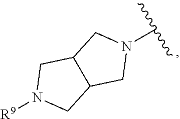

or a group

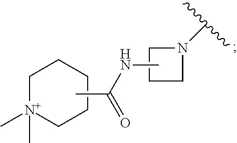

R^{1a} is hydrogen or hydroxy;
R^{1b} is C₁-C₆-alkyl or carbamoyl-C₁-C₆-alkyl-;
R^{1c} is selected from C₁-C₆-alkyl, hydroxy-C₁-C₆-alkyl, hydroxy-C₁-C₆-alkyl-O—C₁-C₆-alkyl-, carbamoyl-C₁-C₆-alkyl, C₁-C₆-alkoxy-C₁-C₆-alkyl-, and C₁-C₆-alkyl-S(O)₂—C₁-C₆-alkyl-;
R^{1d} is C₁-C₆-alkyl or cyano-C₁-C₆-alkyl;
R^{1e} is selected from (C₁-C₆-alkyl)₃N⁺—C₁-C₆-alkyl-NH—, (C₁-C₆-alkyl)₃N⁺—C₁-C₆-alkyl-C(O)—NH—, and carbamoyl-C₁-C₆-alkyl-(C₁-C₆-alkyl)₂N⁺—C₁-C₆-alkyl-C(O)—;
R^{1f} is (C₁-C₆-alkyl)₃N⁺—C₁-C₆-alkyl-C(O)—NH—;
R⁷ is selected from (C₁-C₆-alkyl)₃N⁺—C₁-C₆-alkyl-, (C₁-C₆-alkyl)₃N⁺—C₁-C₆-alkyl-O—C₁-C₆-alkyl-NH—C(O)—, a group

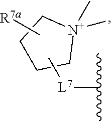

a group

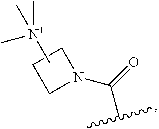

and a group

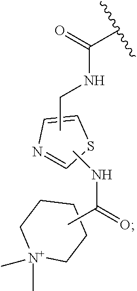

R^{7a} is hydrogen or hydroxy;
R⁸ is selected from (R^{8a})₃N⁺—C₁-C₆-alkyl-L^{8a}- a group

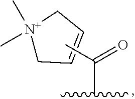

a group

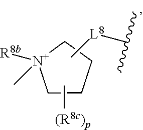

a group

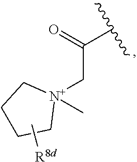

a group

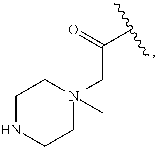 [SMILES: *C(=O)C[N+]1(C)CCNCC1]

a group

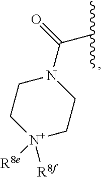

a group

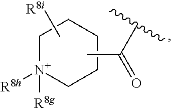

a group

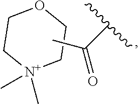

a group

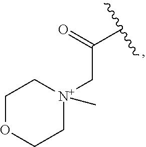 [SMILES: *C(=O)C[N+]1(C)CCOCC1]

a group

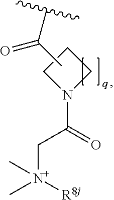

a group

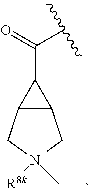

and a group

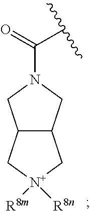

each R^{8a} is independently selected from amino-C₁-C₆-alkyl-, carboxy-C₁-C₆-alkyl-, carbamoyl-C₁-C₆-alkyl-, and C₁-C₆-alkyl-;
R^{8b} is C₁-C₆-alkyl or a group

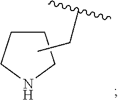

each R^{8c} is independently selected from hydrogen, hydroxy, C₁-C₆-alkyl, hydroxy-C₁-C₆-alkyl, and a group

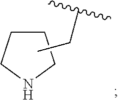

R^{8d} is selected from hydrogen, hydroxy, and carbamoyl;
R^{8e} is carboxy-C₁-C₆-alkyl-;
R^{8f} is C₁-C₆-alkyl or carboxy-C₁-C₆-alkyl-;
R^{8g} is selected from C₁-C₆-alkyl, carboxy-C₁-C₆-alkyl, and carbamoyl-C₁-C₆-alkyl;
R^{8h} is selected from C₁-C₆-alkyl, amino-C₁-C₆-alkyl, carbamoyl-C₁-C₆-alkyl, carboxy-C₁-C₆-alkyl, (C₁-C₆-alkyl)₂N—C₁-C₆-alkyl-, and a group

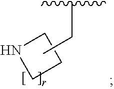

R⁸ⁱ is hydrogen or hydroxy;
R^{8j} is C₁-C₆-alkyl or carbamoyl-C₁-C₆-alkyl;
R^{8k} is carbamoyl-C₁-C₆-alkyl- or carboxy-C₁-C₆-alkyl-;
R^{8m} is carboxy-C₁-C₆-alkyl-;
R⁸ⁿ is amino-C₁-C₆-alkyl-;
R⁹ is (C₁-C₆-alkyl)₃N⁺—C₁-C₆-alkyl-C(O)— or a group

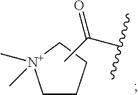

L¹ is carbonyl, —NH—C(O)—;
L⁷ is —NH—C(O)— or —C(O)—NH—;
L⁸ is carbonyl, —CH₂C(O)—, —NH—C(O)—;
L^{8a} is selected from carbonyl, a covalent bond, and —C(O)—NH—;
Y is N or CH;
n and p are each independently 1 or 2;
q is 1 or 3; and
r is 1, 2 or 3.

In a preferred embodiment, there is provided a compound of formula (I) as described herein, or a pharmaceutically acceptable salt thereof, wherein

R¹ is selected from a group

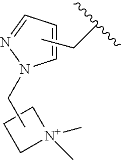

and a group

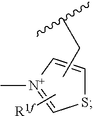

and R² is hydrogen; or
R¹ and R², taken together with the nitrogen atom to which they are attached, form a group

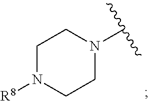

R^{1f} is (C₁-C₆-alkyl)₃N⁺—C₁-C₆-alkyl-C(O)—NH—;
R⁸ is selected from (R^{8a})₃N⁺—C₁-C₆-alkyl-L^{8a}- a group

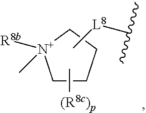

a group

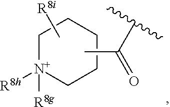

a group

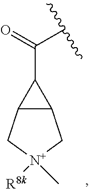

and a group

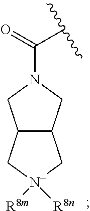

each R^{8a} is independently selected from carbamoyl-C₁-C₆-alkyl- and C₁-C₆-alkyl-;
R^{8b} is C₁-C₆-alkyl;
each R^{8c} is independently selected from hydroxy and C₁-C₆-alkyl;
R^{8g} is selected from C₁-C₆-alkyl, carboxy-C₁-C₆-alkyl, and carbamoyl-C₁-C₆-alkyl;
R^{8h} is C₁-C₆-alkyl or a group

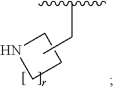

R⁸ⁱ is hydrogen or hydroxy;
R^{8k} is carbamoyl-C₁-C₆-alkyl-;
R^{8m} is carboxy-C₁-C₆-alkyl-;
R⁸ⁿ is amino-C₁-C₆-alkyl-;
L⁸ and L^{8a} are both carbonyl;
p is 1 or 2; and
r is 1.

In a particularly preferred embodiment, there is provided a compound of formula (I) as described herein, or a pharmaceutically acceptable salt thereof, wherein
R¹ is selected from a group

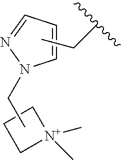

and a group

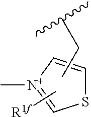

and R² is hydrogen; or
R¹ and R², taken together with the nitrogen atom to which they are attached, form a group

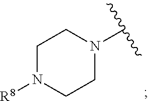

R^{1f} is (CH₃)₃N⁺—CH₂—C(O)—NH—;
R⁸ is selected from (R^{8a})₃N⁺—CH₂-L^{8a}-, a group

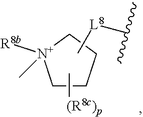

a group

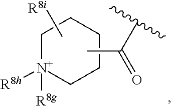

a group

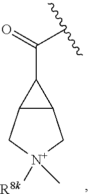

and a group

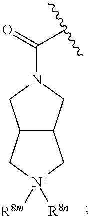

each R^{8a} is independently selected from 2-amino-2-oxo-ethyl and methyl;
R^{8b} is methyl;
each R^{8c} is independently selected from hydroxy and methyl;
R^{8g} is selected from methyl, carboxymethyl, and 2-amino-2-oxo-ethyl;
R^{8h} is methyl or a group

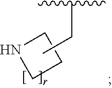

R⁸ⁱ is hydrogen or hydroxy;
R^{8k} is 2-amino-2-oxo-ethyl;
R^{8m} is carboxymethyl;
R⁸ⁿ is 3-aminopropyl;
L⁸ and L⁸a are both carbonyl;
p is 1 or 2; and
r is 1.

In one embodiment, there is provided a compound of formula (I) as described herein, or a pharmaceutically acceptable salt thereof, wherein R³ is halogen or C₁-C₆-alkyl.

In a preferred embodiment, there is provided a compound of formula (I) as described herein, or a pharmaceutically acceptable salt thereof, wherein R³ is halogen.

In a particularly preferred embodiment, there is provided a compound of formula (I) as described herein, or a pharmaceutically acceptable salt thereof, wherein R³ is chloro.

In one embodiment, there is provided a compound of formula (I) as described herein, or a pharmaceutically acceptable salt thereof, wherein:
R⁴ is selected from hydrogen, halogen, and halo-C₁-C₆-alkyl;
R⁵ is selected from hydrogen, halogen, cyano, and halo-C₁-C₆-alkyl; and
R⁶ is selected from C₁-C₆-alkoxy, halo-C₁-C₆-alkoxy, and cyano-C₁-C₆-alkoxy.

In a preferred embodiment, there is provided a compound of formula (I) as described herein, or a pharmaceutically acceptable salt thereof, wherein:
R⁴ and R⁵ are each halogen; and
R⁶ is C₁-C₆-alkoxy or halo-C₁-C₆-alkoxy.

In a particularly preferred embodiment, there is provided a compound of formula (I) as described herein, or a pharmaceutically acceptable salt thereof, wherein:
R⁴ and R⁵ are each independently fluoro or chloro; and
R⁶ is selected from methoxy, fluoromethoxy, and difluoromethoxy.

In one embodiment, there is provided a compound of formula (I) as described herein, or a pharmaceutically acceptable salt thereof, wherein:
R¹ is selected from (C₁-C₆-alkyl)₃N⁺—C₁-C₆-alkyl-O—C₁-C₆-alkyl-, (C₁-C₆-alkyl)₃N⁺—C₁-C₆-alkyl-, (C₁-C₆-alkyl)₃N⁺—C₁-C₆-alkyl-C(O)—NH—C₁-C₆-alkyl-, carbamoyl-C₁-C₆-alkyl-(C₁-C₆-alkyl)₂N⁺—C₁-C₆-alkyl-O—C₁-C₆-alkyl-, carbamoyl-C₁-C₆-alkyl-(C₁-C₆-alkyl)₂N⁺—C₁-C₆-alkyl-, a group

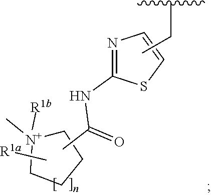

a group

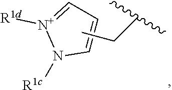

a group

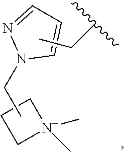

a group

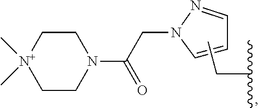

a group

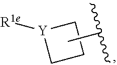

a group

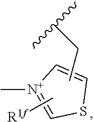

and a group

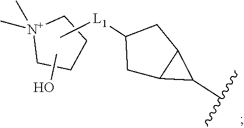

and R² is hydrogen; or
R¹ and R², taken together with the nitrogen atom to which they are attached, form a group

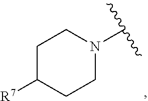

a group

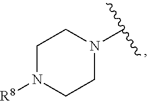

a group

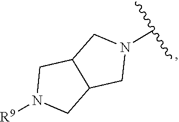

or a group

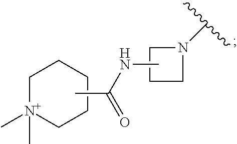

R^{1a} is hydrogen or hydroxy;
R^{1b} is C₁-C₆-alkyl or carbamoyl-C₁-C₆-alkyl-;
R^{1e} is selected from C₁-C₆-alkyl, hydroxy-C₁-C₆-alkyl, hydroxy-C₁-C₆-alkyl-O—C₁-C₆-alkyl-, carbamoyl-C₁-C₆-alkyl, C₁-C₆-alkoxy-C₁-C₆-alkyl-, and C₁-C₆-alkyl-S(O)₂—C₁-C₆-alkyl-;
R^{1d} is C₁-C₆-alkyl or cyano-C₁-C₆-alkyl;
R^{1e} is selected from (C₁-C₆-alkyl)₃N⁺—C₁-C₆-alkyl-NH—, (C₁-C₆-alkyl)₃N⁺—C₁-C₆-alkyl-C(O)—NH—, and carbamoyl-C₁-C₆-alkyl-(C₁-C₆-alkyl)₂N⁺—C₁-C₆-alkyl-C(O)—;
R^{1f} is (C₁-C₆-alkyl)₃N⁺—C₁-C₆-alkyl-C(O)—NH—;
R³ is halogen or C₁-C₆-alkyl;
R⁴ is selected from hydrogen, halogen, and halo-C₁-C₆-alkyl;
R⁵ is selected from hydrogen, halogen, cyano, and halo-C₁-C₆-alkyl;
R⁶ is selected from C₁-C₆-alkoxy, halo-C₁-C₆-alkoxy, and cyano-C₁-C₆-alkoxy;
R⁷ is selected from (C₁-C₆-alkyl)₃N⁺—C₁-C₆-alkyl-, (C₁-C₆-alkyl)₃N⁺—C₁-C₆-alkyl-O—C₁-C₆-alkyl-NH—C(O)—, a group

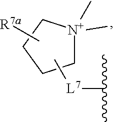

a group

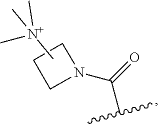

and a group

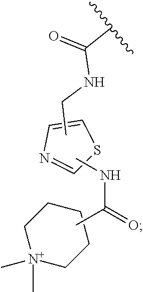

R^{7a} is hydrogen or hydroxy;
R⁸ is selected from (R^{8a})₃N⁺—C₁-C₆-alkyl-L^{8a}- a group

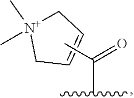

a group

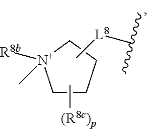

a group

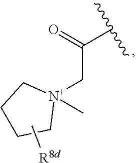

a group

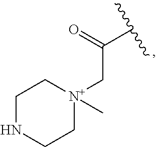 [SMILES: *C(=O)C[N+]1(C)CCNCC1]

a group

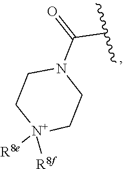

a group

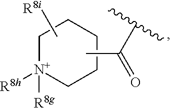

a group

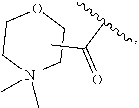

a group

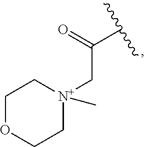 [SMILES: *C(=O)C[N+]1(C)CCOCC1]

a group

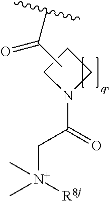

a group

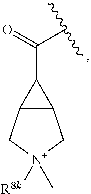

and a group

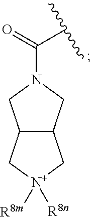

each R^{8a} is independently selected from amino-C₁-C₆-alkyl-, carboxy-C₁-C₆-alkyl-, carbamoyl-C₁-C₆-alkyl-, and C₁-C₆-alkyl-;
R^{8b} is C₁-C₆-alkyl or a group

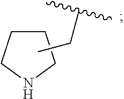

each R^{8e} is independently selected from hydrogen, hydroxy, C₁-C₆-alkyl, hydroxy-C₁-C₆-alkyl, and a group

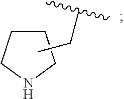

R^{8d} is selected from hydrogen, hydroxy, and carbamoyl;
R^{8e} is carboxy-C₁-C₆-alkyl-;
R^{8f} is C₁-C₆-alkyl or carboxy-C₁-C₆-alkyl-;
R^{8g} is selected from C₁-C₆-alkyl, carboxy-C₁-C₆-alkyl, and carbamoyl-C₁-C₆-alkyl;
R^{8h} is selected from C₁-C₆-alkyl, amino-C₁-C₆-alkyl, carbamoyl-C₁-C₆-alkyl, carboxy-C₁-C₆-alkyl, (C₁-C₆-alkyl)₂N—C₁-C₆-alkyl-, and a group

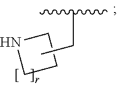

R⁸ⁱ is hydrogen or hydroxy;
R^{8j} is C₁-C₆-alkyl or carbamoyl-C₁-C₆-alkyl;
R^{8k} is carbamoyl-C₁-C₆-alkyl- or carboxy-C₁-C₆-alkyl-;
R^{8m} is carboxy-C₁-C₆-alkyl-;
R⁸ⁿ is amino-C₁-C₆-alkyl-;
R⁹ is (C₁-C₆-alkyl)₃N⁺—C₁-C₆-alkyl-C(O)— or a group

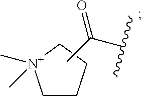

L¹ is carbonyl, —NH—C(O)—;
L⁷ is —NH—C(O)— or —C(O)—NH—;
L⁸ is carbonyl, —CH₂C(O)—, —NH—C(O)—;
L^{8a} is selected from carbonyl, a covalent bond, and —C(O)—NH—;
Y is N or CH;
n and p are each independently 1 or 2;
q is 1 or 3; and
r is 1, 2 or 3.

In a preferred embodiment, there is provided a compound of formula (I) as described herein, or a pharmaceutically acceptable salt thereof, wherein:
R¹ is selected from a group

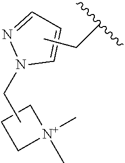

and a group

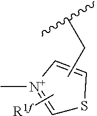

and R² is hydrogen; or
R¹ and R², taken together with the nitrogen atom to which they are attached, form a group

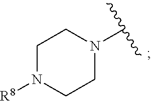

R^{1f} is (C₁-C₆-alkyl)₃N⁺—C₁-C₆-alkyl-C(O)—NH—;
R³, R⁴ and R⁵ are each halogen;
R⁶ is C₁-C₆-alkoxy or halo-C₁-C₆-alkoxy;
R⁸ is selected from (R^{8a})₃N⁺—C₁-C₆-alkyl-L^{8a}- a group

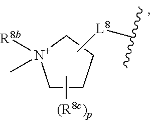

a group

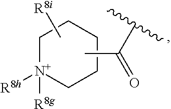

a group

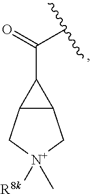

and a group

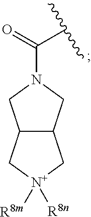

each R^{8a} is independently selected from carbamoyl-C₁-C₆-alkyl- and C₁-C₆-alkyl-;
R^{8b} is C₁-C₆-alkyl;
each R^{8c} is independently selected from hydroxy and C₁-C₆-alkyl;
R^{8g} is selected from C₁-C₆-alkyl, carboxy-C₁-C₆-alkyl, and carbamoyl-C₁-C₆-alkyl;
R^{8h} is C₁-C₆-alkyl or a group

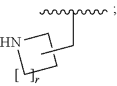

R⁸ⁱ is hydrogen or hydroxy;
R^{8k} is carbamoyl-C₁-C₆-alkyl-;
R^{8m} is carboxy-C₁-C₆-alkyl-;
R⁸ⁿ is amino-C₁-C₆-alkyl-;
L⁸ and L^{8a} are both carbonyl;
p is 1 or 2; and
r is 1.

In a particularly preferred embodiment, there is provided a compound of formula (I) as described herein, or a pharmaceutically acceptable salt thereof, wherein:
R¹ is selected from a group

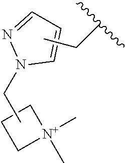

and a group

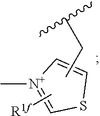

and R² is hydrogen; or
R¹ and R², taken together with the nitrogen atom to which they are attached, form a group

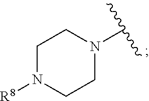

R^{1f} is (CH₃)₃N⁺—CH₂—C(O)—NH—;
R³ is chloro;
R⁴ and R⁵ are each independently fluoro or chloro;
R⁶ is selected from methoxy, fluoromethoxy, and difluoromethoxy;
R⁸ is selected from (R^{8a})₃N⁺—CH₂-L^{8a}-, a group

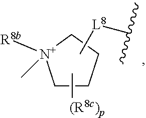

a group

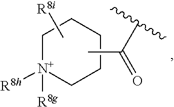

a group

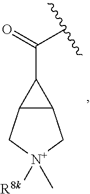

and a group

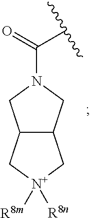

each R^{8a} is independently selected from 2-amino-2-oxo-ethyl and methyl;
R^{8b} is methyl;
each R^{8c} is independently selected from hydroxy and methyl;
R^{8g} is selected from methyl, carboxymethyl, and 2-amino-2-oxo-ethyl;
R^{8h} is methyl or a group

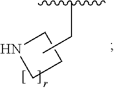

R⁸ⁱ is hydrogen or hydroxy;
R^{8k} is 2-amino-2-oxo-ethyl;
R^{8m} is carboxymethyl;
R⁸ⁿ is 3-aminopropyl;
L⁸ and L⁸a are both carbonyl;
p is 1 or 2; and
r is 1.

In one embodiment, there is provided a compound of formula (I) as described herein, or a pharmaceutically acceptable salt thereof, wherein said compound of formula (I) is a compound of formula (II):

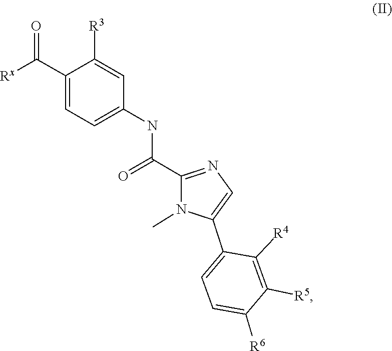

wherein R^{x} is selected from:

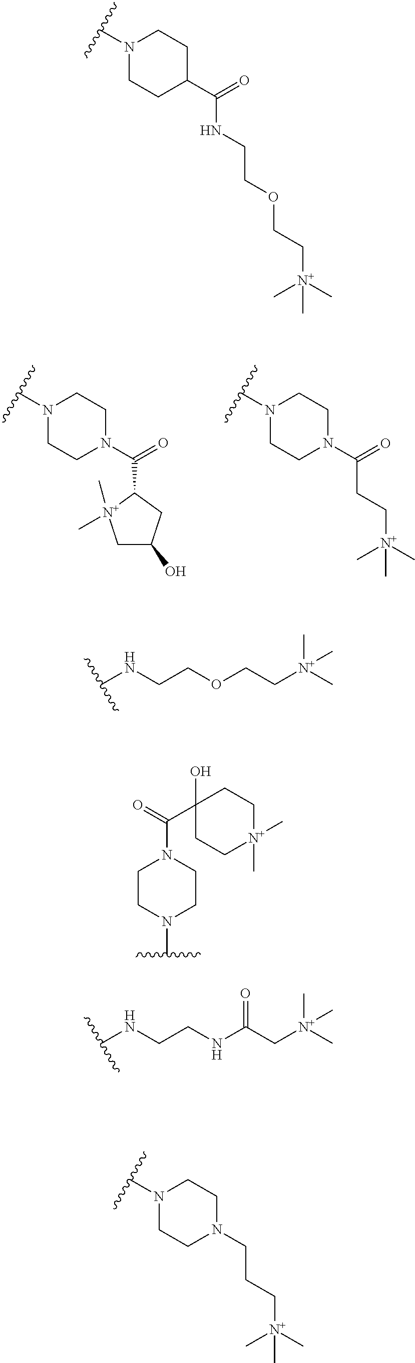

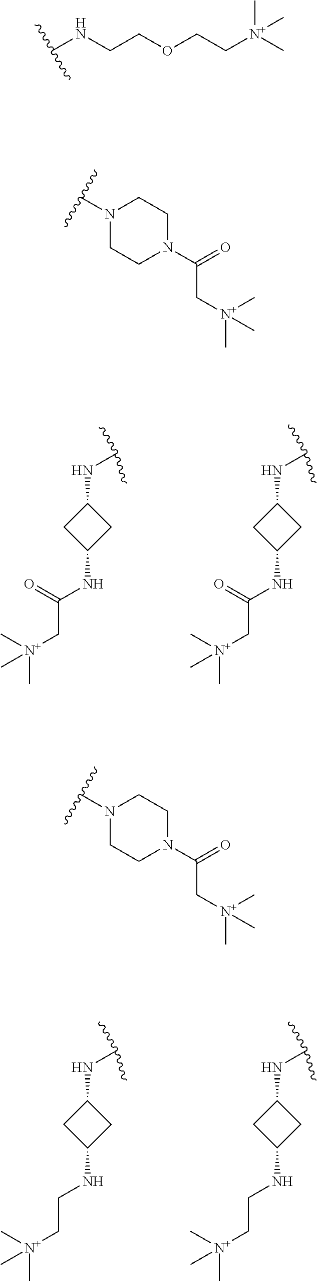

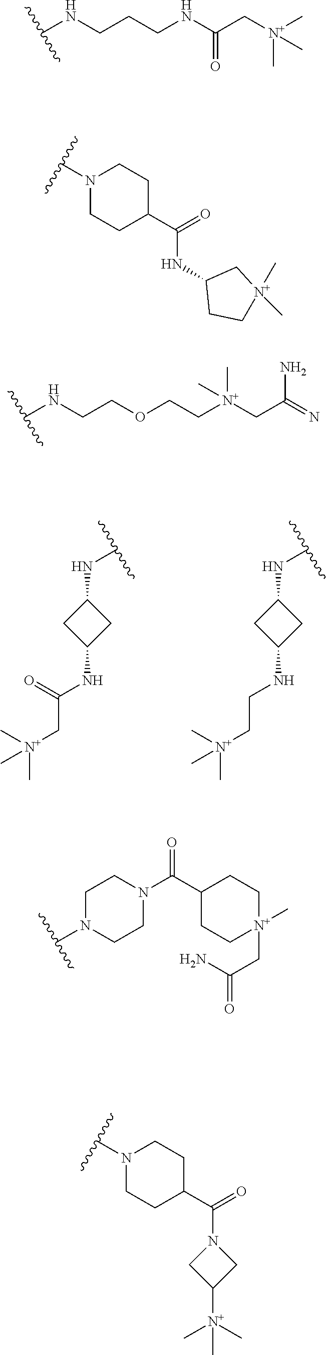

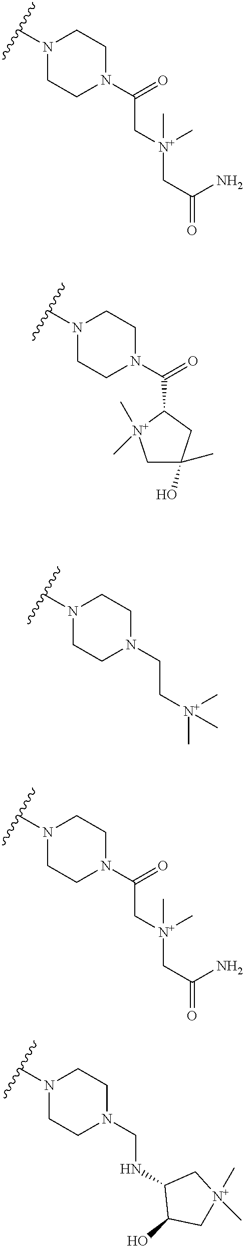 [SMILES: *N1CCN(C(=O)C[N+](C)(C)CC(N)=O)CC1 ; *N1CCN(C(=O)[C@@H]2C[C@](C)(O)C[N+]2(C)C)CC1 ; *N1CCN(CC[N+](C)(C)C)CC1 ; *N1CCN(C(=O)C[N+](C)(C)CC(N)=O)CC1 ; *N1CCN(CN[C@@H]2C[N+](C)(C)C[C@H]2O)CC1]

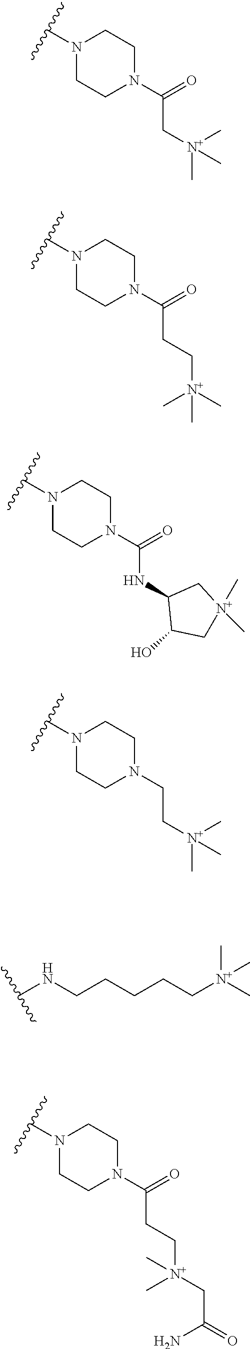 [SMILES: *N1CCN(C(=O)C[N+](C)(C)C)CC1 ; *N1CCN(C(=O)CC[N+](C)(C)C)CC1 ; *N1CCN(C(=O)N[C@H]2C[N+](C)(C)C[C@@H]2O)CC1 ; *N1CCN(CC[N+](C)(C)C)CC1 ; *NCCCCC[N+](C)(C)C ; *N1CCN(C(=O)CC[N+](C)(C)CC(N)=O)CC1]

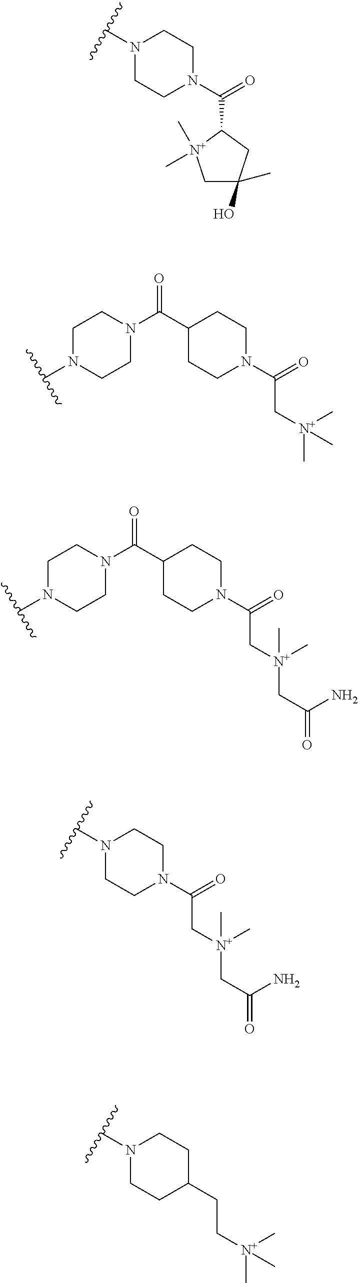 [SMILES: *N1CCN(C(=O)[C@@H]2C[C@@](C)(O)C[N+]2(C)C)CC1 ; *N1CCN(C(=O)C2CCN(C(=O)C[N+](C)(C)C)CC2)CC1 ; *N1CCN(C(=O)C2CCN(C(=O)C[N+](C)(C)CC(N)=O)CC2)CC1 ; *N1CCN(C(=O)C[N+](C)(C)CC(N)=O)CC1 ; *N1CCC(CC[N+](C)(C)C)CC1]

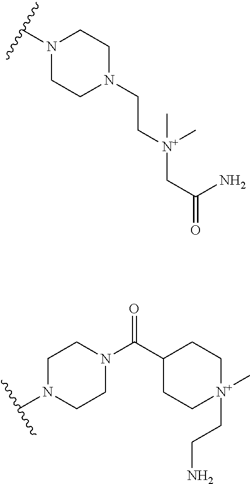 [SMILES: *N1CCN(CC[N+](C)(C)CC(N)=O)CC1 ; *N1CCN(C(=O)C2CC[N+](C)(CCN)CC2)CC1]

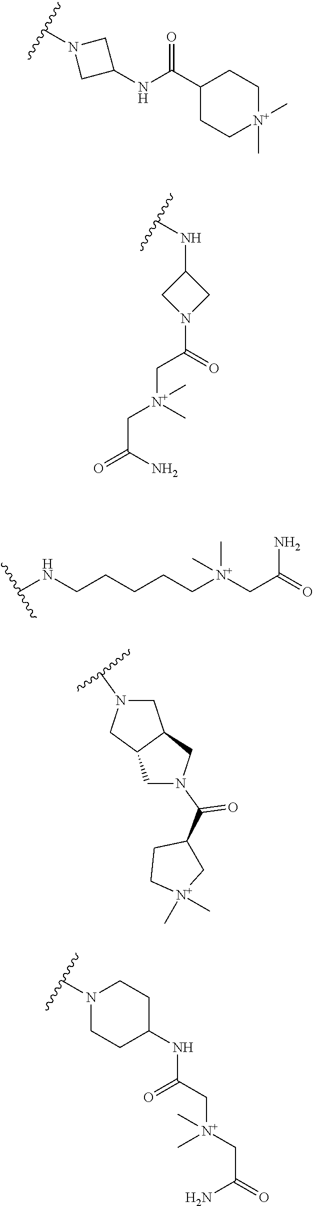 [SMILES: *N1CC(NC(=O)C2CC[N+](C)(C)CC2)C1 ; *NC1CN(C(=O)C[N+](C)(C)CC(N)=O)C1 ; *NCCCCC[N+](C)(C)CC(N)=O ; *N1C[C@H]2CN(C(=O)[C@@H]3CC[N+](C)(C)C3)C[C@@H]2C1 ; *N1CCC(NC(=O)C[N+](C)(C)CC(N)=O)CC1]

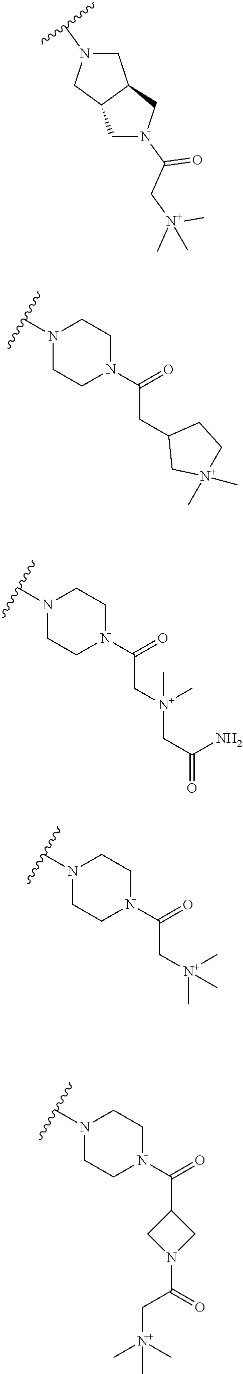 [SMILES: *N1C[C@H]2CN(C(=O)C[N+](C)(C)C)C[C@@H]2C1 ; *N1CCN(C(=O)CC2CC[N+](C)(C)C2)CC1 ; *N1CCN(C(=O)C[N+](C)(C)CC(N)=O)CC1 ; *N1CCN(C(=O)C[N+](C)(C)C)CC1 ; *N1CCN(C(=O)C2CN(C(=O)C[N+](C)(C)C)C2)CC1]

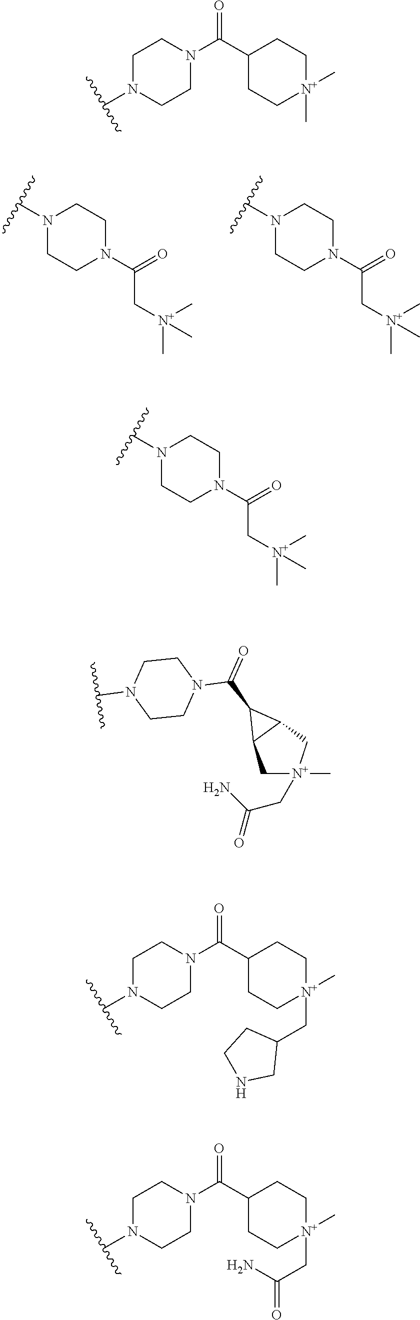

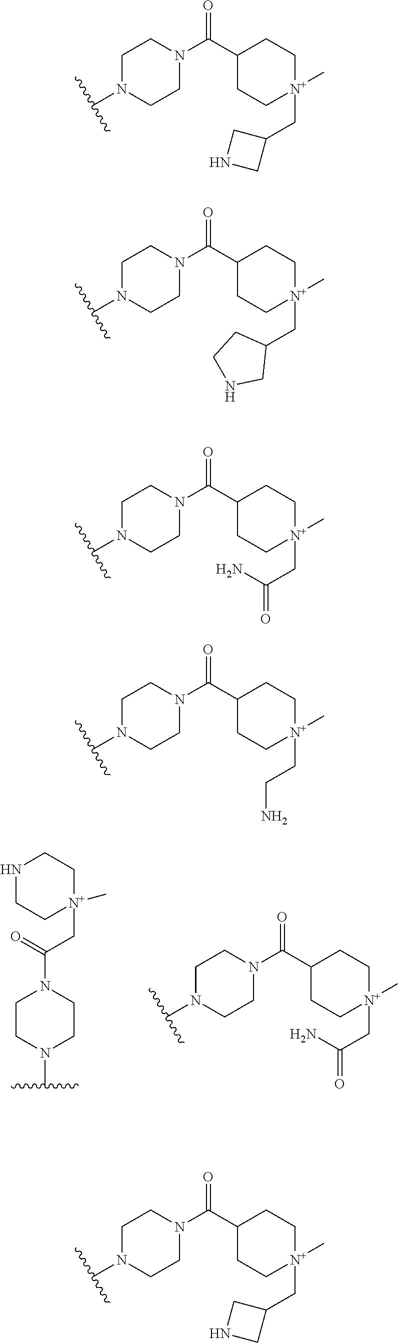

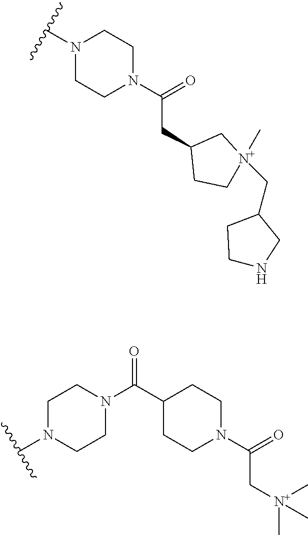 [SMILES: *N1CCN(C(=O)C[C@@H]2CC[N+](C)(CC3CCNC3)C2)CC1 ; *N1CCN(C(=O)C2CCN(C(=O)C[N+](C)(C)C)CC2)CC1]

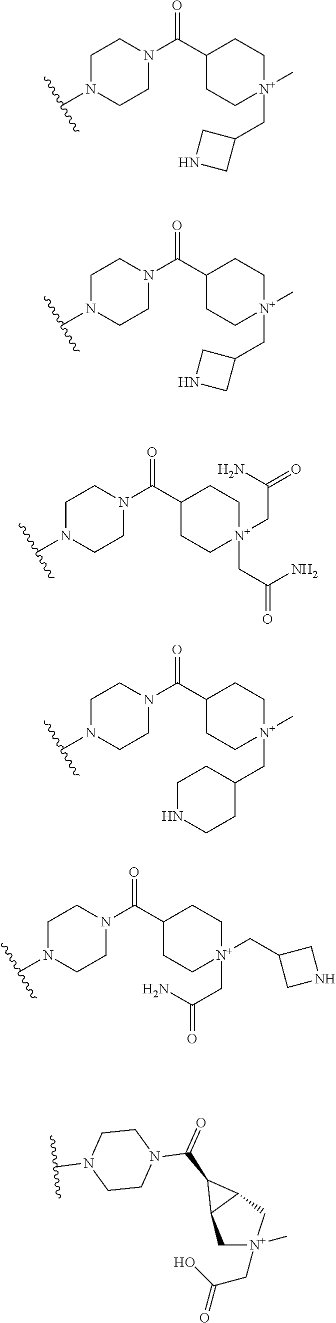

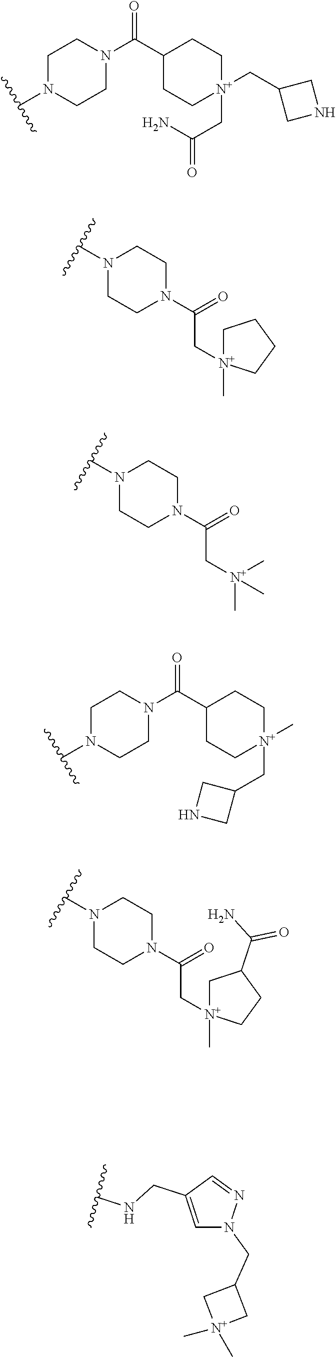 [SMILES: *N1CCN(C(=O)C2CC[N+](CC(N)=O)(CC3CNC3)CC2)CC1 ; *N1CCN(C(=O)C[N+]2(C)CCCC2)CC1 ; *N1CCN(C(=O)C[N+](C)(C)C)CC1 ; *N1CCN(C(=O)C2CC[N+](C)(CC3CNC3)CC2)CC1 ; *N1CCN(C(=O)C[N+]2(C)CCC(C(N)=O)C2)CC1 ; *NCc1cnn(CC2C[N+](C)(C)C2)c1]

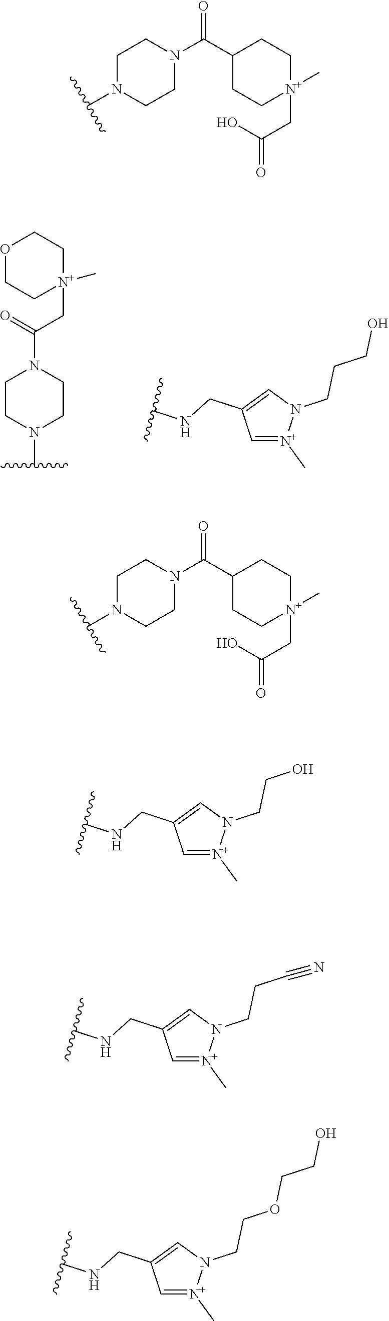

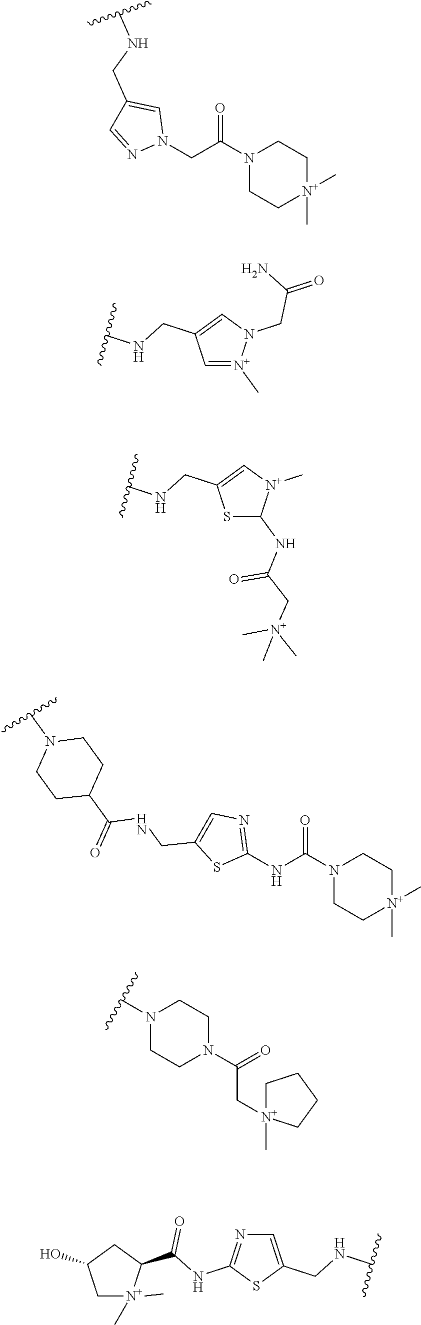 [SMILES: *NCc1cnn(CC(=O)N2CC[N+](C)(C)CC2)c1 ; *NCc1cn(CC(N)=O)[n+](C)c1 ; *NCC1=C[NH+](C)C(NC(=O)C[N+](C)(C)C)S1 ; *N1CCC(C(=O)NCc2cnc(NC(=O)N3CC[N+](C)(C)CC3)s2)CC1 ; *N1CCN(C(=O)C[N+]2(C)CCCC2)CC1 ; *NCc1cnc(NC(=O)[C@@H]2C[C@@H](O)C[N+]2(C)C)s1]

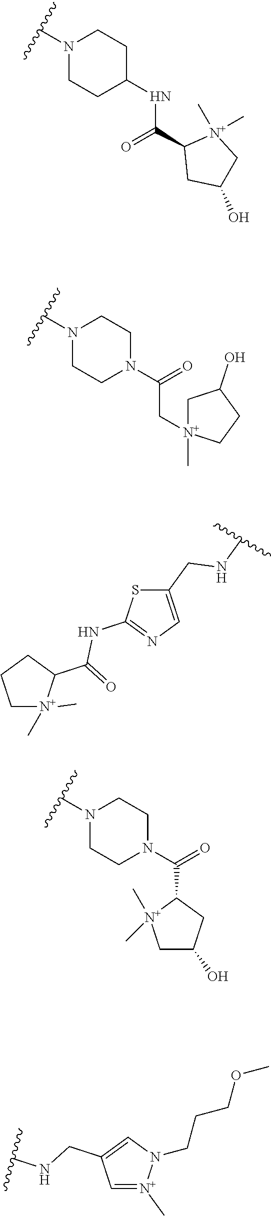 [SMILES: *N1CCC(NC(=O)[C@@H]2C[C@@H](O)C[N+]2(C)C)CC1 ; *N1CCN(C(=O)C[N+]2(C)CCC(O)C2)CC1 ; *NCc1cnc(NC(=O)C2CCC[N+]2(C)C)s1 ; *N1CCN(C(=O)[C@@H]2C[C@H](O)C[N+]2(C)C)CC1 ; *NCc1cn(CCCOC)[n+](C)c1]

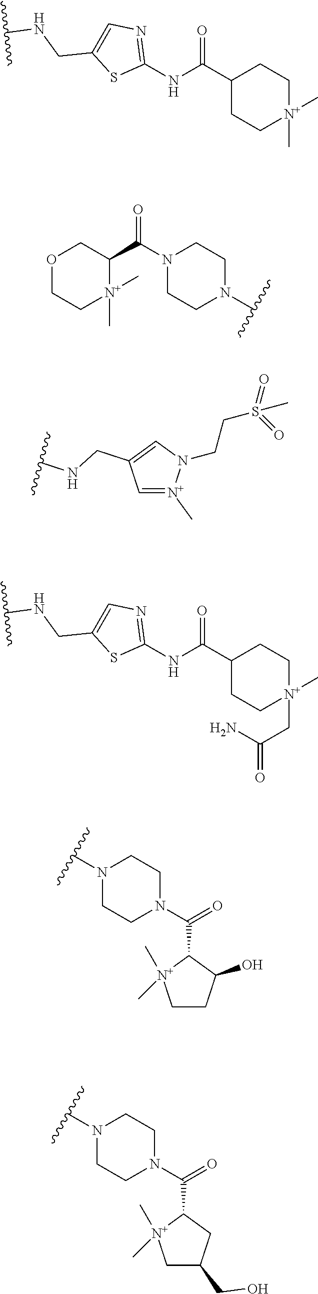 [SMILES: *NCc1cnc(NC(=O)C2CC[N+](C)(C)CC2)s1 ; *N1CCN(C(=O)[C@@H]2COCC[N+]2(C)C)CC1 ; *NCc1cn(CCS(C)(=O)=O)[n+](C)c1 ; *NCc1cnc(NC(=O)C2CC[N+](C)(CC(N)=O)CC2)s1 ; *N1CCN(C(=O)[C@@H]2[C@@H](O)CC[N+]2(C)C)CC1 ; *N1CCN(C(=O)[C@@H]2C[C@@H](CO)C[N+]2(C)C)CC1]

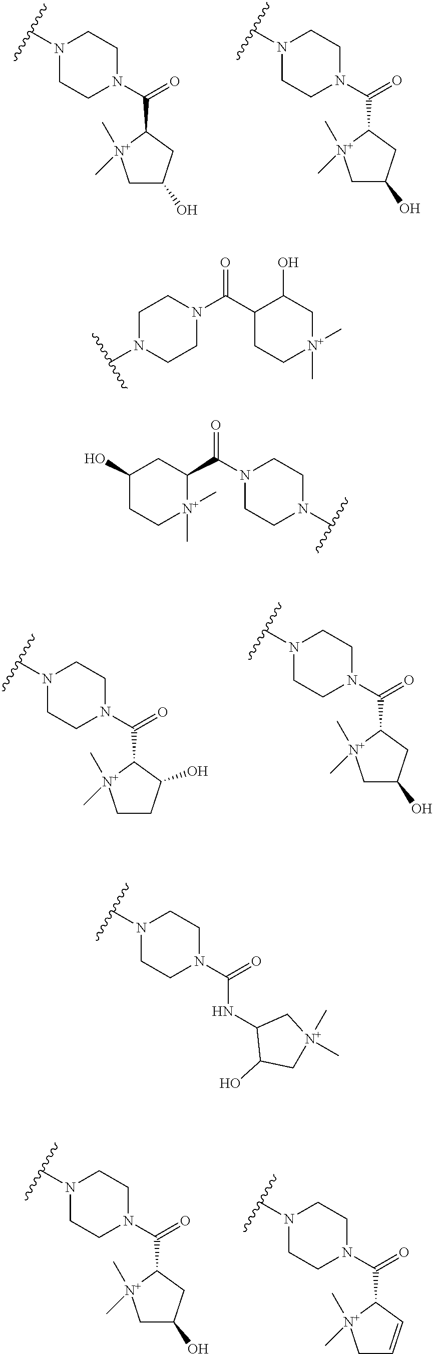

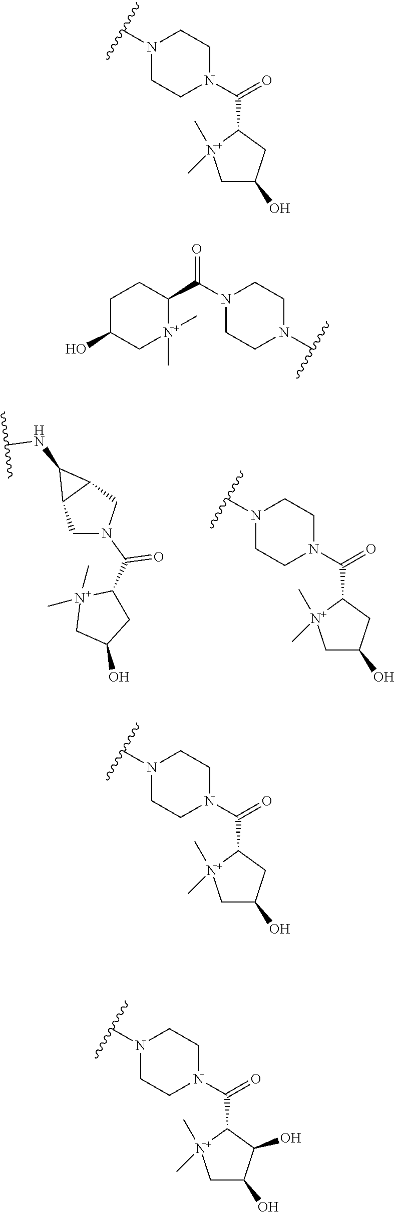 [SMILES: *N1CCN(C(=O)[C@@H]2C[C@@H](O)C[N+]2(C)C)CC1 ; *N1CCN(C(=O)[C@@H]2CC[C@H](O)C[N+]2(C)C)CC1 ; *N[C@H]1[C@@H]2CN(C(=O)[C@@H]3C[C@@H](O)C[N+]3(C)C)C[C@@H]21 ; *N1CCN(C(=O)[C@@H]2C[C@@H](O)C[N+]2(C)C)CC1 ; *N1CCN(C(=O)[C@@H]2C[C@@H](O)C[N+]2(C)C)CC1 ; *N1CCN(C(=O)[C@@H]2[C@@H](O)[C@@H](O)C[N+]2(C)C)CC1]

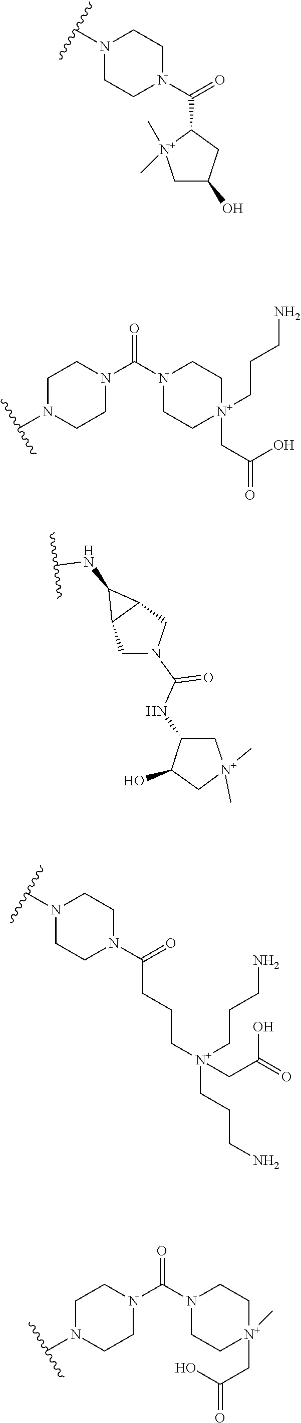 [SMILES: *N1CCN(C(=O)[C@@H]2C[C@@H](O)C[N+]2(C)C)CC1 ; *N1CCN(C(=O)N2CC[N+](CCCN)(CC(=O)O)CC2)CC1 ; *N[C@H]1[C@@H]2CN(C(=O)N[C@@H]3C[N+](C)(C)C[C@H]3O)C[C@@H]21 ; *N1CCN(C(=O)CCC[N+](CCCN)(CCCN)CC(=O)O)CC1 ; *N1CCN(C(=O)N2CC[N+](C)(CC(=O)O)CC2)CC1]

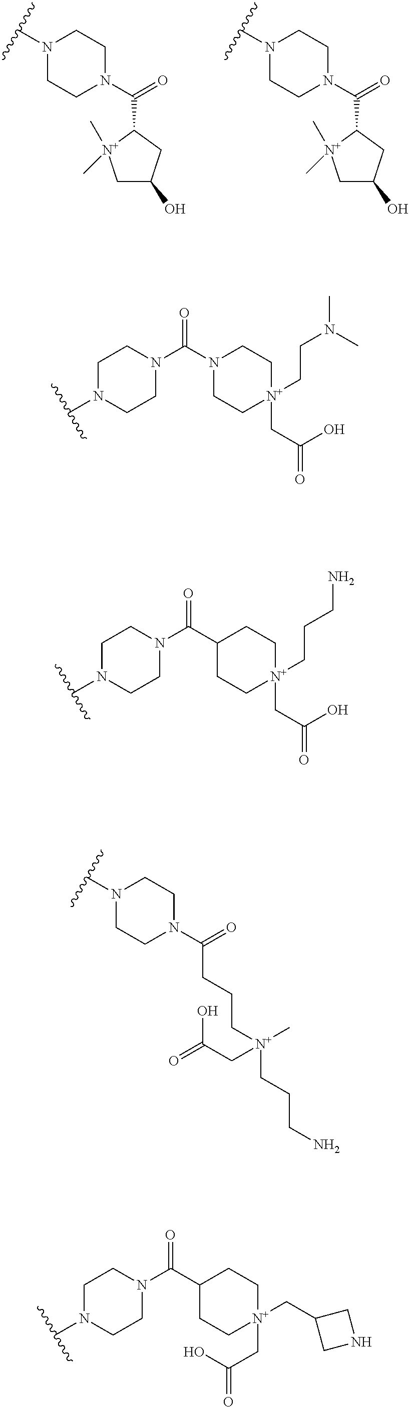 [SMILES: *N1CCN(C(=O)[C@@H]2C[C@@H](O)C[N+]2(C)C)CC1 ; *N1CCN(C(=O)[C@@H]2C[C@@H](O)C[N+]2(C)C)CC1 ; *N1CCN(C(=O)N2CC[N+](CCN(C)C)(CC(=O)O)CC2)CC1 ; *N1CCN(C(=O)C2CC[N+](CCCN)(CC(=O)O)CC2)CC1 ; *N1CCN(C(=O)CCC[N+](C)(CCCN)CC(=O)O)CC1 ; *N1CCN(C(=O)C2CC[N+](CC(=O)O)(CC3CNC3)CC2)CC1]

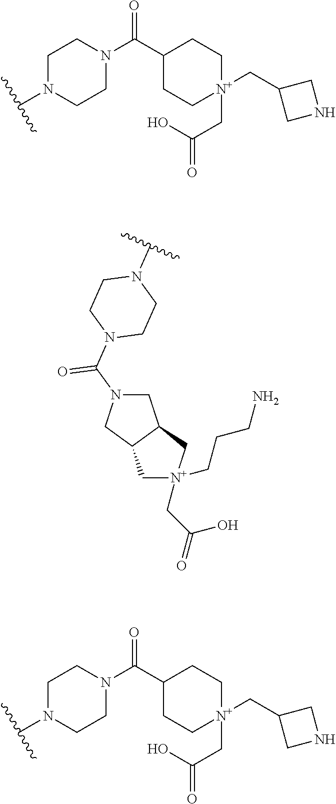 [SMILES: *N1CCN(C(=O)C2CC[N+](CC(=O)O)(CC3CNC3)CC2)CC1 ; *N1CCN(C(=O)N2C[C@H]3C[N+](CCCN)(CC(=O)O)C[C@@H]3C2)CC1 ; *N1CCN(C(=O)C2CC[N+](CC(=O)O)(CC3CNC3)CC2)CC1]

wherein a wavy line indicates the point of attachment of R^{x} to the remainder of formula (II) and wherein R³ to R⁶ are as defined herein.

In one embodiment, there is provided a compound of formula (I) as described herein, or a pharmaceutically acceptable salt thereof, wherein said compound of formula (I) is selected from:
2-[1-[2-Chloro-4-[[5-(2,3-difluoro-4-methoxy-phenyl)-1-methyl-imidazole-2-carbonyl]amino]benzoyl]-4-piperidyl]ethyl-trimethyl-ammonium; formate;
5-[[2-Chloro-4-[[5-(2,3-difluoro-4-methoxy-phenyl)-1-methyl-imidazole-2-carbonyl]amino]benzoyl]amino]pentyl-trimethyl-ammonium;formate;
(2-amino-2-oxo-ethyl)-[5-[[2-chloro-4-[[5-(2,3-difluoro-4-methoxy-phenyl)-1-methyl-imidazole-2-carbonyl]amino]benzoyl]amino]pentyl]-dimethyl-ammonium;formate;
(2-amino-2-oxo-ethyl)-[2-[4-[2-chloro-4-[[5-(2,3-difluoro-4-methoxy-phenyl)-1-methyl-imidazole-2-carbonyl]amino]benzoyl]piperazin-1-yl]ethyl]-dimethyl-ammonium;formate;
2-(2-(4-(5-(2,3-difluoro-4-methoxyphenyl)-1-methyl-1H-imidazole-2-carboxamido)-2-ethylbenzamido)ethoxy)-N,N,N-trimethylethan-1-aminium iodide;
3-(4-(4-(5-(2,3-difluoro-4-methoxyphenyl)-1-methyl-1H-imidazole-2-carboxamido)-2-ethylbenzoyl)piperazin-1-yl)-N,N,N-trimethylpropan-1-aminium iodide;
[2-[4-[2-chloro-4-[[5-[4-(cyanomethoxy)-2,3-difluoro-phenyl]-1-methyl-imidazole-2-carbonyl]amino]benzoyl]piperazin-1-yl]-2-oxo-ethyl]-trimethyl-ammonium;formate;iodide;
[2-[4-[4-[[5-[4-(cyanomethoxy)-2,3-difluoro-phenyl]-1-methyl-imidazole-2-carbonyl]amino]-2-methyl-benzoyl]piperazin-1-yl]-2-oxo-ethyl]-trimethyl-ammonium;iodide;
2-[4-[2-chloro-4-[[5-(2,3-difluoro-4-methoxy-phenyl)-1-methyl-imidazole-2-carbonyl]amino]benzoyl]piperazin-1-yl]ethyl-trimethyl-ammonium;iodide;
[2-[[3-[[2-Chloro-4-[[5-(2,3-difluoro-4-methoxy-phenyl)-1-methyl-imidazole-2-carbonyl]amino]benzoyl]amino]cyclobutyl]amino]-2-oxo-ethyl]-trimethyl-ammonium formate;
2-[[3-[[2-chloro-4-[[5-(2,3-difluoro-4-methoxy-phenyl)-1-methyl-imidazole-2-carbonyl]amino]benzoyl]amino]cyclobutyl]amino]ethyl-trimethyl-ammonium; formic acid; formate;
[2-[3-[[2-chloro-4-[[5-[4-(cyanomethoxy)-2,3-difluoro-phenyl]-1-methyl-imidazole-2-carbonyl]amino]benzoyl]amino]propylamino]-2-oxo-ethyl]-trimethyl-ammonium formate;
(2-amino-2-oxo-ethyl)-[2-[2-[[2-chloro-4-[[5-[4-(cyanomethoxy)-2,3-difluoro-phenyl]-1-methyl-imidazole-2-carbonyl]amino]benzoyl]amino]ethoxy]ethyl]-dimethyl-ammonium formate;
[2-[2-[[2-chloro-4-[[5-[4-(cyanomethoxy)-2,3-difluoro-phenyl]-1-methyl-imidazole-2-carbonyl]amino]benzoyl]amino]ethylamino]-2-oxo-ethyl]-trimethyl-ammonium formate;
2-[2-[[2-chloro-4-[[5-[4-(cyanomethoxy)-2,3-difluoro-phenyl]-1-methyl-imidazole-2-carbonyl]amino]benzoyl]amino]ethoxy]ethyl-trimethyl-ammonium formate;
[2-[[3-[[2-chloro-4-[[5-[4-(cyanomethoxy)-2,3-difluoro-phenyl]-1-methyl-imidazole-2-carbonyl]amino]benzoyl]amino]cyclobutyl]amino]-2-oxo-ethyl]-trimethyl-ammonium formate;
2-[[3-[[2-chloro-4-[[5-[4-(cyanomethoxy)-2,3-difluoro-phenyl]-1-methyl-imidazole-2-carbonyl]amino]benzoyl]amino]cyclobutyl]amino]ethyl-trimethyl-ammonium formate;
[2-[[3-[[4-[[5-[4-(cyanomethoxy)-2,3-difluoro-phenyl]-1-methyl-imidazole-2-carbonyl]amino]-2-methyl-benzoyl]amino]cyclobutyl]amino]-2-oxo-ethyl]-trimethyl-ammonium formate;
2-[[3-[[4-[[5-[4-(cyanomethoxy)-2,3-difluoro-phenyl]-1-methyl-imidazole-2-carbonyl]amino]-2-methyl-benzoyl]amino]cyclobutyl]amino]ethyl-trimethyl-ammonium formate;
N-[3-chloro-4-[[1-(2-hydroxyethyl)-2-methyl-pyrazol-2-ium-4-yl]methylcarbamoyl]phenyl]-5-(2,3-difluoro-4-methoxy-phenyl)-1-methyl-imidazole-2-carboxamide;formate;
N-[3-chloro-4-[[1-(3-hydroxypropyl)-2-methyl-pyrazol-2-ium-4-yl]methylcarbamoyl]phenyl]-5-(2,3-difluoro-4-methoxy-phenyl)-1-methyl-imidazole-2-carboxamide;formate;
N-[3-chloro-4-[[1-(2-cyanoethyl)-2-methyl-pyrazol-2-ium-4-yl]methylcarbamoyl]phenyl]-5-(2,3-difluoro-4-methoxy-phenyl)-1-methyl-imidazole-2-carboxamide;formate;
N-[4-[[1-(2-amino-2-oxo-ethyl)-2-methyl-pyrazol-2-ium-4-yl]methylcarbamoyl]-3-chloro-phenyl]-5-(2,3-difluoro-4-methoxy-phenyl)-1-methyl-imidazole-2-carboxamide;formate;
N-[3-chloro-4-[[1-methyl-2-(2-methylsulfonylethyl)pyrazol-1-ium-4-yl]methylcarbamoyl]phenyl]-5-(2,3-difluoro-4-methoxy-phenyl)-1-methyl-imidazole-2-carboxamide;formate;
N-[3-chloro-4-[[1-(3-methoxypropyl)-2-methyl-pyrazol-2-ium-4-yl]methylcarbamoyl]phenyl]-5-(2,3-difluoro-4-methoxy-phenyl)-1-methyl-imidazole-2-carboxamide;formate;
N-[3-chloro-4-[[1-[2-(2-hydroxyethoxy)ethyl]-2-methyl-pyrazol-2-ium-4-yl]methylcarbamoyl]phenyl]-5-(2,3-difluoro-4-methoxy-phenyl)-1-methyl-imidazole-2-carboxamide;formate;
N-[3-chloro-4-[[2-[[(2S,4R)-4-hydroxy-1,1-dimethyl-pyrrolidin-1-ium-2-carbonyl]amino]thiazol-5-yl]methylcarbamoyl]phenyl]-5-(2,3-difluoro-4-methoxy-phenyl)-1-methyl-imidazole-2-carboxamide;formate;
N-[3-chloro-4-[[2-[(1,1-dimethylpyrrolidin-1-ium-2-carbonyl)amino]thiazol-5-yl]methylcarbamoyl]phenyl]-5-(2,3-difluoro-4-methoxy-phenyl)-1-methyl-imidazole-2-carboxamide;formate;
N-[5-[[[2-chloro-4-[[5-(2,3-difluoro-4-methoxy-phenyl)-1-methyl-imidazole-2-carbonyl]amino]benzoyl]amino]methyl]thiazol-2-yl]-1,1-dimethyl-piperidin-1-ium-4-carboxamide;formate;
1-(2-amino-2-oxo-ethyl)-N-[5-[[[2-chloro-4-[[5-(2,3-difluoro-4-methoxy-phenyl)-1-methyl-imidazole-2-carbonyl]amino]benzoyl]amino]methyl]thiazol-2-yl]-1-methyl-piperidin-1-ium-4-carboxamide;formate;
N-[5-[[[1-[2-chloro-4-[[5-(2,3-difluoro-4-methoxy-phenyl)-1-methyl-imidazole-2-carbonyl]amino]benzoyl]piperidine-4-carbonyl]amino]methyl]thiazol-2-yl]-1,1-dimethyl-piperidin-1-ium-4-carboxamide;formate;
(2-Amino-2-oxo-ethyl)-[2-[4-[4-[[5-(2,3-difluoro-4-methoxy-phenyl)-1-methyl-imidazole-2-carbonyl]amino]-2-methyl-benzoyl]piperazin-1-yl]-2-oxo-ethyl]-dimethyl-ammonium;formate;
(2-amino-2-oxo-ethyl)-[2-[4-[4-[[5-[4-(cyanomethoxy)-2,3-difluoro-phenyl]-1-methyl-imidazole-2-carbonyl]amino]-2-methyl-benzoyl]piperazin-1-yl]-2-oxo-ethyl]-dimethyl-ammonium;formate;
(2-amino-2-oxo-ethyl)-[3-[4-[4-[[5-(2,3-difluoro-4-methoxy-phenyl)-1-methyl-imidazole-2-carbonyl]amino]-2-methyl-benzoyl]piperazin-1-yl]-3-oxo-propyl]-dimethyl-ammonium;2,2,2-trifluoroacetate;
(2-amino-2-oxo-ethyl)-[2-[4-[2-chloro-4-[[5-[4-(cyanomethoxy)-2,3-difluoro-phenyl]-1-methyl-imidazole-2-carbonyl]amino]benzoyl]piperazin-1-yl]-2-oxo-ethyl]-dimethyl-ammonium;formate;
(2-amino-2-oxo-ethyl)-[2-[[1-[2-chloro-4-[[5-(2,3-difluoro-4-methoxy-phenyl)-1-methyl-imidazole-2-carbonyl]amino]benzoyl]-4-piperidyl]amino]-2-oxo-ethyl]-dimethyl-ammonium;formate;
(2-amino-2-oxo-ethyl)-[2-[3-[[2-chloro-4-[[5-(2,3-difluoro-4-methoxy-phenyl)-1-methyl-imidazole-2-carbonyl]amino]benzoyl]amino]azetidin-1-yl]-2-oxo-ethyl]-dimethyl-ammonium;formate;
(2-amino-2-oxo-ethyl)-[2-[4-[2-chloro-4-[[5-(2,3-difluoro-4-methoxy-phenyl)-1-methyl-imidazole-2-carbonyl]amino]benzoyl]piperazin-1-yl]-2-oxo-ethyl]-dimethyl-ammonium;formate;
[3-[4-[4-[[5-(2,3-Difluoro-4-methoxy-phenyl)-1-methyl-imidazole-2-carbonyl]amino]-2-methyl-benzoyl]piperazin-1-yl]-3-oxo-propyl]-trimethyl-ammonium;2,2,2-trifluoroacetate;
[3-[4-[2-chloro-4-[[5-[4-(cyanomethoxy)-2,3-difluoro-phenyl]-1-methyl-imidazole-2-carbonyl]amino]benzoyl]piperazin-1-yl]-3-oxo-propyl]-trimethyl-ammonium;iodide;
N-[3-chloro-4-[4-(1,1-dimethylpiperidin-1-ium-4-carbonyl)piperazine-1-carbonyl]phenyl]-5-(2,3-difluoro-4-methoxy-phenyl)-1-methyl-imidazole-2-carboxamide;formate;
10 [2-[4-[2-chloro-4-[[5-[4-(difluoromethoxy)-2-fluoro-phenyl]-1-methyl-imidazole-2-carbonyl]amino]benzoyl]piperazin-1-yl]-2-oxo-ethyl]-trimethyl-ammonium;formate;
[2-[4-[2-chloro-4-[[5-[4-(difluoromethoxy)-2,3-difluoro-phenyl]-1-methyl-imidazole-2-carbonyl]amino]benzoyl]piperazin-1-yl]-2-oxo-ethyl]-trimethyl-ammonium;formate;
N-[3-chloro-4-[4-[2-(1,1-dimethylpyrrolidin-1-ium-3-yl)acetyl]piperazine-1-carbonyl]phenyl]-5-(2,3-difluoro-4-methoxy-phenyl)-1-methyl-imidazole-2-carboxamide;formate;
[2-[4-[4-[2-chloro-4-[[5-[4-(difluoromethoxy)-2,3-difluoro-phenyl]-1-methyl-imidazole-2-carbonyl]amino]benzoyl]piperazine-1-carbonyl]-1-piperidyl]-2-oxo-ethyl]-trimethyl-ammonium;formate;
[2-[4-[2-chloro-4-[[5-[4-(difluoromethoxy)-3-fluoro-phenyl]-1-methyl-imidazole-2-carbonyl]amino]benzoyl]piperazin-1-yl]-2-oxo-ethyl]-trimethyl-ammonium;formate;
[2-[3-[4-[2-chloro-4-[[5-(2,3-difluoro-4-methoxy-phenyl)-1-methyl-imidazole-2-carbonyl]amino]benzoyl]piperazine-1-carbonyl]azetidin-1-yl]-2-oxo-ethyl]-trimethyl-ammonium;formate;
N-[1-[2-chloro-4-[[5-(2,3-difluoro-4-methoxy-phenyl)-1-methyl-imidazole-2-carbonyl]amino]benzoyl]azetidin-3-yl]-1,1-dimethyl-piperidin-1-ium-4-carboxamide;formate;
N-[4-[(3aS,6aR)-5-[(3R)-1,1-dimethylpyrrolidin-1-ium-3-carbonyl]-1,3,3a,4,6,6a-hexahydropyrrolo[3,4-c]pyrrole-2-carbonyl]-3-chloro-phenyl]-5-(2,3-difluoro-4-methoxy-phenyl)-1-methyl-imidazole-2-carboxamide;formate;
[2-[(3aS,6aR)-5-[2-chloro-4-[[5-(2,3-difluoro-4-methoxy-phenyl)-1-methyl-imidazole-2-carbonyl]amino]benzoyl]-1,3,3a,4,6,6a-hexahydropyrrolo[3,4-c]pyrrol-2-yl]-2-oxo-ethyl]-trimethyl-ammonium;formate;
[2-[4-[2-chloro-4-[[5-(2,3-difluoro-4-methoxy-phenyl)-1-methyl-imidazole-2-carbonyl]amino]benzoyl]piperazin-1-yl]-2-oxo-ethyl]-trimethyl-ammonium;formate;
[2-[4-[4-[[5-(2,3-difluoro-4-methoxy-phenyl)-1-methyl-imidazole-2-carbonyl]amino]-2-methyl-benzoyl]piperazin-1-yl]-2-oxo-ethyl]-trimethyl-ammonium;formate;
[2-[4-[2-chloro-4-[[5-[2-fluoro-4-(fluoromethoxy)phenyl]-1-methyl-imidazole-2-carbonyl]amino]benzoyl]piperazin-1-yl]-2-oxo-ethyl]-trimethyl-ammonium;formate;
2-(2-(1-(4-(5-(2,3-difluoro-4-methoxyphenyl)-1-methyl-1H-imidazole-2-carboxamido)-2-ethylbenzoyl)piperidine-4-carboxamido)ethoxy)-N,N,N-trimethylethan-1-aminium iodide;
(S)-3-(1-(2-chloro-4-(5-(4-(cyanomethoxy)-2,3-difluorophenyl)-1-methyl-1H-imidazole-2-carboxamido)benzoyl)piperidine-4-carboxamido)-1,1-dimethylpyrrolidin-1-ium formate;
(2S,4R)-2-(4-(2-chloro-4-(5-(2,3-difluoro-4-methoxyphenyl)-1-methyl-1H-imidazole-2-carboxamido)benzoyl)piperazine-1-carbonyl)-4-hydroxy-1,1-dimethylpyrrolidin-1-ium formate;
4-(4-(4-(5-(4-(cyanomethoxy)-2,3-difluorophenyl)-1-methyl-1H-imidazole-2-carboxamido)-2-methylbenzoyl)piperazine-1-carbonyl)-4-hydroxy-1,1-dimethylpiperidin-1-ium formate;
(2S,4S)-2-(4-(2-chloro-4-(5-(4-(cyanomethoxy)-2,3-difluorophenyl)-1-methyl-1H-imidazole-2-carboxamido)benzoyl)piperazine-1-carbonyl)-4-hydroxy-1,1,4-trimethylpyrrolidin-1-ium formate;
(3R,4R)-3-(4-(4-(5-(4-(cyanomethoxy)-2,3-difluorophenyl)-1-methyl-1H-imidazole-2-carboxamido)-2-methylbenzoyl)piperazine-1-carboxamido)-4-hydroxy-1,1-dimethylpyrrolidin-1-ium formate;
(3S,4S)-3-(4-(4-(5-(4-(cyanomethoxy)-2,3-difluorophenyl)-1-methyl-1H-imidazole-2-carboxamido)-2-methylbenzoyl)piperazine-1-carboxamido)-4-hydroxy-1,1-dimethylpyrrolidin-1-ium formate;
(2S,4R)-2-(4-(2-chloro-4-(5-(2,3-difluoro-4-methoxyphenyl)-1-methyl-1H-imidazole-2-carboxamido)benzoyl)piperazine-1-carbonyl)-4-hydroxy-1,1,4-trimethylpyrrolidin-1-ium formate;
[1-[1-[2-chloro-4-[[5-[4-(cyanomethoxy)-2,3-difluoro-phenyl]-1-methyl-imidazole-2-carbonyl]amino]benzoyl]piperidine-4-carbonyl]azetidin-3-yl]-trimethyl-ammonium;formate;
N-[3-chloro-4-[[1-[2-(4,4-dimethylpiperazin-4-ium-1-yl)-2-oxo-ethyl]pyrazol-4-yl]methylcarbamoyl]phenyl]-5-(2,3-difluoro-4-methoxy-phenyl)-1-methyl-imidazole-2-carboxamide;formate;
N-[3-chloro-4-[4-(3-hydroxy-1,1-dimethyl-piperidin-1-ium-4-carbonyl)piperazine-1-carbonyl]phenyl]-5-(2,3-difluoro-4-methoxy-phenyl)-1-methyl-imidazole-2-carboxamide;formate;
N-[3-chloro-4-[4-[(2S,4S)-4-hydroxy-1,1-dimethyl-pyrrolidin-1-ium-2-carbonyl]piperazine-1-carbonyl]phenyl]-5-(2,3-difluoro-4-methoxy-phenyl)-1-methyl-imidazole-2-carboxamide;formate;
N-[3-chloro-4-[4-[(3S)-4,4-dimethylmorpholin-4-ium-3-carbonyl]piperazine-1-carbonyl]phenyl]-5-(2,3-difluoro-4-methoxy-phenyl)-1-methyl-imidazole-2-carboxamide;iodide;
N-[3-chloro-4-[4-[(2S,3S)-3-hydroxy-1,1-dimethyl-pyrrolidin-1-ium-2-carbonyl]piperazine-1-carbonyl]phenyl]-5-(2,3-difluoro-4-methoxy-phenyl)-1-methyl-imidazole-2-carboxamide;formate;
N-[3-chloro-4-[4-[(2R,4S)-4-hydroxy-1,1-dimethyl-pyrrolidin-1-ium-2-carbonyl]piperazine-1-carbonyl]phenyl]-5-(2,3-difluoro-4-methoxy-phenyl)-1-methyl-imidazole-2-carboxamide;formate;
N-[3-chloro-4-[4-[(2S,4R)-4-hydroxy-1,1-dimethyl-piperidin-1-ium-2-carbonyl]piperazine-1-carbonyl]phenyl]-5-(2,3-difluoro-4-methoxy-phenyl)-1-methyl-imidazole-2-carboxamide;formate;
4-[2-chloro-4-[[5-(2,3-difluoro-4-methoxy-phenyl)-1-methyl-imidazole-2-carbonyl]amino]benzoyl]-N-(4-hydroxy-1,1-dimethyl-pyrrolidin-1-ium-3-yl)piperazine-1-carboxamide;formate;
N-[3-chloro-4-[4-[(2S,4R)-4-hydroxy-1,1-dimethyl-pyrrolidin-1-ium-2-carbonyl]piperazine-1-carbonyl]phenyl]-5-[2-(difluoromethyl)-3-fluoro-4-methoxy-phenyl]-1-methyl-imidazole-2-carboxamide;formate;
N-[3-chloro-4-[4-[(2S,4R)-4-hydroxy-1,1-dimethyl-pyrrolidin-1-ium-2-carbonyl]piperazine-1-carbonyl]phenyl]-5-[4-(difluoromethoxy)-2,3-difluoro-phenyl]-1-methyl-imidazole-2-carboxamide;formate;
N-[3-chloro-4-[4-[(2S,4R)-4-(hydroxymethyl)-1,1-dimethyl-pyrrolidin-1-ium-2-carbonyl]piperazine-1-carbonyl]phenyl]-5-(2,3-difluoro-4-methoxy-phenyl)-1-methyl-imidazole-2-carboxamide;formate;
5-(2-chloro-3-fluoro-4-methoxy-phenyl)-N-[3-chloro-4-[4-[(2S,4R)-4-hydroxy-1,1-dimethyl-pyrrolidin-1-ium-2-carbonyl]piperazine-1-carbonyl]phenyl]-1-methyl-imidazole-2-carboxamide;formate;
N-[3-chloro-4-[4-[(2S,3R)-3-hydroxy-1,1-dimethyl-pyrrolidin-1-ium-2-carbonyl]piperazine-1-carbonyl]phenyl]-5-(2,3-difluoro-4-methoxy-phenyl)-1-methyl-imidazole-2-carboxamide;formate;
N-[3-chloro-4-[4-[(2S,4R)-4-hydroxy-1,1-dimethyl-pyrrolidin-1-ium-2-carbonyl]piperazine-1-carbonyl]phenyl]-5-[2,3-difluoro-4-(fluoromethoxy)phenyl]-1-methyl-imidazole-2-carboxamide;formate;
N-[3-chloro-4-[4-[(2S,5S)-5-hydroxy-1,1-dimethyl-piperidin-1-ium-2-carbonyl]piperazine-1-carbonyl]phenyl]-5-(2,3-difluoro-4-methoxy-phenyl)-1-methyl-imidazole-2-carboxamide;formate;
5-[3-chloro-4-(difluoromethoxy)-2-fluoro-phenyl]-N-[3-chloro-4-[4-[(2S,4R)-4-hydroxy-1,1-dimethyl-pyrrolidin-1-ium-2-carbonyl]piperazine-1-carbonyl]phenyl]-1-methyl-imidazole-2-carboxamide;iodide;
5-(3-chloro-2-fluoro-4-methoxy-phenyl)-N-[3-chloro-4-[4-[(2S,4R)-4-hydroxy-1,1-dimethyl-pyrrolidin-1-ium-2-carbonyl]piperazine-1-carbonyl]phenyl]-1-methyl-imidazole-2-carboxamide;formate;
N-[3-chloro-4-[4-[(2S)-1,1-dimethyl-2,5-dihydropyrrol-1-ium-2-carbonyl]piperazine-1-carbonyl]phenyl]-5-(2,3-difluoro-4-methoxy-phenyl)-1-methyl-imidazole-2-carboxamide;formate;
N-[3-chloro-4-[4-[(2S,3R,4S)-3,4-dihydroxy-1,1-dimethyl-pyrrolidin-1-ium-2-carbonyl]piperazine-1-carbonyl]phenyl]-5-(2,3-difluoro-4-methoxy-phenyl)-1-methyl-imidazole-2-carboxamide;formate;
N-[3-chloro-4-[4-[(2S,4R)-4-hydroxy-1,1-dimethyl-pyrrolidin-1-ium-2-carbonyl]piperazine-1-carbonyl]phenyl]-5-(3-cyano-2-fluoro-4-methoxy-phenyl)-1-methyl-imidazole-2-carboxamide;formate;
N-[3-chloro-4-[4-[(2S,4R)-4-hydroxy-1,1-dimethyl-pyrrolidin-1-ium-2-carbonyl]piperazine-1-carbonyl]phenyl]-5-[2-fluoro-4-methoxy-3-(trifluoromethyl)phenyl]-1-methyl-imidazole-2-carboxamide;formate;
5-[2-chloro-4-(difluoromethoxy)-3-fluoro-phenyl]-N-[3-chloro-4-[4-[(2S,4R)-4-hydroxy-1,1-dimethyl-pyrrolidin-1-ium-2-carbonyl]piperazine-1-carbonyl]phenyl]-1-methyl-imidazole-2-carboxamide;formate;
N-[3-chloro-4-[[(1R,5S)-3-[(2S,4R)-4-hydroxy-1,1-dimethyl-pyrrolidin-1-ium-2-carbonyl]-3-azabicyclo[3.1.0]hexan-6-yl]carbamoyl]phenyl]-5-(2,3-difluoro-4-methoxy-phenyl)-1-methyl-imidazole-2-carboxamide;formate;
(1S,5R)-6-[[2-chloro-4-[[5-(2,3-difluoro-4-methoxy-phenyl)-1-methyl-imidazole-2-carbonyl]amino]benzoyl]amino]-N-[(3R,4R)-4-hydroxy-1,1-dimethyl-pyrrolidin-1-ium-3-yl]-3-azabicyclo[3.1.0]hexane-3-carboxamide;formate;
10 [2-[4-[2-chloro-4-[[5-[2,3-difluoro-4-(fluoromethoxy)phenyl]-1-methyl-imidazole-2-carbonyl]amino]benzoyl]piperazin-1-yl]-2-oxo-ethyl]-trimethyl-ammonium;formate;
N-[4-[4-[1-(2-Amino-2-oxo-ethyl)-1-methyl-piperidin-1-ium-4-carbonyl]piperazine-1-carbonyl]-3-chloro-phenyl]-5-[2-chloro-4-(difluoromethoxy)-3-fluoro-phenyl]-1-methyl-imidazole-2-carboxamide;formate;
N-[4-[4-[1-(2-amino-2-oxo-ethyl)-1-methyl-piperidin-1-ium-4-carbonyl]piperazine-1-carbonyl]-3-chloro-phenyl]-5-[4-(difluoromethoxy)-2,3-difluoro-phenyl]-1-methyl-imidazole-2-carboxamide;formate;
N-[4-[4-[1-(2-amino-2-oxo-ethyl)-1-methyl-piperidin-1-ium-4-carbonyl]piperazine-1-carbonyl]-3-chloro-phenyl]-5-(2-chloro-3-fluoro-4-methoxy-phenyl)-1-methyl-imidazole-2-carboxamide;formate;
N-[4-[4-[(1R,5S)-3-(2-amino-2-oxo-ethyl)-3-methyl-3-azoniabicyclo[3.1.0]hexane-6-carbonyl]piperazine-1-carbonyl]-3-chloro-phenyl]-5-[4-(difluoromethoxy)-2,3-difluoro-phenyl]-1-methyl-imidazole-2-carboxamide;formate;
N-[4-[4-[1-(2-amino-2-oxo-ethyl)-1-methyl-piperidin-1-ium-4-carbonyl]piperazine-1-carbonyl]-3-chloro-phenyl]-5-(2,3-difluoro-4-methoxy-phenyl)-1-methyl-imidazole-2-carboxamide;formate;
[2-[4-[4-[2-chloro-4-[[5-(2,3-difluoro-4-methoxy-phenyl)-1-methyl-imidazole-2-carbonyl]amino]benzoyl]piperazine-1-carbonyl]-1-piperidyl]-2-oxo-ethyl]-trimethyl-ammonium;formate;
(2-amino-2-oxo-ethyl)-[2-[4-[4-[2-chloro-4-[[5-(2,3-difluoro-4-methoxy-phenyl)-1-methyl-imidazole-2-carbonyl]amino]benzoyl]piperazine-1-carbonyl]-1-piperidyl]-2-oxo-ethyl]-dimethyl-ammonium;formate;
2-[4-[4-[2-chloro-4-[[5-(2,3-difluoro-4-methoxy-phenyl)-1-methyl-imidazole-2-carbonyl]amino]benzoyl]piperazine-1-carbonyl]-1-methyl-piperidin-1-ium-1-yl]acetic acid;formate;
N-[4-[4-[1,1-bis(2-amino-2-oxo-ethyl)piperidin-1-ium-4-carbonyl]piperazine-1-carbonyl]-3-chloro-phenyl]-5-[2-chloro-4-(difluoromethoxy)-3-fluoro-phenyl]-1-methyl-imidazole-2-carboxamide;formate;
N-[4-[4-[1-(2-amino-2-oxo-ethyl)-1-(azetidin-3-ylmethyl)piperidin-1-ium-4-carbonyl]piperazine-1-carbonyl]-3-chloro-phenyl]-5-[2-chloro-4-(difluoromethoxy)-3-fluoro-phenyl]-1-methyl-imidazole-2-carboxamide;2,2,2-trifluoroacetate;2,2,2-trifluoroacetic acid;
N-[4-[4-[1-(2-Aminoethyl)-1-methyl-piperidin-1-ium-4-carbonyl]piperazine-1-carbonyl]-3-chloro-phenyl]-5-(2,3-difluoro-4-methoxy-phenyl)-1-methyl-imidazole-2-carboxamide;formate;
N-[4-[4-[1-(2-aminoethyl)-1-methyl-piperidin-1-ium-4-carbonyl]piperazine-1-carbonyl]-3-chloro-phenyl]-5-[2-chloro-4-(difluoromethoxy)-3-fluoro-phenyl]-1-methyl-imidazole-2-carboxamide;formic acid;formate;
N-[4-[4-[1-(azetidin-3-ylmethyl)-1-methyl-piperidin-1-ium-4-carbonyl]piperazine-1-carbonyl]-3-chloro-phenyl]-5-[4-(difluoromethoxy)-2,3-difluoro-phenyl]-1-methyl-imidazole-2-carboxamide;2,2,2-trifluoroacetate;2,2,2-trifluoroacetic acid;
N-[3-chloro-4-[4-[1-methyl-1-(pyrrolidin-3-ylmethyl)piperidin-1-ium-4-carbonyl]piperazine-1-carbonyl]phenyl]-5-[4-(difluoromethoxy)-2,3-difluoro-phenyl]-1-methyl-imidazole-2-carboxamide;formic acid;formate;
N-[4-[4-[1-(azetidin-3-ylmethyl)-1-methyl-piperidin-1-ium-4-carbonyl]piperazine-1-carbonyl]-3-chloro-phenyl]-5-[2-chloro-4-(difluoromethoxy)-3-fluoro-phenyl]-1-methyl-imidazole-2-carboxamide;formic acid;formate;
N-[4-[4-[1-(azetidin-3-ylmethyl)-1-methyl-piperidin-1-ium-4-carbonyl]piperazine-1-carbonyl]-3-chloro-phenyl]-5-(2,3-difluoro-4-methoxy-phenyl)-1-methyl-imidazole-2-carboxamide;formic acid;formate;
5-[4-(difluoromethoxy)-2,3-difluoro-phenyl]-N-[3-ethyl-4-[4-[1-methyl-1-(pyrrolidin-3-ylmethyl)piperidin-1-ium-4-carbonyl]piperazine-1-carbonyl]phenyl]-1-methyl-imidazole-2-carboxamide;2,2,2-trifluoroacetate;2,2,2-trifluoroacetic acid;
N-[3-chloro-4-[4-[2-[1-methyl-1-(pyrrolidin-3-ylmethyl)pyrrolidin-1-ium-3-yl]acetyl]piperazine-1-carbonyl]phenyl]-5-[4-(difluoromethoxy)-2,3-difluoro-phenyl]-1-methyl-imidazole-2-carboxamide;2,2,2-trifluoroacetate;2,2,2-trifluoroacetic acid;
N-[4-[4-[1-(azetidin-3-ylmethyl)-1-methyl-piperidin-1-ium-4-carbonyl]piperazine-1-carbonyl]-3-ethyl-phenyl]-5-[4-(difluoromethoxy)-2,3-difluoro-phenyl]-1-methyl-imidazole-2-carboxamide;2,2,2-trifluoroacetate;2,2,2-trifluoroacetic acid;
5-[4-(difluoromethoxy)-2,3-difluoro-phenyl]-N-[3-ethyl-4-[4-[1-methyl-1-(4-piperidylmethyl)piperidin-1-ium-4-carbonyl]piperazine-1-carbonyl]phenyl]-1-methyl-imidazole-2-carboxamide;2,2,2-trifluoroacetate;2,2,2-trifluoroacetic acid;
N-[4-[4-[1-(2-amino-2-oxo-ethyl)-1-(azetidin-3-ylmethyl)piperidin-1-ium-4-carbonyl]piperazine-1-carbonyl]-3-ethyl-phenyl]-5-[4-(difluoromethoxy)-2,3-difluoro-phenyl]-1-methyl-imidazole-2-carboxamide;2,2,2-trifluoroacetate;2,2,2-trifluoroacetic acid;
N-[3-chloro-4-[[1-[(1,1-dimethylazetidin-1-ium-3-yl)methyl]pyrazol-4-yl]methylcarbamoyl]phenyl]-5-(2,3-difluoro-4-methoxy-phenyl)-1-methyl-imidazole-2-carboxamide;formate;
[2-[[5-[[[2-chloro-4-[[5-(2,3-difluoro-4-methoxy-phenyl)-1-methyl-imidazole-2-carbonyl]amino]benzoyl]amino]methyl]-3-methyl-thiazol-3-ium-2-yl]amino]-2-oxo-ethyl]-trimethyl-ammonium;formate;
N-[3-chloro-4-[4-[2-(1-methylpiperazin-1-ium-1-yl)acetyl]piperazine-1-carbonyl]phenyl]-5-[4-(difluoromethoxy)-2,3-difluoro-phenyl]-1-methyl-imidazole-2-carboxamide;2,2,2-trifluoroacetate;2,2,2-trifluoroacetic acid;
N-[3-chloro-4-[4-[2-(1-methylpyrrolidin-1-ium-1-yl)acetyl]piperazine-1-carbonyl]phenyl]-5-[4-(difluoromethoxy)-2,3-difluoro-phenyl]-1-methyl-imidazole-2-carboxamide;formate;
N-[4-[4-[2-(3-carbamoyl-1-methyl-pyrrolidin-1-ium-1-yl)acetyl]piperazine-1-carbonyl]-3-chloro-phenyl]-5-[4-(difluoromethoxy)-2,3-difluoro-phenyl]-1-methyl-imidazole-2-carboxamide;formate;
N-[3-chloro-4-[4-[2-(4-methylmorpholin-4-ium-4-yl)acetyl]piperazine-1-carbonyl]phenyl]-5-(2,3-difluoro-4-methoxy-phenyl)-1-methyl-imidazole-2-carboxamide;formate;
N-[3-chloro-4-[4-[2-(1-methylpyrrolidin-1-ium-1-yl)acetyl]piperazine-1-carbonyl]phenyl]-5-(2,3-difluoro-4-methoxy-phenyl)-1-methyl-imidazole-2-carboxamide;formate;
N-[3-chloro-4-[4-[2-(3-hydroxy-1-methyl-pyrrolidin-1-ium-1-yl)acetyl]piperazine-1-carbonyl]phenyl]-5-(2,3-difluoro-4-methoxy-phenyl)-1-methyl-imidazole-2-carboxamide;formate;
2-[(1R,5S)-6-[4-[2-chloro-4-[[5-[2,3-difluoro-4-(fluoromethoxy)phenyl]-1-methyl-imidazole-2-carbonyl]amino]benzoyl]piperazine-1-carbonyl]-3-methyl-3-azoniabicyclo[3.1.0]hexan-3-yl]acetic acid;2,2,2-trifluoroacetate;
2-[4-[4-[2-chloro-4-[[5-[4-(difluoromethoxy)-2,3-difluoro-phenyl]-1-methyl-imidazole-2-carbonyl]amino]benzoyl]piperazine-1-carbonyl]-1-methyl-piperidin-1-ium-1-yl]acetic acid;2,2,2-trifluoroacetate;
2-[1-(3-aminopropyl)-4-[4-[2-chloro-4-[[5-(2,3-difluoro-4-methoxy-phenyl)-1-methyl-imidazole-2-carbonyl]amino]benzoyl]piperazine-1-carbonyl]piperidin-1-ium-1-yl]acetic acid;formate;
2-[1-(azetidin-3-ylmethyl)-4-[4-[2-chloro-4-[[5-(2,3-difluoro-4-methoxy-phenyl)-1-methyl-imidazole-2-carbonyl]amino]benzoyl]piperazine-1-carbonyl]piperidin-1-ium-1-yl]acetic acid;formate;
bis(3-aminopropyl)-(carboxymethyl)-[4-[4-[2-chloro-4-[[5-(2,3-difluoro-4-methoxy-phenyl)-1-methyl-imidazole-2-carbonyl]amino]benzoyl]piperazin-1-yl]-4-oxo-butyl]ammonium;formate;
3-aminopropyl-(carboxymethyl)-[4-[4-[2-chloro-4-[[5-(2,3-difluoro-4-methoxy-phenyl)-1-methyl-imidazole-2-carbonyl]amino]benzoyl]piperazin-1-yl]-4-oxo-butyl]-methyl-ammonium;formate;
2-[4-[4-[2-chloro-4-[[5-(2,3-difluoro-4-methoxy-phenyl)-1-methyl-imidazole-2-carbonyl]amino]benzoyl]piperazine-1-carbonyl]-1-methyl-piperazin-1-ium-1-yl]acetic acid formate;
2-[(3aS,6aR)-5-(3-aminopropyl)-2-[4-[2-chloro-4-[[5-(2,3-difluoro-4-methoxy-phenyl)-1-methyl-imidazole-2-carbonyl]amino]benzoyl]piperazine-1-carbonyl]-1,3,3a,4,6,6a-hexahydropyrrolo[3,4-c]pyrrol-5-ium-5-yl]acetic acid chloride hydrochloride;
2-[1-(3-aminopropyl)-4-[4-[2-chloro-4-[[5-(2,3-difluoro-4-methoxy-phenyl)-1-methyl-imidazole-2-carbonyl]amino]benzoyl]piperazine-1-carbonyl]piperazin-1-ium-1-yl]acetic acid formate;
2-[4-[4-[2-chloro-4-[[5-(2,3-difluoro-4-methoxy-phenyl)-1-methyl-imidazole-2-carbonyl]amino]benzoyl]piperazine-1-carbonyl]-1-[2-(dimethylamino)ethyl]piperidin-1-ium-1-yl]acetic acid bromide;
2-[1-(azetidin-3-ylmethyl)-4-[4-[2-chloro-4-[[5-(2-chloro-3-fluoro-4-methoxy-phenyl)-1-methyl-imidazole-2-carbonyl]amino]benzoyl]piperazine-1-carbonyl]piperidin-1-ium-1-yl]acetic acid;formate;
2-[1-(azetidin-3-ylmethyl)-4-[4-[2-chloro-4-[[5-[2-chloro-3-fluoro-4-(fluoromethoxy)phenyl]-1-methyl-imidazole-2-carbonyl]amino]benzoyl]piperazine-1-carbonyl]piperidin-1-ium-1-yl]acetic acid;formate;
N-[4-[4-[1-(azetidin-3-ylmethyl)-1-methyl-piperidin-1-ium-4-carbonyl]piperazine-1-carbonyl]-3-fluoro-phenyl]-5-[4-(difluoromethoxy)-2,3-difluoro-phenyl]-1-methyl-imidazole-2-carboxamide;2,2,2-trifluoroacetate;2,2,2-trifluoroacetic acid; and
N-[3-chloro-4-[4-[[(2S,4R)-4-hydroxy-1,1-dimethyl-pyrrolidin-1-ium-2-carbonyl]amino]piperidine-1-carbonyl]phenyl]-5-(2,3-difluoro-4-methoxy-phenyl)-1-methyl-imidazole-2-carboxamide;formate.

In a preferred embodiment, there is provided a compound of formula (I) as described herein, or a pharmaceutically acceptable salt thereof, wherein said compound of formula (I) is selected from:
(2-amino-2-oxo-ethyl)-[2-[4-[2-chloro-4-[[5-(2,3-difluoro-4-methoxy-phenyl)-1-methyl-imidazole-2-carbonyl]amino]benzoyl]piperazin-1-yl]-2-oxo-ethyl]-dimethyl-ammonium;formate;
N-[3-chloro-4-[4-(1,1-dimethylpiperidin-1-ium-4-carbonyl)piperazine-1-carbonyl]phenyl]-5-(2,3-difluoro-4-methoxy-phenyl)-1-methyl-imidazole-2-carboxamide;formate;
[2-[4-[2-chloro-4-[[5-[4-(difluoromethoxy)-2,3-difluoro-phenyl]-1-methyl-imidazole-2-carbonyl]amino]benzoyl]piperazin-1-yl]-2-oxo-ethyl]-trimethyl-ammonium;formate;
[2-[4-[2-chloro-4-[[5-(2,3-difluoro-4-methoxy-phenyl)-1-methyl-imidazole-2-carbonyl]amino]benzoyl]piperazin-1-yl]-2-oxo-ethyl]-trimethyl-ammonium;formate;
(2S,4R)-2-(4-(2-chloro-4-(5-(2,3-difluoro-4-methoxyphenyl)-1-methyl-1H-imidazole-2-carboxamido)benzoyl)piperazine-1-carbonyl)-4-hydroxy-1,1-dimethylpyrrolidin-1-ium formate;
(2S,4R)-2-(4-(2-chloro-4-(5-(2,3-difluoro-4-methoxyphenyl)-1-methyl-1H-imidazole-2-carboxamido)benzoyl)piperazine-1-carbonyl)-4-hydroxy-1,1,4-trimethylpyrrolidin-1-ium formate;
N-[3-chloro-4-[4-(3-hydroxy-1,1-dimethyl-piperidin-1-ium-4-carbonyl)piperazine-1-carbonyl]phenyl]-5-(2,3-difluoro-4-methoxy-phenyl)-1-methyl-imidazole-2-carboxamide;formate;
N-[3-chloro-4-[4-[(2S,4R)-4-hydroxy-1,1-dimethyl-pyrrolidin-1-ium-2-carbonyl]piperazine-1-carbonyl]phenyl]-5-[4-(difluoromethoxy)-2,3-difluoro-phenyl]-1-methyl-imidazole-2-carboxamide;formate;
5-(2-chloro-3-fluoro-4-methoxy-phenyl)-N-[3-chloro-4-[4-[(2S,4R)-4-hydroxy-1,1-dimethyl-pyrrolidin-1-ium-2-carbonyl]piperazine-1-carbonyl]phenyl]-1-methyl-imidazole-2-carboxamide;formate;
N-[3-chloro-4-[4-[(2S,4R)-4-hydroxy-1,1-dimethyl-pyrrolidin-1-ium-2-carbonyl]piperazine-1-carbonyl]phenyl]-5-[2,3-difluoro-4-(fluoromethoxy)phenyl]-1-methyl-imidazole-2-carboxamide;formate;
N-[3-chloro-4-[4-[(2S,5S)-5-hydroxy-1,1-dimethyl-piperidin-1-ium-2-carbonyl]piperazine-1-carbonyl]phenyl]-5-(2,3-difluoro-4-methoxy-phenyl)-1-methyl-imidazole-2-carboxamide;formate;
5-[3-chloro-4-(difluoromethoxy)-2-fluoro-phenyl]-N-[3-chloro-4-[4-[(2S,4R)-4-hydroxy-1,1-dimethyl-pyrrolidin-1-ium-2-carbonyl]piperazine-1-carbonyl]phenyl]-1-methyl-imidazole-2-carboxamide;iodide;
5-(3-chloro-2-fluoro-4-methoxy-phenyl)-N-[3-chloro-4-[4-[(2S,4R)-4-hydroxy-1,1-dimethyl-pyrrolidin-1-ium-2-carbonyl]piperazine-1-carbonyl]phenyl]-1-methyl-imidazole-2-carboxamide;formate;
N-[4-[4-[1-(2-Amino-2-oxo-ethyl)-1-methyl-piperidin-1-ium-4-carbonyl]piperazine-1-carbonyl]-3-chloro-phenyl]-5-[2-chloro-4-(difluoromethoxy)-3-fluoro-phenyl]-1-methyl-imidazole-2-carboxamide;formate;
N-[4-[4-[1-(2-amino-2-oxo-ethyl)-1-methyl-piperidin-1-ium-4-carbonyl]piperazine-1-carbonyl]-3-chloro-phenyl]-5-[4-(difluoromethoxy)-2,3-difluoro-phenyl]-1-methyl-imidazole-2-carboxamide;formate;
N-[4-[4-[1-(2-amino-2-oxo-ethyl)-1-methyl-piperidin-1-ium-4-carbonyl]piperazine-1-carbonyl]-3-chloro-phenyl]-5-(2-chloro-3-fluoro-4-methoxy-phenyl)-1-methyl-imidazole-2-carboxamide;formate;
N-[4-[4-[(1R,5S)-3-(2-amino-2-oxo-ethyl)-3-methyl-3-azoniabicyclo[3.1.0]hexane-6-carbonyl]piperazine-1-carbonyl]-3-chloro-phenyl]-5-[4-(difluoromethoxy)-2,3-difluoro-phenyl]-1-methyl-imidazole-2-carboxamide;formate;
N-[4-[4-[1-(2-amino-2-oxo-ethyl)-1-methyl-piperidin-1-ium-4-carbonyl]piperazine-1-carbonyl]-3-chloro-phenyl]-5-(2,3-difluoro-4-methoxy-phenyl)-1-methyl-imidazole-2-carboxamide;formate;
N-[3-chloro-4-[[1-[(1,1-dimethylazetidin-1-ium-3-yl)methyl]pyrazol-4-yl]methylcarbamoyl]phenyl]-5-(2,3-difluoro-4-methoxy-phenyl)-1-methyl-imidazole-2-carboxamide;formate;
[2-[[5-[[[2-chloro-4-[[5-(2,3-difluoro-4-methoxy-phenyl)-1-methyl-imidazole-2-carbonyl]amino]benzoyl]amino]methyl]-3-methyl-thiazol-3-ium-2-yl]amino]-2-oxo-ethyl]-trimethyl-ammonium;formate;
2-[1-(azetidin-3-ylmethyl)-4-[4-[2-chloro-4-[[5-(2,3-difluoro-4-methoxy-phenyl)-1-methyl-imidazole-2-carbonyl]amino]benzoyl]piperazine-1-carbonyl]piperidin-1-ium-1-yl]acetic acid;formate;
2-[(3aS,6aR)-5-(3-aminopropyl)-2-[4-[2-chloro-4-[[5-(2,3-difluoro-4-methoxy-phenyl)-1-methyl-imidazole-2-carbonyl]amino]benzoyl]piperazine-1-carbonyl]-1,3,3a,4,6,6a-hexahydropyrrolo[3,4-c]pyrrol-5-ium-5-yl]acetic acid chloride hydrochloride; and
2-[1-(azetidin-3-ylmethyl)-4-[4-[2-chloro-4-[[5-[2-chloro-3-fluoro-4-(fluoromethoxy)phenyl]-1-methyl-imidazole-2-carbonyl]amino]benzoyl]piperazine-1-carbonyl]piperidin-1-ium-1-yl]acetic acid;formate.

In some embodiments, the compounds of formula (I) are isotopically-labeled by having one or more atoms therein replaced by an atom having a different atomic mass or mass number. Such isotopically-labeled (i.e., radiolabeled) compounds of formula (I) are considered to be within the scope of this disclosure. Examples of isotopes that can be incorporated into the compounds of formula (I) include isotopes of hydrogen, carbon, nitrogen, oxygen, phosphorous, sulfur, fluorine, chlorine, and iodine, such as, but not limited to, ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. Certain isotopically-labeled compounds of formula (I), for example, those incorporating a radioactive isotope, are useful in drug and/or substrate tissue distribution studies. The radioactive isotopes tritium, i.e. ³H, and carbon-14, i.e., ¹⁴C, are particularly useful for this purpose in view of their ease of incorporation and ready means of detection. For example, a compound of formula (I) can be enriched with 1, 2, 5, 10, 25, 50, 75, 90, 95, or 99 percent of a given isotope.

Substitution with heavier isotopes such as deuterium, i.e. ²H, may afford certain therapeutic advantages resulting from greater metabolic stability, for example, increased in vivo half-life or reduced dosage requirements.

Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of formula (I) can generally be prepared by conventional techniques known to those skilled in the art or by processes analogous to those described in the Examples as set out below using an appropriate isotopically-labeled reagent in place of the non-labeled reagent previously employed.

### Processes of Manufacturing

The preparation of compounds of formula (I) of the present invention may be carried out in sequential or convergent synthetic routes. Syntheses of the compounds of the invention are shown in the following schemes. The skills required for carrying out the reactions and purifications of the resulting products are known to those skilled in the art. The substituents and indices used in the following description of the processes have the significance given herein before unless indicated to the contrary. In more detail, the compounds of formula (I) can be manufactured by the methods given below, by the methods given in the examples or by analogous methods. Appropriate reaction conditions for the individual reaction steps are known to a person skilled in the art. Also, for reaction conditions described in literature affecting the described reactions see for example: Comprehensive Organic Transformations: A Guide to Functional Group Preparations, 3rd Edition, Richard C. Larock. John Wiley & Sons, New York, NY. 2018). We find it convenient to carry out the reactions in the presence or absence of a solvent. There is no particular restriction on the nature of the solvent to be employed, provided that it has no adverse effect on the reaction or the reagents involved and that it can dissolve the reagents, at least to some extent. The described reactions can take place over a wide range of temperatures, and the precise reaction temperature is not critical to the invention. It is convenient to carry out the described reactions in a temperature range between −78° C. to reflux temperature. The time required for the reaction may also vary widely, depending on many factors, notably the reaction temperature and the nature of the reagents. However, a period of from 0.5 h to several days will usually suffice to yield the described intermediates and compounds. The reaction sequence is not limited to the one displayed in the schemes, however, depending on the starting materials and their respective reactivity the sequence of reaction steps can be freely altered. Starting materials are either commercially available or can be prepared by methods analogous to the methods given below, by methods described in references cited in the description or in the examples, or by methods known in the art.

All substituents have the meanings as defined above and in the claims, unless otherwise indicated. Furthermore, and unless explicitly otherwise stated, all reactions, reaction conditions, abbreviations and symbols have the meanings well known to a person of ordinary skill in organic chemistry.

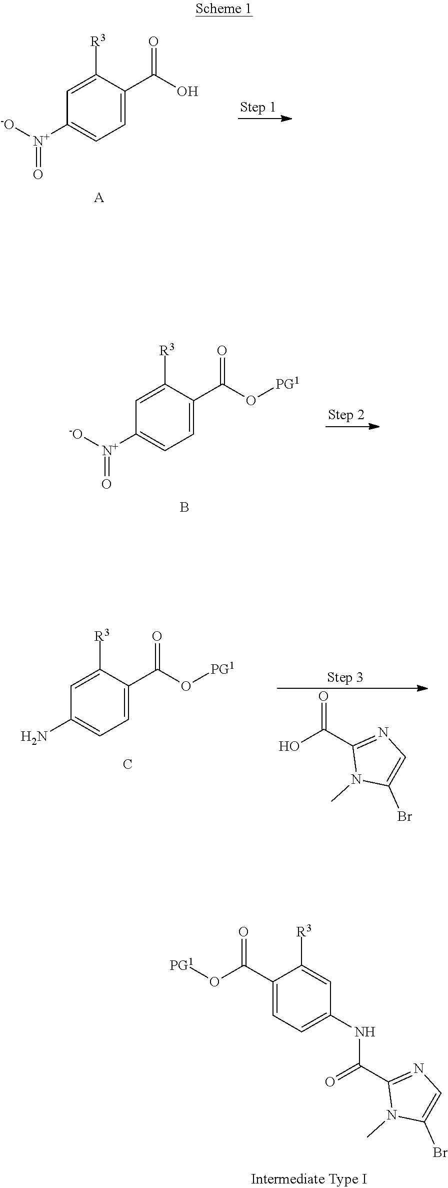

Wherein PG¹ is a protective group, e.g. a Boc protective group.

Intermediates of Type I can be prepared according to Scheme 1. Protection of substituted 4-nitrobenzoic acid A with e.g. (Boc)₂O gives compounds B. Reduction of the nitro group of compounds B can be achieved under reductive conditions, e.g. using the well-known ammonium chloride/iron system at room temperature, to give amines C. Coupling of carboxylic acid D with amines C using a condensing agent, such as HATU or DIPEA, in a solvent like DMSO, affords intermediates of Type I.

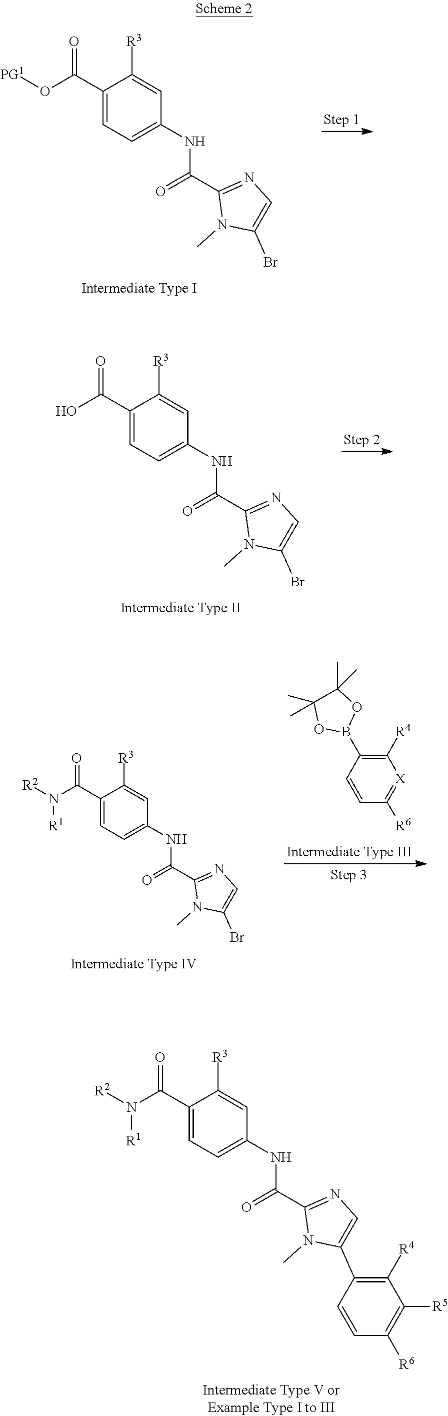

Wherein PG¹ is a protective group, e.g. a Boc protective group.

Intermediates of Type V or examples of Type I to III can be prepared according to Scheme 2. Hydrolysis of intermediates of Type I gives carboxylic acid intermediates of Type II, which can be coupled with diverse amines in the presence of a condensing agent, such as HATU or DIPEA, in solvents like DMSO, to afford intermediates of Type IV. Suzuki coupling of intermediates of Type IV with bronic acid ester intermediates of Type III in the presence of a transition metal catalyst, preferably a palladium catalyst having phosphine ligands, yields intermediates of Type V. Subsequent alkylation, e.g. methylation, of intermediates of Type V can be achieved using an alkylating agent like Mel, in the presence of a base, such as DIPEA, in a solvent, like acetonitrile, at room temperature to afford examples of Type I to III. The removal of the protective group can occur before or after the alkylation step, based on different substitution.

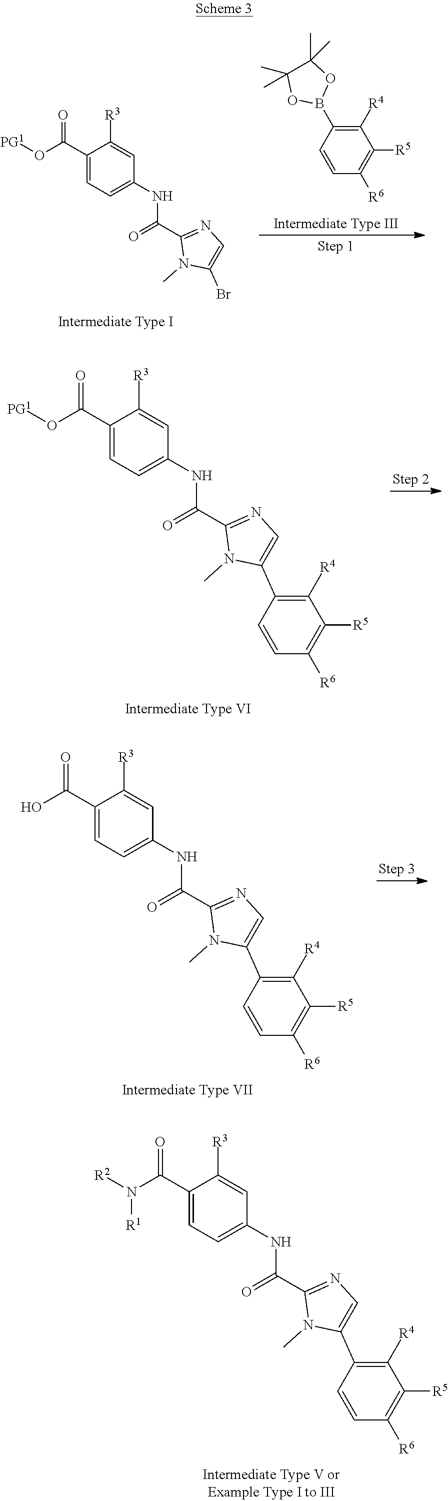

Wherein PG¹ is a protective group, e.g. a Boc protective group.

Intermediates of Type V or examples of Type I to III can also be prepared according to Scheme 3. Suzuki coupling of intermediates of Type I with bronic acid ester intermediates of Type III can be achieved using transition metal catalysts, preferably a palladium catalyst having phosphine ligands, to give intermediates of Type VI. Hydrolysis of intermediates of Type VI affords carboxylic acid intermediates of Type VII, which can be coupled with diverse amines in the presence of condensing agents, such as HATU or DIPEA, in solvents like DMSO, to afford intermediates of Type V. Subsequent alkylation, e.g. methylation, of intermediates of Type V can be achieved using an alkylating agent like Mel, in the presence of a base, such as DIPEA, in a solvent, like acetonitrile, at room temperature to afford examples of Type I to III. The removal of the protective group can occur before or after the methylation step, based on different substitution.

In one aspect, the present invention provides a process of manufacturing the compounds of formula (I) described herein, wherein said process is as described in any one of Schemes 1 to 4 above.

In a further aspect, the present invention provides a compound of formula (I) as described herein, or a pharmaceutically acceptable salt thereof, when manufactured according to the processes disclosed herein.

### Using the Compounds of the Invention

As illustrated in the experimental section, the compounds of formula (I) and their pharmaceutically acceptable salts possess valuable pharmacological properties for the treatment or prevention of infections and resulting diseases, particularly bacteremia, pneumonia, meningitis, urinary tract infection, and wound infection, caused by pathogens, particularly by bacteria, more particularly by Acinetobacter species, most particularly by Acinetobacter baumannii.

The compounds of formula (I) and their pharmaceutically acceptable salts exhibit activity as antibiotics, particularly as antibiotics against Acinetobacter species, more particularly as antibiotics against Acinetobacter baumannii, most particularly as pathogen-specific antibiotics against Acinetobacter baumannii.

The compounds of formula (I) and their pharmaceutically acceptable salts can be used as antibiotics, i.e. as antibacterial pharmaceutical ingredients suitable in the treatment and prevention of bacterial infections, particularly in the treatment and prevention of bacterial infections caused by Acinetobacter species, more particularly in the treatment and prevention of bacterial infections caused by Acinetobacter baumannii.

The compounds of the present invention can be used, either alone or in combination with other drugs, for the treatment or prevention of infections and resulting diseases, particularly bacteremia, pneumonia, meningitis, urinary tract infection, and wound infection, caused by pathogens, particularly by bacteria, more particularly caused by Acinetobacter species, most particularly by Acinetobacter baumannii.

In one aspect, the present invention provides compounds of formula (I) or their pharmaceutically acceptable salts as described herein for use as therapeutically active substances.

In a further aspect, the present invention provides a compound of formula (I) as described herein, or a pharmaceutically acceptable salt thereof, for use as antibiotic.

In a further aspect, the present invention provides a compound of formula (I) as described herein, or a pharmaceutically acceptable salt thereof, for use in the treatment or prevention of nosocomial infections and resulting diseases.

In a particular embodiment, said nosocomial infections and resulting diseases are selected from bacteremia, pneumonia, meningitis, urinary tract infection and wound infection, or a combination thereof.

In a further aspect, the present invention provides a compound of formula (I) as described herein, or a pharmaceutically acceptable salt thereof, for use in the treatment or prevention of infections and resulting diseases caused by Gram-negative bacteria.

In a particular embodiment, said infections and resulting diseases caused by Gram-negative bacteria are selected from bacteremia, pneumonia, meningitis, urinary tract infection and wound infection, or a combination thereof.

In a further aspect, the present invention provides a compound of formula (I) as described herein, or a pharmaceutically acceptable salt thereof, for use in the treatment or prevention of infections and resulting diseases caused by Enterococcus faecium, Staphylococcus aureus, Klebsiella pneumoniae, Acinetobacter baumannii, Pseudomonas aeruginosa, Enterobacter species or E. coli, or a combination thereof.

In a further aspect, the present invention provides a method for the treatment or prevention of infections and resulting diseases caused by Enterococcus faecium, Staphylococcus aureus, Klebsiella pneumoniae, Acinetobacter baumannii, Pseudomonas aeruginosa, Enterobacter species or E. coli, or a combination thereof, which method comprises administering a compound of formula (I) as described herein, or a pharmaceutically acceptable salt thereof, to a mammal.

In a further aspect, the present invention provides the use of a compound of formula (I) as described herein, or a pharmaceutically acceptable salt thereof, as an antibiotic.

In a further aspect, the present invention provides the use of a compound of formula (I) as described herein, or a pharmaceutically acceptable salt thereof, for the treatment or prevention of infections and resulting diseases caused by Enterococcus faecium, Staphylococcus aureus, Klebsiella pneumoniae, Acinetobacter baumannii, Pseudomonas aeruginosa, Enterobacter species or E. coli, or a combination thereof.

In a further aspect, the present invention provides the use of a compound of formula (I) as described herein, or a pharmaceutically acceptable salt thereof, for the preparation of medicaments useful for the treatment or prevention of infections and resulting diseases caused by Enterococcus faecium, Staphylococcus aureus, Klebsiella pneumoniae, Acinetobacter baumannii, Pseudomonas aeruginosa, Enterobacter species or E. coli, or a combination therof.

In a particular embodiment, said infections and resulting diseases caused by Enterococcus faecium, Staphylococcus aureus, Klebsiella pneumoniae, Acinetobacter baumannii, Pseudomonas aeruginosa, Enterobacter species or E. coli, or a combination thereof, are selected from bacteremia, pneumonia, meningitis, urinary tract infection and wound infection, or a combination thereof.

In a further aspect, the present invention provides compounds of formula (I) or their pharmaceutically acceptable salts as defined above for use in the treatment or prevention of infections and resulting diseases, particularly bacteremia, pneumonia, meningitis, urinary tract infection, and wound infection, caused by pathogens, particularly by bacteria, more particularly caused by Acinetobacter species, most particularly by Acinetobacter baumannii.

In a further aspect, the present invention provides a method for the treatment or prevention of infections and resulting diseases, particularly bacteremia, pneumonia, meningitis, urinary tract infection, and wound infection, caused by pathogens, particularly by bacteria, more particularly caused by Acinetobacter species, most particularly by Acinetobacter baumannii, which method comprises administering a compound of formula (I) or a pharmaceutically acceptable salt thereof as defined above to a mammal.

In a further aspect, the present invention provides the use of compounds of formula (I) or their pharmaceutically acceptable salts as defined above for the treatment or prevention of infections and resulting diseases, particularly bacteremia, pneumonia, meningitis, urinary tract infection, and wound infection, caused by pathogens, particularly by bacteria, more particularly caused by Acinetobacter species, most particularly by Acinetobacter baumannii.

In a further aspect, the present invention provides the use of compounds of formula (I) or their pharmaceutically acceptable salts as defined above for the preparation of medicaments for the treatment or prevention of infections and resulting diseases, particularly bacteremia, pneumonia, meningitis, urinary tract infection, and wound infection, caused by pathogens, particularly by bacteria, more particularly caused by Acinetobacter species, most particularly by Acinetobacter baumannii. Such medicaments comprise compounds of formula (I) or their pharmaceutically acceptable salts as defined above.

### Pharmaceutical Compositions and Administration

In one aspect, the present invention provides pharmaceutical compositions comprising compounds of formula (I) or their pharmaceutically acceptable salts as defined above and one or more pharmaceutically acceptable excipients. Exemplary pharmaceutical compositions are described in Examples 1-4.

In a further aspect, the present invention relates to pharmaceutical compositions comprising compounds of formula (I) or their pharmaceutically acceptable salts as defined above and one or more pharmaceutically acceptable excipients for the treatment or prevention of infections and resulting diseases, particularly bacteremia, pneumonia, meningitis, urinary tract infection, and wound infection, caused by pathogens, particularly by bacteria, more particularly caused by Acinetobacter species, most particularly by Acinetobacter baumannii.

The compounds of formula (I) and their pharmaceutically acceptable salts can be used as medicaments (e.g. in the form of pharmaceutical preparations). The pharmaceutical preparations can be administered internally, such as orally (e.g. in the form of tablets, coated tablets, dragées, hard and soft gelatin capsules, solutions, emulsions or suspensions), nasally (e.g. in the form of nasal sprays) or rectally (e.g. in the form of suppositories). However, the administration can also be effected parentally, such as intramuscularly or intravenously (e.g. in the form of injection solutions or infusion solutions).

The compounds of formula (I) and their pharmaceutically acceptable salts can be processed with pharmaceutically inert, inorganic or organic excipients for the production of tablets, coated tablets, dragées and hard gelatin capsules. Lactose, corn starch or derivatives thereof, talc, stearic acid or its salts etc. can be used, for example, as such excipients for tablets, dragées and hard gelatin capsules.

Suitable excipients for soft gelatin capsules are, for example, vegetable oils, waxes, fats, semi-solid substances and liquid polyols, etc.

Suitable excipients for the production of solutions and syrups are, for example, water, polyols, saccharose, invert sugar, glucose, etc.

Suitable excipients for injection solutions are, for example, water, alcohols, polyols, glycerol, vegetable oils, etc.

Suitable excipients for suppositories are, for example, natural or hardened oils, waxes, fats, semi-solid or liquid polyols, etc.

Moreover, the pharmaceutical preparations can contain preservatives, solubilizers, viscosity-increasing substances, stabilizers, wetting agents, emulsifiers, sweeteners, colorants, flavorants, salts for varying the osmotic pressure, buffers, masking agents or antioxidants. They can also contain still other therapeutically valuable substances.

The dosage can vary in wide limits and will, of course, be fitted to the individual requirements in each particular case. In general, in the case of oral administration a daily dosage of about 0.1 mg to 20 mg per kg body weight, preferably about 0.5 mg to 4 mg per kg body weight (e.g. about 300 mg per person), divided into preferably 1-3 individual doses, which can consist, for example, of the same amounts, should be appropriate. It will, however, be clear that the upper limit given herein can be exceeded when this is shown to be indicated.

### Co-Administration of Compounds of Formula (I) and Other Agents

The compounds of formula (I) or salts thereof or a compound disclosed herein or a pharmaceutically acceptable salt thereof may be employed alone or in combination with other agents for treatment. For example, the second agent of the pharmaceutical combination formulation or dosing regimen may have complementary activities to the compound of formula (I) such that they do not adversely affect each other. The compounds may be administered together in a unitary pharmaceutical composition or separately. In one embodiment a compound or a pharmaceutically acceptable salt can be co-administered with an antibiotic, in particular with an antibiotic for the treatment or prevention of infections and resulting diseases caused by Enterococcus faecium, Staphylococcus aureus, Klebsiella pneumoniae, Acinetobacter baumannii, Pseudomonas aeruginosa, Enterobacter species or E. coli, or a combination thereof.

The term “co-administering” refers to either simultaneous administration, or any manner of separate sequential administration, of a compound of formula (I) or a salt thereof or a compound disclosed herein or a pharmaceutically acceptable salt thereof and a further active pharmaceutical ingredient or ingredients, including antibiotic agents. If the administration is not simultaneous, the compounds are administered in a close time proximity to each other. Furthermore, it does not matter if the compounds are administered in the same dosage form, e.g. one compound may be administered intravenously and another compound may be administered orally.

Typically, any agent that has antimicrobial activity may be co-administered. Particular examples of such agents are Carbapenems (meropenem), Fluoroquinolone (Ciprofloxacin), Aminoglycoside (amikacin), Tetracyclines (tigecycline), Colistin, Sulbactam, Sulbactam+Durlobactam, Cefiderocol (Fetroja), macrocyclic peptides as exemplified e.g. in WO 2017072062 A1, WO 2019185572 A1 and WO 2019206853 A1, and Macrolides (erythromycin).

In one aspect, the present invention provides a pharmaceutical composition described herein, further comprising an additional therapeutic agent.

In one embodiment, said additional therapeutic agent is an antibiotic agent.

In one embodiment, said additional therapeutic agent is an antibiotic agent that is useful for the treatment or prevention of infections and resulting diseases caused by Enterococcus faecium, Staphylococcus aureus, Klebsiella pneumoniae, Acinetobacter baumannii, Pseudomonas aeruginosa, Enterobacter species or E. coli, or a combination thereof.

In one embodiment, said additional therapeutic agent is an antibiotic agent selected from Carbapenems (meropenem), Fluoroquinolone (Ciprofloxacin), Aminoglycoside (amikacin), Tetracyclines (tigecycline), Colistin, Sulbactam, Sulbactam+Durlobactam, Cefiderocol (Fetroja), macrocyclic peptides as exemplified in WO 2017072062 A1, WO 2019185572 A1 and WO 2019206853 A1, and Macrolides (erythromycin).

### EXAMPLES

The invention will be more fully understood by reference to the following examples. The claims should not, however, be construed as limited to the scope of the examples.

In case the preparative examples are obtained as a mixture of enantiomers, the pure enantiomers can be separated by methods described herein or by methods known to the man skilled in the art, such as e.g., chiral chromatography (e.g., chiral SFC) or crystallization.

All reaction examples and intermediates were prepared under an argon atmosphere if not specified otherwise.

Abbreviations used herein are as follows:
ACN or MeCN Acetonitrile
BINAP 2,2′-Bis(diphenylphosphino)-1,1′-binaphthalene
CFU colony-forming unit
d day
DCM dichloromethane
DIPEA N,N-diisopropylethylamine
EtOAc or EA Ethyl acetate
FA Formic acid
h(s) or hr(s) hour
HATU: 1-[Bis(dimethylamino)methylene]-1H-1,2,3-triazolo[4,5-b]pyridinium 3-oxid hexafluorophosphate
HPLC: high performance liquid chromatography
HPLC-UV: high performance liquid chromatography with ultraviolet detector
IC50 half maximal inhibitory concentration
IC90 90% inhibitory concentration
NaBH3CN Sodium cyanoborohydride
PE petroleum ether
PdCl2(DPPF) [1,1′-Bis(diphenylphosphino)ferrocene]dichloropalladium(II)
Pd2(dba)3 Tris(dibenzylideneacetone)dipalladium(0)
PG Protective group
Precat precatalyst
prep-HPLC preparative high performance liquid chromatography
rt room temperature
sat saturated
SEM 2-methoxyethyl(trimethyl)silane
FA Formic acid
TEMPO (2,2,6,6-Tetramethylpiperidin-1-yl)oxyl
TFA Trifluoroacetic Acid
wt weight
X—PHOS 2-Dicyclohexylphosphino-2′,4′,6′-triisopropylbiphenyl

### Intermediate A1

### Methyl 4-[(5-bromo-1-methyl-imidazole-2-carbonyl)amino]-2-chloro-benzoate

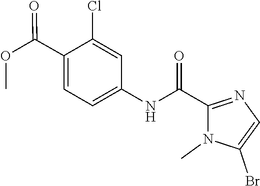 [SMILES: COC(=O)c1ccc(NC(=O)c2ncc(Br)n2C)cc1Cl]

### Step 1: isobutyl 5-bromo-1-methyl-imidazole-2-carboxylate

To a solution of 5-bromo-1-methyl-imidazole (20 g, 124 mmol) and DIPEA (32.1 g, 43.4 mL, 248 mmol) in DCM (140 mL) at −70° C. was added slowly a solution of isobutyl carbonochloridate (22.1 g, 161 mmol) in DCM (60 mL). The dropwise time lasted about 30 mins. The mixture was stirred at −70° C. for 2 h. Then the mixture was slowly warmed to room temperature. Then the solution was washed with water and concentrated in vacuo. The crude was purified by flash column chromatography to afford isobutyl 5-bromo-1-methyl-1H-imidazole-2-carboxylate (29 g, 89.4% yield) as a yellow oil.

### Step 2: 5-bromo-1-methyl-imidazole-2-carboxylic acid

To a solution of isobutyl 5-bromo-1-methyl-1H-imidazole-2-carboxylate (29 g, 111 mmol) in MeOH (5 mL) and THE (120 mL) was added a solution of Lithium hydroxide monohydrate (9.32 g, 222 mmol) in water (60 mL). The mixture was stirred at rt for 3 hrs. The organic solvent was removed under reduced pressure. 12 N HCl aqueous solution was added under stirring until PH 4-5. The white solid was filtered, washed with MeOH and dried to afford 5-bromo-1-methyl-imidazole-2-carboxylic acid (20.5 g, 90% yield) as a white solid.

### Step 3: methyl 4-[(5-bromo-1-methyl-imidazole-2-carbonyl)amino]-2-chloro-benzoate

A mixture of 5-bromo-1-methyl-1H-imidazole-2-carboxylic acid (13 g, 63.4 mmol), methyl 2-chloro-4-(methylamino)benzoate (11.8 g, 63.4 mmol), 2-(3H-[1,2,3]triazolo[4,5-b]pyridin-3-yl)-1,1,3,3-tetramethylisouronium hexafluorophosphate(V) (24.1 g, 63.4 mmol) and DIPEA (24.6 g, 33.2 mL) in DMF (30 mL) was stirred at room temperature overnight. Then the mixture was poured into water. The water phase was extracted with DCM (3×50 mL). The combined organic phases were washed with water, dried over anhydrous Na₂SO₄ and concentrated in vacuum. The solids precipitated from the concentrated solution. The solids were collected, washed with MeOH and dried to afford methyl 4-[(5-bromo-1-methyl-imidazole-2-carbonyl) amino]-2-chloro-benzoate (18 g, 76% yield) as a light yellow solid. The following Type I Intermediates was prepared in analogy to Intermediate A1:

| | | | MS | |
|---|---|---|---|---|
| | | | ESI | |
| Int. | Name | Structure | [M + H]⁺ | Starting Material |
| Intermediate A2 | tert-butyl 4-[4-[(5- bromo-1-methyl- imidazole-2- carbonyl)amino]-2- fluoro- benzoyl]piperazine- 1-carboxylate | 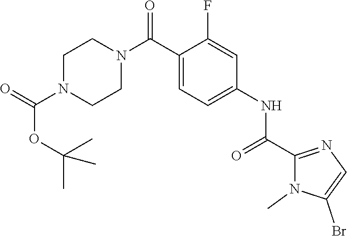 [SMILES: Cn1c(Br)cnc1C(=O)Nc1ccc(C(=O)N2CCN(C(=O)OC(C)(C)C)CC2)c(F)c1] | 509.9 | 5-bromo-1-methyl- imidazole and Intermediate 6c |

### Intermediate A3

### tert-butyl 4-[4-[4-[(5-bromo-1-methyl-imidazole-2-carbonyl)amino]-2-fluoro-benzoyl]piperazine-1-carbonyl]piperidine-1-carboxylate

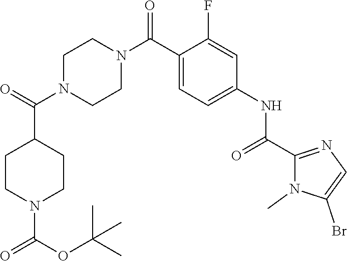 [SMILES: Cn1c(Br)cnc1C(=O)Nc1ccc(C(=O)N2CCN(C(=O)C3CCN(C(=O)OC(C)(C)C)CC3)CC2)c(F)c1]

### Step 1: 5-bromo-N-[3-fluoro-4-(piperazine-1-carbonyl)phenyl]-1-methyl-imidazole-2-carboxamide

A solution of tert-butyl 4-[4-[(5-bromo-1-methyl-imidazole-2-carbonyl)amino]-2-fluoro-benzoyl]piperazine-1-carboxylate (1 g, 1.96 mmol) in DCM (10 mL) and TFA (6 mL) was stirred at room temperature for 30 mins. Then the solution was concentrated and the residue was dissolved in DCM (20 mL). The organic layer was basified to PH 8-9 with NH₃·H₂O. The organic layer was washed with water, dried and concentrated. The residue was used into next step reaction without further purification. ESI MS [M+H]⁺ 409.9.

### Step 2: tert-butyl 4-[4-[4-[(5-bromo-1-methyl-imidazole-2-carbonyl)amino]-2-fluoro-benzoyl]piperazine-1-carbonyl]piperidine-1-carboxylate

At room temperature, a mixture of 1-tert-butoxycarbonylpiperidine-4-carboxylic acid (503 mg, 2.19 mmol), 5-bromo-N-[3-fluoro-4-(piperazine-1-carbonyl)phenyl]-1-methyl-imidazole-2-carboxamide (900 mg, 2.19 mmol), HATU (834 mg, 2.19 mmol) and DIPEA (284 mg, 2.19 mmol) in DMF (10 mL) was stirred for 1 h. Then the mixture was poured into water. The solid was collected and dried to give the title compound (1.2 g) as a yellow solid.

ESI MS [M+H]+ 620.9.

### Intermediate B1

### 4-[(5-Bromo-1-methyl-imidazole-2-carbonyl)amino]-2-chloro-benzoic acid

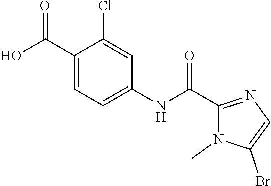 [SMILES: Cn1c(Br)cnc1C(=O)Nc1ccc(C(=O)O)c(Cl)c1]

To a solution of methyl 4-[(5-bromo-1-methyl-imidazole-2-carbonyl)amino]-2-chloro-benzoate (7.6 g, 20.4 mmol) in MeOH (2 mL) and THE (48 mL) was added a solution of Lithium hydroxide monohydrate (2.57 g, 61.2 mmol) in water (24 mL). The mixture was stirred at r.t overnight. Then the mixture was concentrated and the water layer was acidified by HCl. The solids precipitated from the concentrated solution. The solids were collected, washed with water and dried to afford 4-[(5-bromo-1-methyl-imidazole-2-carbonyl)amino]-2-chloro-benzoic acid as a white solid (6 g, 8200 yield). ESI MS [M+H]⁺:358.0.

The following Type 11 Intermediates was prepared in analogy to Intermediate B1:

| | | | MS | |
|---|---|---|---|---|
| | | | ESI | |
| Int. | Name | Structure | [M + H]⁺ | Starting Material |
| Intermediate B2 | 4-[(5-bromo-1-methyl- imidazole-2- carbonyl)amino]-2- methyl-benzoic acid | 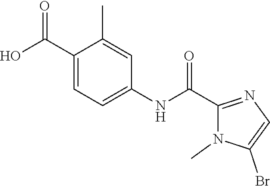 [SMILES: Cc1cc(NC(=O)c2ncc(Br)n2C)ccc1C(=O)O] | 338.0 | 5-bromo-1-methyl- 1H-imidazole-2- carboxylic acid and methyl 4-amino-2- methylbenzoate |
| Intermediate B3 | 4-(5-(2,3-difluoro-4- methoxyphenyl)-1- methyl-1H-imidazole- 2-carboxamido)-2- ethylbenzoic acid | 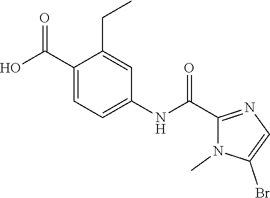 [SMILES: CCc1cc(NC(=O)c2ncc(Br)n2C)ccc1C(=O)O] | 414.3 | 5-bromo-1-methyl- 1H-imidazole-2- carboxylic acid and methyl 4-amino-2- ethylbenzoate |

### Intermediate C1

### 2-[2,3-Difluoro-4-(4,4,5,5-tetramethyl-1,3,2-dioxaborolan-2-yl)phenoxy]acetonitrile

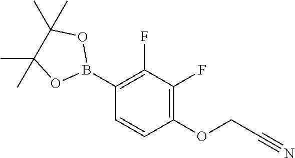 [SMILES: CC1(C)OB(c2ccc(OCC#N)c(F)c2F)OC1(C)C]

### Step 1: 2-(4-bromo-2,3-difluoro-phenoxy)acetonitrile

To a solution of 4-bromo-2,3-difluorophenol (5.2 g, 25 mmol),bromoacetonitrile (6.0 g, 50 mmol) in DMF (25 mL) was added potassium carbonate (6.9 g, 50 mmol) and then the resultant mixture was stirred overnight at room temperature. The mixture was poured into water (50 mL) and the aqueous solution was extracted with EA (100 mL×2). The organic layers were combined and washed with water and brine, dried over anhydrous Na₂SO₄ and concentrated under reduced pressure. The residue was purified by column chromatography to give the title compound (5.2 g, 84% yield) as a white solid.

### Step 2: 2-(2,3-difluoro-4-(4,4,5,5-tetramethyl-1,3,2-dioxaborolan-2-yl)phenoxy acetonitrile

To a solution of 2-(4-bromo-2,3-difluorophenoxy)acetonitrile (6.2 g, 25 mmol) in dioxane (50 mL) and was added (4,4,4,4′,5,5,5′,5′-octamethyl-2,2′-bi(1,3,2-dioxaborolane) (6.35 g, 25 mmol), Pd(dppf)Cl₂ (1.6 g, 2 mmol) and potassium acetate (4.9 g, 50 mmol) and then the resultant mixture was degassed for 5 min with nitrogen and then stirred overnight at 80° C. After cooling to room temperature, the mixture was poured into water (100 mL) and the aqueous solution was extracted with EA (100 mL×2). The organic layers were combined and washed with water and brine, dried over anhydrous Na₂SO₄ and concentrated under reduced pressure to give a red oil which was purified by Flash to provide the desired compound (4 g, 5400 yield) as an off-white solid.

The following Type III Intermediates were prepared in analogy to Intermediate C1:

| | | | MS | |
|---|---|---|---|---|
| | | | ESI | |
| Int. | Name | Structure | [M + H]⁺ | Starting Material |
| Intermediate C2 | 2-(2-chloro-3-fluoro-4- methoxy-phenyl)- 4,4,5,5-tetramethyl- 1,3,2-dioxaborolane | 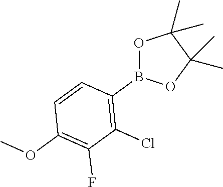 | NA | 4-bromo-3-chloro-2- fluoro-phenol and iodomethane; bis(pinacolato) diboron |
| Intermediate C7 | 2-[2-fluoro-4- (fluoromethoxy) phenyl]-4,4,5,5- tetramethyl- 1,3,2-dioxaborolane | 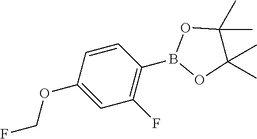 [SMILES: CC1(C)OB(c2ccc(OCF)cc2F)OC1(C)C] | NA | 4-bromo-3-fluoro- phenol and fluoromethyl 4- methylbenzene- sulfonate |
| Intermediate C8 | 2-[2,3-difluoro-4- (4,4,5,5-tetramethyl- 1,3,2-dioxaborolan-2- yl)phenoxy]acetonitrile | 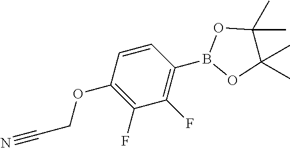 [SMILES: CC1(C)OB(c2ccc(OCC#N)c(F)c2F)OC1(C)C] | NA | 4-bromo-2,3- difluorophenol and 2- bromoacetonitrile |
| Intermediate C9 | 2-[2,3-difluoro-4- (fluoromethoxy) phenyl]-4,4,5,5- tetramethyl- 1,3,2-dioxaborolane | 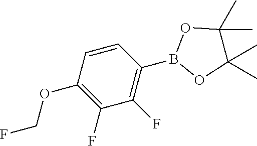 [SMILES: CC1(C)OB(c2ccc(OCF)c(F)c2F)OC1(C)C] | NA | 4-bromo-2,3- difluoro-phenol and fluoromethyl 4- methylbenzene- sulfonate |
| Intermediate C11 | 2-(3-chloro-2-fluoro-4- methoxy-phenyl)- 4,4,5,5-tetramethyl- 1,3,2-dioxaborolane | 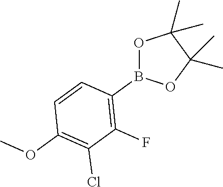 | NA | 4-bromo-2-chloro-3- fluoro-phenol and iodomethane; bis(pinacolato) diboron |
| Intermediate C14 | 2-[2-chloro-3-fluoro-4- (fluoromethoxy) phenyl]-4,4,5,5- tetramethyl- 1,3,2-dioxaborolane | 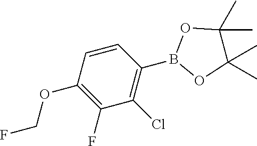 [SMILES: CC1(C)OB(c2ccc(OCF)c(F)c2Cl)OC1(C)C] | NA | 4-bromo-3-chloro-2- fluoro-phenol and fluoromethyl 4- methylbenzene- sulfonate; bis(pinacolato) diboron |
| Intermediate C15 | 2-[2-(difluoromethyl)- 3-fluoro-4-methoxy- phenyl]-4,4,5,5- tetramethyl-1,3,2- dioxaborolane | 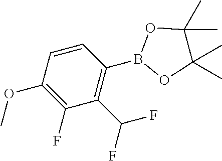 | NA | 4-bromo-3- (difluoromethyl)-2- fluoro-phenol and iodomethane; bis(pinacolato) diboron |

### Intermediate C3

### 2-(4-(Difluoromethoxy)-2,3-difluorophenyl)-4,4,5,5-tetramethyl-1,3,2-dioxaborolane

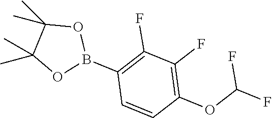 [SMILES: CC1(C)OB(c2ccc(OC(F)F)c(F)c2F)OC1(C)C]

### Step 1: 1-bromo-4-(difluoromethoxy)-2,3-difluorobenzene

To a solution of 4-bromo-2,3-difluorophenol (25 g, 120 mmol), sodium 2-chloro-2,2-difluoroacetate (36.5 g, 239 mmol) and K₂CO₃ (19.8 g, 144 mmol) in DMF (250 mL) and Water (57 mL). The reaction mixture was stirred for 3 h at 100° C. under N₂. The reaction mixture was poured into 1.5 L 1 H₂O and extracted with EtOAc (3×250 mL). The organic layers were combined, washed with sat NaCl (1×200 mL). The organic layers were dried over Na₂SO₄ and concentrated in vacuum. The crude material was purified by flash chromatography to afford 1-bromo-4-(difluoromethoxy)-2,3-difluorobenzene (25.2 g, 97.3 mmol, 81.3% yield)

### Step 2: 2-(4-(difluoromethoxy)-2,3-difluorophenyl)-4,4,5,5-tetramethyl-1,3,2-dioxaborolane

To a 250 mL RBF was added 4,4,4′,4′,5,5,5′,5′-octamethyl-2,2′-bi(1,3,2-dioxaborolane) (24.5 g, 96.5 mmol), 1-bromo-4-(difluoromethoxy)-2,3-difluorobenzene (25 g, 96.5 mmol), PdCl₂(DPPF)—CH₂Cl₂ adduct (3.53 g, 4.83 mmol) and potassium acetate (18.9 g, 193 mmol) in Dioxane (150 mL). The vial was capped and heated at 80° C. for 15 h under N₂. The crude reaction mixture was concentrated in vacuum. The reaction mixture was poured into 50 mL H₂O and extracted with EtOAc (3×50 mL). The organic layers were combined, washed with sat NaCl (1×50 mL). The organic layers were dried over Na₂SO₄ and concentrated in vacuum. The crude material was purified by flash chromatography to afford 2-(4-(difluoromethoxy)-2,3-difluorophenyl)-4,4,5,5-tetramethyl-1,3,2-dioxaborolane (25 g, 81.7 mmol, 84.6% yield).

The following Type III Intermediates were prepared in analogy to Intermediate C3:

| | | | MS | |
|---|---|---|---|---|
| | | | ESI | |
| Int | Name | Structure | [M + H]⁺ | Starting Material |
| Intermediate C4 | 2-[4-(difluoromethoxy)- 2-fluoro-phenyl]- 4,4,5,5-tetramethyl- 1,3,2-dioxaborolane | 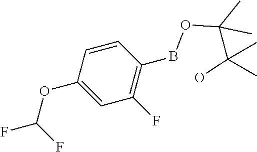 | NA | 4-bromo-3-fluoro- phenol and sodium 2- chloro-2,2- difluoroacetate |
| Intermediate C5 | 2-[4-(difluoromethoxy)- 3-fluoro-phenyl]- 4,4,5,5-tetramethyl- 1,3,2-dioxaborolane | 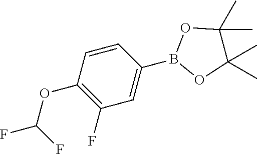 [SMILES: CC1(C)OB(c2ccc(OC(F)F)c(F)c2)OC1(C)C] | NA | 4-bromo-2-fluoro- phenol and sodium 2- chloro-2,2- difluoroacetate |
| Intermediate C6 | 2-[2-chloro-4- (difluoromethoxy)-3- fluoro-phenyl]-4,4,5,5- tetramethyl-1,3,2- dioxaborolane | 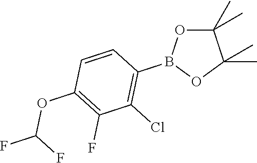 [SMILES: CC1(C)OB(c2ccc(OC(F)F)c(F)c2Cl)OC1(C)C] | NA | 4-bromo-3-chloro-2- fluoro-phenol and sodium 2-chloro-2,2- difluoroacetate |
| Intermediate C10 | 2-[3-chloro-4- difluoromethoxy)-2- fluoro-phenyl]-4,4,5,5- tetramethyl-1,3,2- dioxaborolane | 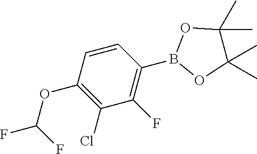 [SMILES: CC1(C)OB(c2ccc(OC(F)F)c(Cl)c2F)OC1(C)C] | NA | 4-bromo-2-chloro-3- fluoro-phenol and sodium 2- chloro-2,2- difluoroacetate |

### Intermediate C12

### (3-cyano-2-fluoro-4-methoxy-phenyl)boronic acid

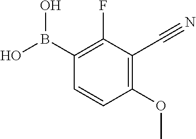

The title compound was prepared in analogy to intermediate C13 step 2 from 2-fluoro-6-methoxybenzonitrile (1.0 g, 6.62 mmol) and was obtained as light yellow oil (900 mg, 4.62 mmol, 69.77% yield) which was used without further purification. ESI MS [M+H]⁺: 196.1.

### Intermediate C13

### [2-fluoro-4-methoxy-3-(trifluoromethyl)phenyl]boronic acid

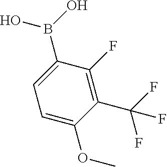 [SMILES: COc1ccc(B(O)O)c(F)c1C(F)(F)F]

### Step 1: 1-fluoro-3-methoxy-2-(trifluoromethyl)benzene

A solution of 2,6-difluorobenzotrifluoride (4.0 g, 21.97 mmol, 1 eq) in methanol (40 mL) was added 5M sodium methoxide in MeOH (4.39 mL, 21.97 mmol, 1 eq) at 20° C. and stirred at 50° C. for 16 h. The solution was concentrated in vacuum and the residue was suspended with DCM (100 mL), filtered and the filtrate concentrated to afford the title compound (1.1 g, 5.67 mmol, 25.79% yield) as colorless oil, which was used without further purification.

### Step 2: [2-fluoro-4-methoxy-3-(trifluoromethyl)phenyl]boronic acid

To solution of 1-fluoro-3-methoxy-2-(trifluoromethyl)benzene (500.0 mg, 2.58 mmol, 1 eq) in THE (10 mL) was added dropwise lithium diisopropylamide (1.24 mL, 3.09 mmol, 1.2 eq) and stirred at −70° C. for 30 min under N2. Then the mixture was added boron isopropoxide (0.77 mL, 3.35 mmol, 1.3 eq) and stirred at −70° C. for 3 h under N2. The mixture was poured into NH4Cl solution (80 mL), extracted with EtOAc (50 mL×2). The combined organic layers were dried over Na₂SO₄ and concentrated to afford the title compound (600 mg, 2.52 mmol, 97.91% yield) as light yellow solid, which used without purification.

### Intermediate E1

### N-(3-Chloro-4-(4-(dimethylglycyl)piperazine-1-carbonyl)phenyl)-5-(4-(cyanomethoxy)-2,3-difluorophenyl)-1-methyl-1H-imidazole-2-carboxamide

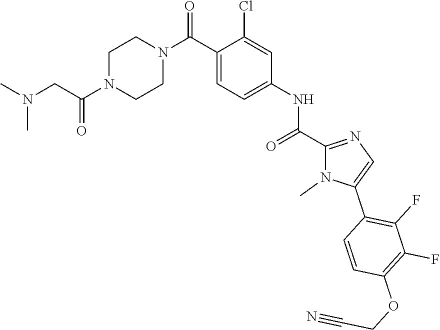 [SMILES: CN(C)CC(=O)N1CCN(C(=O)c2ccc(NC(=O)c3ncc(-c4ccc(OCC#N)c(F)c4F)n3C)cc2Cl)CC1]

### Step 1: 5-bromo-N-(3-chloro-4-(4-(dimethylglycyl)piperazine-1-carbonyl)phenyl)-1-methyl-1H-imidazole-2-carboxamide

1-(4-(4-amino-2-chlorobenzoyl)piperazin-1-yl)-2-(dimethylamino)ethan-1-one (1.65 g, 5.08 mmol), 5-bromo-1-methyl-1H-imidazole-2-carboxylic acid (1.04 g, 5.08 mmol) and HATU (2.32 g, 6.1 mmol) were dissolved in DMF (25 mL). The reaction mixture was cooled to 0° C. and DIPEA (2.63 g, 3.55 mL, 20.3 mmol) was added. It was stirred at room temperature overnight. Water was added to the reaction after cooling at 0° C. and it was extracted with DCM. The organic layers were combined, dried with MgSO₄, concentrated under vacuum and DMF was removed using HV for 1 h. The crude was purified by flash chromatography to afford the title compound (2.27 g, 3.99 mmol, 79.6% yield) as an orange solid. ESI MS [M+H]⁺: 511.2/513.3.

### Step 2: N-(3-chloro-4-(4-(dimethylglycyl)piperazine-1-carbonyl)phenyl)-5-(4-(cyanomethoxy)-2,3-difluorophenyl)-1-methyl-1H-imidazole-2-carboxamide

5-bromo-N-(3-chloro-4-(4-(dimethylglycyl)piperazine-1-carbonyl)phenyl)-1-methyl-1H-imidazole-2-carboxamide (768 mg, 1.5 mmol), 2-(2,3-difluoro-4-(4,4,5,5-tetramethyl-1,3,2-dioxaborolan-2-yl)phenoxy)acetonitrile (620 mg, 2.1 mmol), sodium carbonate (366 mg, 3.45 mmol), 1,1′-bis(di-tert-butylphospino)ferrocene palladium dichloride (196 mg, 300 μmol) were mixed in Dioxane (5 mL) and water (500 l). N₂ was bubbled through for 15 min. The reaction mixture was heated in the microwave to 100° C. for 1.5 h. The reaction mixture was diluted with dichloromethane and purified by flash chromatography to afford the title compound (1 g, 1 0.33 mmol, 88.900 yield) as a dark brown viscous oil (purity 80 0%). ESI MS [M+H]⁺: 600.4.

The following Type V Intermediates were prepared in analogy to Intermediate E1:

| | | | MS | |
|---|---|---|---|---|
| | | | ESI | |
| Int. | Name | Structure | [M + H]⁺ | Starting Material |
| Intermediate E2 | tert-butyl 4-[4-[4-[[5-[4- (difluoromethoxy)- 2,3-difluoro-phenyl]- 1-methyl-imidazole- 2-carbonyl]amino]-2- ethyl-benzoyl] piperazine-1- carbonyl]piperidine- 1-carboxylate | 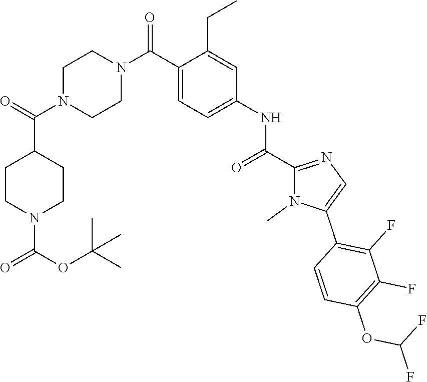 [SMILES: CCc1cc(NC(=O)c2ncc(-c3ccc(OC(F)F)c(F)c3F)n2C)ccc1C(=O)N1CCN(C(=O)C2CCN(C(=O)OC(C)(C)C)CC2)CC1] | 731.6 | 5-bromo-1-methyl- 1H-imidazole-2- carboxylic acid and Intermediate F2; Intermediate C3 |
| Intermediate E37 | 5-[4- (cyanomethoxy)-2,3- difluoro-phenyl]-N- [4-[4-[2- (dimethylamino) acetyl]piperazine-1- carbonyl]-3-methyl- phenyl]-1-methyl- imidazole-2- carboxamide | 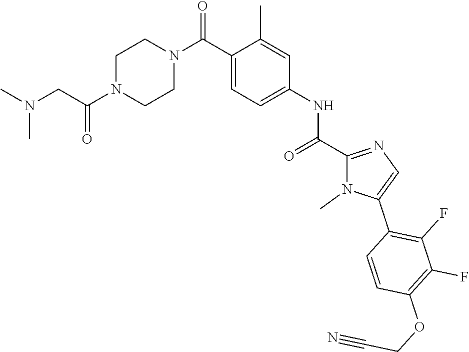 [SMILES: Cc1cc(NC(=O)c2ncc(-c3ccc(OCC#N)c(F)c3F)n2C)ccc1C(=O)N1CCN(C(=O)CN(C)C)CC1] | 780.3 | 5-bromo-1-methyl- 1H-imidazole-2- carboxylic acid and Intermediate F4; Intermediate C1 |

### Intermediate E3

### 5-(2,3-Difluoro-4-methoxy-phenyl)-1-methyl-N-[3-methyl-4-(piperazine-1-carbonyl)phenyl]imidazole-2-carboxamide

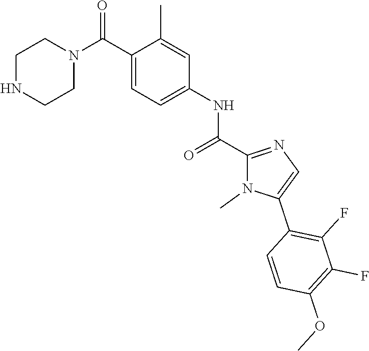 [SMILES: COc1ccc(-c2cnc(C(=O)Nc3ccc(C(=O)N4CCNCC4)c(C)c3)n2C)c(F)c1F]

### Step 1: tert-butyl 4-[4-[(5-bromo-1-methyl-imidazole-2-carbonyl)amino]-2-methyl-benzoyl]piperazine-1-carboxylate

To a solution of 4-[(5-bromo-1-methyl-imidazole-2-carbonyl)amino]-2-methyl-benzoic acid (2.0 g, 6 mmol), tert-butyl piperazine-1-carboxylate (1.34 g, 7.2 mmol) in anhydrous DMF (20 mL) was added TEA (1.2 g, 12 mmol) and then the resultant mixture was stirred for 10 min at room temperature, T₃P (6 mL, 9.0 mmol) was added in the mixture and stirred for extra 10 hr. The mixture was poured into water (50 mL) and the aqueous solution was extracted with DCM (50 mL×2). The organic layers were combined and washed with water and brine, dried over anhydrous Na₂SO₄ and concentrated under reduced pressure to give a red oil which was used in next step without purification. ESI MS [M+H]⁺: 506.3.

### Step 2: tert-butyl 4-[4-[[5-(2,3-difluoro-4-methoxy-phenyl)-1-methyl-imidazole-2-carbonyl]amino]-2-methyl-benzoyl]piperazine-1-carboxylate

A mixture of tert-butyl 4-[4-[(5-bromo-1-methyl-imidazole-2-carbonyl)amino]-2-methyl-benzoyl]piperazine-1-carboxylate (2.0 g, 4.0 mmol), (2,3-difluoro-4-methoxyphenyl)boronic acid (2.26 g, 12 mmol), Na₂CO₃ (1.28 g, 12 mmol) and 1,1′-Bis (di-t-butylphosphino) ferrocene palladium dichloride (260 mg, 0.4 mmol) in 1,4-Dioxane (15 mL) and water (1.5 mL) was irritated at 100° C. for 4 h. Then the solution was filtered and concentrated. The water layer was extracted with DCM. The organic layer was dried and concentrated. The residue was purified by Flash to give tert-butyl 4-[4-[[5-(2,3-difluoro-4-methoxy-phenyl)-1-methyl-imidazole-2-carbonyl]amino]-2-methyl-benzoyl]piperazine-1-carboxylate (1.95 g, 85.60 yield) as a white solid. ESI MS [M+H]⁺: 570.2.

### Step 3: 5-(2,3-difluoro-4-methoxy-phenyl)-1-methyl-N-[3-methyl-4-(piperazine-1-carbonyl)phenyl]imidazole-2-carboxamide

To a solution of tert-butyl 4-(4-(5-(2,3-difluoro-4-methoxyphenyl)-1-methyl-H-imidazole-2-carboxamido)-2-methylbenzoyl)piperazine-1-carboxylate (1.42 g, 2.5 mmol) in DCM (50 m) was added TFA (5 mL) at room temperature. The resultant mixture was stirred for 10 h and then adjusted to pH=7-8 with aqueous ammonia. The mixture was poured into water (50 mL) and then extracted with dichloromethane/isopropanol (100/10 mL), the organic layer was concentrated to give a red oil, which was purified by Flash to afford 5-(2,3-difluoro-4-methoxy-phenyl)-1-methyl-N-[3-methyl-4-(piperazine-1-carbonyl)phenyl]imidazole-2-carboxamide (0.75 g, 64.0 00 yield). ESI MS [M+H]⁺: 470.2.

The following Type V Intermediates were prepared in analogy to Intermediate E3:

| | | | MS | |
|---|---|---|---|---|
| | | | ESI | |
| Int. | Name | Structure | [M + H]⁺ | Starting Material |
| Intermediate E4 | 5-[4- (cyanomethoxy)-2,3- difluoro-phenyl]-1- methyl-N-[3-methyl- 4-(piperazine-1- carbonyl)phenyl] imidazole-2- carboxamide | 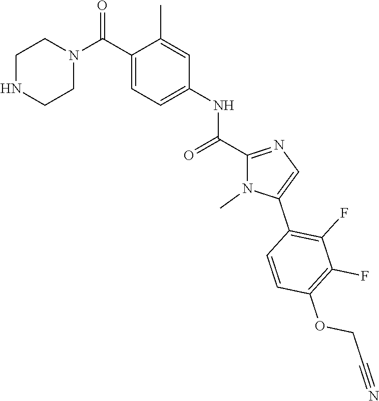 [SMILES: Cc1cc(NC(=O)c2ncc(-c3ccc(OCC#N)c(F)c3F)n2C)ccc1C(=O)N1CCNCC1] | 495.1 | Intermediate B2, tert-butyl piperazine-1- carboxylate and Intermediate C1 |
| Intermediate E5 | N-[3-chloro-4-(piperazine- 1-carbonyl)phenyl]-5- [4-(cyanomethoxy)- 2,3-difluoro-phenyl]- 1-methyl-imidazole- 2-carboxamide | 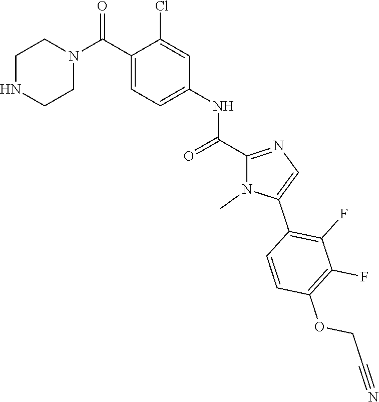 [SMILES: Cn1c(-c2ccc(OCC#N)c(F)c2F)cnc1C(=O)Nc1ccc(C(=O)N2CCNCC2)c(Cl)c1] | 515.2 | Intermediate B1 and tert-butyl piperazine-1- carboxylate and Intermediate C1 |
| Intermediate E6 | N-[3-chloro-4- (piperazine-1- carbonyl)phenyl]-5- (2,3-difluoro-4- methoxy-phenyl)-1- methyl-imidazole-2- carboxamide | 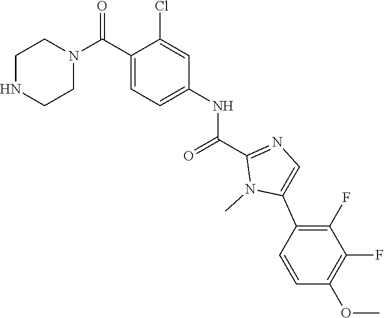 [SMILES: COc1ccc(-c2cnc(C(=O)Nc3ccc(C(=O)N4CCNCC4)c(Cl)c3)n2C)c(F)c1F] | 480.1 | Intermediate B1 and tert-butyl piperazine-1- carboxylate and (2,3-difluoro-4- methoxy-phenyl) boronic acid |
| Intermediate E7 | 5-(2-chloro-3-fluoro- 4-methoxy-phenyl)- N-[3-chloro-4- (piperazine-1- carbonyl)phenyl]-1- methyl-imidazole-2- carboxamide | 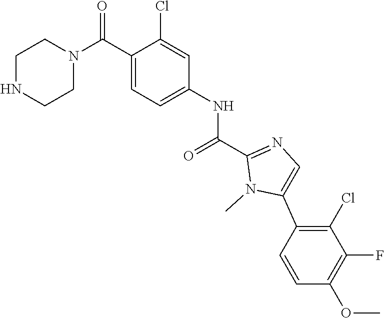 [SMILES: COc1ccc(-c2cnc(C(=O)Nc3ccc(C(=O)N4CCNCC4)c(Cl)c3)n2C)c(Cl)c1F] | 506.0 | Intermediate B1 and tert-butyl piperazine-1- carboxylate and Intermediate C2 |
| Intermediate E8 | N-[4-[4-(2- aminoethyl)piperidine- 1-carbonyl]-3- chloro-phenyl]-5- (2,3-difluoro-4- methoxy-phenyl)-1- methyl-imidazole-2- carboxamide | 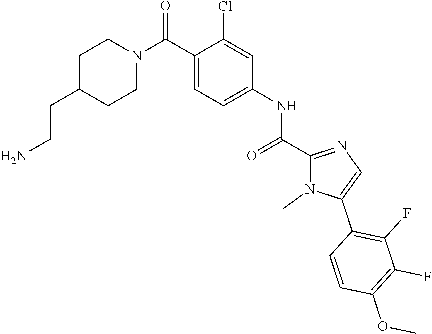 [SMILES: COc1ccc(-c2cnc(C(=O)Nc3ccc(C(=O)N4CCC(CCN)CC4)c(Cl)c3)n2C)c(F)c1F] | 532.2 | Intermediate B1 and tert-butyl (2- (piperidin-4- yl)ethyl)carbamate and (2,3-difluoro- 4-methoxy- phenyl)boronic acid |
| Intermediate E9 | N-[4-(3- aminoazetidine-1- carbonyl)-3-chloro- phenyl]-5-(2,3- difluoro-4-methoxy- phenyl)-1-methyl- imidazole-2- carboxamide | 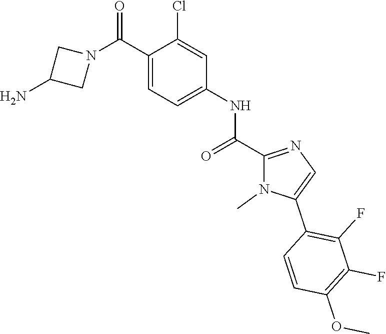 [SMILES: COc1ccc(-c2cnc(C(=O)Nc3ccc(C(=O)N4CC(N)C4)c(Cl)c3)n2C)c(F)c1F] | 476.3 | Intermediate B1 and tert-butyl N- (azetidin-3- yl)carbamate and (2,3-difluoro-4- methoxy- phenyl) boronic acid |
| Intermediate E10 | N-[4-(azetidin-3- ylcarbamoyl)-3- chloro-phenyl]-5- (2,3-difluoro-4- methoxy-phenyl)-1- methyl-imidazole-2- carboxamide | 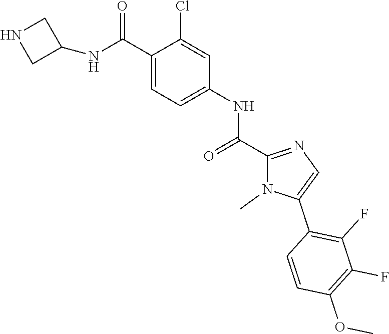 [SMILES: COc1ccc(-c2cnc(C(=O)Nc3ccc(C(=O)NC4CNC4)c(Cl)c3)n2C)c(F)c1F] | 476.1 | Intermediate B1 and tert-butyl 3- aminoazetidine-1- carboxylate and (2,3-difluoro-4- methoxy-phenyl) boronic acid |
| Intermediate E11 | N-[4-[(3aR,6aS)- 2,3,3a,4,6,6a- hexahydro-1H- pyrrolo[3,4- c]pyrrole-5- carbonyl]-3-chloro- phenyl]-5-(2,3- difluoro-4-methoxy- phenyl)-1-methyl- imidazole-2- carboxamide | 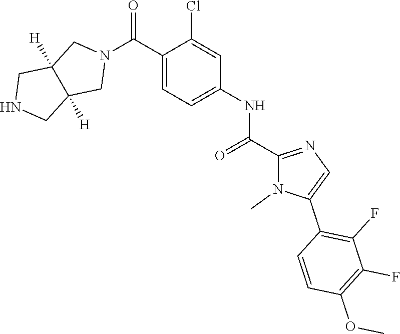 [SMILES: COc1ccc(-c2cnc(C(=O)Nc3ccc(C(=O)N4C[C@H]5CNC[C@H]5C4)c(Cl)c3)n2C)c(F)c1F] | 516.3 | Intermediate B1 and tert-butyl rac- (3aR,6aS)- 2,3,3a,4,6,6a- hexahydro-1H- pyrrolo[3,4- c]pyrrole-5- carboxylate and (2,3-difluoro-4- methoxy-phenyl) boronic acid |
| Intermediate E12 | 5-[2-chloro-4- (difluoromethoxy)-3- fluoro-phenyl]-N-[3- chloro-4-(piperazine- 1-carbonyl)phenyl]- 1-methyl-imidazole- 2-carboxamide | 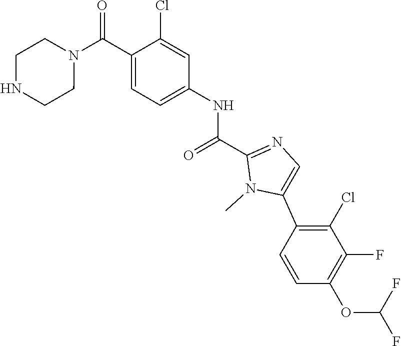 [SMILES: Cn1c(-c2ccc(OC(F)F)c(F)c2Cl)cnc1C(=O)Nc1ccc(C(=O)N2CCNCC2)c(Cl)c1] | 542.0 | Intermediate B1 and tert-butyl piperazine-1- carboxylate and Intermediate C6 |
| Intermediate E13 | N-[3-chloro-4- (piperazine-1- carbonyl)phenyl]-5- [4-(difluoromethoxy)- 2,3-difluoro-phenyl]- 1-methyl-imidazole- 2-carboxamide | 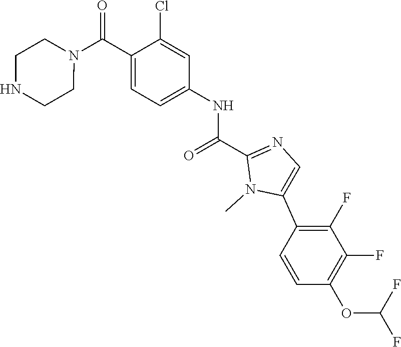 [SMILES: Cn1c(-c2ccc(OC(F)F)c(F)c2F)cnc1C(=O)Nc1ccc(C(=O)N2CCNCC2)c(Cl)c1] | 526.2 | Intermediate B1 and tert-butyl piperazine-1- carboxylate and Intermediate C3 |
| Intermediate E14 | N-[3-chloro-4- (piperazine-1- carbonyl)phenyl]-5- [4-(difluoromethoxy)- 2-fluoro-phenyl]-1- methyl-imidazole-2- carboxamide | 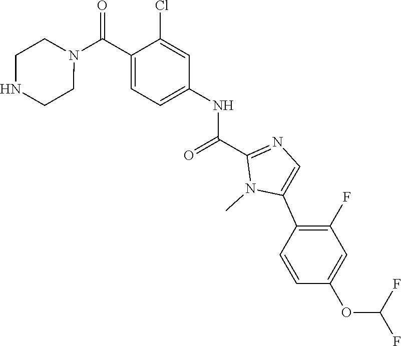 [SMILES: Cn1c(-c2ccc(OC(F)F)cc2F)cnc1C(=O)Nc1ccc(C(=O)N2CCNCC2)c(Cl)c1] | 508.3 | Intermediate B1 and tert-butyl piperazine-1- carboxylate and Intermediate C4 |
| Intermediate E15 | N-[3-chloro-4- (piperazine-1- carbonyl)phenyl]-5- [4- (difluoromethoxy)-3- fluoro-phenyl]-1- methyl-imidazole-2- carboxamide | 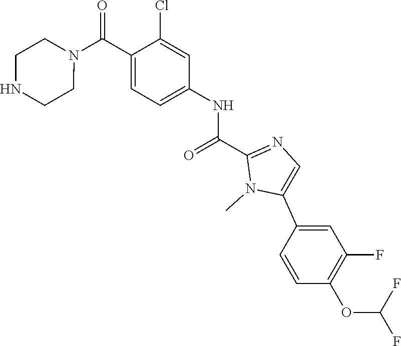 [SMILES: Cn1c(-c2ccc(OC(F)F)c(F)c2)cnc1C(=O)Nc1ccc(C(=O)N2CCNCC2)c(Cl)c1] | 508.2 | Intermediate B1 and tert-butyl piperazine-1- carboxylate and Intermediate C5 |
| Intermediate E16 | N-[4-(4- aminopiperidine-1- carbonyl)-3-chloro- phenyl]-5-(2,3- difluoro-4-methoxy- phenyl)-1-methyl- imidazole-2- carboxamide | 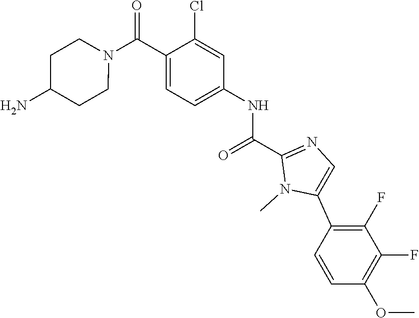 [SMILES: COc1ccc(-c2cnc(C(=O)Nc3ccc(C(=O)N4CCC(N)CC4)c(Cl)c3)n2C)c(F)c1F] | 504.1 | Intermediate B1 and tert-butyl piperazine-1- carboxylate and (2,3-difluoro-4- methoxy-phenyl) boronic acid |
| Intermediate E17 | N-[3-chloro-4- (piperazine-1- carbonyl)phenyl]-5- [2-fluoro-4- (fluoromethoxy) phenyl]-1-methyl- imidazole-2- carboxamide | 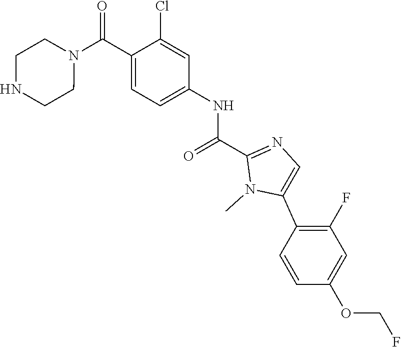 [SMILES: Cn1c(-c2ccc(OCF)cc2F)cnc1C(=O)Nc1ccc(C(=O)N2CCNCC2)c(Cl)c1] | 490.1 | Intermediate B1 and tert-butyl piperazine-1- carboxylate and C7 |
| Intermediate E18 | N-[3-chloro-4-[5- (dimethylamino) pentylcarbamoyl] phenyl]- 5-(2,3-difluoro-4- methoxy-phenyl)-1- methyl-imidazole-2- carboxamide | 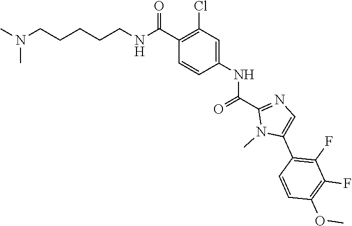 [SMILES: COc1ccc(-c2cnc(C(=O)Nc3ccc(C(=O)NCCCCCN(C)C)c(Cl)c3)n2C)c(F)c1F] | 561.4 | Intermediate B1 and N′,N′- dimethylpentane- 1,5-diamine and (2,3-difluoro-4- methoxy-phenyl) boronic acid |
| Intermediate E19 | N-[3-chloro-4-[4-[2- (dimethylamino)ethyl] piperazine-1- carbonyl]phenyl]-5- (2,3-difluoro-4- methoxy-phenyl)-1- methyl-imidazole-2- carboxamide | 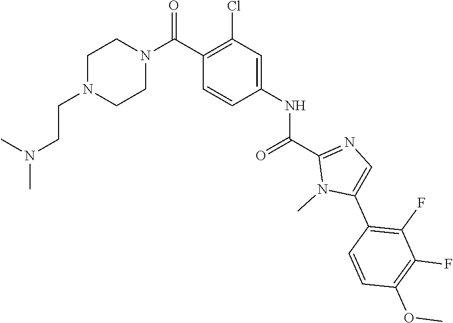 [SMILES: COc1ccc(-c2cnc(C(=O)Nc3ccc(C(=O)N4CCN(CCN(C)C)CC4)c(Cl)c3)n2C)c(F)c1F] | 561.4 | Intermediate B1 and N,N-dimethyl- 2-(piperazin-1- yl)ethan-1-amine and (2,3-difluoro- 4-methoxy- phenyl) boronic acid |
| Intermediate E20 | 5-(2,3-difluoro-4- methoxyphenyl)-N- (4-(4-(3- (dimethylamino) propyl)piperazine- 1-carbonyl)-3- ethylphenyl)-1- methyl-1H- imidazole-2- carboxamide | 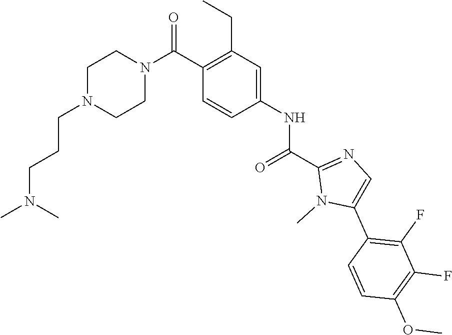 [SMILES: CCc1cc(NC(=O)c2ncc(-c3ccc(OC)c(F)c3F)n2C)ccc1C(=O)N1CCN(CCCN(C)C)CC1] | 569.4 | Intermediate B3, N,N-dimethyl-3- (piperazin-1- yl)propan-1-amine and (2,3-difluoro- 4-methoxy- phenyl) boronic acid |
| Intermediate E21 | 5-(2,3-difluoro-4- methoxyphenyl)-N- (4-((2-(2- (dimethylamino) ethoxy)ethyl) carbamoyl)- 3-ethylphenyl)-1- methyl-1H- imidazole-2- carboxamide | 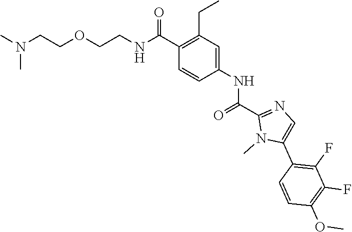 [SMILES: CCc1cc(NC(=O)c2ncc(-c3ccc(OC)c(F)c3F)n2C)ccc1C(=O)NCCOCCN(C)C] | 530.3 | Intermediate B3, 2-(2-aminoethoxy)- N,N-dimethylethan- 1-amine and (2,3- difluoro-4- methoxy-phenyl) boronic acid |
| Intermediate E22 | 1-(4-(5-(2,3-difluoro- 4-methoxyphenyl)-1- methyl-1H- imidazole-2- carboxamido)-2- ethylbenzoyl) piperidine-4- carboxylic acid | 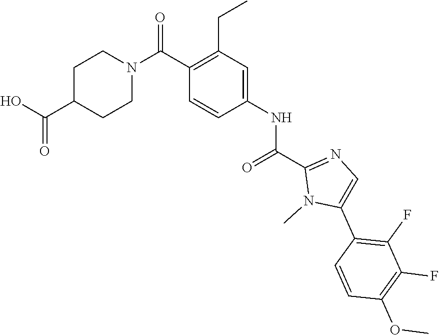 [SMILES: CCc1cc(NC(=O)c2ncc(-c3ccc(OC)c(F)c3F)n2C)ccc1C(=O)N1CCC(C(=O)O)CC1] | 525.4 | Intermediate B3, ethyl piperidine-4- carboxylate and (2,3-difluoro-4- methoxy-phenyl) boronic acid |
| Intermediate E23 | 1-[2-chloro-4-[[5-[4- (cyanomethoxy)-2,3- difluoro-phenyl]-1- methyl-imidazole-2- carbonyl]amino] benzoyl]piperidine- 4-carboxylic acid | 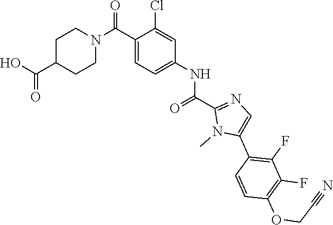 [SMILES: Cn1c(-c2ccc(OCC#N)c(F)c2F)cnc1C(=O)Nc1ccc(C(=O)N2CCC(C(=O)O)CC2)c(Cl)c1] | 558.1 | Intermediate B2, methyl piperidine- 4-carboxylate and Intermediate C1 |
| Intermediate E24 | N-[4-[(2- aminothiazol-5- yl)methylcarbamoyl]- 3-chloro-phenyl]-5- (2,3-difluoro-4- methoxy-phenyl)-1- methyl-imidazole-2- carboxamide | 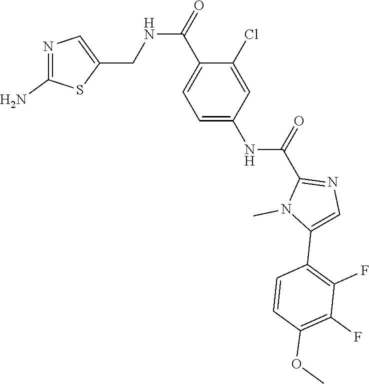 [SMILES: COc1ccc(-c2cnc(C(=O)Nc3ccc(C(=O)NCc4cnc(N)s4)c(Cl)c3)n2C)c(F)c1F] | 533.1 | Intermediate B2 and tert-butyl (5- (aminomethyl) thiazol-2- yl)carbamate |
| Intermediate E25 | 1-[2-chloro-4-[[5- (2,3-difluoro-4- methoxy-phenyl)-1- methyl-imidazole-2- carbonyl]amino] benzoyl]piperidine- 4-carboxylic acid | 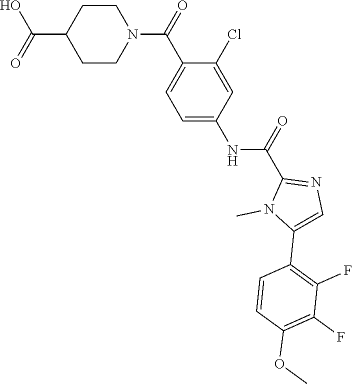 [SMILES: COc1ccc(-c2cnc(C(=O)Nc3ccc(C(=O)N4CCC(C(=O)O)CC4)c(Cl)c3)n2C)c(F)c1F] | 533.1 | Intermediate B2 and methyl piperidine-4- carboxylate and (2,3-difluoro- 4-methoxy- phenyl) boronic acid |
| Intermediate E26 | N-[(2-aminothiazol- 5-yl)methyl]-1-[2- chloro-4-[[5-(2,3- difluoro-4-methoxy- phenyl)-1-methyl- imidazole-2- carbonyl]amino] benzoyl]piperidine- 4-carboxamide | 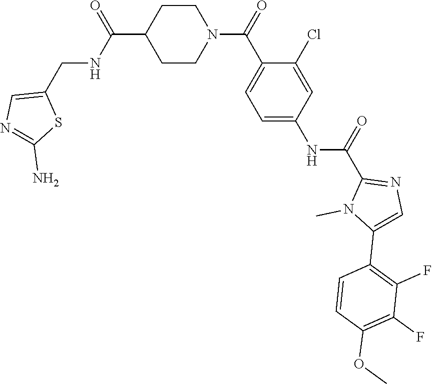 [SMILES: COc1ccc(-c2cnc(C(=O)Nc3ccc(C(=O)N4CCC(C(=O)NCc5cnc(N)s5)CC4)c(Cl)c3)n2C)c(F)c1F] | 644.2 | Intermediate B2 and tert-butyl (5- (aminomethyl) thiazol-2-yl) carbamate and (2,3-difluoro- 4-methoxy- phenyl) boronic acid |
| Intermediate E27 | 2-[4-[[[2-chloro-4- [5-(2,3-difluoro-4- methoxy-phenyl)-1- methyl-imidazole-2- carbonyl]amino] benzoyl]amino] methyl]pyrazol- 1-yl]acetic acid | 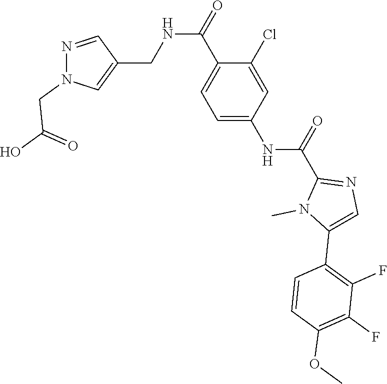 [SMILES: COc1ccc(-c2cnc(C(=O)Nc3ccc(C(=O)NCc4cnn(CC(=O)O)c4)c(Cl)c3)n2C)c(F)c1F] | 559.1 | Intermediate B2 and (1H-pyrazol- 4-yl)methanamine dihydrochloride and tert-butyl 2- bromoacetate and (2,3-difluoro-4- methoxy-phenyl) boronic acid |
| Intermediate E28 | 5-[4- (difluoromethoxy)- 2,3-difluoro-phenyl]- N-[3-fluoro-4-[4- (piperidine-4- carbonyl)piperazine- 1-carbonyl]phenyl]- 1-methyl-imidazole- 2-carboxamide | 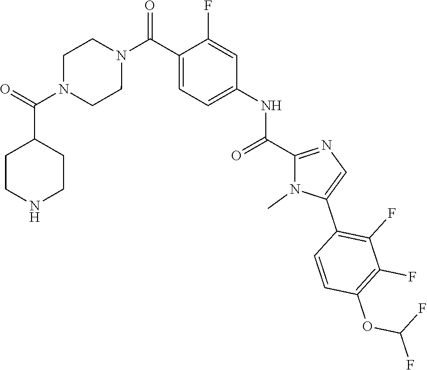 [SMILES: Cn1c(-c2ccc(OC(F)F)c(F)c2F)cnc1C(=O)Nc1ccc(C(=O)N2CCN(C(=O)C3CCNCC3)CC2)c(F)c1] | 621.1 | Intermediate A3 and Intermediate C3 |
| Intermediate E29 | N-[3-chloro-4- (piperazine-1- carbonyl)phenyl]-5- [2-(difluoromethyl)- 3-fluoro-4-methoxy- phenyl]-1-methyl- imidazole-2- carboxamide | 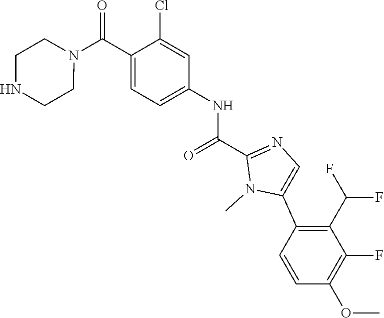 [SMILES: COc1ccc(-c2cnc(C(=O)Nc3ccc(C(=O)N4CCNCC4)c(Cl)c3)n2C)c(C(F)F)c1F] | 522.2 | Intermediate B1 and tert-butyl piperazine-1- carboxylate; Intermediate C15 |
| Intermediate E30 | N-[3-chloro-4- (piperazine-1- carbonyl)phenyl]-5- [2,3-difluoro-4- (fluoromethoxy) phenyl]-1-methyl- imidazole-2- carboxamide | 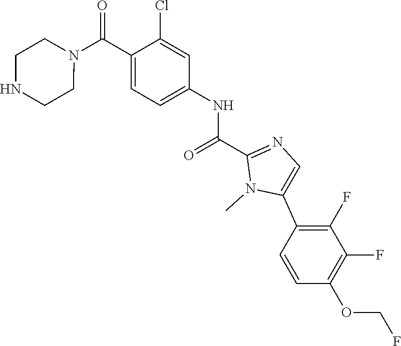 [SMILES: Cn1c(-c2ccc(OCF)c(F)c2F)cnc1C(=O)Nc1ccc(C(=O)N2CCNCC2)c(Cl)c1] | 508.2 | Intermediate B1 and tert-butyl piperazine-1- carboxylate; Intermediate C9 |
| Intermediate E31 | 5-[3-chloro-4- (difluoromethoxy)-2- fluoro-phenyl]-N-[3- fluoro-4-[4- (piperidine-4- carbonyl)piperazine- 1-carbonyl]phenyl]- 1-methyl-imidazole- 2-carboxamide | 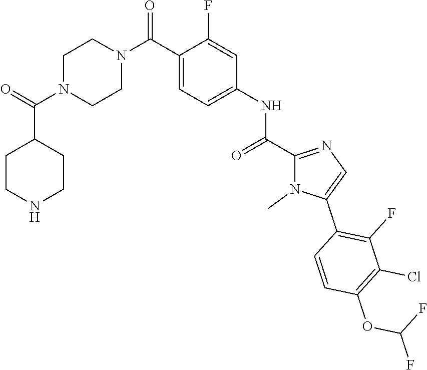 [SMILES: Cn1c(-c2ccc(OC(F)F)c(Cl)c2F)cnc1C(=O)Nc1ccc(C(=O)N2CCN(C(=O)C3CCNCC3)CC2)c(F)c1] | 637.0 | Intermediate A3 and Intermediate C10 |
| Intermediate E32 | 5-(3-chloro-2-fluoro- 4-methoxy-phenyl)- N-[3-chloro-4- (piperazine-1- carbonyl)phenyl]-1- methyl-imidazole-2- carboxamide | 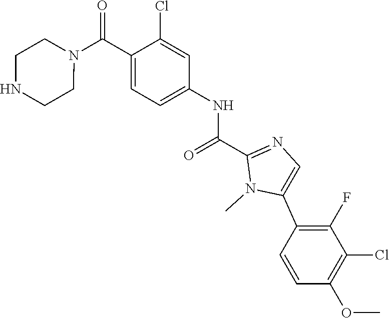 [SMILES: COc1ccc(-c2cnc(C(=O)Nc3ccc(C(=O)N4CCNCC4)c(Cl)c3)n2C)c(F)c1Cl] | 506.0 | Intermediate B1 and tert-butyl piperazine-1- carboxylate; Intermediate C11 |
| Intermediate E33 | N-[3-chloro-4- (piperazine-1- carbonyl)phenyl]-5- (3-cyano-2-fluoro-4- methoxy-phenyl)-1- methyl-imidazole-2- carboxamide | 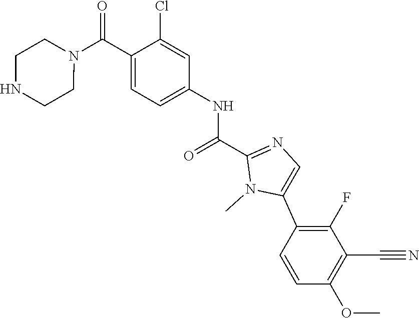 [SMILES: COc1ccc(-c2cnc(C(=O)Nc3ccc(C(=O)N4CCNCC4)c(Cl)c3)n2C)c(F)c1C#N] | 497.2 | Intermediate B1 and tert-butyl piperazine-1- carboxylate; Intermediate C12 |
| Intermediate E34 | N-[3-chloro-4- (piperazine-1- carbonyl)phenyl]-5- [2-fluoro-4- methoxy-3- (trifluoromethyl) phenyl]-1-methyl- imidazole-2- | 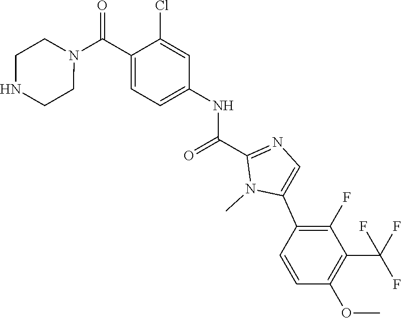 [SMILES: COc1ccc(-c2cnc(C(=O)Nc3ccc(C(=O)N4CCNCC4)c(Cl)c3)n2C)c(F)c1C(F)(F)F] | 540.2 | Intermediate B1 and tert-butyl piperazine-1- carboxylate; Intermediate C13 |
| Intermediate E35 | N-[4-[(1S,5R)-3- azabicyclo[3.1.0] hexan-6-yl]carbamoyl]- 3-chloro-phenyl]-5- (2,3-difluoro-4- methoxy-phenyl)-1- methyl-imidazole-2- carboxamide | 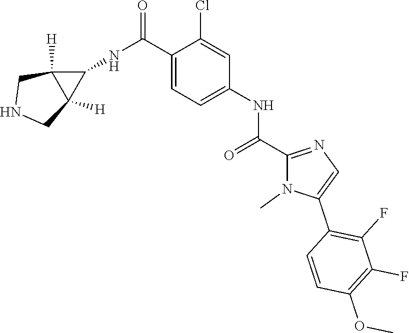 [SMILES: COc1ccc(-c2cnc(C(=O)Nc3ccc(C(=O)N[C@H]4[C@@H]5CNC[C@@H]54)c(Cl)c3)n2C)c(F)c1F] | 502.2 | Intermediate B1 and tert-butyl (1R,5S)-6-amino- 3-azabicyclo[3.1.0] hexane-3-carboxylate and (2,3-difluoro-4- methoxy-phenyl) boronic acid |
| Intermediate E36 | 5-[2-chloro-3-fluoro- 4-(fluoromethoxy) phenyl]-N-[3-chloro- 4-(piperazine-1- carbonyl)phenyl]-1- methyl-imidazole-2- carboxamide | 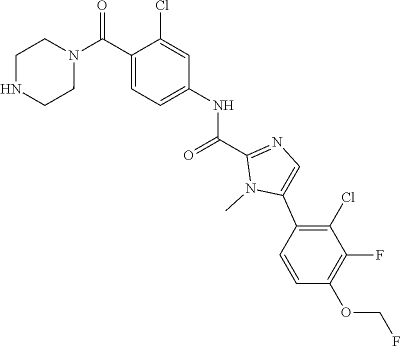 [SMILES: Cn1c(-c2ccc(OCF)c(F)c2Cl)cnc1C(=O)Nc1ccc(C(=O)N2CCNCC2)c(Cl)c1] | 524.3 | Intermediate B1 and tert-butyl piperazine-1- carboxylate; Intermediate C14 |

### Intermediate F1

### 1-(4-(4-Amino-2-chlorobenzoyl)piperazin-1-yl)-2-(dimethylamino)ethan-1-one

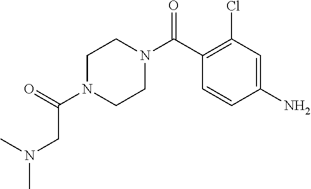 [SMILES: CN(C)CC(=O)N1CCN(C(=O)c2ccc(N)cc2Cl)CC1]

4-amino-2-chlorobenzoic acid (1 g, 5.83 mmol), 2-(dimethylamino)-1-(piperazin-1-yl)ethan-1-one (998 mg, 5.83 mmol), 2-(3H-[1,2,3]triazolo[4,5-b]pyridin-3-yl)-1,1,3,3-tetramethylisouronium hexafluorophosphate(V) (2.66 g, 6.99 mmol) were dissolved in DMF (25 mL). DIPEA (3.01 g, 4.07 mL, 23.3 mmol) was added. After 3 h, no reaction was observed so 2-(dimethylamino)-1-(piperazin-1-yl)ethan-1-one (200 mg, 1.17 mmol) was added and the reaction mixture was stirred overnight. 25 mL of water were added after cooling at 0° C. to the reaction mixture. It was extracted with 3×50 mL of DCM. The organic layers were combined, dried with MgSb₄, concentrated under vacuum and DMF was removed with HV for 1 h. The crude was purified by flash chormatography to afford the title compound (1.65 g, 4.57 mmol, 78.40%) as a white solid. ES4MS[M+H]⁺:325.5.

The following Type VI Intermediates were prepared in analogy to Intermediate F1:

| | | | MS | |
|---|---|---|---|---|
| | | | ESI | |
| Int. | Name | Structure | [M + H]⁺ | Starting Material |
| Intermediate F2 | tert-butyl 4-[4-(4-amino- 2-ethyl- benzoyl)piperazine-1- carbonyl]piperidine-1- carboxylate | 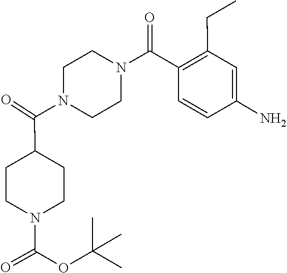 [SMILES: CCc1cc(N)ccc1C(=O)N1CCN(C(=O)C2CCN(C(=O)OC(C)(C)C)CC2)CC1] | 345.4 | tert-butyl 4- (piperazine-1- carbonyl)piperidine- 1-carboxylate and 4- amino-2-ethyl- benzoic acid |
| Intermediate F3 | tert-butyl 4-(4-amino-2- fluoro- benzoyl)piperazine-1- carboxylate | 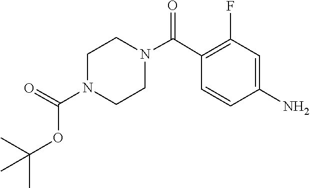 [SMILES: CC(C)(C)OC(=O)N1CCN(C(=O)c2ccc(N)cc2F)CC1] | 323.9 | tert-butyl piperazine-1- carboxylate and 4- amino-2-fluoro- benzoic acid |
| Intermediate F4 | 1-[4-(4-amino-2-methyl- benzoyl)piperazin-1-yl]- 2- (dimethylamino)ethanone | 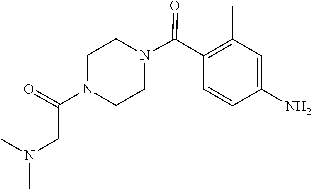 [SMILES: Cc1cc(N)ccc1C(=O)N1CCN(C(=O)CN(C)C)CC1] | 305.3 | 4-amino-2-methyl- benzoic acid and 2- (dimethylamino)-1- piperazin-1-yl- ethanone |

### Intermediate G1

### 2-Chloro-4-[[5-(2,3-difluoro-4-methoxy-phenyl)-1-methyl-imidazole-2-carbonyl]amino]benzoic acid

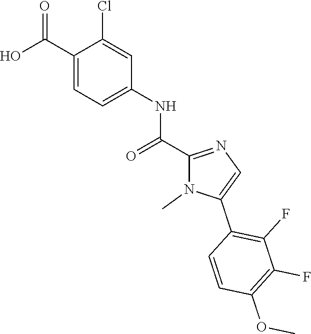 [SMILES: COc1ccc(-c2cnc(C(=O)Nc3ccc(C(=O)O)c(Cl)c3)n2C)c(F)c1F]

### Step 1: methyl 2-chloro-4-[[5-(2,3-difluoro-4-methoxy-phenyl)-1-methyl-imidazole-2-carbonyl]amino]benzoate

A mixture of methyl 4-[(5-bromo-1-methyl-imidazole-2-carbonyl)amino]-2-chloro-benzoate (1 g, 2.68 mmol), (2,3-difluoro-4-methoxyphenyl)boronic acid (504 mg, 2.68 mmol), Na₂CO₃ (853 mg, 8.05 mmol) and 1,1′-bis(di-tert-butylphosphino) ferrocene palladium dichloride (350 mg, 537 μmol) in 1,4-Dioxane (15 mL) and water (1.5 mL) was irritated under microwave at 100° C. for 60 min. Repeat this reaction for eight times. The combined reaction solutions were concentrated. Water (40 mL) was added. The water phase was extracted with DCM. The combined organic phases were washed with water, dried over anhydrous Na₂SO₄ and concentrated. The concentrated organic phase was filtrated and the solid was dried to give the crude title compound (8.3 g, 88.7% yield) as a brown solid. ESI MS [M+H]⁺: 435.9.

### Step 2: 2-chloro-4-[[5-(2,3-difluoro-4-methoxy-phenyl)-1-methyl-imidazole-2-carbonyl]amino]benzoic acid

To a solution of methyl 2-chloro-4-[[5-(2,3-difluoro-4-methoxy-phenyl)-1-methyl-imidazole-2-carbonyl]amino]benzoate (8.3 g, 19 mmol) in MeOH (2 mL), THE (48 mL) and water (24 mL) was added a solution of Lithium hydroxide monohydrate (3.2 g, 76.2 mmol) in water (24 mE). The mixture was stirred at room temperature overnight. Then the mixture was concentrated and acidified by 6N HCl under stirring until PH 3-4. Some solids precipitated from the concentrated solution. The solids were filtered and dried to give the title compound (7 g, 8700 yield) as a brown solid. ESI MS [M+H]⁺: 422.3.

The following Type VII Intermediates were prepared in analogy to Intermediate G1:

| | | | MS | |
|---|---|---|---|---|
| | | | ESI | |
| Int. | Name | Structure | [M + H]⁺ | Starting Material |
| Intermediate G2 | 2-chloro-4-[[5-[4- (cyanomethoxy)-2,3- difluoro-phenyl]-1- methyl-imidazole-2- carbonyl]amino]benzoic acid | 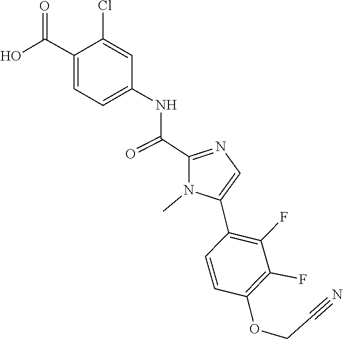 [SMILES: Cn1c(-c2ccc(OCC#N)c(F)c2F)cnc1C(=O)Nc1ccc(C(=O)O)c(Cl)c1] | 447 | (4- methoxyphenyl)methyl 4-[(5-bromo-1-methyl- imidazole-2- carbonyl)amino]-2- chloro-benzoate and C8 |
| Intermediate G3 | 4-[[5-[4- (cyanomethoxy)-2,3- difluoro-phenyl]-1- methyl-imidazole-2- carbonyl]amino]-2- methyl-benzoic acid | 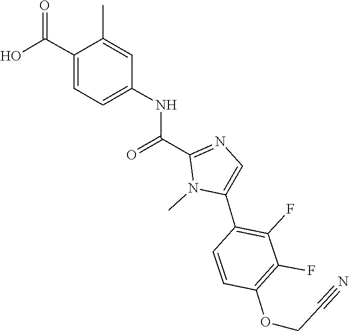 [SMILES: Cc1cc(NC(=O)c2ncc(-c3ccc(OCC#N)c(F)c3F)n2C)ccc1C(=O)O] | 427.1 | (4- methoxyphenyl)methyl 4-[(5-bromo-1-methyl- imidazole-2- carbonyl)amino]-2- methyl-benzoate and C8 |
| Intermediate G4 | 2-chloro-4-[[5-(2- chloro-3-fluoro-4- methoxy-phenyl)-1- methyl-imidazole-2- carbonyl]amino]benzoic acid | 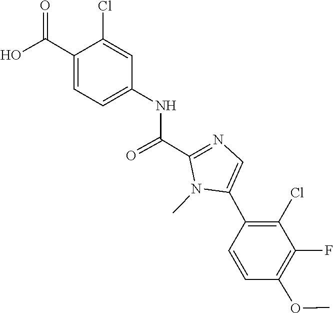 [SMILES: COc1ccc(-c2cnc(C(=O)Nc3ccc(C(=O)O)c(Cl)c3)n2C)c(Cl)c1F] | 437.1 | methyl 4-[(5-bromo-1- methyl-imidazole-2- carbonyl)amino]-2- chloro-benzoate and C2 |

### Intermediate H1

### [2-[(3-Aminocyclobutyl)amino]-2-oxo-ethyl]-trimethyl-ammonium iodide hydrochloride

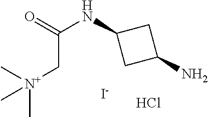

### Step 1: tert-butyl N-[3-[[2-(dimethylamino)acetyl]amino]cyclobutyl]carbamate

To a solution of N,N-dimethylglycine (500.0 mg, 4.85 mmol) in DMF (5 mL) was added 1,1′-carbonyldiimidazole (786.22 mg, 4.85 mmol). The reaction mixture was stirred at 50° C. for 1 h. Then tert-butyl N-(3-aminocyclobutyl)carbamate (903.07 mg, 4.85 mmol) was added to the mixture and stirred at 50° C. for 12 h. The reaction mixture was diluted with water and extracted with DCM/MeOH=10:1. The combined organic layer was washed with brine, dried over Na₂SO₄, filtered and concentrated to afford the title compound (1.31 g, 4.83 mmol, 99% yield) as a colorless oil. ESI MS [M+H]⁺: 272.3.

### Step 2: [2-[[3-(tert-butoxycarbonylamino)cyclobutyl]amino]-2-oxo-ethyl]-trimethyl-ammonium iodide

To a solution of tert-butyl N-[3-[[2-(dimethylamino)acetyl]amino]cyclobutyl]carbamate (1.31 g, 4.83 mmol) in ethyl acetate (15 mL) was added iodomethane (822.26 mg, 5.79 mmol). The reaction was stirred at 30° C. for 16 h. The reaction mixture was filtered and the filter cake was collected to afford the title compound (1.3 g, 3.15 mmol, 65% yield) as a yellow solid. ESI MS [M+H]⁺: 286.3.

### Step 3: [2-[(3-aminocyclobutyl)amino]-2-oxo-ethyl]-trimethyl-ammonium iodide hydrochloride

[2-[[3-(tert-butoxycarbonylamino)cyclobutyl]amino]-2-oxo-ethyl]-trimethyl-ammonium iodide (1.3 g, 3.15 mmol) was added to hydrogen chloride in methanol (15 mL, 60 mmol). The reaction was stirred at 30° C. for 1 h. The reaction mixture was concentrated to afford the title compound (1.1 g, 3.15 mmol, 100% yield) as a yellow oil. ESI MS [M+H]⁺: 186.2.

### Intermediate H2

### 2-[(3-Aminocyclobutyl)amino]ethyl-trimethyl-ammonium chloride

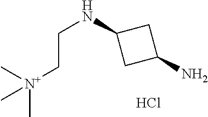

### Step 1: 2-[[3-(tert-butoxycarbonylamino)cyclobutyl]amino]ethyl-trimethyl-ammonium formate

To a solution of tert-butyl N-(3-aminocyclobutyl)carbamate (500 mg, 2.68 mmol) and 2-bromoethyl(trimethyl)ammonium bromide (2.98 g, 12.1 mmol) in ACN (5 mL) was added sodium iodide (0.12 g, 0.81 mmol) and diisopropyl ethylamine (2.11 mL, 12.08 mmol) then the reaction was stirred at 80° C. for 24 h. The reaction mixture was concentrated under reduce pressure. The crude product was purified by prep-HPLC(FA) and lyophilized to afford the title compound (500 mg, 1.58 mmol, 68.37% yield) as a dark green oil. ESI MS [M+H]⁺: 272.2.

### Step 2: 2-[(3-aminocyclobutyl)amino]ethyl-trimethyl-ammonium chloride

To a solution of 2-[[3-(tert-butoxycarbonylamino)cyclobutyl]amino]ethyl-trimethyl-ammonium (500 mg, 1.84 mmol) was added HCl in MeOH (1.97 mL, 80 mmol) and then the reaction was stirred at 20° C. for 12 h. The reaction mixture was concentrated under reduced pressure to afford the title compound (500 mg, 2.41 mmol, 95% yield) as a light yellow oil. ESI MS [M+H]⁺: 172.2.

### Intermediate H3

### [2-(3-Aminopropylamino)-2-oxo-ethyl]-trimethyl-ammonium iodide hydrochloride

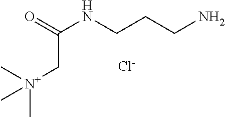

### Step 1: tert-butyl N-[3-[[2-(dimethylamino)acetyl]amino]propyl]carbamate

To a solution of N,N-dimethylglycine (500 mg, 4.85 mmol) in DMF (5 mL) was added 1,1′-carbonyldiimidazole (786.22 mg, 4.85 mmol). The mixture was stirred at 50° C. for 1 h. Then N-Boc-1,3-diaminopropane (929 mg, 5.33 mmol) was added to the mixture and it was stirred for 12 h. The reaction mixture was diluted with water and extracted with DCM/MeOH=10:1. The combined organic layer was washed with brine, dried over Na₂SO₄, filtered and concentrated to afford the title compound (1.05 g, 4.05 mmol, 83% yield) as a colorless oil. ESI MS [M+H]⁺: 260.3.

### Step 2: tert-butyl N-ethylcarbamate; [2-(ethylamino)-2-oxo-ethyl]-trimethyl-ammonium; iodide

To a solution of tert-butyl N-[3-[[2-(dimethylamino)acetyl]amino]propyl]carbamate (1.05 g, 4.05 mmol) in ethyl acetate (8.33 mL) was added iodomethane (632.12 mg, 4.45 mmol). The reaction was stirred at 30° C. for 16 h. The reaction mixture was filtered and the filter cake was collected to afford the title compound (1.08 g, 2.59 mmol, 63% yield) as a white solid. ESI MS [M+H]⁺: 274.2.

### Step 3: [2-(3-aminopropylamino)-2-oxo-ethyl]-trimethyl-ammonium iodide hydrochloride

[2-[3-(tert-butoxycarbonylamino)propylamino]-2-oxo-ethyl]-trimethyl-ammonium iodide (1.08 g, 2.69 mmol) was added to hydrogen chloride in methanol (10 mL, 40 mmol). The reaction was stirred at 30° C. for 1 h. The reaction mixture was concentrated to afford the title compound (900 mg, 2.67 mmol, 99% yield) as a yellow oil. ESI MS [M+H]⁺: 174.1.

The following Intermediates were prepared in analogy to Intermediate H3:

| | | | MS | |
|---|---|---|---|---|
| | | | ESI | Starting |
| Int. | Name | Structure | [M + H]⁺ | Material |
| Intermediate H4 | [2-(2- aminoethylamino)- 2-oxo-ethyl]- trimethyl- ammonium iodide hydrochloride | 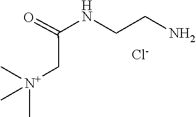 | 160.1 | N,N- dimethylglycine and N-Boc- ethylenediamine |

### Intermediate H5

### (2-Amino-2-oxo-ethyl)-[2-[2-(tert-butoxycarbonylamino)ethoxy]ethyl]-dimethyl-ammonium bromide

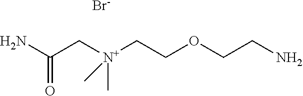

### Step 1: 2-[2-(tert-butoxycarbonylamino)ethoxy]ethyl 4-methylbenzenesulfonate

To a solution of 2-(2-Boc-aminoethoxy)ethanol (5 g, 24.36 mmol), 4-dimethylaminopyridine (1.79 g, 14 mmol), and N,N-diisopropylethylamine (12.73 mL, 73 mmol) in DCM (50 mL) was added p-toluenesulfonyl chloride (9.29 g, 48.mmol) at 0° C. 1 h later, the reaction was warmed to 20° C. and stirred for 15 h. The reaction mixture was diluted with water (150 mL) and extracted with DCM (100 mL×3). The combined organic layer was washed with brine (100 mL), dried over Na₂SO₄, filtered and concentrated. The residue was purified by column chromatography to afford the title compound (6.17 g, 17.17 mmol, 70% yield) as a light yellow oil. ESI MS [M+H-Boc]⁺: 260.0.

### Step 2: tert-butyl N-[2-[2-(dimethylamino)ethoxy]ethyl]carbamate

To a solution of 2-[2-(tert-butoxycarbonylamino)ethoxy]ethyl 4-methylbenzenesulfonate (6.07 g, 16.89 mmol) in ACN (60 mL) was added potassium carbonate (7 g, 50.66 mmol) and dimethylamine hydrochloride (2.75 g, 33.77 mmol). The reaction was stirred at 40° C. for 16 h. The reaction mixture was diluted with water (50 mL) and extracted with DCM/MeOH=10:1 (50 mL×3). The combined organic layer was washed with brine (100 mL), dried over Na₂SO₄, filtered and concentrated to afford the title compound (4 g, 17.22 mmol, 101.96% yield) as a yellow oil. ESI MS [M+H]⁺: 233.2.

### Step 3: (2-amino-2-oxo-ethyl)-[2-[2-(tert-butoxycarbonylamino)ethoxy]ethyl]-dimethyl-ammonium bromide

To a solution of tert-butyl N-[2-[2-(dimethylamino)ethoxy]ethyl]carbamate (500 mg, 2.15 mmol) in ethyl acetate (5 mL) was added 2-bromoacetamide (445.38 mg, 3.23 mmol). The reaction was stirred at 50° C. for 16 h. The reaction mixture was concentrated. The residue was purified by prep-HPLC (FA) to afford the title compound (480 mg, 1.3 mmol, 60% yield) as a colorless oil. ESI MS [M+H]⁺: 290.3.

### Step 4: 2-(2-aminoethoxy)ethyl-(2-amino-2-oxo-ethyl)-dimethyl-ammonium iodide hydrochloride

(2-amino-2-oxo-ethyl)-[2-[2-(tert-butoxycarbonylamino)ethoxy]ethyl]-dimethyl-ammonium bromide (480 mg, 1.3 mmol) was added to hydrogen chloride in methanol (5 mL, 20 mmol). The reaction was stirred at 30° C. for 1 h. The reaction mixture was concentrated to afford the title compound (397 mg, 1.29 mmol, 99% yield) as a yellow oil. ESI MS [M+H]⁺: 190.2.

The following Intermediates were prepared in analogy to Intermediate H5:

| | | | MS | |
|---|---|---|---|---|
| | | | ESI | |
| Int. | Name | Structure | [M + H]⁺ | Starting Material |
| Intermediate H6 | 2-(2- aminoethoxy)ethyl- trimethyl- ammonium iodide hydrochloride | 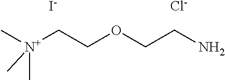 | 147.1 | iodomethane |

### Intermediate H7

### 2-[4-(aminomethyl)-2-methyl-pyrazol-2-ium-1-yl]ethanol;chloride

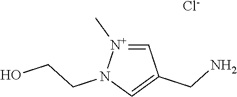

### Step 1: tert-butyl N-[[1-[2-[tert-butyl(dimethyl)silyl]oxyethyl]pyrazol-4-yl]methyl]carbamate

tert-butyl ((1H-pyrazol-4-yl)methyl)carbamate (300 mg, 1.52 mmol), cesium carbonate (991 mg, 3.04 mmol) and tert-butyl(2-iodoethoxy)dimethylsilane (653 mg, 2.28 mmol) in Acetonitrile (7.61 mL) were stirred at rt overnight. Silica gel was added to absorb the product, which was then purified by flash chromatography to give the product as colorless oil (540 mg, 98%). ESI MS [M+H]⁺: 356.2.

### Step 2: tert-butyl 2-[4-(aminomethyl)-2-methyl-pyrazol-2-ium-1-yl]ethanol;chloride

tert-butyl ((1-(2-((tert-butyldimethylsilyl)oxy)ethyl)-1H-pyrazol-4-yl)methyl)carbamate (0.54 g, 1.52 mmol) and iodomethane (1.08 g, 7.59 mmol) in MeCN (6.9 mL) were stirred overnight at rt. The temperature was raised to 80° C., and the stirring was continued for 7 h. The solvent was removed in vacuum, and the residue was dissolved in 4M HCl/MeOH solution. After 2 h stirring at rt, the solvent was removed in vacuum, the residue was azeotroped with toluene (25 mL×3), and the brown oil was used in the next step without purification, 230 mg, 97%. ESI MS [M+H]⁺: 156.0.

The following Intermediates were prepared in analogy to Intermediate 117:

| | | | MS | |
|---|---|---|---|---|
| | | | ESI | |
| Int. | Name | Structure | [M]⁺ | Starting Material |
| Intermediate H8 | 3-[4-(aminomethyl)-2- methyl-pyrazol-2-ium-1- yl]propan-1-ol; chloride | 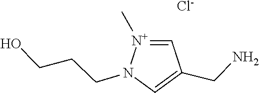 | 170.0 | tert-butyl ((1H-pyrazol-4- yl)methyl)carbamate, (3- bromopropoxy)(tert- butyl)dimethylsilane and iodomethane |
| Intermediate H9 | 3-[4-(aminomethyl)-2- methyl-pyrazol-2-ium-1- yl]propanenitrile; chloride | 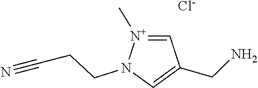 | 165.1 | tert-butyl ((1H-pyrazol-4- yl)methyl)carbamate, 3- bromopropanenitrile and iodomethane |
| Intermediate H10 | 3-[4-(aminomethyl)-2- methyl-pyrazol-2-ium-1- yl]propanamide; chloride | 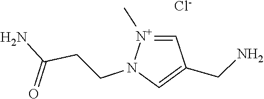 | 169.1 | tert-butyl ((1H-pyrazol-4- yl)methyl)carbamate, 2- iodoacetamide and iodomethane |
| Intermediate H11 | [1-(3-methoxypropyl)-2- methyl-pyrazol-2-ium-4- yl]methanamine; chloride | 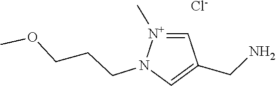 | 184.1 | tert-butyl ((1H-pyrazol-4- yl)methyl)carbamate, 1-bromo-3- methoxypropane and iodomethane |
| Intermediate H12 | [1-methyl-2-(2- methylsulfonylethyl)pyrazol- 1-ium-4-yl]methanamine; chloride | 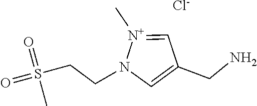 | 218.3 | tert-butyl ((1H-pyrazol-4- yl)methyl)carbamate, 1-bromo-2- (methylsulfonyl)ethane and iodomethane |
| Intermediate H13 | 2-[2-[4-(aminomethyl)-2- methyl-pyrazol-2-um-1- yl]ethoxy]ethanol; chloride | 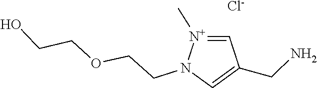 | 200.1 | tert-butyl ((1H-pyrazol-4- yl)methyl)carbamate, tert-butyl(2- (2- chloroethoxy)ethoxy)diphenylsilane and iodomethane |
| Intermediate H14 | 4-hydroxy-1,1-dimethyl- pyrrolidin-1-ium-2- carboxylic acid; chloride | 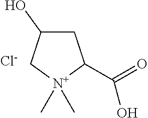 | 160.1 | 4-hydroxypyrrolidine-2-carboxylic acid and iodomethane |
| Intermediate H15 | 1,1-dimethylpyrrolidin-1- ium-2-carboxylic acid; chloride | 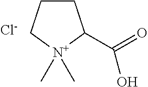 | 144.1 | pyrrolidine-2-carboxylic acid and iodomethane |
| Intermediate H16 | 1,1-dimethylpiperidin-1- ium-4-carboxylic acid; chloride | 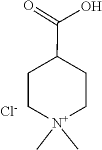 | 158.1 | 1-methylpiperidine-4-carboxylic acid and iodomethane |
| Intermediate H17 | 1-(2-amino-2-oxo-ethyl)-1- methyl-piperidin-1-ium-4- carboxylic acid; chloride | 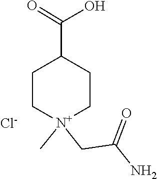 | 201.1 | 1-methylpiperidine-4-carboxylic acid and 2-iodoacetamide |

### Intermediate H19

### Tert-butyl 3-[[1-(2-tert-butoxy-2-oxo-ethyl)-4-(piperazine-1-carbonyl)piperidin-1-ium-1-yl]methyl]azetidine-1-carboxylate formate

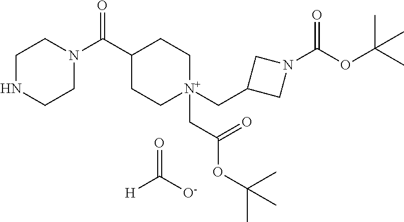

### Step 1: benzyl 4-[1-[(1-tert-butoxycarbonylazetidin-3-yl)methyl]piperidine-4-carbonyl]piperazine-1-carboxylate

To a mixture of benzyl 4-(piperidine-4-carbonyl)piperazine-1-carboxylate (1.2 g, 3.62 mmol, 1 eq) in DMF (10 mL) was added 1-BOC-3-(bromomethyl)azetidine (1.0 g, 4 mmol, 1.1 eq) and N,N-diisopropylethylamine (1.89 mL, 10.86 mmol, 3 eq). The reaction mixture was stirred at 50° C. for 16 h. The mixture was purified by reversed phase-HPLC (FA condition) to afford the title compound (750 mg, 1.5 mmol, 41.37% yield) as yellow solid. ESI MS [M+H]⁺: 501.4.

### Step 2: benzyl 4-[1-[(1-tert-butoxycarbonylazetidin-3-yl)methyl]-1-(2-tert-butoxy-2-oxo-ethyl)piperidin-1-ium-4-carbonyl]piperazine-1-carboxylate formate

To a mixture of benzyl 4-[1-[(1-tert-butoxycarbonylazetidin-3-yl)methyl]piperidine-4-carbonyl]piperazine-1-carboxylate (470.0 mg, 0.940 mmol, 1 eq) in ACN (10 mL) was added triethylamine (0.39 mL, 2.82 mmol, 3 eq) and tert-butyl bromoacetate (0.45 mL, 2.82 mmol, 3 eq). The reaction mixture was stirred at 80° C. for 16 h. The mixture was concentrated in vacuum. The crude material was purified by reversed phase-HPLC (FA condition) to afford the title compound (250 mg, 0.410 mmol, 43.24% yield) as yellow solid. ESI MS [M+H]⁺: 615.5.

### Step 3: tert-butyl 3-[[1-(2-tert-butoxy-2-oxo-ethyl)-4-(piperazine-1-carbonyl)piperidin-1-ium-1-yl]methyl]azetidine-1-carboxylate formate

To a mixture of benzyl 4-[1-[(1-tert-butoxycarbonylazetidin-3-yl)methyl]-1-(2-tert-butoxy-2-oxo-ethyl)piperidin-1-ium-4-carbonyl]piperazine-1-carboxylate formate (250.0 mg, 0.410 mmol, 1 eq) in methanol (5 mL) was added 10% palladium/C (43 mg) under N₂. The reaction mixture was stirred at 25° C. for 16 h under hydrogen atmosphere. The mixture was filtered and concentrated in vacuum to afford the title compound (150 mg, 0.310 mmol, 76.7% yield) as colorless oil. ESI MS [M+H]⁺: 418.3.

The following Intermediates were prepared in analogy to Intermediate H19:

| | | | MS | |
|---|---|---|---|---|
| | | | ESI | |
| Int. | Name | Structure | [M]⁺ | Starting Material |
| Intermediate H18 | tert-butyl 2-[1-[3-(tert- butoxycarbonylamino)propyl]- 4-(piperazine-1- carbonyl)piperidin-1-ium-1- yl]acetate | 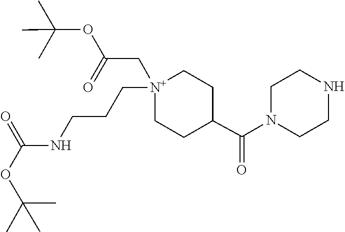 [SMILES: CC(C)(C)OC(=O)C[N+]1(CCCNC(=O)OC(C)(C)C)CCC(C(=O)N2CCNCC2)CC1] | 469.3 | 3-(BOC- amino)propyl bromide |

### Intermediate H20

### Bis[3-(tert-butoxycarbonylamino)propyl]-(2-tert-butoxy-2-oxo-ethyl)-(4-oxo-4-piperazin-1-yl-butyl)ammonium formate

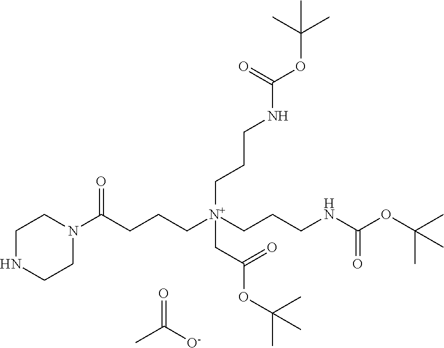

### Step 1: benzyl 4-[4-(tert-butoxycarbonylamino)butanoyl]piperazine-1-carboxylate

A mixture of BOC-gamma-abu-OH (2.31 g, 11.35 mmol), 1-CBZ-piperazine (2.5 g, 11.35 mmol), triethylamine (2.37 mL, 17.02 mmol) in THE (70 mL) was added propylphosphonic anhydride (8.1 mL, 13.62 mmol) at 25° C. and stirred for 2 h. The solution was poured into HCl solution (0.1N, 150 mL), extracted with EtOAc (50 mL×2), washed by brine (100 mL), dried over Na2SO4, concentrated and purified by reversed phase-HPLC (FA) to afford the title compound (3.5 g, 8.63 mmol, 76.05% yield) as colorless oil. ESI MS [M+H]⁺: 406.4.

### Step 2: benzyl 4-(4-aminobutanoyl)piperazine-1-carboxylate hydrochloride

A mixture of benzyl 4-[4-(tert-butoxycarbonylamino)butanoyl]piperazine-1-carboxylate (3.5 g, 8.63 mmol in 4M hydrochloric acid in EtOAc (38.9 mL, 155.56 mmol,) was stirred at 25° C. for 1 h. The solution was concentrated to afford the title compound (2.9 g, 8.48 mmol, 98.29% yield) as white solid, which was used without further purification. ESI MS [M+H]⁺: 306.2.

### Step 3: benzyl 4-[4-[bis[3-(tert-butoxycarbonylamino)propyl]amino]butanoyl]piperazine-1-carboxylate

A mixture of tert-butyl N-(3-oxopropyl)carbamate (2.0 g, 6.93 mmol, 2.37 eq), benzyl 4-(4-aminobutanoyl)piperazine-1-carboxylate hydrochloride (1.0 g, 2.93 mmol), sodium acetate (480 mg, 5.85 mmol) in methanol was added sodium triacetoxyborohydride (1860 mg, 8.78 mmol) and stirred at 25° C. for 16 h. The solution was filtered, concentrated and purified by reversed phase-HPLC (FA) to afford the title compound (900 mg, 1.45 mmol, 49.64% yield) as colorless oil. ESI MS [M+H]⁺: 620.6.

### Step 4: [4-(4-benzyloxycarbonylpiperazin-1-yl)-4-oxo-butyl]-bis[3-(tert-butoxycarbonylamino)propyl]-(2-tert-butoxy-2-oxo-ethyl)ammonium formate

To a mixture of benzyl 4-[4-[bis[3-(tert-butoxycarbonylamino)propyl]amino]butanoyl]piperazine-1-carboxylate (500.0 mg, 0.810 mmol) in ACN (10 mL) was added triethylamine (0.34 mL, 2.42 mmol) and tert-butyl bromoacetate (0.39 mL, 2.42 mmol). The reaction mixture was stirred at 70° C. for 16 h. The mixture was concentrated in vacuum. The crude material was purified by reversed phase-HPLC (FA condition) to afford the title compound (200 mg, 0.320 mmol, 40% yield) as yellow solid. ESI MS [M+H]⁺: 734.3.

### Step 5: bis[3-(tert-butoxycarbonylamino)propyl]-(2-tert-butoxy-2-oxo-ethyl)-(4-oxo-4-piperazin-1-yl-butyl)ammonium formate

To a mixture of [4-(4-benzyloxycarbonylpiperazin-1-yl)-4-oxo-butyl]-bis[3-(tert-butoxycarbonylamino)propyl]-(2-tert-butoxy-2-oxo-ethyl)ammonium formate (60.0 mg, 0.080 mmol) in methanol (5 mL) was added 10% palladium/C (9 mg) under N2. The reaction mixture was stirred at 25° C. for 16 h under hydrogen atmosphere. The mixture was filtered and concentrated in vacuum to afford bis[3-(tert-butoxycarbonylamino)propyl]-(2-tert-butoxy-2-oxo-ethyl)-(4-oxo-4-piperazin-1-yl-butyl)ammonium formate (45 mg, 0.070 mmol, 91.74% yield) as yellow solid. ESI MS [M+H]⁺: 600.5.

### Intermediate H21

### 1-[(1-tert-butoxycarbonylazetidin-3-yl)methyl]-1-(2-tert-butoxy-2-oxo-ethyl)piperidin-1-ium-4-carboxylate

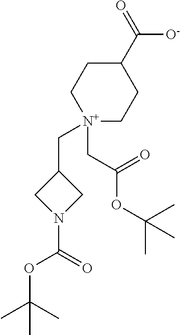 [SMILES: CC(C)(C)OC(=O)C[N+]1(CC2CN(C(=O)OC(C)(C)C)C2)CCC(C(=O)[O-])CC1]

### Step 1: benzyl 1-[(1-tert-butoxycarbonylazetidin-3-yl)methyl]piperidine-4-carboxylate

To a solution of benzyl piperidine-4-carboxylate hydrochloride (500.0 mg, 1.96 mmol) and potassium carbonate (811 mg, 5.87 mmol) in DMF (10 mL) was added 1-BOC-3-(bromomethyl)azetidine (587 mg, 2.35 mmol). The mixture was stirred at 25° C. for 16 h. Then the mixture was stirred at 50° C. for another 16 h. The mixture was diluted with EtOAc (100 mL) and then washed with brine (30 mL×3). The organic layer was dried over sodium sulfate, filtered and the filtrate was concentrated under vacuum. The residue was purified by column chromatography (PE:EA=2:1˜0:1) to give the title compound (710 mg, 1.83 mmol, 74.17% yield) as light yellow gum. ESI MS [M+H]⁺: 389.1.

### Step 2: benzyl 1-[(1-tert-butoxycarbonylazetidin-3-yl)methyl]-1-(2-tert-butoxy-2-oxo-ethyl)piperidin-1-ium-4-carboxylate formate

To a solution of benzyl 1-[(1-tert-butoxycarbonylazetidin-3-yl)methyl]piperidine-4-carboxylate (4.7 g, 12.1 mmol) and sodium iodide (181 mg, 1.21 mmol) in DMF (50 mL) was added tert-butyl bromoacetate (4.72 g, 24.2 mmol) and N,N-diisopropylethylamine (6.32 mL, 36.29 mmol). The mixture was stirred at 60° C. for 16 h. The mixture was concentrated under vacuum. The residue was purified twice by prep-HPLC (FA condition) to give the title compound (5 g, 9.93 mmol, 82.06% yield) as a yellow solid. ESI MS [M+H]⁺: 503.2.

### Step 3: 1-[(1-tert-butoxycarbonylazetidin-3-yl)methyl]-1-(2-tert-butoxy-2-oxo-ethyl)piperidin-1-ium-4-carboxylate

To a solution of benzyl 1-[(1-tert-butoxycarbonylazetidin-3-yl)methyl]-1-(2-tert-butoxy-2-oxo-ethyl)piperidin-1-ium-4-carboxylate formate (4.7 g, 8.57 mmol) in methanol (150 mL) was added palladium on charcoal (400 mg, 10% purity) and palladium hydroxide on charcoal (400.0 mg, 10% wt) under nitrogen atmosphere. The mixture was degassed and then stirred at 15° C. for 4 h under hydrogen (760 mmHg). The mixture was filtered through celit pad, the solid was washed with MeOH (20 mL×4). The combined filtrate was concentrated under vacuum. The solid was dissolved in water (100 mL) and then lyophilized to give 1-[(1-tert-butoxycarbonylazetidin-3-yl)methyl]-1-(2-tert-butoxy-2-oxo-ethyl)piperidin-1-ium-4-carboxylate (3.5 g, 8.48 mmol, 93.86% yield) as a white solid. ESI MS [M+H]⁺: 413.2.

### Intermediate H22

### Tert-butyl 2-[1-[3-(tert-butoxycarbonylamino)propyl]piperazin-1-ium-1-yl]acetate formate

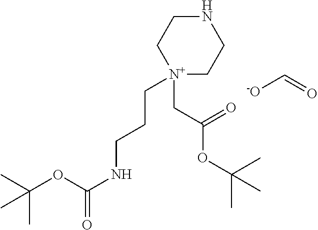

### Step 1: benzyl 4-[3-(tert-butoxycarbonylamino)propyl]piperazine-1-carboxylate

To a solution of 1-CBZ-piperazine (5.0 g, 22.7 mmol) in MeCN (100 mL) was added triethylamine (3.16 mL, 22.7 mmol) and 3-(BOC-amino)propyl bromide (5.68 g, 23.83 mmol), then the mixture was stirred at 25° C. for 16 h. The mixture was concentrated in vacuum and purified by silica gel column (PE/EA=100:1˜1:2) to obtain the title compound (5.2 g, 13.78 mmol, 60.69% yield) as light brown solid. ESI MS [M+H]⁺: 378.3.

### Step 2: benzyl 4-[3-(tert-butoxycarbonylamino)propyl]-4-(2-tert-butoxy-2-oxo-ethyl)piperazin-4-ium-1-carboxylate formate

To a solution of benzyl 4-[3-(tert-butoxycarbonylamino)propyl]piperazine-1-carboxylate (5.2 g, 13.78 mmol) in MeCN (100 mL) was added triethylamine (1.92 mL, 13.78 mmol) and tert-butyl bromoacetate (5.37 g, 27.55 mmol), then the mixture was stirred at 50° C. for 16 h. The mixture was concentrated in vacuum and purified by prep-HPLC (0.1% FA)-MeOH to obtain the title compound (4 g, 59% yield) as light yellow solid. ESI MS [M+H]⁺: 492.4.

### Step 3: tert-butyl 2-[1-[3-(tert-butoxycarbonylamino)propyl]piperazin-1-ium-1-yl]acetate formate

To a solution of benzyl 4-[3-(tert-butoxycarbonylamino)propyl]-4-(2-tert-butoxy-2-oxo-ethyl)piperazin-4-ium-1-carboxylate formate (4.0 g, 8.12 mmol) in THF (40 mL) was added 10% palladium on charcoal (400 mg) and the reaction stirred under hydrogen atmosphere at 25° C. for 16 h. The mixture was concentrated in vacuum and purified by prep-HPLC (0.1% FA)-ACN to obtain tert-butyl 2-[1-[3-(tert-butoxycarbonylamino)propyl]piperazin-1-ium-1-yl]acetate formate (1.5 g, 4.18 mmol, 51.53% yield) as white solid. ESI MS [M+H]⁺: 358.3.

### Example A1

### 2-[1-[2-Chloro-4-[[5-(2,3-difluoro-4-methoxy-phenyl)-1-methyl-imidazole-2-carbonyl]amino]benzoyl]-4-piperidyl]ethyl-trimethyl-ammonium;formate

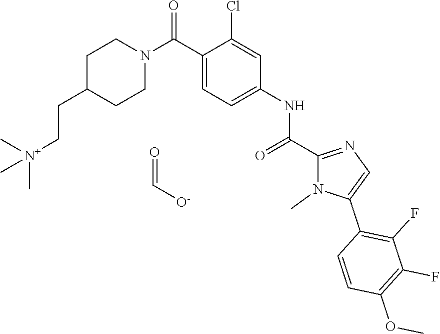

### Step 1: N-[3-chloro-4-[4-[2-(dimethylamino)ethyl]piperidine-1-carbonyl]phenyl]-5-(2,3-difluoro-4-methoxy-phenyl)-1-methyl-imidazole-2-carboxamide

In a 100 mL round-bottomed flask, N-(4-(4-(2-aminoethyl)piperidine-1-carbonyl)-3-chlorophenyl)-5-(2,3-difluoro-4-methoxyphenyl)-1-methyl-1H-imidazole-2-carboxamide (160 mg, 301 μmol), formaldehyde (274 mg, 3.01 mmol) and NaBH₃CN (113 mg, 1.8 mmol) were combined with MeOH (20 mL) to give a light brown solution. The reaction was stirred at room temperature for overnight. The crude reaction mixture was concentrated in vacuum. The reaction mixture was poured into 25 mL sat NaHCO₃ and extracted with EtOAc (3×25 mL). The organic layers were combined, washed with sat NaCl (1×25 mL). The organic layers were dried over Na₂SO₄ and concentrated in vacuo. to afford N-(3-chloro-4-(4-(2-(dimethylamino)ethyl) piperidine-1-carbonyl)phenyl)-5-(2,3-difluoro-4-methoxyphenyl)-1-methyl-1H-imidazole-2-carboxamide (134 mg, 239 μmol, 79% yield). ESI MS [M+H]⁺: 560.6.

### Step 2: 2-[1-[2-chloro-4-[[5-(2,3-difluoro-4-methoxy-phenyl)-1-methyl-imidazole-2-carbonyl]amino]benzoyl]-4-piperidyl]ethyl-trimethyl-ammonium;formate

In a 100 mL round-bottomed flask, N-(3-chloro-4-(4-(2-(dimethylamino)ethyl)piperidine-1-carbonyl)phenyl)-5-(2,3-difluoro-4-methoxyphenyl)-1-methyl-1H-imidazole-2-carboxamide (134 mg, 239 μmol), Mel (170 mg, 1.2 mmol) and DIEA (155 mg, 1.2 mmol) were combined with EtOH (6 mL) to give a light brown solution. The reaction was stirred at room temperature for 2 h. The crude reaction mixture was concentrated in vacuum. The crude material was purified by preparative HPLC. to afford 2-[1-[2-chloro-4-[[5-(2,3-difluoro-4-methoxy-phenyl)-1-methyl-imidazole-2-carbonyl]amino]benzoyl]-4-piperidyl]ethyl-trimethyl-ammonium;formate (48 mg, 75.9 μmol, 31.7% yield). ESI MS [M+H]⁺: 574.3.

### Example A2

### 5-[[2-Chloro-4-[[5-(2,3-difluoro-4-methoxy-phenyl)-1-methyl-imidazole-2-carbonyl]amino]benzoyl]amino]pentyl-trimethyl-ammonium;formate

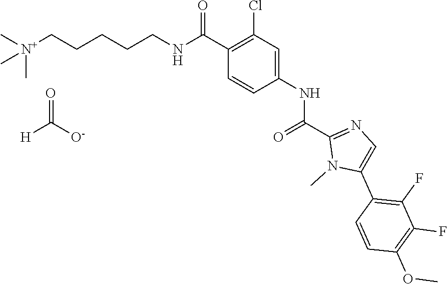

### Step 1:5-[[2-chloro-4-[[5-(2,3-difluoro-4-methoxy-phenyl)-1-methyl-imidazole-2-carbonyl]amino]benzoyl]amino]pentyl-trimethyl-ammonium;formate

To a solution of N-(3-chloro-4-((5-(dimethylamino)pentyl)carbamoyl)phenyl)-5-(2,3-difluoro-4-methoxyphenyl)-1-methyl-1H-imidazole-2-carboxamide (75 mg, 140 μmol) in DCM (2 mL) was added methyl iodide (26.3 μl, 421 μmol), the reaction was stirred for 15 hours at room temperature. The reaction mixture was concentrated in vacuum. The residue was purified by preparative HPLC to give 5-[[2-chloro-4-[[5-(2,3-difluoro-4-methoxy-phenyl)-1-methyl-imidazole-2-carbonyl]amino]benzoyl]amino]pentyl-trimethyl-ammonium;formate. ESI MS [M+H]⁺: 548.5.

The following Type I Examples were prepared in analogy to Example A2:

| | | | MS | |
|---|---|---|---|---|
| | | | ESI | Starting |
| | Name | Structure | [M + H]⁺ | Material |
| Exam- ple A3 | (2-amino-2-oxo- ethyl)-[5-[[2-chloro- 4-[[5-(2,3-difluoro-4- methoxy-phenyl)-1- methyl-imidazole-2- carbonyl]amino] benzoyl]amino] pentyl]-dimethyl- ammonium; formate | 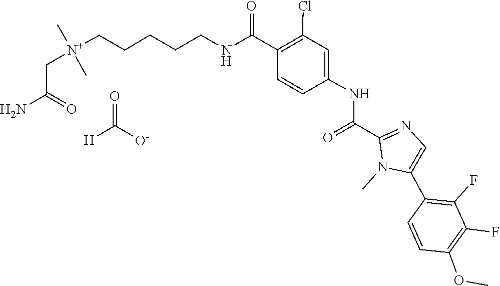 | 591.1 | Intermediate E18 and 2- iodoacetamide |
| Exam- ple A4 | (2-amino-2-oxo- ethyl)-[2-[4-[2- chloro-4-[[5-(2,3- difluoro-4-methoxy- phenyl)-1-methyl- imidazole-2- carbonyl]amino] benzoyl]piperazin-1- yl]ethyl]-dimethyl- ammonium; formate | 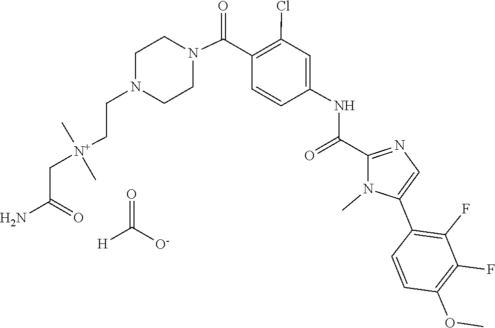 | 618.4 | Intermediate E19 and 2- iodoacetamide |
| Exam- ple A5 | 2-(2-(4-(5-(2,3- difluoro-4- methoxyphenyl)-1- methyl-1H-imidazole- 2-carboxamido)-2- ethylbenzamido) ethoxy)-N,N,N- trimethylethan-1- aminium iodide | 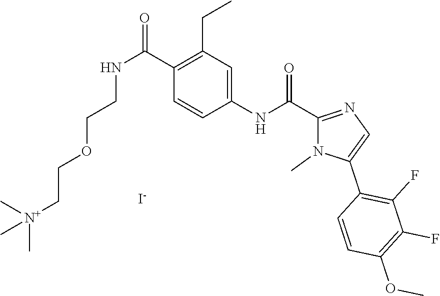 [SMILES: CCc1cc(NC(=O)c2ncc(-c3ccc(OC)c(F)c3F)n2C)ccc1C(=O)NCCOCC[N+](C)(C)C ; [I-]] | 544.3 | Intermediate E21 and methyl iodide |
| Exam- ple A6 | 3-(4-(4-(5-(2,3- difluoro-4- methoxyphenyl)-1- methyl-1H-imidazole- 2-carboxamido)-2- ethylbenzoyl) piperazin-1-yl)- N,N,N- trimethylpropan-1- aminium iodide | 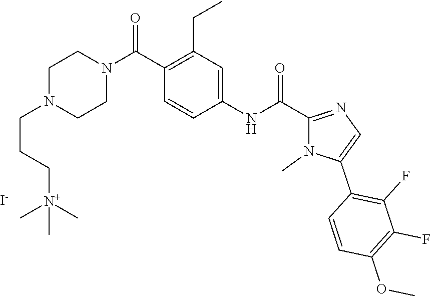 [SMILES: [I-] ; CCc1cc(NC(=O)c2ncc(-c3ccc(OC)c(F)c3F)n2C)ccc1C(=O)N1CCN(CCC[N+](C)(C)C)CC1] | 583.3 | Intermediate E20 and methyl iodide |
| Exam- ple A7 | [2-[4-[2-chloro-4-[[5- [4-(cyanomethoxy)- 2,3-difluoro-phenyl]- 1-methyl-imidazole- 2-carbonyl]amino] benzoyl]piperazin-1- yl]-2-oxo-ethyl]- trimethyl-ammonium; formate; iodide | 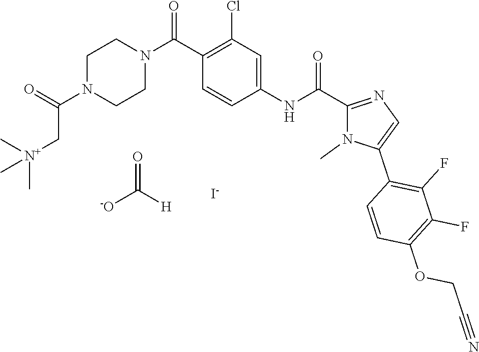 | 614.4 | Intermediate E1 and methyl iodide |
| Exam- ple A8 | [2-[4-[4-[[5-[4- (cyanomethoxy)-2,3- difluoro-phenyl]-1- methyl-imidazole-2- carbonyl]amino]-2- methyl- benzoyl]piperazin-1- yl]-2-oxo-ethyl]- trimethyl-ammonium; iodide | 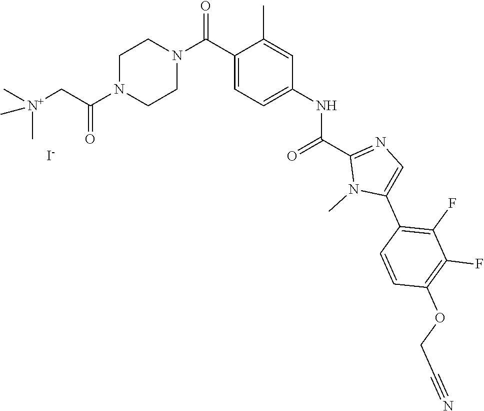 [SMILES: Cc1cc(NC(=O)c2ncc(-c3ccc(OCC#N)c(F)c3F)n2C)ccc1C(=O)N1CCN(C(=O)C[N+](C)(C)C)CC1 ; [I-]] | 594.3 | Intermediate E37 and methyl iodide |
| Exam- ple A9 | 2-[4-[2-chloro-4-[[5- (2,3-difluoro-4- methoxy-phenyl)-1- methyl-imidazole-2- carbonyl]amino] benzoyl]piperazin-1- yl]ethyl-trimethyl- ammonium; iodide | 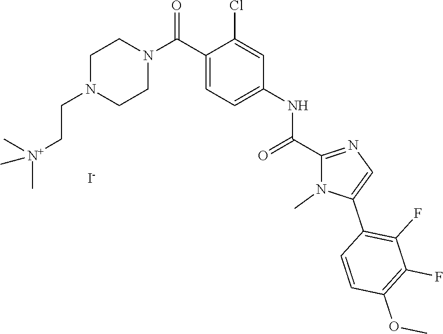 [SMILES: COc1ccc(-c2cnc(C(=O)Nc3ccc(C(=O)N4CCN(CC[N+](C)(C)C)CC4)c(Cl)c3)n2C)c(F)c1F ; [I-]] | 575.2 | Intermediate E19 and methyl iodide |

### Example A10

### [2-[[3-[[2-Chloro-4-[[5-(2,3-difluoro-4-methoxy-phenyl)-1-methyl-imidazole-2-carbonyl]amino]benzoyl]amino]cyclobutyl]amino]-2-oxo-ethyl]-trimethyl-ammonium formate

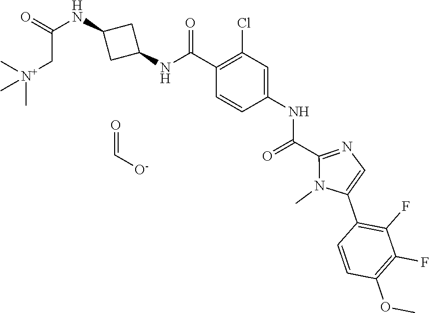

To a solution of 2-chloro-4-[[5-(2,3-difluoro-4-methoxy-phenyl)-1-methyl-imidazole-2-carbonyl]amino]benzoic acid (50 mg, 0.12 mmol) in DMF (0.47 mL) was added [2-[(3-aminocyclobutyl)amino]-2-oxo-ethyl]-trimethyl-ammonium iodide hydrochloride (62.2 mg, 0.180 mmol), 0-(7-azabenzotriazol-1-yl)-N,N,N′,N′-tetramethyluronium hexafluorophosphate (90.2 mg, 0.24 mmol) and N,N-diisopropylethylamine (0.06 mL, 0.36 mmol). The mixture was stirred at 30° C. for 12 h. The mixture was purified by prep-HPLC (FA), and the purified solution was lyophilized to afford the title compound (50.4 mg, 0.08 mmol, 67% yield) as a yellow solid. ESI MS [M+H]⁺: 589.3.

The following Type I Examples were prepared in analogy to Example A10:

| | | | MS | |
|---|---|---|---|---|
| | | | ESI | Starting |
| Int. | Name | Structure | [M + H]⁺ | Material |
| Exa- mple A11 | 2-[[3-[[2-chloro-4-[[5- (2,3-difluoro-4- methoxy-phenyl)-1- methyl-imidazole-2- carbonyl]amino] benzoyl]amino] cyclobutyl]amino]ethyl- trimethyl-ammonium; formic acid; formate | 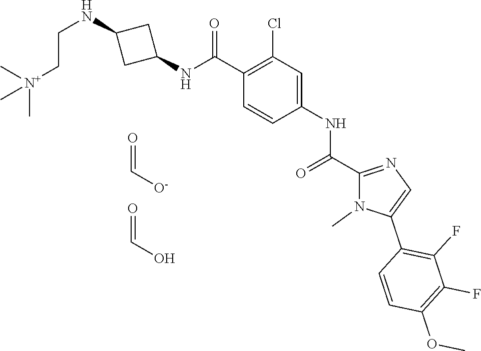 | 575.3 | Intermediate G1 and H2 |
| Exam- ple A12 | [2-[3-[[2-chloro-4-[[5- [4-(cyanomethoxy)-2,3- difluoro-phenyl]-1- methyl-imidazole-2- carbonyl]amino] benzoyl]amino] propylamino]-2-oxo- ethyl]-trimethyl- ammonium formate | 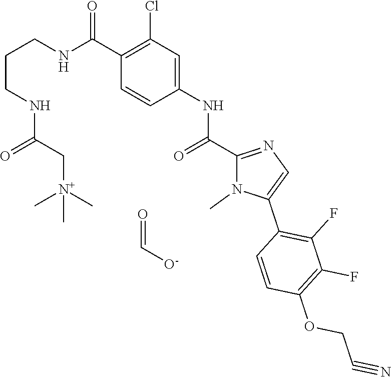 | 602.2 | Intermediate G2 and H3 |
| Exam- ple A13 | (2-amino-2-oxo-ethyl)- [2-[2-[[2-chloro-4-[[5- [4-(cyanomethoxy)-2,3- difluoro-phenyl]-1- methyl-imidazole-2- carbonyl]amino] benzoyl]amino]ethoxy] ethyl]-dimethyl- ammonium formate | 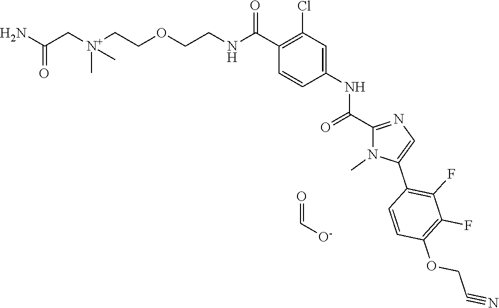 | 618.2 | Intermediate G2 and H5 |
| Exam- ple A14 | [2-[2-[2-chloro-4-[[5-[4- (cyanomethoxy)-2,3- difluoro-phenyl]-1- methyl-imidazole-2- carbonyl]amino] benzoyl]amino] ethylamino]-2-oxo- ethyl]-trimethyl- ammonium formate | 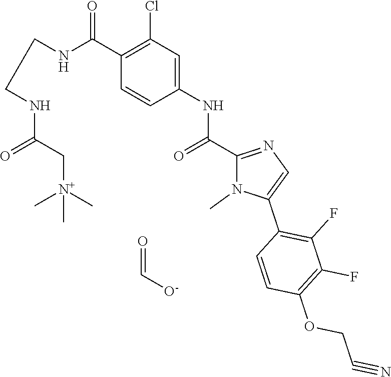 | 588.1 | Intermediate G2 and H4 |
| Exam- ple A15 | 2-[2-[[2-chloro-4-[[5-[4- (cyanomethoxy)-2,3- difluoro-phenyl]-1- methyl-imidazole-2- carbonyl]amino] benzoyl]amino]ethoxy] ethyl-trimethyl- ammonium formate | 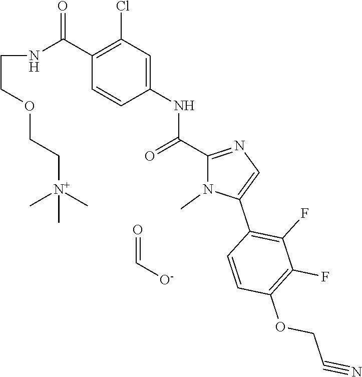 | 575.2 | Intermediate G2 and H6 |
| Exam- ple A16 | [2-[[3-[2-chloro-4-[[5- [4-(cyanomethoxy)-2,3- difluoro-phenyl]-1- methyl-imidazole-2- carbonyl]amino] benzoyl]amino] cyclobutyl]amino]-2- oxo-ethyl]-trimethyl- ammonium formate | 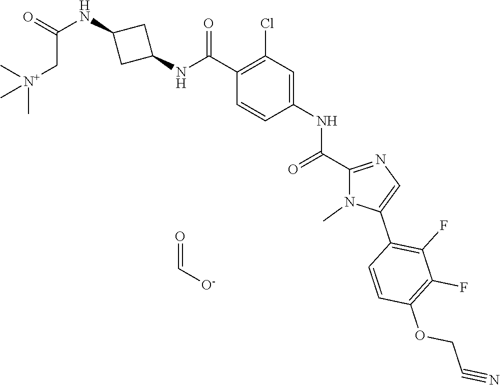 | 614.2 | Intermediate G2 and H1 |
| Exam- ple A17 | 2-[[3-[[2-chloro-4-[[5- [4-(cyanomethoxy)-2,3- difluoro-phenyl]-1- methyl-imidazole-2- carbonyl]amino] benzoyl]amino] cyclobutyl]amino]ethyl- trimethyl-ammonium formate | 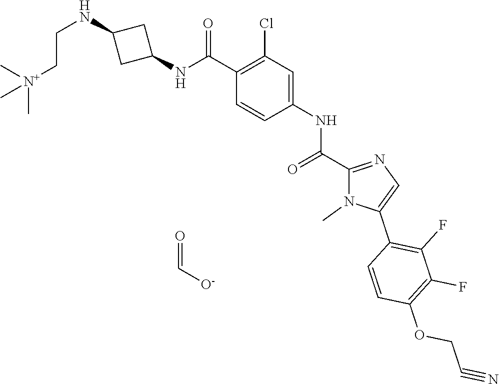 | 600.2 | Intermediate G2 and H2 |
| Exam- ple A18 | [2-[[3-[[4-[5-[4- (cyanomethoxy)-2,3- difluoro-phenyl]-1- methyl-imidazole-2- carbonyl]amino]-2- methyl-benzoyl]amino] cyclobutyl]amino]-2- oxo-ethyl]-trimethyl- ammonium formate | 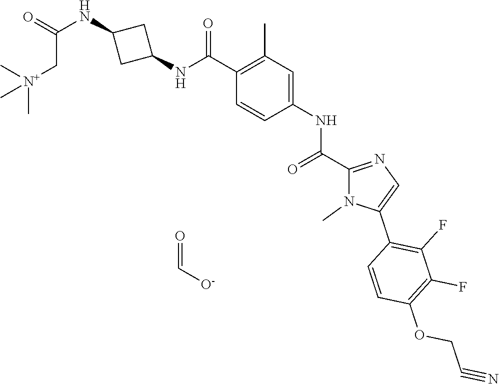 | 594.2 | Intermediate G3 and H1 |
| Exam- ple A19 | 2-[[3-[[4-[[5-[4- (cyanomethoxy)-2,3- difluoro-phenyl]-1- methyl-imidazole-2- carbonyl]amino]-2- methyl-benzoyl]amino] cyclobutyl]amino]ethyl- trimethyl-ammonium formate | 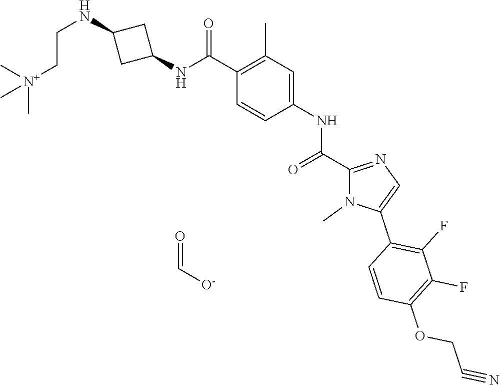 | 580.2 | Intermediate G3 and H2 |
| Exam- ple C32 | 2-[1-(3-aminopropyl)-4- [4-[2-chloro-4-[[5-(2,3- difluoro-4-methoxy- phenyl)-1-methyl- imidazole-2- carbonyl]amino] benzoyl]piperazine-1- carbonyl]piperidin-1- ium-1-yl]acetic acid; formate | 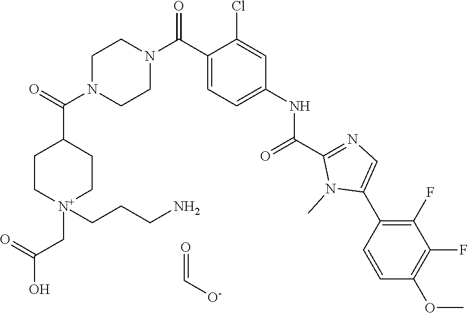 | 716.3 | Intermediate G1 and H18; remove protection group with TFA |
| Exam- ple C33 | 2-[1-(azetidin-3- ylmethyl)-4-[4-[2- chloro-4-[[5-(2,3- difluoro-4-methoxy- phenyl)-1-methyl- imidazole-2- carbonyl]amino] benzoyl]piperazine-1- carbonyl]piperidin-1- ium-1-yl]acetic acid; formate | 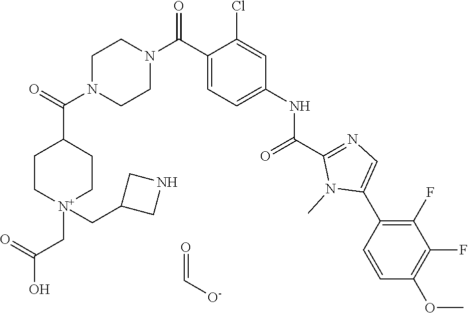 | 728.3 | Intermediate G1 and H19; remove protection group with TFA |
| Exam- ple C34 | bis(3-aminopropyl)- (carboxymethyl)-[4-[4- [2-chloro-4-[[5-(2,3- difluoro-4-methoxy- phenyl)-1-methyl- imidazole-2- carbonyl]amino] benzoyl]piperazin-1-yl]- 4-oxo-butyl]ammonium; formate | 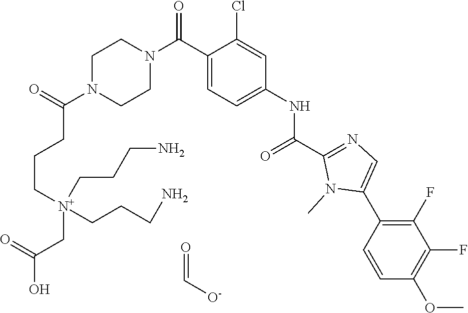 | 747.3 | Intermediate G1 and H20; remove protection group with TFA |
| Exam- ple C40 | 2-[1-(azetidin-3- ylmethyl)-4-[4-[2- chloro-4-[[5-(2-chloro- 3-fluoro-4-methoxy- phenyl)-1-methyl- imidazole-2- carbonyl]amino] benzoyl]piperazine-1- carbonyl]piperidin-1- ium-1-yl]acetic acid; formate | 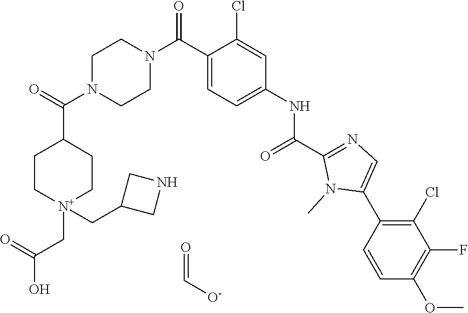 | 744.3 | Intermediate E7 and H21; remove protection group with TFA |

### Example A20

### N-[3-chloro-4-[[1-(2-hydroxyethyl)-2-methyl-pyrazol-2-ium-4-yl]methylcarbamoyl]phenyl]-5-(2,3-difluoro-4-methoxy-phenyl)-1-methyl-imidazole-2-carboxamide;formate

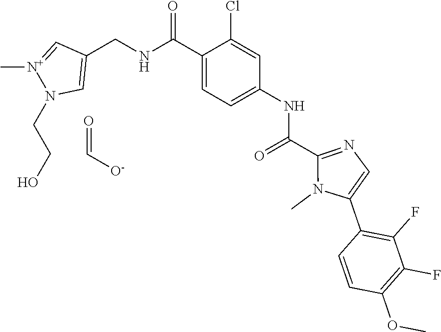

3-(((ethylimino)methylene)amino)-N,N-dimethylpropan-1-amine hydrochloride (54.5 mg, 285 μmol) and N-ethyl-N-isopropylpropan-2-amine (153 mg, 207 μl, 1.19 mmol, Eq: 5) were added to a solution of Intermediate G1 (55.6 mg, 356 μmol), Intermediate H7 (100 mg, 237 μmol) and HOBt (38.4 mg, 285 μmol, Eq: 1.2) in DMA (4.74 mL) and stirred for 2 hours at room temperature under nitrogen. The crude product was purified by preparative HPLC. ESI MS [M+H]⁺: 559.4.

The following Type I Examples were prepared in analogy to example A20:

| | | | MS | |
|---|---|---|---|---|
| | | | ESI | Starting |
| Int. | Name | Structure | [M]+ | Material |
| Example A21 | N-[3-chloro-4-[1-(3- hydroxypropyl)-2- methyl-pyrazol-2-ium- 4-yl]methylcarbamoyl] phenyl]-5-(2,3-difluoro- 4-methoxy-phenyl)-1- methyl-imidazole-2- carboxamide; formate | 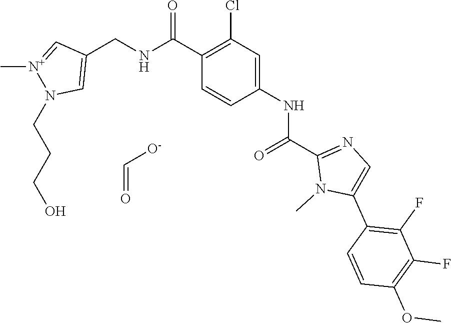 | 573.2 | Intermediate G1 and H8 |
| Example A22 | N-[3-chloro-4-[1-(2- cyanoethyl)-2-methyl- pyrazol-2-ium-4- yl]methylcarbamoyl] phenyl]-5-(2,3-difluoro- 4-methoxy-phenyl)-1- methyl-imidazole-2- carboxamide; formate | 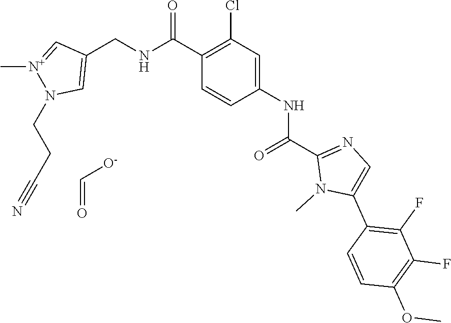 | 568.4 | Intermediate G1 and H9 |
| Example A23 | N-[4-[1-(2-amino-2- oxo-ethyl)-2-methyl- pyrazol-2-ium-4- yl]methylcarbamoyl]-3- chloro-phenyl]-5-(2,3- difluoro-4-methoxy- phenyl)-1-methyl- imidazole-2- carboxamide; formate | 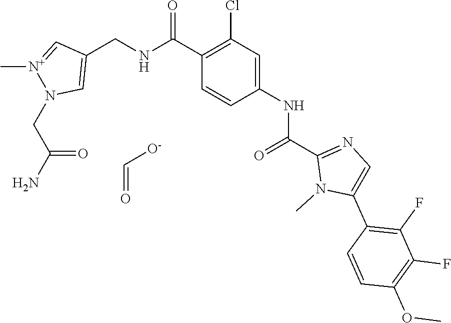 | 572.3 | Intermediate G1 and H10 |
| Example A24 | N-[3-chloro-4-[[1- methyl-2-(2- methylsulfonylethyl) pyrazol-1-ium-4- yl]methylcarbamoyl] phenyl]-5-(2,3-difluoro- 4-methoxy-phenyl)-1- methyl-imidazole-2- carboxamide; formate | 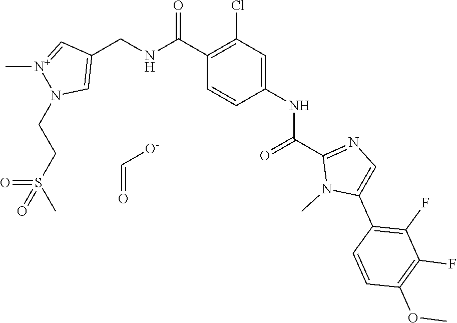 | 621.3 | Intermediate G1 and H12 |
| Example A25 | N-[3-chloro-4-[1-(3- methoxypropyl)-2- methyl-pyrazol-2-ium- 4-yl]methylcarbamoyl] phenyl]-5-(2,3-difluoro- 4-methoxy-phenyl)-1- methyl-imidazole-2- carboxamide; formate | 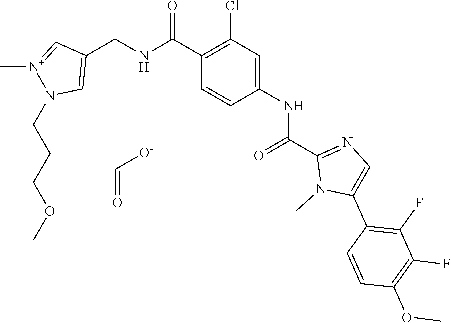 | 587.4 | Intermediate G1 and H11 |
| Example A26 | N-[3-chloro-4-[1-[2-(2- hydroxyethoxy)ethyl]- 2-methyl-pyrazol-2- ium-4- yl]methylcarbamoyl] phenyl]-5-(2,3-difluoro- 4-methoxy-phenyl)-1- methyl-imidazole-2- carboxamide; formate | 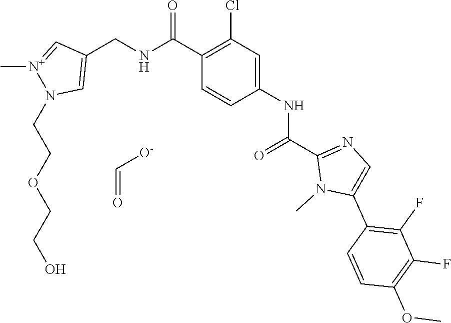 | 603.2 | Intermediate G1 and H13 |
| Example A27 | N-[3-chloro-4-[[2- [[(2S,4R)-4-hydroxy- 1,1-dimethyl- pyrrolidin-1-ium-2- carbonyl]amino]thiazol- 5-yl]methylcarbamoyl] phenyl]-5-(2,3-difluoro- 4-methoxy-phenyl)-1- methyl-imidazole-2- carboxamide; formate | 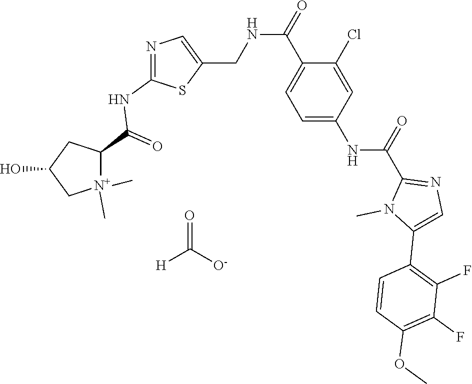 | 674.2 | Intermediate E24 and Intermediate H14 |
| Example A28 | N-[3-chloro-4-[2-[(1,1- dimethylpyrrolidin-1- ium-2- carbonyl)amino]thiazol- 5-yl]methylcarbamoyl] phenyl]-5-(2,3-difluoro- 4-methoxy-phenyl)-1- methyl-imidazole-2- carboxamide; formate | 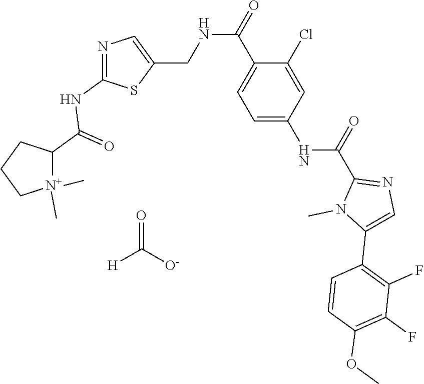 | 658.2 | Intermediate E24 and Intermediate H15 |
| Example A29 | N-[5-[2-chloro-4-[[5- (2,3-difluoro-4- methoxy-phenyl)-1- methyl-imidazole-2- carbonyl]amino] benzoyl]amino]methyl] thiazol-2-yl]-1,1- dimethyl-piperidin-1- ium-4-carboxamide; formate | 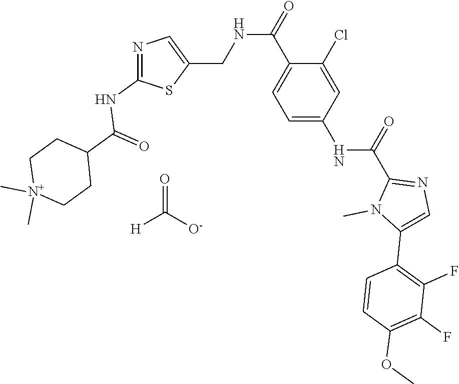 | 672.2 | Intermediate E24 and Intermediate H16 |
| Example A30 | 1-(2-amino-2-oxo- ethyl)-N-[5-[[[2-chloro- 4-[[5-(2,3-difluoro-4- methoxy-phenyl)-1- methyl-imidazole-2- carbonyl]amino] benzoyl]amino]methyl] thiazol-2-yl]-1-methyl- piperidin-1-ium-4- carboxamide; formate | 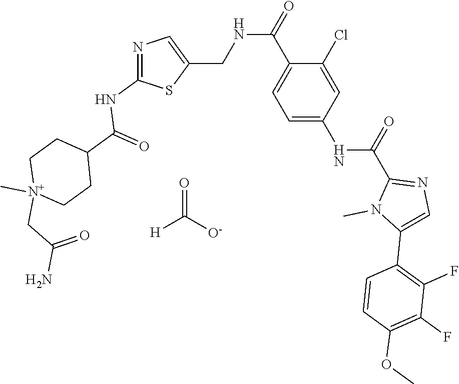 | 715.2 | Intermediate E24 and Intermediate H17 |
| Example A31 | N-[5-[[1-[2-chloro-4- [[5-(2,3-difluoro-4- methoxy-phenyl)-1- methyl-imidazole-2- carbonyl]amino] benzoyl]piperidine-4- carbonyl]amino]methyl] thiazol-2-yl]-1,1- dimethyl-piperidin-1- ium-4-carboxamide; formate | 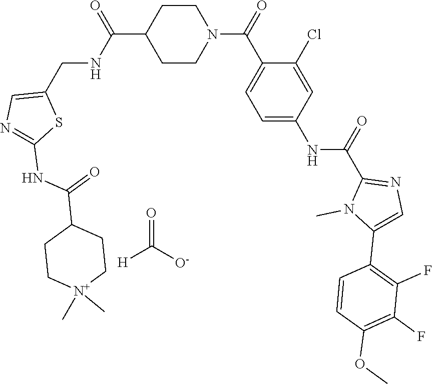 | 783.3 | Intermediate E26 and Intermediate H16 |

### Example B1

### (2-Amino-2-oxo-ethyl)-[2-[4-[4-[[5-(2,3-difluoro-4-methoxy-phenyl)-1-methyl-imidazole-2-carbonyl]amino]-2-methyl-benzoyl]piperazin-1-yl]-2-oxo-ethyl]-dimethyl-ammonium;formate

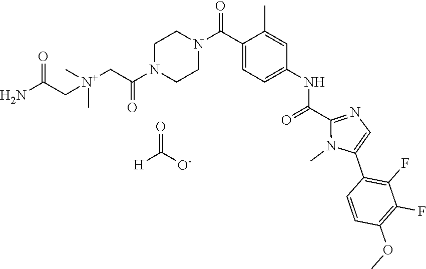

### Step 1: 5-(2,3-difluoro-4-methoxy-phenyl)-N-[4-[4-[2-(dimethylamino)acetyl]piperazine-1-carbonyl]-3-methyl-phenyl]-1-methyl-imidazole-2-carboxamide

To a solution of 5-(2,3-Difluoro-4-methoxy-phenyl)-1-methyl-N-[3-methyl-4-(piperazine-1-carbonyl)phenyl]imidazole-2-carboxamide (940 mg, 2.0 mmol), dimethylglycine (260 mg, 2.5 mmol) in anhydrous DMF (15 mL) was added TEA (505 mg, 5 mmol) and then the resultant mixture was stirred for 10 min at room temperature, T₃P (2.0 mL, 3.0 mmol) was added in the mixture and stirred for extra 10 hr. The mixture was poured into water (50 mL) and the aqueous solution was extracted with DCM/isopropanol (100/10 mL). The organic layers were combined and washed with water and brine, dried over anhydrous Na₂SO₄ and concentrated under reduced pressure to give a red oil, which was purified by column to afford 5-(2,3-difluoro-4-methoxy-phenyl)-N-[4-[4-[2-(dimethylamino)acetyl]piperazine-1-carbonyl]-3-methyl-phenyl]-1-methyl-imidazole-2-carboxamide (1.0 g, 90% yield). ESI MS [M+H]⁺: 555.2.

### Step 2: (2-amino-2-oxo-ethyl)-[2-[4-[4-[[5-(2,3-difluoro-4-methoxy-phenyl)-1-methyl-imidazole-2-carbonyl]amino]-2-methyl-benzoyl]piperazin-1-yl]-2-oxo-ethyl]-dimethyl-ammonium;formate

To a solution of 5-(2,3-difluoro-4-methoxy-phenyl)-N-[4-[4-[2-(dimethylamino)acetyl]piperazine-1-carbonyl]-3-methyl-phenyl]-1-methyl-imidazole-2-carboxamide (220 mg, 0.4 mmol) in DCM (10 mL) and EtOH (2 mL) was added DIPEA (110 mg, 0.8 mmol), 2-iodoacetamide (370 mg, 2.0 mmol) at room temperature. The resultant mixture was stirred overnight. The mixture was poured into water (15 mL), extracted with dichloromethane/isopropanol (50/5 mL), the organic layer was concentrated to give a red oil, which was purified by Prep-HPLC to afford (2-amino-2-oxo-ethyl)-[2-[4-[4-[[5-(2,3-difluoro-4-methoxy-phenyl)-1-methyl-imidazole-2-carbonyl]amino]-2-methyl-benzoyl]piperazin-1-yl]-2-oxo-ethyl]-dimethyl-ammonium;formate (54 mg, 20% yield) as a white powder. ESI MS [M+H]⁺: 612.2.

The following Type II Examples were prepared in analogy to Example B1:

| | | | MS | |
|---|---|---|---|---|
| | | | ESI | |
| Int. | Name | Structure | [M + H]⁺ | Starting Material |
| Example B2 | (2-amino-2-oxo- ethyl)-[2-[4-[4-[[5-[4- (cyanomethoxy)-2,3- difluoro-phenyl]-1- methyl-imidazole-2- carbonyl]amino]-2- methyl- benzoyl]piperazin-1- yl]-2-oxo-ethyl]- dimethyl-mmonium; formate | 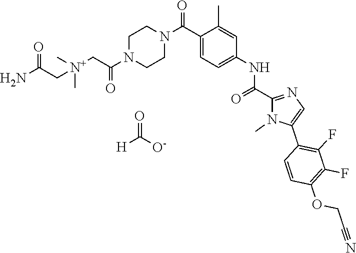 | 637.3 | Intermediate E4 and 2- (dimethylamino) acetic acid and 2-iodoacetamide |
| Example B3 | (2-amino-2-oxo- ethyl)-[3-[4-[4-[[5- (2,3-difluoro-4- methoxy-phenyl)-1- methyl-imidazole-2- carbonyl]amino]-2- methyl- benzoyl]piperazin-1- yl]-3-oxo-propyl]- dimethyl-ammonium; 2,2,2-trifluoroacetate | 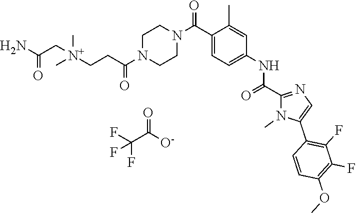 [SMILES: COc1ccc(-c2cnc(C(=O)Nc3ccc(C(=O)N4CCN(C(=O)CC[N+](C)(C)CC(N)=O)CC4)c(C)c3)n2C)c(F)c1F ; O=C([O-])C(F)(F)F] | 626.2 | Intermediate E3 and 3- (dimethylamino) propanoic acid hydrochloride and 2- iodoacetamide |
| Example B4 | (2-amino-2-oxo- ethyl)-[2-[4-[2-chloro- 4-[[5-[4- (cyanomethoxy)-2,3- difluoro-phenyl]-1- methyl-imidazole-2- carbonyl]amino] benzoyl] piperazin-1- yl]-2-oxo-ethyl]- dimethyl-ammonium; formate | 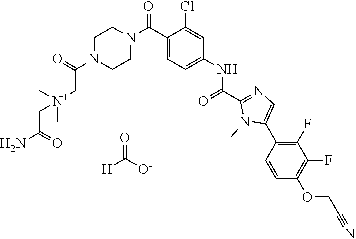 | 657.9 | Intermediate E5 and 2- (dimethylamino) acetic acid and 2-iodoacetamide |
| Example B5 | (2-amino-2-oxo- ethyl)-[2-[1-[2-chloro- 4-[5-(2,3-difluoro-4- methoxy-phenyl)-1- methyl-imidazole-2- carbonyl]amino] benzoyl]-4- piperidyl]amino]-2- oxo-ethyl]-dimethyl- ammonium; formate | 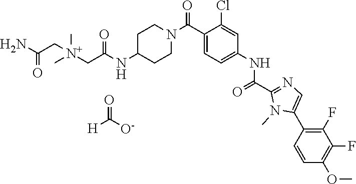 | 646.3 | Intermediate E16 and 2- (dimethylamino) acetic acid and 2-iodoacetamide |
| Example B6 | (2-amino-2-oxo- ethyl)-[2-[3-[[2- chloro-4-[[5-(2,3- difluoro-4-methoxy- phenyl)-1-methyl- imidazole-2- carbonyl]amino] benzoyl] aminoJazetidin-1-yl]- 2-oxo-ethyl]- dimethyl-ammonium; formate | 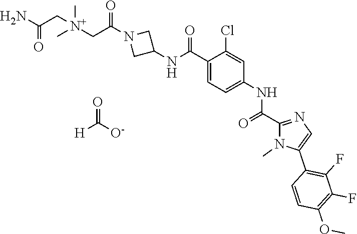 | 618.4 | Intermediate E10 and 2- iodoacetamide and 2- (dimethylamino) acetic acid |
| Example B7 | (2-amino-2-oxo- ethyl)-[2-[4-[2-chloro- 4-[[5-(2,3-difluoro-4- methoxy-phenyl)-1- methyl-imidazole-2- carbonyl]amino] benzoyl] piperazin-1- yl]-2-oxo-ethyl]- dimethyl-ammonium; formate | 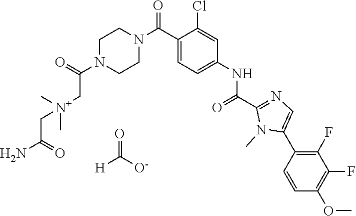 | 632.2 | Intermediate E6 and 2- iodoacetamide and 2- (dimethylamino) acetic acid |

### Example B8

### [3-[4-[4-[[5-(2,3-Difluoro-4-methoxy-phenyl)-1-methyl-imidazole-2-carbonyl]amino]-2-methyl-benzoyl]piperazin-1-yl]-3-oxo-propyl]-trimethyl-ammonium;2,2,2-trifluoroacetate

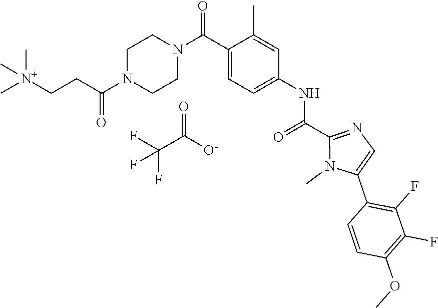 [SMILES: COc1ccc(-c2cnc(C(=O)Nc3ccc(C(=O)N4CCN(C(=O)CC[N+](C)(C)C)CC4)c(C)c3)n2C)c(F)c1F ; O=C([O-])C(F)(F)F]

### Step 1: 5-(2,3-difluoro-4-methoxy-phenyl)-N-[4-[4-[3-(dimethylamino)propanoyl]piperazine-1-carbonyl]-3-methyl-phenyl]-1-methyl-imidazole-2-carboxamide

To a solution of in 5-(2,3-difluoro-4-methoxy-phenyl)-1-methyl-N-[3-methyl-4-(piperazine-1-carbonyl)phenyl]imidazole-2-carboxamide (940 mg, 2.0 mmol), 3-(dimethylamino) propanoic acid hydrochloride (385 mg, 2.5 mmol) in anhydrous DMF (15 mL) was added TEA (505 mg, 5 mmol) and then the resultant mixture was stirred for 10 min at room temperature. T₃P (2.5 mL, 4.0 mmol) was added in the mixture and stirred for extra 10 hr. The mixture was poured into water (50 mL) and the aqueous solution was extracted with DCM (50 mL×2). The organic layers were combined and washed with water and brine, dried over anhydrous Na₂SO₄ and concentrated under reduced pressure to give a red oil which was purified by column chromatography to afford 5-(2,3-difluoro-4-methoxy-phenyl)-N-[4-[4-[3-(dimethylamino) propanoyl]piperazine-1-carbonyl]-3-methyl-phenyl]-1-methyl-imidazole-2-carboxamide (1.0 g, 83% yield). ESI MS [M+H]⁺: 569.3.

### Step 2: [3-[4-[4-[[5-(2,3-difluoro-4-methoxy-phenyl)-1-methyl-imidazole-2-carbonyl]amino]-2-methyl-benzoyl]piperazin-1-yl]-3-oxo-propyl]-trimethyl-ammonium;2,2,2-trifluoroacetate

To a solution of 5-(2,3-difluoro-4-methoxyphenyl)-N-(4-(4-(3-(dimethylamino)propanoyl) piperazine-1-carbonyl)-3-methylphenyl)-1-methyl-1H-imidazole-2-carboxamide (115 mg, 0.2 mmol) in DCM (5 mL) was added methyl iodide (84 mg, 0.6 mmol) at room temperature. The resultant mixture was stirred overnight. The mixture was poured into water (25 mL) and then extracted with dichloromethane/isopropanol (50/5 mL), the organic layer was concentrated to give a red oil, which was purified by Prep-HPLC to provide [3-[4-[4-[[5-(2,3-difluoro-4-methoxy-phenyl)-1-methyl-imidazole-2-carbonyl]amino]-2-methyl-benzoyl]piperazin-1-yl]-3-oxo-propyl]-trimethyl-ammonium; 2,2,2-trifluoroacetate (30 mg, 21% yield) as a white powder. ESI MS [M+H]⁺: 583.2.

The following Type II Examples were prepared in analogy to Example B8:

| | | | MS | |
|---|---|---|---|---|
| | | | ESI | |
| | Name | Structure | [M + H]⁺ | Starting Material |
| Example B9 | [3-[4-[2-chloro-4- [[5-[4- (cyanomethoxy)-2,3- difluoro-phenyl]-1- methyl-imidazole-2- carbonyl]amino] benzoyl]piperazin-1- yl]-3-oxo-propyl]- trimethyl- ammonium; iodide | 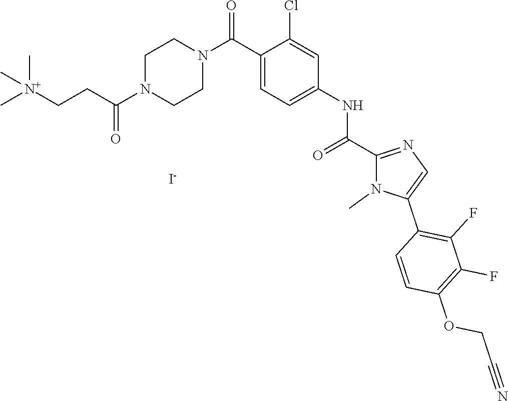 [SMILES: Cn1c(-c2ccc(OCC#N)c(F)c2F)cnc1C(=O)Nc1ccc(C(=O)N2CCN(C(=O)CC[N+](C)(C)C)CC2)c(Cl)c1 ; [I-]] | 628.1 | Intermediate E23 and 3- (dimethylamino) propanoic acid hydrochloride and methyl iodide |
| Example B10 | N-[3-chloro-4-[4- (1,1- dimethylpiperidin-1- ium-4- carbonyl)piperazine- 1-carbonyl]phenyl]- 5-(2,3-difluoro-4- methoxy-phenyl)-1- methyl-imidazole-2- carboxamide; formate | 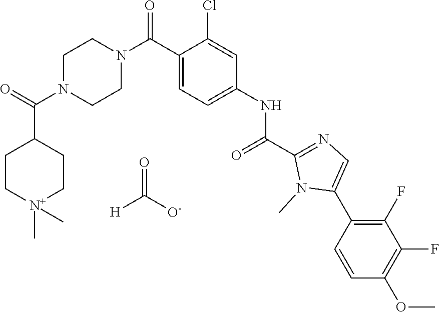 | 629.3 | Intermediate E5 and 1-tert- butoxycarbonyl- piperidine-4- carboxylic acid and methyl iodide |
| Example B11 | [2-[4-[2-chloro-4-[5- [4- (difluoromethoxy)- 2-fluoro-phenyl]-1- methyl-imidazole-2- carbonyl]amino] benzoyl]piperazin-1- yl]-2-oxo-ethyl]- trimethyl- ammonium; formate | 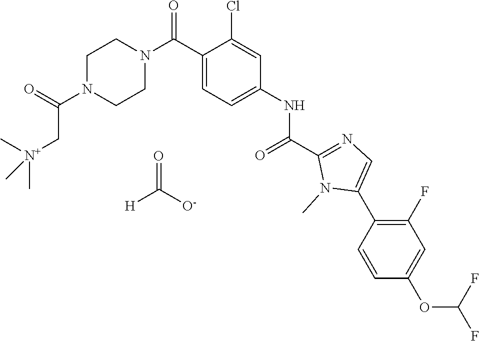 | 608.1 | Intermediate E14 and 2- (dimethylamino) acetic acid and methyl iodide |
| Example B12 | [2-[4-[2-chloro-4- [[5-[4- (difluoromethoxy)- 2,3-difluoro- phenyl]-1-methyl- imidazole-2- carbonyl]amino] benzoyl]piperazin-1- yl]-2-oxo-ethyl]- trimethyl- ammonium; formate | 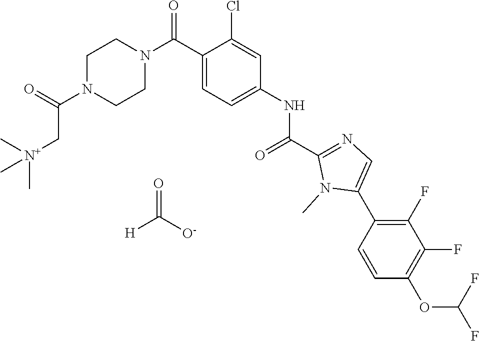 | 625.2 | Intermediate E13 and 2- (dimethylamino) acetic acid and methyl iodide |
| Example B13 | N-[3-chloro-4-[4-[2- (1,1- dimethylpyrrolidin- 1-ium-3- yl)acetyl]piperazine- 1-carbonyl]phenyl]- 5-(2,3-difluoro-4- methoxy-phenyl)-1- methyl-imidazole-2- carboxamide; formate | 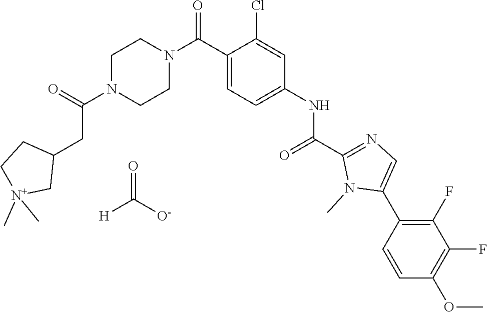 | 629.4 | Intermediate E6 and 2-(1-tert- butoxycarbonyl- pyrrolidin-3- yl)acetic acid and methyl iodide |
| Example B14 | [2-[4-[4-[2-chloro-4- [[5-[4- (difluoromethoxy)- 2,3-difluoro- phenyl]-1-methyl- imidazole-2- carbonyl]amino] benzoyl]piperazine- 1-carbonyl]-1- piperidyl]-2-oxo- ethyl]-trimethyl- ammonium; formate | 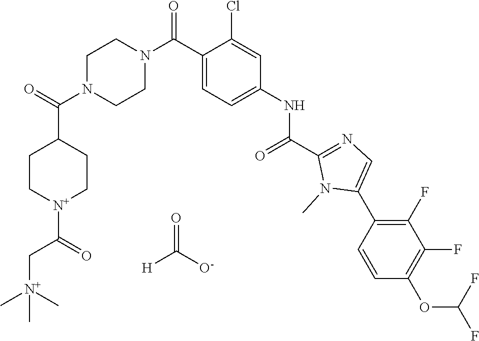 | 736.3 | Intermediate E13 and 1-tert- butoxycarbonyl- piperidine-4- carboxylic acid and 2- (dimethylamino) acetic acid methyl iodide |
| Example B15 | [2-[4-[2-chloro-4- [[5-[4- (difluoromethoxy)- 3-fluoro-phenyl]-1- methyl-imidazole-2- carbonyl]amino] benzoyl]piperazin-1- yl]-2-oxo-ethyl]- trimethyl- ammonium; formate | 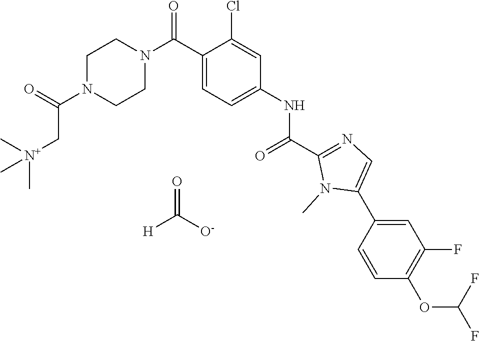 | 607.4 | Intermediate E14 and 2- (dimethylamino) acetic acid and methyl iodide |
| Example B16 | [2-[3-[4-[2-chloro-4- [[5-(2,3-difluoro-4- methoxy-phenyl)-1- methyl-imidazole-2- carbonyl]amino] benzoyl]piperazine- 1-carbonyl]azetidin- 1yl]-2-oxo-ethyl]- trimethyl- ammonium; formate | 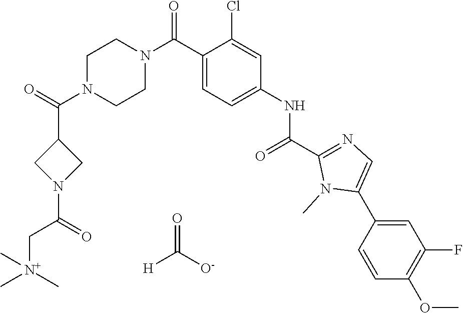 | 672.5 | Intermediate E6 and 1-tert- butoxycarbonyl- azetidine-3- carboxylic acid and 2- (dimethylamino) acetic acid and methyl iodide |
| Example B17 | N-[1-[2-chloro-4- [[5-(2,3-difluoro-4- methoxy-phenyl)-1- methyl-imidazole-2- carbonyl]amino] benzoyl]azetidin-3- yl]-1,1-dimethyl- piperidin-1-ium-4- carboxamide; formate | 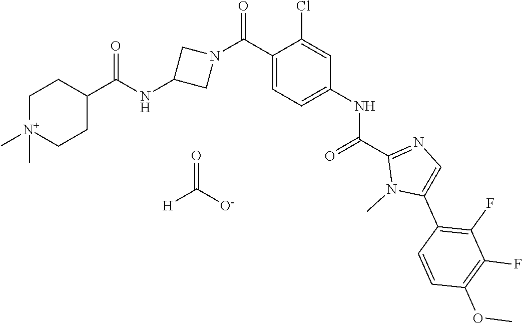 | 615.3 | Intermediate E9 and 1-tert- butoxycarbonyl- piperidine-4- carboxylic acid and methyl iodide |
| Example B18 | N-[4-[(3aS,6aR)-5- [(3R)-1,1- dimethylpyrrolidin- 1-ium-3-carbonyl]- 1,3,3a,4,6,6a- hexahydropyrrolo[3, 4-c]pyrrole-2- carbonyl]-3-chloro- phenyl]-5-(2,3- difluoro-4-methoxy- phenyl)-1-methyl- imidazole-2- carboxamide; formate | 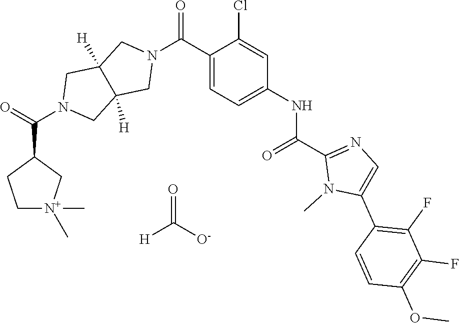 | 641.3 | Intermediate E11 and 1-tert- butoxycarbonyl- pyrrolidine-3- carboxylic acid and methyl iodide |
| Example B19 | [2-[(3aS,6aR)-5-[2- chloro-4-[[5-(2,3- difluoro-4-methoxy- phenyl)-1-methyl- imidazole-2- carbonyl]amino] benzoyl]- 1,3,3a,4,6,6a- hexahydropyrrolo[3, 4-c]pyrrol-2-yl]-2- oxo-ethyl]- trimethyl- ammonium; formate | 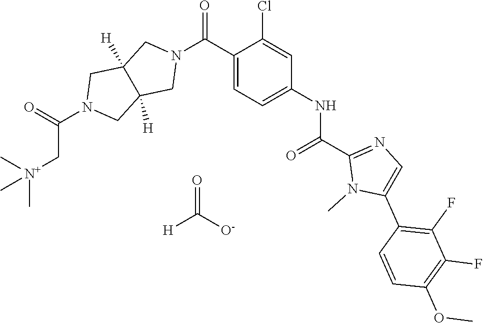 | 615.4 | Intermediate E11 and 2- (dimethylamino) acetic acid and methyl iodide |
| Example B20 | [2-[4-[2-chloro-4- [[5-(2,3-difluoro-4- methoxy-phenyl)-1- methyl-imidazole-2- carbonyl]amino] benzoyl]piperazin-1- yl]-2-oxo-ethyl]- trimethyl- ammonium; formate | 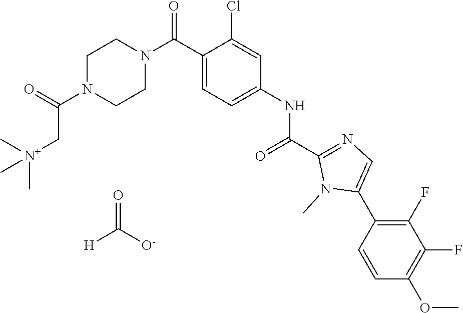 | 590.2 | Intermediate E6 and 2- (dimethylamino) acetic acid and methyl iodide |
| Example B21 | [2-[4-[4-[5-(2,3- difluoro-4-methoxy- phenyl)-1-methyl- imidazole-2- carbonyl]amino]-2- methyl- benzoyl]piperazin-1- yl]-2-oxo-ethyl]- trimethyl- ammonium; formate | 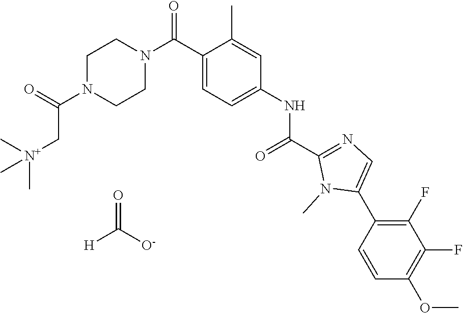 | 570.2 | Intermediate E3 and 2- (dimethylamino) acetic acid and methyl iodide |
| Example B22 | [2-[4-[2-chloro-4-[5- [2-fluoro-4- (fluoromethoxy) phenyl]-1-methyl- imidazole-2- carbonyl]amino] benzoyl]piperazin-1- yl]-2-oxo-ethyl]- trimethyl- ammonium; formate | 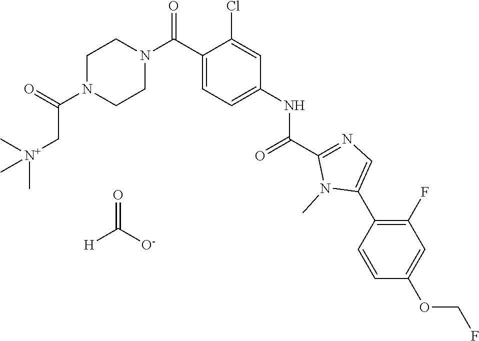 | 590.2 | Intermediate E17 and 2- (dimethylamino) acetic acid and methyl iodide |
| Example B23 | 2-(2-(1-(4-(5-(2,3- difluoro-4- methoxyphenyl)-1- methyl-1H- imidazole-2- carboxamido)-2- ethylbenzoyl) piperidine-4- carboxamido) ethoxy)-N,N,N- trimethylethan-1- aminium iodide | 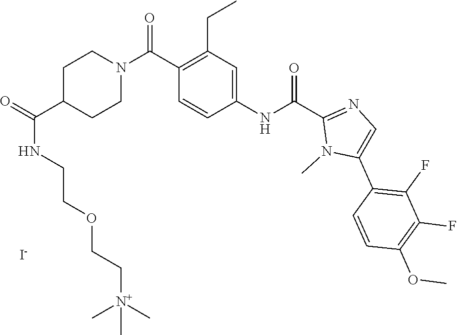 [SMILES: CCc1cc(NC(=O)c2ncc(-c3ccc(OC)c(F)c3F)n2C)ccc1C(=O)N1CCC(C(=O)NCCOCC[N+](C)(C)C)CC1 ; [I-]] | 655.3 | Intermediate E22 and 2-(2- aminoethoxy)- N,N- dimethylethan-1- amine and methyl iodide |
| Example B24 | (S)-3-(1-(2-chloro-4- (5-(4- (cyanomethoxy)-2,3- difluorophenyl)-1- methyl-1H- imidazole-2- carboxamido) benzoyl)piperidine- 4-carboxamido)-1,1- dimethylpyrrolidin- 1-ium formate | 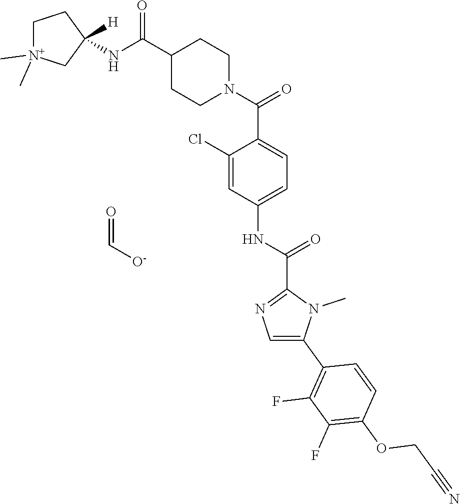 | 654.4 | Intermediate E23 and (3S)-1- methylpyrrolidin- 3-amine and methyl iodide |
| Example B25 | (2S,4R)-2-(4-(2- chloro-4-(5-(2,3- difluoro-4- methoxyphenyl)-1- methyl-1H- imidazole-2- carboxamido) benzoyl)piperazine- 1-carbonyl)-4- hydroxy-1,1- dimethylpyrrolidin- 1-ium formate | 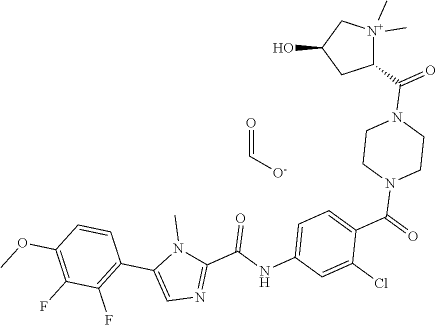 | 631.3 | Intermediate E6 and (2S,4R)-4- hydroxy-1- methyl- pyrrolidine-2- carboxylic acid and methyl iodide |
| Example B26 | 4-(4-(4-(5-(4- (cyanomethoxy)-2,3- difluorophenyl)-1- methyl-1H- imidazole-2- carboxamido)-2- methylbenzoyl) piperazine-1- carbonyl)-4- hydroxy-1,1- dimethylpiperidin-1- ium formate | 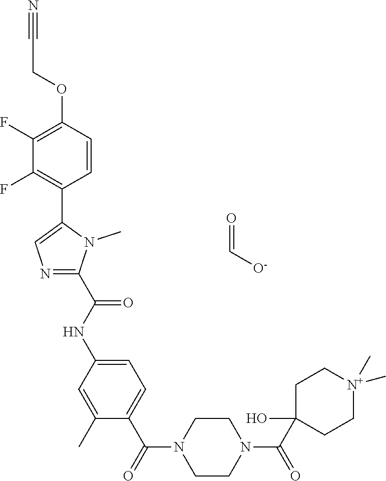 | 650.4 | Intermediate E4 and 4-hydroxy-1- methyl- piperidine-4- carboxylic acid and methyl iodide |
| Example B27 | (2S,4S)-2-(4-(2- chloro-4-(5-(4- (cyanomethoxy)-2,3- difluorophenyl)-1- methyl-1H- imidazole-2- carboxamido) benzoyl)piperazine- 1-carbonyl)-4- hydroxy-1,1,4- trimethylpyrrolidin- 1-ium formate | 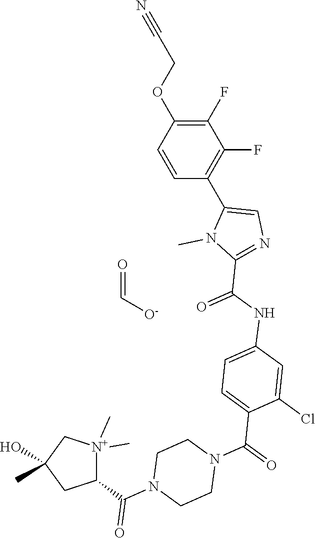 | 670.4 | Intermediate E5 and (2S,4S)-4- hydroxy-1,4- dimethyl- pyrrolidine-2- carboxylic acid and methyl iodide |
| Example B28 | (3R,4R)-3-(4-(4-(5- (4-(cyanomethoxy)- 2,3-difluorophenyl)- 1-methyl-1H- imidazole-2- carboxamido)-2- methylbenzoyl) piperazine-1- carboxamido)-4- hydroxy-1,1- dimethylpyrrolidin- 1-ium formate | 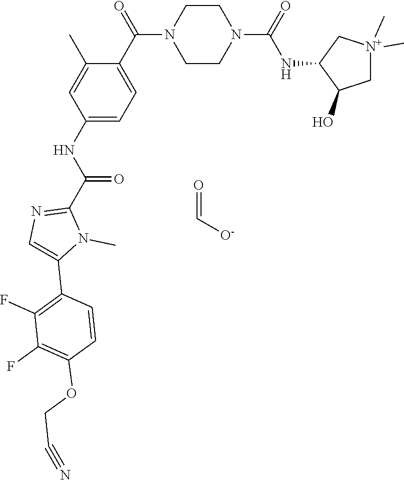 | 651.3 | Intermediate E4 and (3R,4R)-4- amino-1-methyl- pyrrolidin-3-ol and methyl iodide |
| Example B29 | (3S,4S)-3-(4-(4-(5- (4-(cyanomethoxy)- 2,3-difluorophenyl)- 1-methyl-1H- imidazole-2- carboxamido)-2- methylbenzoyl) piperazine-1- carboxamido)-4- hydroxy-1,1- dimethylpyrrolidin- 1-ium formate | 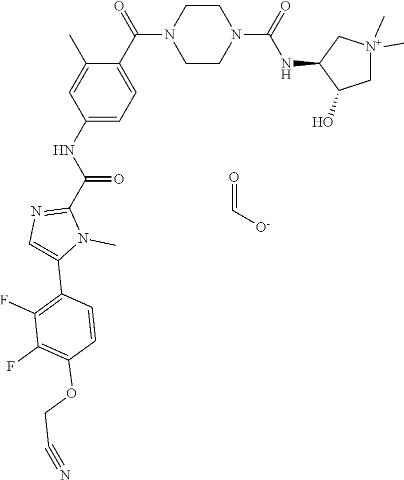 | 651.4 | Intermediate E4 and (3S,4S)-4- amino-1-methyl- pyrrolidin-3-ol and methyl iodide |
| Example B30 | (2S,4R)-2-(4-(2- chloro-4-(5-(2,3- difluoro-4- methoxyphenyl)-1- methyl-1H- imidazole-2- carboxamido) benzoyl)piperazine- 1-carbonyl)-4- hydroxy-1,1,4- trimethylpyrrolidin- 1-ium formate | 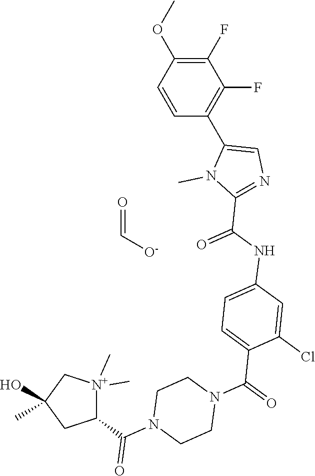 | 645.3 | Intermediate E6 and (2S,4R)-4- hydroxy-1,4- dimethyl- pyrrolidine-2- carboxylic acid and methyl iodide |
| Example B31 | [1-[1-[2-chloro-4- [[5-[4- (cyanomethoxy)-2,3- difluoro-phenyl]-1- methyl-imidazole-2- carbonyl]amino] benzoyl]piperidine- 4-carbonyl]azetidin- 3-yl]-trimethyl- ammonium; formate | 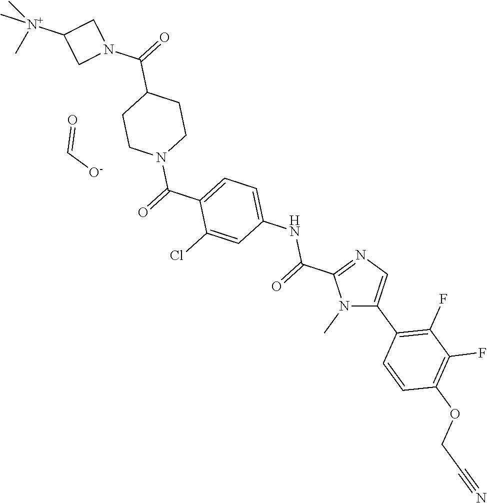 | 654.4 | Intermediate E23 and N,N- dimethylazetidin- 3-amine and methyl iodide |
| Example B32 | N-[3-chloro-4-[1-[2- (4,4- dimethylpiperazin-4- ium-1-yl)-2-oxo- ethyl]pyrazol-4- yl]methylcarbamoyl] phenyl]-5-(2,3- difluoro-4-methoxy- phenyl)-1-methyl- imidazole-2- carboxamide; formate | 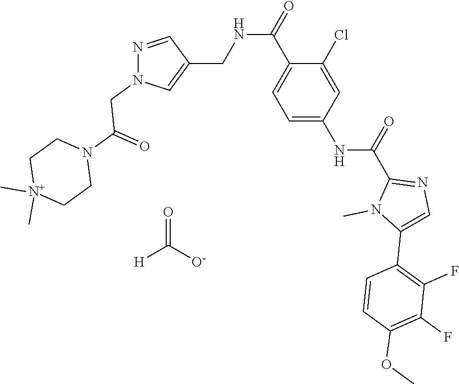 | 655.2 | Intermediate E24 and tert-butyl piperazine-1- carboxylate and methyl iodide |
| Example B33 | N-[3-chloro-4-[4-(3- hydroxy-1,1- dimethyl-piperidin- 1-ium-4- carbonyl)piperazine- 1-carbonyl]phenyl]- 5-(2,3-difluoro-4- methoxy-phenyl)-1- methyl-imidazole-2- carboxamide; formate | 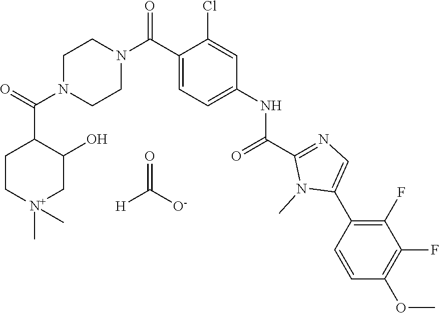 | 645.9 | Intermediate E6 and 1-tert- butoxycarbonyl- 3-hydroxy- piperidine-4- carboxylic acid and methyl iodide |
| Example B34 | N-[3-chloro-4-[4- [(2S,4S)-4-hydroxy- 1,1-dimethyl- pyrrolidin-1-ium-2- carbonyl]piperazine- 1-carbonyl]phenyl]- 5-(2,3-difluoro-4- methoxy-phenyl)-1- methyl-imidazole-2- carboxamide; formate | 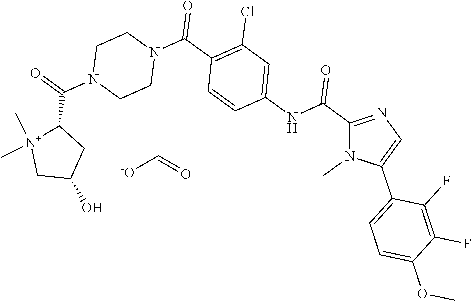 | 631.3 | Intermediate E6, (2S,4S)-4- hydroxy-1- methyl- pyrrolidine-2- carboxylic acid and methyl iodide |
| Example B35 | N-[3-chloro-4-[4- [(3S)-4,4- dimethylmorpholin- 4-ium-3- carbonyl]piperazine- 1-carbonyl]phenyl]- 5-(2,3-difluoro-4- methoxy-phenyl)-1- methyl-imidazole-2- carboxamide; iodide | 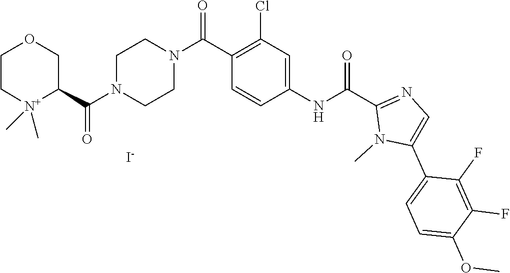 | 631.2 | Intermediate E6, (3S)-morpholine- 3-carboxylic acid and methyl iodide |
| Example B36 | N-[3-chloro-4-[4- [(2S,3S)-3-hydroxy- 1,1-dimethyl- pyrrolidin-1-ium-2- carbonyl]piperazine- 1-carbonyl]phenyl]- 5-(2,3-difluoro-4- methoxy-phenyl)-1- methyl-imidazole-2- carboxamide; formate | 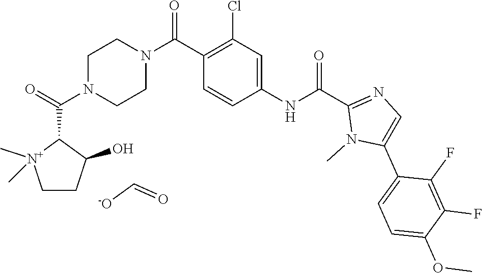 | 631.2 | Intermediate E6, (2S,3S)-3- hydroxy- pyrrolidine-2- carboxylic acid and methyl iodide |
| Example B37 | N-[3-chloro-4-[4- [(2R,4S)-4-hydroxy- 1,1-dimethyl- pyrrolidin-1-ium-2- carbonyl]piperazine- 1-carbonyl]phenyl]- 5-(2,3-difluoro-4- methoxy-phenyl)-1- methyl-imidazole-2- carboxamide; formate | 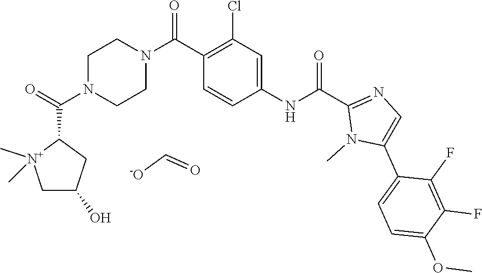 | 631.2 | Intermediate E6, (2R,4S)-4- hydroxy- pyrrolidine-2- carboxylic acid and methyl iodide |
| Example B38 | N-[3-chloro-4-[4- [(2S,4R)-4-hydroxy- 1,1-dimethyl- piperidin-1-ium-2- carbonyl]piperazine- 1-carbonyl]phenyl]- 5-(2,3-difluoro-4- methoxy-phenyl)-1- methyl-imidazole-2- carboxamide; formate | 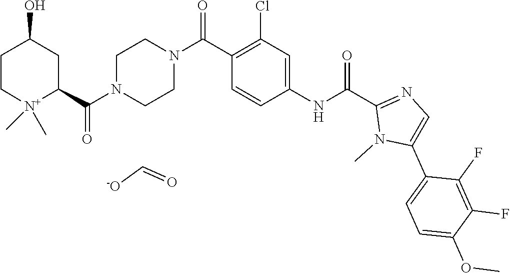 | 645.3 | Intermediate E6, (2S,4R)-4- hydroxy- piperidine-2- carboxylic acid and methyl iodide |
| Example B39 | 4-[2-chloro-4-[[5- (2,3-difluoro-4- methoxy-phenyl)-1- methyl-imidazole-2- carbonyl]amino] benzoyl]-N-(4- hydroxy-1,1- dimethyl-pyrrolidin- 1-ium-3- yl)piperazine-1- carboxamide; formate | 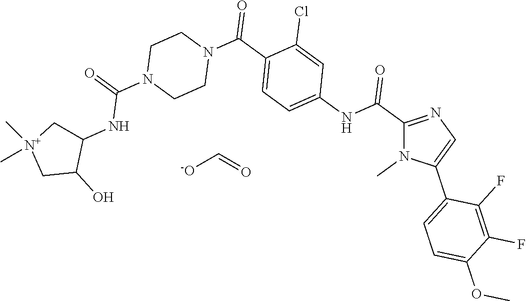 | 631.2 | Intermediate E6, 4-hydroxy- pyrrolidine-2- carboxylic acid and methyl iodide |
| Example B40 | N-[3-chloro-4-[4- [(2S,4R)-4-hydroxy- 1,1-dimethyl- pyrrolidin-1-ium-2- carbonyl]piperazine- 1-carbonyl]phenyl]- 5-[2- (difluoromethyl)-3- fluoro-4-methoxy- phenyl]-1-methyl- imidazole-2- carboxamide; formate | 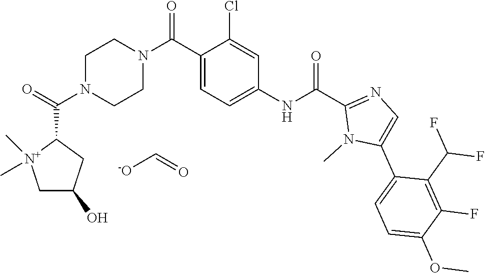 | 663.3 | Intermediate E6, (2S,4R)-4- hydroxy- pyrrolidine-2- carboxylic acid and methyl iodide |
| Example B41 | N-[3-chloro-4-[4- [(2S,4R)-4-hydroxy- 1,1-dimethyl- pyrrolidin-1-ium-2- carbonyl]piperazine- 1-carbonyl]phenyl]- 5-[4- (difluoromethoxy)- 2,3-difluoro- phenyl]-1-methyl- imidazole-2- carboxamide; formate | 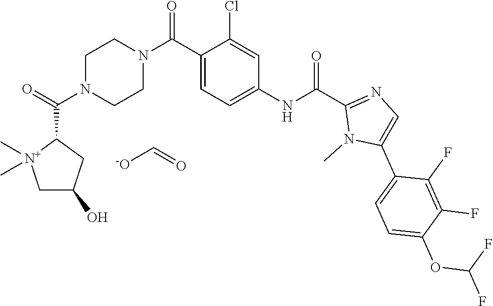 | 667.3 | Intermediate E13, (2S,4R)-4- hydroxy- pyrrolidine-2- carboxylic acid and methyl iodide |
| Example B42 | N-[3-chloro-4-[4- [(2S,4R)-4- (hydroxymethyl)- 1,1-dimethyl- pyrrolidin-1-ium-2- carbonyl]piperazine- 1-carbonyl]phenyl]- 5-(2,3-difluoro-4- methoxy-phenyl)-1- methyl-imidazole-2- carboxamide; formate | 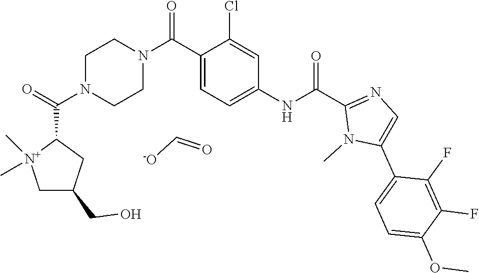 | 645.3 | Intermediate E6, (2S,4R)-4- (hydroxymethyl) pyrrolidine-2- carboxylic acid and methyl iodide |
| Example B43 | 5-(2-chloro-3- fluoro-4-methoxy- phenyl)-N-[3- chloro-4-[4- [(2S,4R)-4-hydroxy- 1,1-dimethyl- pyrrolidin-1-ium-2- carbonyl]piperazine- 1-carbonyl]phenyl]- 1-methyl-imidazole- 2-carboxamide; formate | 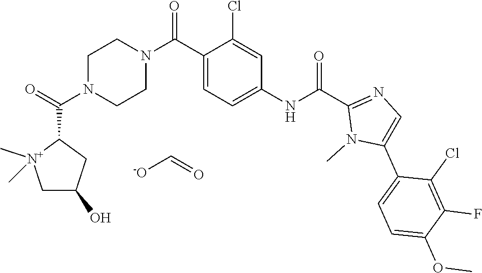 | 647.2 | Intermediate E7, (2S,4R)-4- hydroxy- pyrrolidine-2- carboxylic acid and methyl iodide |
| Example B44 | N-[3-chloro-4-[4- [(2S,3R)-3-hydroxy- 1,1-dimethyl- pyrrolidin-1-ium-2- carbonyl]piperazine- 1-carbonyl]phenyl]- 5-(2,3-difluoro-4- methoxy-phenyl)-1- methyl-imidazole-2- carboxamide; formate | 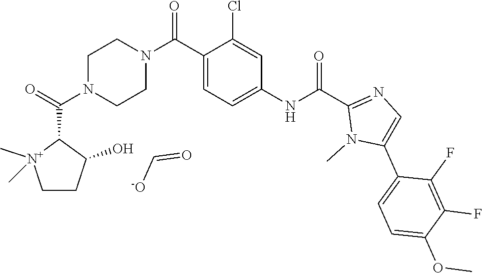 | 631.2 | Intermediate E6, (2S,3R)-3- hydroxy- pyrrolidine-2- carboxylic acid and methyl iodide |
| Example B45 | N-[3-chloro-4-[4- [(2S,4R)-4-hydroxy- 1,1-dimethyl- pyrrolidin-1-ium-2- carbonyl]piperazine- 1-carbonyl]phenyl]- 5-[2,3-difluoro-4- (fluoromethoxy) phenyl]-1-methyl- imidazole-2- carboxamide; formate | 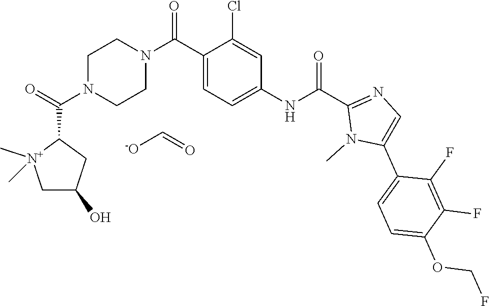 | 649.2 | Intermediate E30, (2S,4R)-4- hydroxy- pyrrolidine-2- carboxylic acid and methyl iodide |
| Example B46 | N-[3-chloro-4-[4- [(2S,5S)-5-hydroxy- 1,1-dimethyl- piperidin-1-ium-2- carbonyl]piperazine- 1-carbonyl]phenyl]- 5-(2,3-difluoro-4- methoxy-phenyl)-1- methyl-imidazole-2- carboxamide; formate | 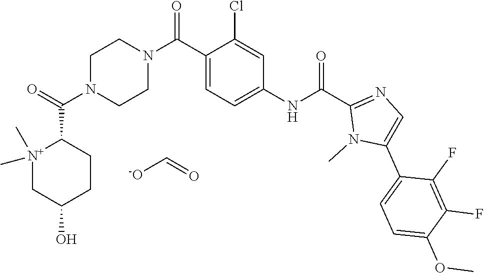 | 645.2 | Intermediate E6, (2S,5S)-5- hydroxy- piperidine-2- carboxylic acid and methyl iodide |
| Example B47 | 5-[3-chloro-4- (difluoromethoxy)- 2-fluoro-phenyl]-N- [3-chloro-4-[4- [(2S,4R)-4-hydroxy- 1,1-dimethyl- pyrrolidin-1-ium-2- carbonyl]piperazine- 1-carbonyl]phenyl]- 1-methyl-imidazole- 2-carboxamide; iodide | 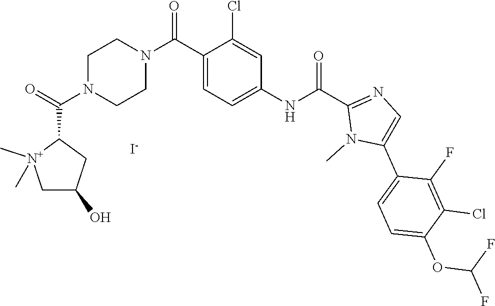 [SMILES: Cn1c(-c2ccc(OC(F)F)c(Cl)c2F)cnc1C(=O)Nc1ccc(C(=O)N2CCN(C(=O)[C@@H]3C[C@@H](O)C[N+]3(C)C)CC2)c(Cl)c1 ; [I-]] | 483.2 | Intermediate E31, (2S,4R)-4- hydroxy- pyrrolidine-2- carboxylic acid and methyl iodide |
| Example B56 | [2-[4-[2-chloro-4- [[5-[2,3-difluoro-4- (fluoromethoxy) phenyl]-1-methyl- imidazole-2- carbonyl]amino] benzoyl]piperazin-1- yl]-2-oxo-ethyl]- trimethyl- ammonium; formate | 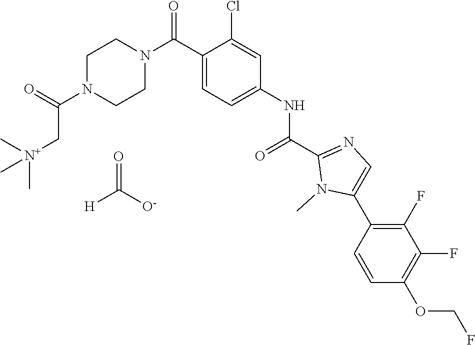 | 607.2 | Intermediate E36 and 2- (dimethylamino) aceticacid and methyl iodide |

### Example B48

### 5-(3-chloro-2-fluoro-4-methoxy-phenyl)-N-[3-chloro-4-[4-[(2S,4R)-4-hydroxy-1,1-dimethyl-pyrrolidin-1-ium-2-carbonyl]piperazine-1-carbonyl]phenyl]-1-methyl-imidazole-2-carboxamide formate

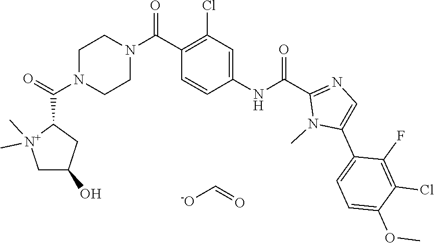

### Step 1: 5-bromo-N-[3-chloro-4-(piperazine-1-carbonyl)phenyl]-1-methyl-imidazole-2-carboxamide

To a mixture of tert-butyl 4-[4-[(5-bromo-1-methyl-imidazole-2-carbonyl)amino]-2-chloro-benzoyl]piperazine-1-carboxylate (1200 mg, 2.28 mmol) in DCM (30 mL) was added trifluoroacetic acid (3.51 mL, 45.56 mmol). The reaction mixture was stirred at 30° C. for 4 h. The reaction mixture was concentrated in vacuum. The residue was purified by prep-HPLC (FA) to the title compound (1000 mg, 2.34 mmol, 79.64% yield) as yellow solid. ESI MS [M+H]⁺: 428.0.

### Step 2: tert-butyl (2S,4R)-2-[4-[4-[(5-bromo-1-methyl-imidazole-2-carbonyl)amino]-2-chloro-benzoyl]piperazine-1-carbonyl]-4-hydroxy-pyrrolidine-1-carboxylate

To a mixture of BOC—HYP—OH (379 mg, 1.64 mmol) and 5-bromo-N-[3-chloro-4-(piperazine-1-carbonyl)phenyl]-1-methyl-imidazole-2-carboxamide (700 mg, 1.64 mmol) in THE (10 mL) was added N,N-diisopropylethylamine (635 mg, 4.92 mmol) and propylphosphonic anhydride (1357 mg, 2.13 mmol). The reaction mixture was stirred at 30° C. for 4 h. The reaction mixture was concentrated in vacuum and the residue was purified by prep-HPLC (FA) to afford the title compound (350 mg, 0.550 mmol, 33.34% yield) as yellow solid. ESI MS [M+H]⁺: 641.1.

### Step 3: tert-butyl (2S,4R)-2-[4-[2-chloro-4-[[5-(3-chloro-2-fluoro-4-methoxy-phenyl)-1-methyl-imidazole-2-carbonyl]amino]benzoyl]piperazine-1-carbonyl]-4-hydroxy-pyrrolidine-1-carboxylate

To a mixture of (3-chloro-2-fluoro-4-methoxy-phenyl)boronic acid (192 mg, 0.940 mmol) and tert-butyl (2S,4R)-2-[4-[4-[(5-bromo-1-methyl-imidazole-2-carbonyl)amino]-2-chloro-benzoyl]piperazine-1-carbonyl]-4-hydroxy-pyrrolidine-1-carboxylate (300 mg, 0.470 mmol) in 1,4-dioxane (10 mL) was added [1,1′-bis(diphenylphosphino)ferrocene]dichloropalladium(II) (34.3 mg, 0.050 mmol) and sodium carbonate (99 mg, 0.940 mmol). The reaction mixture was stirred at 85° C. for 16 h. The mixture was concentrated to remove solvent, purified by silica column (DCM/MeOH=50:1 to 20:1) and reversed phase-HPLC (FA) to afford the title compound (150 mg, 0.210 mmol, 44.46% yield) as white solid. ESI MS [M+H]⁺: 619.2.

### Step 4+5: 5-(3-chloro-2-fluoro-4-methoxy-phenyl)-N-[3-chloro-4-[4-[(2S,4R)-4-hydroxy-1,1-dimethyl-pyrrolidin-1-ium-2-carbonyl]piperazine-1-carbonyl]phenyl]-1-methyl-imidazole-2-carboxamide formate

The title compound was prepared in analogy to Example B54 steps 2 and 3 from tert-butyl (2S,4R)-2-[4-[2-chloro-4-[[5-(3-chloro-2-fluoro-4-methoxy-phenyl)-1-methyl-imidazole-2-carbonyl]amino]benzoyl]piperazine-1-carbonyl]-4-hydroxy-pyrrolidine-1-carboxylate and was obtained as white solid (38.8 mg). ESI MS [M+H]⁺: 647.2.

The following Type II Examples were prepared in analogy to Example B48:

| | | | MS | |
|---|---|---|---|---|
| | | | ESI | |
| | Name | Structure | [M + H]⁺ | Starting Material |
| Example B51 | N-[3-chloro-4- [4-[(2S,4R)-4- hydroxy-1,1- dimethyl-pyr- rolidin-1-ium- 2-carbonyl]- piperazine-1- carbonyl]phen- yl]-5-(3-cyano- 2-fluoro-4- methoxy-phen- yl)-1-methyl- imidazole-2- carboxamide; | 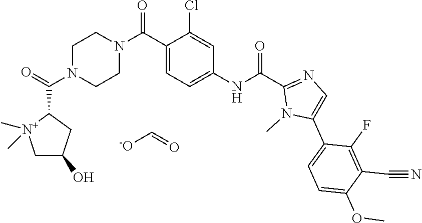 | 638.1 | (3-cyano-2-fluoro- 4-methoxy-phenyl)- boronic acid |
| | formate | | | |
| Example B52 | N-[3-chloro-4- [4-[(2S,4R)-4- hydroxy-1,1- dimethyl-pyr- rolidin-1-ium- 2-carbonyl]- piperazine-1- carbonyl]- phenyl]-5-[2- fluoro-4-meth- oxy-3-(tri- fluoromethyl)- phenyl]-1- methyl-imid- | 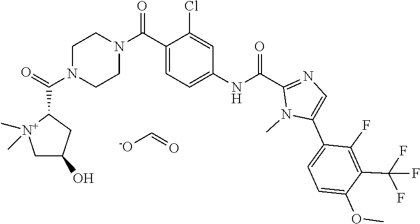 | 681.1 | [2-fluoro-4-meth- oxy-3-(trifluoro- methyl)phenyl]- boronic acid |
| | azole-2-carbox- | | | |
| | amide; formate | | | |

### Example B49

### N-[3-chloro-4-[4-[(2S)-1,1-dimethyl-2,5-dihydropyrrol-1-ium-2-carbonyl]piperazine-1-carbonyl]phenyl]-5-(2,3-difluoro-4-methoxy-phenyl)-1-methyl-imidazole-2-carboxamide formate

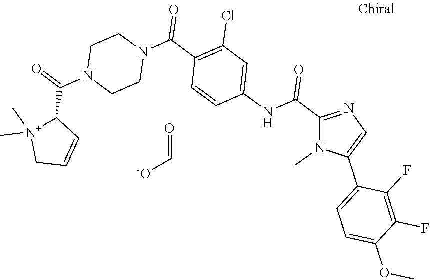

### Step: 1 tert-butyl (2S)-2-[4-[2-chloro-4-[[5-(2,3-difluoro-4-methoxy-phenyl)-1-methyl-imidazole-2-carbonyl]amino]benzoyl]piperazine-1-carbonyl]-2,5-dihydropyrrole-1-carboxylate

A mixture of N-[3-chloro-4-(piperazine-1-carbonyl)phenyl]-5-(2,3-difluoro-4-methoxy-phenyl)-1-methyl-imidazole-2-carboxamide hydrochloride (0.5 g, 0.950 mmol), (2S)-1-tert-butoxycarbonyl-2,5-dihydropyrrole-2-carboxylic acid (243.06 mg, 1.14 mmol), triethylamine (0.4 mL, 2.85 mmol) and propylphosphonic anhydride (0.73 mL, 1.23 mmol) in DMF (5 mL) was stirred at 20° C. for 16 h. The mixture was purified by reversed phase-HPLC to afford the title compound (420 mg, 0.610 mmol, 64.54% yield) as light yellow solid.

### Step 2: N-[3-chloro-4-[4-[(2S)-2,5-dihydro-1H-pyrrole-2-carbonyl]piperazine-1-carbonyl]phenyl]-5-(2,3-difluoro-4-methoxy-phenyl)-1-methyl-imidazole-2-carboxamide 2,2,2-trifluoroacetate

A solution of tert-butyl rac-(2S)-2-[4-[2-chloro-4-[[5-(2,3-difluoro-4-methoxy-phenyl)-1-methyl-imidazole-2-carbonyl]amino]benzoyl]piperazine-1-carbonyl]-2,5-dihydropyrrole-1-carboxylate (100.0 mg, 0.150 mmol) in DCM (3 mL) was added trifluoroacetic acid (1.0 mL, 12.98 mmol) and stirred at 20° C. for 16 h. The mixture was concentrated to afford the crude title compound (50 mg, 0.070 mmol, 49.01% yield) as light yellow oil, which used for next step without purification.

### Step 3: N-[3-chloro-4-[4-[(2S)-1,1-dimethyl-2,5-dihydropyrrol-1-ium-2-carbonyl]piperazine-1-carbonyl]phenyl]-5-(2,3-difluoro-4-methoxy-phenyl)-1-methyl-imidazole-2-carboxamide formate

A solution of N-[3-chloro-4-[4-[(2S)-2,5-dihydro-1H-pyrrole-2-carbonyl]piperazine-1-carbonyl]phenyl]-5-(2,3-difluoro-4-methoxy-phenyl)-1-methyl-imidazole-2-carboxamide;2,2,2-trifluoroacetic acid (50.0 mg, 0.070 mmol) and triethylamine (0.1 mL, 0.720 mmol) in ACN (3 mL)/water (0.30 mL) was added iodomethane (0.07 mL, 0.720 mmol) and stirred at 20° C. for 16 h. The mixture was concentrated and purified by prep-HPLC (FA) to afford the title compound (19.1 mg, 0.030 mmol, 32.17% yield) as white solid. ESI MS [M+H]⁺: 613.1.

### Example B50

### N-[3-chloro-4-[4-[(2S,3R,4S)-3,4-dihydroxy-1,1-dimethyl-pyrrolidin-1-ium-2-carbonyl]piperazine-1-carbonyl]phenyl]-5-(2,3-difluoro-4-methoxy-phenyl)-1-methyl-imidazole-2-carboxamide formate

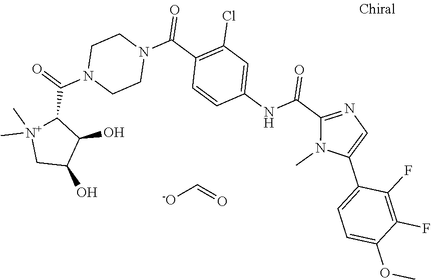

### Step 1: tert-butyl (2S,3R,4S)-2-[4-[2-chloro-4-[[5-(2,3-difluoro-4-methoxy-phenyl)-1-methyl-imidazole-2-carbonyl]amino]benzoyl]piperazine-1-carbonyl]-3,4-dihydroxy-pyrrolidine-1-carboxylate

A mixture of tert-butyl (2S)-2-[4-[2-chloro-4-[[5-(2,3-difluoro-4-methoxy-phenyl)-1-methyl-imidazole-2-carbonyl]amino]benzoyl]piperazine-1-carbonyl]-2,5-dihydropyrrole-1-carboxylate (0.3 g, 0.440 mmol), dioxo(dipotassiooxy)osmium dihydrate (16.13 mg, 0.040 mmol), 4-methylmorpholine N-oxide (0.14 mL, 1.31 mmol, 3 eq) and in tert-butanol (5 mL) was stirred at 0° C. for 16 h. The mixture was purified by reversed phase-HPLC (FA) to afford the title compound (50 mg, 0.070 mmol, 15.88% yield) as light yellow solid. ESI MS [M+H]⁺: 719.3.

### Step 2: N-[3-chloro-4-[4-[(2S,3R,4S)-3,4-dihydroxypyrrolidine-2-carbonyl]piperazine-1-carbonyl]phenyl]-5-(2,3-difluoro-4-methoxy-phenyl)-1-methyl-imidazole-2-carboxamide

A solution of tert-butyl (2S,3R,4S)-2-[4-[2-chloro-4-[[5-(2,3-difluoro-4-methoxy-phenyl)-1-methyl-imidazole-2-carbonyl]amino]benzoyl]piperazine-1-carbonyl]-3,4-dihydroxy-pyrrolidine-1-carboxylate (60.0 mg, 0.080 mmol) in methanol (5 mL) was added hydrochloric acid in MeOH (3.0 mL, 12 mmol) and stirred at 20° C. for 16 h. The mixture was concentrated and purified by reversed phase-HPLC (FA) to afford the title compound (45 mg, 0.070 mmol, 87.13% yield) as light yellow solid. ESI MS [M+H]⁺: 619.2.

### Step 3: N-[3-chloro-4-[4-[(2S,3R,4S)-3,4-dihydroxy-1,1-dimethyl-pyrrolidin-1-ium-2-carbonyl]piperazine-1-carbonyl]phenyl]-5-(2,3-difluoro-4-methoxy-phenyl)-1-methyl-imidazole-2-carboxamide formate

To a solution of N-[3-chloro-4-[4-[(2S,3R,4S)-3,4-dihydroxypyrrolidine-2-carbonyl]piperazine-1-carbonyl]phenyl]-5-(2,3-difluoro-4-methoxy-phenyl)-1-methyl-imidazole-2-carboxamide;hydrochloride (45.0 mg, 0.070 mmol) and triethylamine (0.05 mL, 0.340 mmol) in ACN (5 mL)/Water (0.500 mL) was added iodomethane (0.1 g, 0.690 mmol) at 25° C. and stirred for 16 h at 25° C. The mixture was concentrated and purified by prep-HPLC (FA) to afford the title compound (22.8 mg, 0.030 mmol, 46.05% yield) as light yellow solid. ESI MS [M+H]⁺: 647.2.

### Example B54

### N-[3-chloro-4-[[(exo)-3-[(2S,4R)-4-hydroxy-1,1-dimethyl-pyrrolidin-1-ium-2-carbonyl]-3-azabicyclo[3.1.0]hexan-6-yl]carbamoyl]phenyl]-5-(2,3-difluoro-4-methoxy-phenyl)-1-methyl-imidazole-2-carboxamide formate

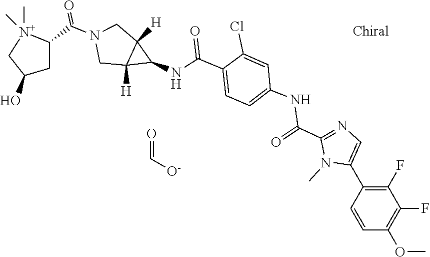

### Step 1: (2S,4R)-2-[(exo)-6-[[2-chloro-4-[[5-(2,3-difluoro-4-methoxy-phenyl)-1-methyl-imidazole-2-carbonyl]amino]benzoyl]amino]-3-azabicyclo[3.1.0]hexane-3-carbonyl]-4-hydroxy-pyrrolidine-1-carboxylic acid tert-butyl ester

In a 10 ml round-bottomed flask (2S,4R)-1-tert-butoxycarbonyl-4-hydroxy-proline (25 mg, 0.109 mmol, 1.5 eq) and N-[4-[[(exo)-3-azabicyclo[3.1.0]hexan-6-yl]carbamoyl]-3-chloro-phenyl]-5-(2,3-difluoro-4-methoxy-phenyl)-1-methyl-imidazole-2-carboxamide hydrochloride (50 mg, 0.072 mmol) were combined with N,N-dimethylformamide (1 mL) to give a light brown solution. N,N-diisopropylethylamine (47 mg, 63 μL, 0.362 mmol) and HATU (41 mg, 0.109 mmol) were added, and the reaction mixture was stirred at RT for 1.5 hours. Water was added to the reaction mixture. The mixture was extracted with DCM and the organic layer was then washed twice with a 5% LiCl solution, brine and dried over Na2SO4. After filtration and evaporation of the volatiles, the residue was purified by column chromatography on silica gel using DCM:MeOH as eluent to give the title compound (28.4 mg, 50.98%) as white solid. ESI MS [M+H]⁺: 715.2.

### Step 2: N-[3-chloro-4-[[(exo)-3-[(2S,4R)-4-hydroxyprolyl]-3-azabicyclo[3.1.0]hexan-6-yl]carbamoyl]phenyl]-5-(2,3-difluoro-4-methoxy-phenyl)-1-methyl-imidazole-2-carboxamide hydrochloride

In a 10 ml round-bottomed flask, (2S,4R)-2-[(exo)-6-[[2-chloro-4-[[5-(2,3-difluoro-4-methoxy-phenyl)-1-methyl-imidazole-2-carbonyl]amino]benzoyl]amino]-3-azabicyclo[3.1.0]hexane-3-carbonyl]-4-hydroxy-pyrrolidine-1-carboxylic acid tert-butyl ester (28.4 mg, 0.037 mmol) was combined with dichloromethane (300 uL) to give a light brown solution. 4 M HCl in dioxane (55.4 mg, 46.17 uL, 0.185 mmol) was added, and the reaction mixture was stirred at RT for 1.5 hours. The reaction mixture was concentrated to dryness to afford the title compound (31.7 mg, quant) as off-white solid and was used without further purification. ESI MS [M+H]⁺: 615.2.

### Step 3: N-[3-chloro-4-[(exo)-3-[(2 S,4R)-4-hydroxy-1,1-dimethyl-pyrrolidin-1-ium-2-carbonyl]-3-azabicyclo[3.1.01 hexan-6-yl]carbamoyl]phenyl]-5-(2,3-difluoro-4-methoxy-phenyl)-1-methyl-imidazole-2-carboxamide formate

In a 5 ml round-bottomed flask, N-[3-chloro-4-[[(exo)-3-[(2S,4R)-4-hydroxyprolyl]-3-azabicyclo[3.1.0]hexan-6-yl]carbamoyl]phenyl]-5-(2,3-difluoro-4-methoxy-phenyl)-1-methyl-imidazole-2-carboxamide hydrochloride (25.3 mg, 0.035 mmol) was combined with acetonitrile (0.500 mL) to give a white suspension. DIPA (13.7 mg, 18.52 uL, 0.106 mmol) and iodomethane (12.5 mg, 5.52 uL, 0.088 mmol) were added, and the reaction mixture was stirred at for 1 h. After concentration to dryness, the crude material was purified by prep HPLC to give the title compound (12.2 mg, 49.6%) as white lyoph solid. ESI MS [M+H]⁺: 643.2.

The following Type 11 Examples were prepared in analogy to Example B48:

| | | | MS | |
|---|---|---|---|---|
| | | | ESI | |
| | Name | Structure | [M + H]⁺ | Starting Material |
| Example B53 | 5-[2-chloro-4- (difluorometh- oxy)-3-fluoro- phenyl]-N-[3- chloro-4-[4- [(2S,4R)-4- hydroxy-1,1- dimethyl- pyrrolidin-1- ium-2-carbon- yl]piperazine- 1-carbonyl]- phenyl]-1- methyl-imid- azole-2-carbox- amide; formate | 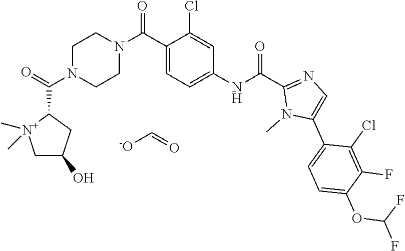 | 683.2 | Intermediate E13 |

### Example B55

### (exo)-6-[[2-chloro-4-[[5-(2,3-difluoro-4-methoxy-phenyl)-1-methyl-imidazole-2-carbonyl]amino]benzoyl]amino]-N-[(trans)-4-hydroxy-1,1-dimethyl-pyrrolidin-1-ium-3-yl]-3-azabicyclo[3.1.0]hexane-3-carboxamide formate

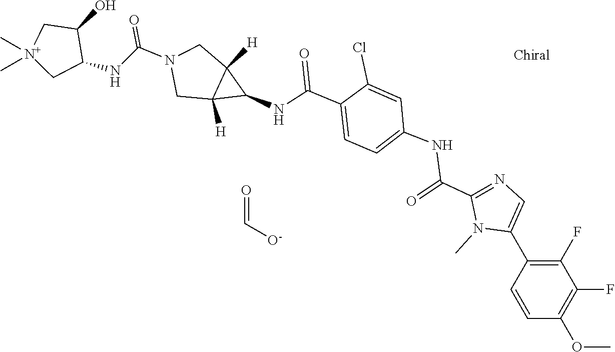

### Step 1: (trans)-3-[[(exo)-6-[[2-chloro-4-[[5-(2,3-difluoro-4-methoxy-phenyl)-1-methyl-imidazole-2-carbonyl]amino]benzoyl]amino]-3-azabicyclo[3.1.0]hexane-3-carbonyl]amino]-4-hydroxy-pyrrolidine-1-carboxylic acid tert-butyl ester

To a solution of (trans)-3-amino-4-hydroxy-pyrrolidine-1-carboxylic acid tert-butyl ester (44 mg, 0.217 mmol) in N,N-dimethylformamide were added triethylamine (37 mg, 50.48 uL, 0.362 mmol) and 1,1′-carbonyldiimidazole (29 mg, 0.181 mmol) and the reaction mixture was stirred at RT for 20 min. Then N-[4-[[(exo)-3-azabicyclo[3.1.0]hexan-6-yl]carbamoyl]-3-chloro-phenyl]-5-(2,3-difluoro-4-methoxy-phenyl)-1-methyl-imidazole-2-carboxamide hydrochloride (50 mg, 0.072 mmol, 1 eq) was added and the stirring was continued at RT for 1.5 hours. Water was added to the reaction mixture. The mixture was extracted with DCM and the organic layer was then washed twice with a 5% LiCl solution, brine and dried over Na₂SO₄. After filtration and evaporation of the volatiles, the residue was purified by column chromatography on silica gel using DCM:MeOH as eluent) to give the title compound (51.1 mg, 87.91%) as white solid. ESI MS [M+H]⁺:730.3.

### Step 2: (exo)-6-[[2-chloro-4-[[5-(2,3-difluoro-4-methoxy-phenyl)-1-methyl-imidazole-2-carbonyl]amino]benzoyl]amino]-N-[(trans)-4-hydroxy-1,1-dimethyl-pyrrolidin-1-ium-3-yl]-3-azabicyclo[3.1.0]hexane-3-carboxamide formate

The title compound was prepared in analogy to Example B54, steps 2, 3 and was obtained as white solid (5.2 mg). ESI MS [M+H]⁺: 658.2.

### Example C1

### N-[4-[4-[1-(2-Amino-2-oxo-ethyl)-1-methyl-piperidin-1-ium-4-carbonyl]piperazine-1-carbonyl]-3-chloro-phenyl]-5-[2-chloro-4-(difluoromethoxy)-3-fluoro-phenyl]-1-methyl-imidazole-2-carboxamide;formate

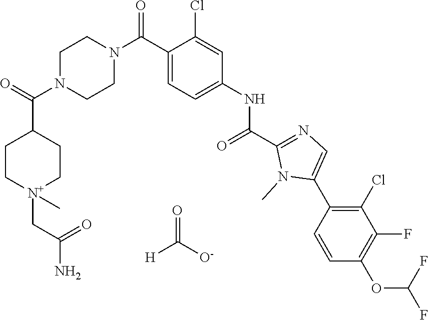

### Step 1: tert-butyl 4-[4-[2-chloro-4-[[5-[2-chloro-4-(difluoromethoxy)-3-fluoro-phenyl]-1-methyl-imidazole-2-carbonyl]amino]benzoyl]piperazine-1-carbonyl]piperidine-1-carboxylate

In a 100 mL round-bottomed flask, 5-(2-chloro-4-(difluoromethoxy)-3-fluorophenyl)-N-(3-chloro-4-(piperazine-1-carbonyl)phenyl)-1-methyl-1H-imidazole-2-carboxamide (100 mg, 184 μmol), 1-(tert-butoxycarbonyl)piperidine-4-carboxylic acid (59.2 mg, 258 μmol), DIEA (71.5 mg, 96.6 μl, 553 μmol) and 2-(3H-[1,2,3]triazolo[4,5-b]pyridin-3-yl)-1,1,3,3-tetramethylisouronium hexafluorophosphate(V) (98.2 mg, 258 μmol) were combined with DMF (4 mL) to give a light brown solution. The reaction was stirred at room temperature for 30 min. The crude reaction mixture was concentrated in vacuum. The crude product was directly used to the next step, to afford tert-butyl 4-(4-(2-chloro-4-(5-(2-chloro-4-(difluoromethoxy)-3-fluorophenyl)-1-methyl-1H-imidazole-2carboxamido)benzoyl)piperazine-1-carbonyl)piperidine-1-carboxylate (138 mg, 183 μmol, 99% yield). ESI MS [M+H]⁺: 653.1.

### Step 2: 5-[2-chloro-4-(difluoromethoxy)-3-fluoro-phenyl]-N-[3-chloro-4-[4-(piperidine-4-carbonyl)piperazine-1-carbonyl]phenyl]-1-methyl-imidazole-2-carboxamide

In a 100 mL round-bottomed flask, tert-butyl 4-(4-(2-chloro-4-(5-(2-chloro-4-(difluoromethoxy)-3-fluorophenyl)-1-methyl-1H-imidazole-2carboxamido)benzoyl)piperazine-1-carbonyl)piperidine-1-carboxylate (138 mg, 183 μmol) was combined with THE (3 mL) to give a light brown solution. HCl (1.22 mL, 14.6 mmol) was added. The reaction was stirred at room temperature for 30 min. The crude reaction mixture was concentrated in vacuo. the crude product was directly used to the next step, to afford N-(3-chloro-4-(4-(piperidine-4-carbonyl)piperazine-1-carbonyl)phenyl)-5-(2-chloro-4-(difluoromethoxy)-3-fluorophenyl)-1-methyl-1H-imidazole-2-carboxamide (120 mg, 184 μmol, 100% yield). ESI MS [M+H]⁺: 655.1.

### Step 3: 5-[2-chloro-4-(difluoromethoxy)-3-fluoro-phenyl]-N-[3-chloro-4-[4-(1-methylpiperidine-4-carbonyl)piperazine-1-carbonyl]phenyl]-1-methyl-imidazole-2-carboxamide

In a 100 mL round-bottomed flask, N-(3-chloro-4-(4-(piperidine-4-carbonyl)piperazine-1-carbonyl)phenyl)-5-(2-chloro-4-(difluoromethoxy)-3-fluorophenyl)-1-methyl-1H-imidazole-2-carboxamide (120 mg, 184 μmol), formaldehyde (59.6 mg, 735 μmol) and sodium cyanoborohydride (69.2 mg, 1.1 mmol) were combined with MeOH (8 mL) to give a light brown solution. The reaction was stirred at room temperature for overnight. The reaction mixture was poured into 25 mL sat NaHCO₃ and extracted with EtOAc (3×75 mL). The organic layers were combined, washed with sat NaCl (1×25 mL), The crude reaction mixture was concentrated in vacuum. The crude product was directly used to the next step, to afford N-(3-chloro-4-(4-(1-methylpiperidine-4-carbonyl)piperazine-1-carbonyl)phenyl)-5-(2-chloro-4-(difluoromethoxy)-3-fluorophenyl)-1-methyl-1H-imidazole-2-carboxamide (103 mg, 154 μmol, 84% yield). ESI MS [M+H]⁺: 669.0.

### Step 4: N-[4-[4-[1-(2-amino-2-oxo-ethyl)-1-methyl-piperidin-1-ium-4-carbonyl]piperazine-1-carbonyl]-3-chloro-phenyl]-5-[2-chloro-4-(difluoromethoxy)-3-fluoro-phenyl]-1-methyl-imidazole-2-carboxamide;formate

In a 100 mL round-bottomed flask, N-(3-chloro-4-(4-(1-methylpiperidine-4-carbonyl) piperazine-1-carbonyl)phenyl)-5-(2-chloro-4-(difluoromethoxy)-3-fluorophenyl)-1-methyl-1H-imidazole-2-carboxamide (103 mg, 154 μmol), 2-iodoacetamide (114 mg, 617 μmol) and DIEA (79.8 mg, 108 μl, 617 μmol) were combined with EtOH (5 mL) to give a light brown solution. The reaction was stirred at room temperature for 3 h. The crude reaction mixture was concentrated in vacuum. The crude material was purified by preparative HPLC to afford N-[4-[4-[1-(2-amino-2-oxo-ethyl)-1-methyl-piperidin-1-ium-4-carbonyl]piperazine-1-carbonyl]-3-chloro-phenyl]-5-[2-chloro-4-(difluoromethoxy)-3-fluoro-phenyl]-1-methyl-imidazole-2-carboxamide;formate (24 mg, 30.4 μmol, 20% yield). ESI MS [M+H]⁺: 725.7.

The following Type III Examples were prepared in analogy to Example C1:

| | | | MS | |
|---|---|---|---|---|
| | | | ESI | |
| Int. | Name | Structure | [M + H]⁺ | Starting Material |
| Example C2 | N-[4-[4-[1-(2- amino-2-oxo- ethyl)-1-methyl- piperidin-1-ium- 4-carbonyl]- piperazine-1- carbonyl]-3- chloro-phenyl]- 5-[4-(difluoro- methoxy)-2,3- difluoro-phen- yl]-1-methyl- imidazole-2- carboxamide; formate | 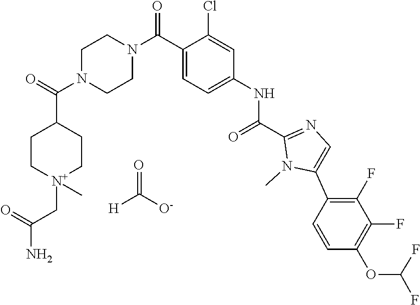 | 708.2 | Intermediate E13 and 1-tert-butoxy- carbonylpiperidine- 4-carboxylic acid and 2-iodoacet- amide |
| Example C3 | N-[4-[4-[1-(2- amino-2-oxo- ethyl)-1-methyl- piperidin-1-ium- 4-carbonyl]- piperazine-1- carbonyl]-3- chloro-phenyl]- 5-(2-chloro-3- fluoro-4-meth- oxy-phenyl)-1- methyl-imid- azole-2-carbox- amide; formate | 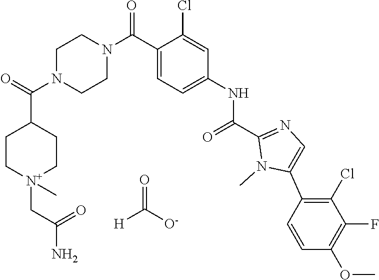 | 688.2 | Intermediate E7 and 1-tert-butoxy- carbonylpiperidine- 4-carboxylic acid and 2-iodoacet- amide |
| Example C4 | N-[4-[4- [(1R,5S)-3-(2- amino-2-oxo- ethyl)-3-methyl- 3-azoniabi- cyclo[3.1.0]- hexane-6- carbonyl]- piperazine-1- carbonyl]-3- chloro-phenyl]- 5-[4-(difluoro- methoxy)-2,3- difluoro-phen- yl]-1-methyl- imidazole-2- carboxamide; formate | 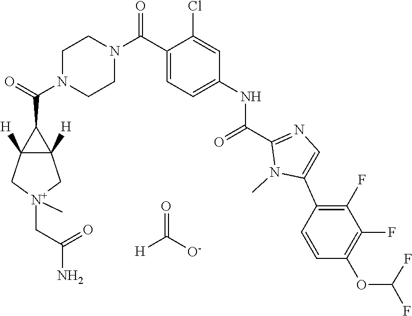 | 706.1 | Intermediate E12 and rac-(1R,5S)-3- tert-butoxycarbon- yl-3-azabicyclo- [3.1.0]hexane-6- carboxylic acid and 2-iodoacet- amide |
| Example C5 | N-[4-[4-[1-(2- amino-2-oxo- ethyl)-1-methyl- piperidin-1-ium- 4-carbonyl]- piperazine-1- carbonyl]-3- chloro-phenyl]- 5-(2,3-difluoro- 4-methoxy- phenyl)-1-meth- yl-imidazole-2- carboxamide; formate | 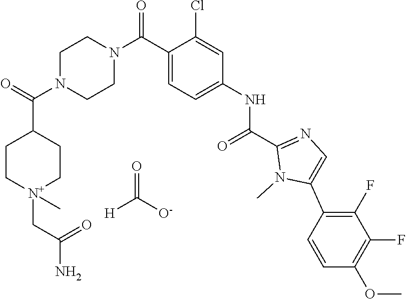 | 672.5 | Intermediate E6 and 1-tert-butoxy- carbonylpiperi- dine-4-carboxylic acid and 2-iodo- acetamide |
| Example C6 | [2-[4-[4-[2- chloro-4-[5- (2,3-difluoro-4- methoxy-phen- yl)-1-methyl- imidazole-2- carbonyl]amino]- benzoyl]piper- azine-1-carbon- yl]-1-piperidyl]- 2-oxo-ethyl]- trimethyl- ammonium; formate | 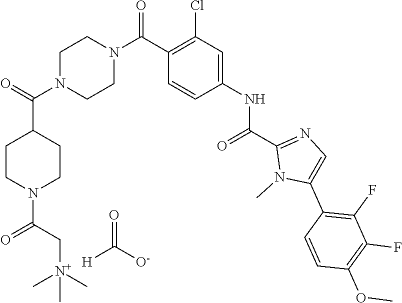 | 700.5 | Intermediate E6 and 1-tert-butoxy- carbonylpiperi- dine-4-carboxylic acid and 2- (dimethylamino)- acetic acid and methyl iodide |
| Example C7 | (2-amino-2-oxo- ethyl)-[2-[4-[4- [2-chloro-4- [[5-(2,3-difluoro- 4-methoxy- phenyl)-1-meth- yl-imidazole-2- carbonyl]amino]- benzoyl]piper- azine-1-carbon- yl]-1-piperidyl]- 2-oxo-ethyl]- dimethyl-ammo- nium; formate | 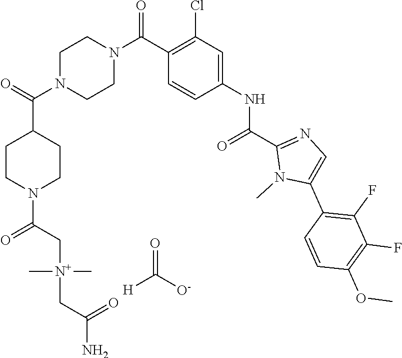 | 743.5 | Intermediate E6 and 1-tert-butoxy- carbonylpiperi- dine-4-carboxylic acid and 2- (dimethylamino)- acetic acid and 2- iodoacetamide |
| Example C8 | 2-[4-[4-[2- chloro-4-[[5- (2,3-difluoro-4- methoxy-phen- yl)-1-methyl- imidazole-2- carbonyl]amino]- benzoyl]piper- azine-1-carbon- yl]-1-methyl- piperidin-1-ium- 1-yl]acetic acid; formate | 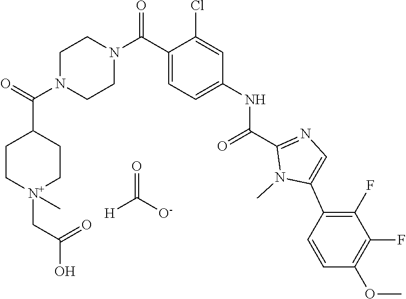 | 673.2 | Intermediate E6 and 1-tert-butoxy- carbonylpiperi- dine-4-carboxylic acid and tert-butyl 2-bromoacetate; HCl |
| Example C9 | N-[4-[4-[1,1- bis(2-amino-2- oxo-ethyl)piper- idin-1-ium-4- carbonyl]piper- azine-1-carbon- yl]-3-chloro- phenyl]-5-[2- chloro-4- (difluorometh- oxy)-3-fluoro- phenyl]-1- methyl-imid- azole-2-carbox- amide; formate | 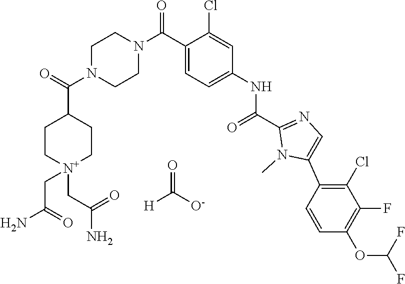 | 769.7 | Intermediate E12 and 1-tert-butoxy- carbonylpiperi- dine-4-carboxylic acid and 2-iodo- acetamide |
| Example C10 | N-[4-[4-[1-(2- amino-2-oxo- ethyl)-1-azetidin- 3-ylmethyl)- piperidin-1-ium- 4-carbonyl]- piperazine-1- carbonyl]-3- chloro-phenyl]- 5-[2-chloro-4- (difluorometh- oxy)-3-fluoro- phenyl]-1-meth- yl-imidazole-2- carboxamide; 2,2,2-trifluoro- acetate; 2,2,2- | 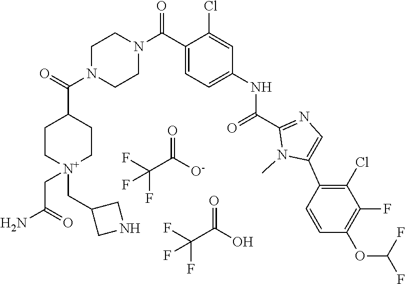 [SMILES: Cn1c(-c2ccc(OC(F)F)c(F)c2Cl)cnc1C(=O)Nc1ccc(C(=O)N2CCN(C(=O)C3CC[N+](CC(N)=O)(CC4CNC4)CC3)CC2)c(Cl)c1 ; O=C([O-])C(F)(F)F ; O=C(O)C(F)(F)F] | 781.6 | Intermediate E12 and 1-tert-butoxy- carbonylpiperi- dine-4-carboxylic acid; tert-butyl 3- formylazetidine-1- carboxylate and 2- iodoacetamide |
| | trifluoroacetic | | | |
| | acid | | | |
| Example C30 | 2-[(1R,5S)-6-[4- [2-chloro-4-[[5- [2,3-difluoro-4- (fluoromethoxy) phenyl]-1-meth- yl-imidazole-2- carbonyl]amino]- benzoyl]piper- azine-1-carbon- yl]-3-methyl-3- azoniabicyclo- [3.1.0]hexan-3- yl]acetic acid; 2,2,2-trifluoro- acetate | 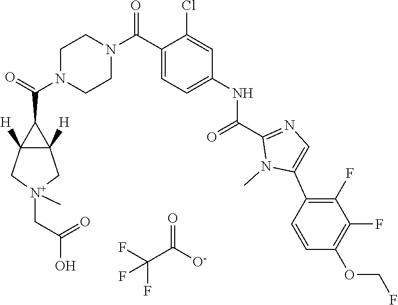 [SMILES: Cn1c(-c2ccc(OCF)c(F)c2F)cnc1C(=O)Nc1ccc(C(=O)N2CCN(C(=O)[C@H]3[C@@H]4C[N+](C)(CC(=O)O)C[C@@H]43)CC2)c(Cl)c1 ; O=C([O-])C(F)(F)F] | 689.4 | Intermediate E30 and rac-(1R,5S)-3- tert-butoxycarbon- yl-3-azabicyclo- [3.1.0]hexane-6- carboxylic acid and tert-butyl 2- bromoacetate |
| Example C31 | 2-[4-[4-[2- chloro-4-[[5-[4- (difluorometh- oxy)-2,3- difluoro-phen- yl]-1-methyl- imidazole-2- carbonyl]amino]- benzoyl]piper- azine-1-carbon- yl]-1-methyl- piperidin-1-ium- 1-yl]acetic acid; 2,2,2-trifluoro- acetate | 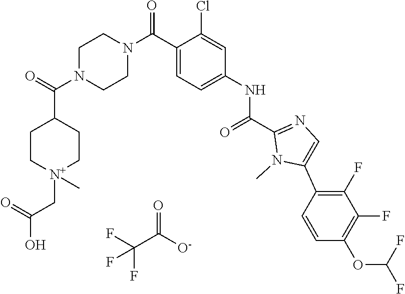 [SMILES: Cn1c(-c2ccc(OC(F)F)c(F)c2F)cnc1C(=O)Nc1ccc(C(=O)N2CCN(C(=O)C3CC[N+](C)(CC(=O)O)CC3)CC2)c(Cl)c1 ; O=C([O-])C(F)(F)F] | 709.7 | Intermediate E13 and 1-tert-butoxy- carbonylpiperidine- 4-carboxylic acid and tert-butyl 2- bromoacetate |

### Example C11

### N-[4-[4-[1-(2-Aminoethyl)-1-methyl-piperidin-1-ium-4-carbonyl]piperazine-1-carbonyl]-3-chloro-phenyl]-5-(2,3-difluoro-4-methoxy-phenyl)-1-methyl-imidazole-2-carboxamide;formate

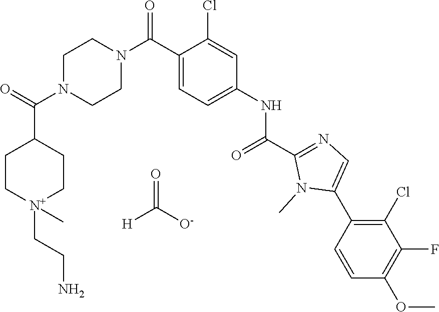

### Step 1: tert-butyl 4-[4-[2-chloro-4-[[5-(2,3-difluoro-4-methoxy-phenyl)-1-methyl-imidazole-2-carbonyl]amino]benzoyl]piperazine-1-carbonyl]piperidine-1-carboxylate

In a 100 mL round-bottomed flask, 1-(tert-butoxycarbonyl)piperidine-4-carboxylic acid (243 mg, 1.06 mmol), N-(3-chloro-4-(piperazine-1-carbonyl)phenyl)-5-(2,3-difluoro-4-methoxyphenyl)-1-methyl-1H-imidazole-2-carboxamide (400 mg, 816 μmol) and TEA (248 mg, 341 μl, 2.45 mmol) were combined with DMF (10 mL) to give a light yellow solution. 1-propanephosphonoc anhydride (779 mg, 1.22 mmol) was added. The reaction was stirred at room temperature for 1 h. The reaction mixture was poured into 50 mL H₂O and extracted with EtOAc (3×50 mL). The organic layers were combined, washed with sat NaCl (1×50 mL). The organic layers were dried over Na₂SO₄ and concentrated in vacuum to afford tert-butyl 4-(4-(2-chloro-4-(5-(2,3-difluoro-4-methoxyphenyl)-1-methyl-1H-imidazole-2-carboxamido)benzoyl)piperazine-1-carbonyl)piperidine-1-carboxylate (570 mg, 813 μmol, 99% yield). ESI MS [M+H]⁺: 701.2.

### Step 2: N-[3-chloro-4-[4-(piperidine-4-carbonyl)piperazine-1-carbonyl]phenyl]-5-(2,3-difluoro-4-methoxy-phenyl)-1-methyl-imidazole-2-carboxamide

In a 100 mL round-bottomed flask, tert-butyl 4-(4-(2-chloro-4-(5-(2,3-difluoro-4-methoxyphenyl)-1-methyl-1H-imidazole-2-carboxamido)benzoyl)piperazine-1-carbonyl)piperidine-1-carboxylate (570 mg, 813 μmol) was combined with THE (5 mL) to give a colorless solution. HCl (8.13 mL, 32.5 mmol) in dioxane was added. The reaction was stirred at room temperature for 10 min. The crude reaction mixture was concentrated in vacuum. The crude product was directly used to the next step to afford N-(3-chloro-4-(4-(piperidine-4-carbonyl)piperazine-1-carbonyl)phenyl)-5-(2,3-difluoro-4-methoxyphenyl)-1-methyl-1H-imidazole-2-carboxamide (489 mg, 814 μmol, 100% yield). ESI MS [M+H]⁺: 601.1.

### Step 3: tert-butyl N-[2-[4-[4-[2-chloro-4-[[5-(2,3-difluoro-4-methoxy-phenyl)-1-methyl-imidazole-2-carbonyl]amino]benzoyl]piperazine-1-carbonyl]-1-piperidyl]ethyl]carbamate

In a 100 mL round-bottomed flask, N-(3-chloro-4-(4-(piperidine-4-carbonyl)piperazine-1-carbonyl)phenyl)-5-(2,3-difluoro-4-methoxyphenyl)-1-methyl-1H-imidazole-2-carboxamide (90 mg, 150 μmol), tert-butyl (2-oxoethyl)carbamate (95.3 mg, 599 μmol) and sodium cyanoborohydride (47 mg, 749 μmol) were combined with MeOH (5 mL) to give a colorless solution. The reaction mixture was heated to 40° C. and stirred for 1 h. The crude product was directly used to the next step to afford tert-butyl (2-(4-(4-(2-chloro-4-(5-(2,3-difluoro-4-methoxyphenyl)-1-methyl-1H-imidazole-2-carboxamido)benzoyl)piperazine-1-carbonyl)piperidin-1-yl)ethyl)carbamate (111 mg, 149 μmol, 99% yield). ESI MS [M+H]⁺: 744.6.

### Step 4: tert-butyl N-[2-[4-[4-[2-chloro-4-[[5-(2,3-difluoro-4-methoxy-phenyl)-1-methyl-imidazole-2-carbonyl]amino]benzoyl]piperazine-1-carbonyl]-1-methyl-piperidin-1-ium-1-yl]ethyl]carbamate

To a 25 mL microwave vial was added tert-butyl (2-(4-(4-(2-chloro-4-(5-(2,3-difluoro-4-methoxyphenyl)-1-methyl-1H-imidazole-2-carboxamido)benzoyl)piperazine-1-carbonyl)piperidin-1-yl)ethyl)carbamate (111 mg, 149 μmol), Mel (212 mg, 93.3 μl, 1.49 mmol) and DIEA (289 mg, 391 μl, 2.24 mmol) in MeOH. The vial was capped and heated in the microwave at 50° C. for 2 h. The crude reaction mixture was concentrated in vacuum. The crude product was directly used to the next step to afford 1-(2-((tert-butoxycarbonyl)amino)ethyl)-4-(4-(2-chloro-4-(5-(2,3-difluoro-4-methoxyphenyl)-1-methyl-1H-imidazole-2-carboxamido)benzoyl)piperazine-1-carbonyl)-1-methylpiperidin-1-ium (113 mg, 149 μmol, 99% yield). ESI MS [M+H]⁺: 758.5.

### Step 5: N-[4-[4-[1-(2-aminoethyl)-1-methyl-piperidin-1-ium-4-carbonyl]piperazine-1-carbonyl]-3-chloro-phenyl]-5-(2,3-difluoro-4-methoxy-phenyl)-1-methyl-imidazole-2-carboxamide;formate

In a 100 mL round-bottomed flask, 1-(2-((tert-butoxycarbonyl)amino)ethyl)-4-(4-(2-chloro-4-(5-(2,3-difluoro-4-methoxyphenyl)-1-methyl-1H-imidazole-2-carboxamido)benzoyl)piperazine-1-carbonyl)-1-methylpiperidin-1-ium (113 mg, 149 μmol) was combined with THE (3 mL) to give a light yellow solution. HCl (1.12 mL, 4.46 mmol) in dioxane was added. The reaction was stirred at room temperature for 30 min. The crude reaction mixture was concentrated in vacuum. The crude material was purified by preparative PLC to afford N-[4-[4-[1-(2-aminoethyl)-1-methyl-piperidin-1-ium-4-carbonyl]piperazine-1-carbonyl]-3-chloro-phenyl]-5-(2,3-difluoro-4-methoxy-phenyl)-1-methyl-imidazole-2-carboxamide;formate (28 mg, 37.4 μmol, 25% yield). ESI MS [M+H]⁺: 658.4.

The following Type III Examples were prepared in analogy to Example C11:

| | | | MS | |
|---|---|---|---|---|
| | | | ESI | |
| Int. | Name | Structure | [M + H]⁺ | Starting Material |
| Example C12 | N-[4-[4-[1-(2- aminoethyl)-1- methyl-piperidin- 1-ium-4-carbon- yl]piperazine-1- carbonyl]-3- chloro-phenyl]-5- [2-chloro-4- (difluorometh- oxy)-3-fluoro- phenyl]-1-methyl- imidazole-2- carboxamide; formic acid; formate | 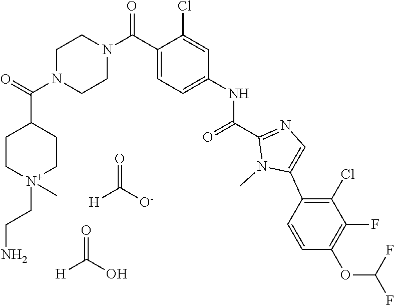 | 710.0 | Intermediate E12 and tert-butyl (2- oxoethyl)carbamate and methyl iodide |
| Example C13 | N-[4-[4-[1-(azet- idin-3-ylmeth-yl)- 1-methyl-piper- idin-1-ium-4- carbonyl]piper- azine-1-carbon- yl]-3-chloro-phen- yl]-5-[4-(difluoro- methoxy)-2,3- difluoro-phenyl]- 1-methyl-imid- azole-2-carbox- amide; 2,2,2- trifluoroacetate; 2,2,2-trifluoro- acetic acid | 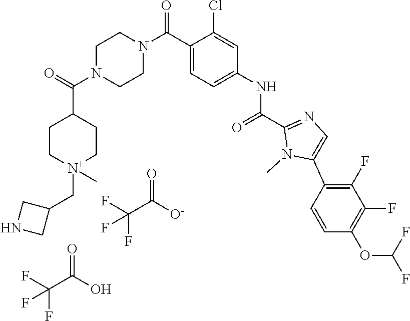 [SMILES: Cn1c(-c2ccc(OC(F)F)c(F)c2F)cnc1C(=O)Nc1ccc(C(=O)N2CCN(C(=O)C3CC[N+](C)(CC4CNC4)CC3)CC2)c(Cl)c1 ; O=C(O)C(F)(F)F ; O=C([O-])C(F)(F)F] | 720.1 | Intermediate E13 and tert-butyl 3- formylazetidine-1- carboxylate and methyl iodide |
| Example C14 | N-[3-chloro-4-[4- [1-methyl-1- (pyrrolidin-3-yl- methyl)piperidin- 1-ium-4-carbon- yl]piperazine-1- carbonyl]phenyl]- 5-[4-(difluoro- methoxy)-2,3- difluoro-phenyl]- 1-methyl-imid- azole-2-carbox- amide; formic acid; formate | 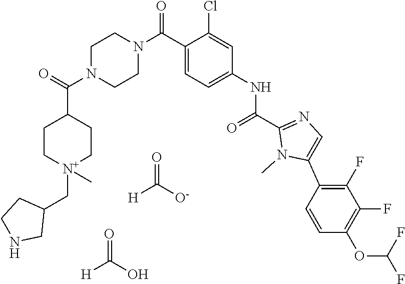 | 734.3 | Intermediate E13 and tert-butyl 3- formylpyrrolidine- 1-carboxylate and methyl iodide |
| Example C15 | N-[4-[4-[1- (azetidin-3-yl- methyl)-1-meth- yl-piperidin-1- ium-4-carbonyl]- piperazine-1-car- bonyl]-3-chloro- phenyl]-5-[2- chloro-4-(difluoro- methoxy)-3- fluoro-phenyl]-1- methyl-imidazole- 2-carboxamide; formic acid; formate | 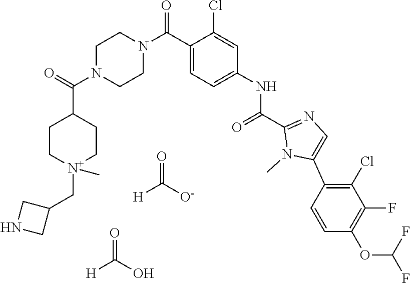 | 736.3 | Intermediate E12 and tert-butyl 3- formylazetidine-1- carboxylate and methyl iodide |
| Example C16 | N-[4-[4-[1-(azet- idin-3-ylmethyl)- 1-methyl-piper- idin-1-ium-4- carbonyl]piper- azine-1-carbon- yl]-3-chloro- phenyl]-5-(2,3- difluoro-4-meth- oxy-phenyl)-1- methyl-imid- azole-2-carbox- amide; formic acid; formate | 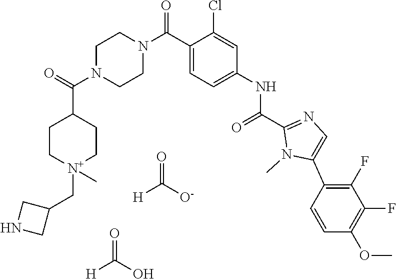 | 684.1 | Intermediate E6 and tert-butyl 3- formylazetidine-1- carboxylate and methyl iodide |
| Example C17 | 5-[4-(difluoro- methoxy)-2,3- difluoro-phenyl]- N-[3-ethyl-4-[4- [1-methyl-1-(pyr- rolidin-3-ylmeth- yl)piperidin-1- ium-4-carbonyl]- piperazine-1- carbonyl]phenyl]- 1-methyl-imid- azole-2-carbox- amide; 2,2,2- trifluoroacetate; 2,2,2-trifluoro- acetic acid | 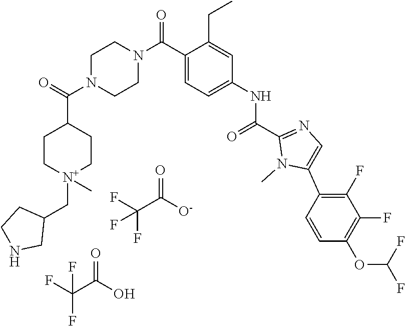 [SMILES: CCc1cc(NC(=O)c2ncc(-c3ccc(OC(F)F)c(F)c3F)n2C)ccc1C(=O)N1CCN(C(=O)C2CC[N+](C)(CC3CCNC3)CC2)CC1 ; O=C(O)C(F)(F)F ; O=C([O-])C(F)(F)F] | 728.2 | Intermediate E2 and tert-butyl 3- formylpyrrolidine- 1-carboxylate and methyl iodide |
| Example C18 | N-[3-chloro-4-[4- [2-[1-methyl-1- (pyrrolidin-3-yl- methyl)pyrrol- idin-1-ium-3-yl]- acetyl]piperazine- 1-carbonyl]phen- yl]-5-[4-(difluoro- methoxy)-2,3- difluoro-phenyl]- 1-methyl-imid- azole-2-carbox- amide; 2,2,2- trifluoroacetate; 2,2,2-trifluoro- acetic acid | 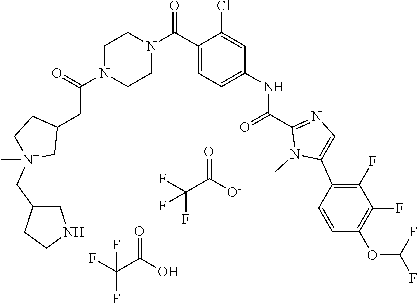 [SMILES: Cn1c(-c2ccc(OC(F)F)c(F)c2F)cnc1C(=O)Nc1ccc(C(=O)N2CCN(C(=O)CC3CC[N+](C)(CC4CCNC4)C3)CC2)c(Cl)c1 ; O=C(O)C(F)(F)F ; O=C([O-])C(F)(F)F] | 735.1 | Intermediate E13 and 2-(1-tert- butoxycarbonylpyr- rolidin-3-yl)acetic acid; tert-butyl 3- formylpyrrolidine- 1-carboxylate and methyl iodide |
| Example C19 | N-[4-[4-[1-(azet- idin-3-ylmethyl)- 1-methyl-piper- idin-1-ium-4- carbonyl]piper- azine-1-carbonyl]- 3-ethyl-phenyl]- 5-[4-difluoro- methoxy)-2,3- difluoro-phenyl]- 1-methyl-imid- azole-2-carbox- amide; 2,2,2- trifluoroacetate; 2,2,2-trifluoro- acetic acid | 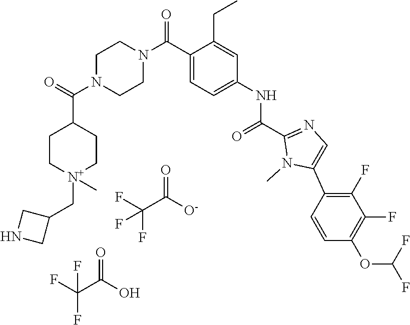 [SMILES: CCc1cc(NC(=O)c2ncc(-c3ccc(OC(F)F)c(F)c3F)n2C)ccc1C(=O)N1CCN(C(=O)C2CC[N+](C)(CC3CNC3)CC2)CC1 ; O=C(O)C(F)(F)F ; O=C([O-])C(F)(F)F] | 714.3 | Intermediate E2 and tert-butyl 3- formylazetidine-1- carboxylate and methyl iodide |
| Example C20 | 5-[4-(difluoro- methoxy)-2,3- difluoro-phenyl]- N-[3-ethyl-4-[4- [1-methyl-1-(4- piperidylmethyl)- piperidin-1-ium- 4-carbonyl]- piperazine-1- carbonyl]phenyl]- 1-methyl-imid- azole-2-carbox- amide; 2,2,2- trifluoroacetate; 2,2,2-trifluoro- acetic acid | 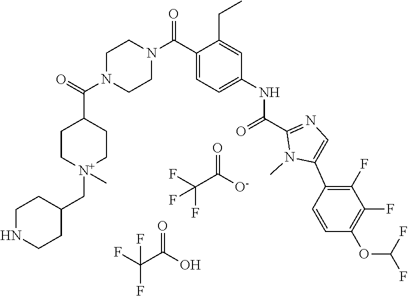 [SMILES: CCc1cc(NC(=O)c2ncc(-c3ccc(OC(F)F)c(F)c3F)n2C)ccc1C(=O)N1CCN(C(=O)C2CC[N+](C)(CC3CCNCC3)CC2)CC1 ; O=C(O)C(F)(F)F ; O=C([O-])C(F)(F)F] | 742.3 | Intermediate E2 and tert-butyl 4- formylpiperidine- 1-carboxylate and methyl iodide |
| Example C21 | N-[4-[4-[1-(2- amino-2-oxo- ethyl)-1-(azetidin- 3-ylmethyl)piper- idin-1-ium-4- carbonyl]piper- azine-1-carbonyl]- 3-ethyl-phenyl]- 5-[4-difluoro- methoxy)-2,3- difluoro-phenyl]- 1-methyl-imid- azole-2-carbox- amide; 2,2,2- trifluoroacetate; 2,2,2-trifluoro- acetic acid | 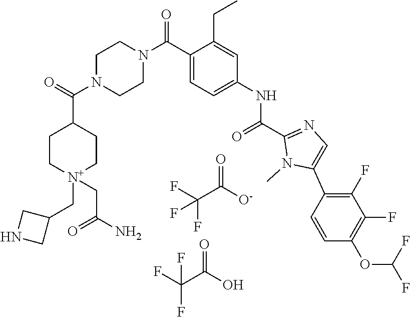 [SMILES: CCc1cc(NC(=O)c2ncc(-c3ccc(OC(F)F)c(F)c3F)n2C)ccc1C(=O)N1CCN(C(=O)C2CC[N+](CC(N)=O)(CC3CNC3)CC2)CC1 ; O=C(O)C(F)(F)F ; O=C([O-])C(F)(F)F] | 757.1 | Intermediate E2 and tert-butyl 3- formylazetidine-1- carboxylate and 2- iodoacetamide |
| Example C42 | N-[4-[4-[1-(azet- idin-3-ylmethyl)- 1-methyl-piper- idin-1-ium-4- carbonyl]piper- azine-1-carbon- yl]-3-fluoro- phenyl]-5-[4- (difluorometh- oxy)-2,3-difluoro- phenyl]-1-methyl- imidazole-2-car- boxamide; 2,2,2- trifluoroacetate; 2,2,2-trifluoro- acetic acid | 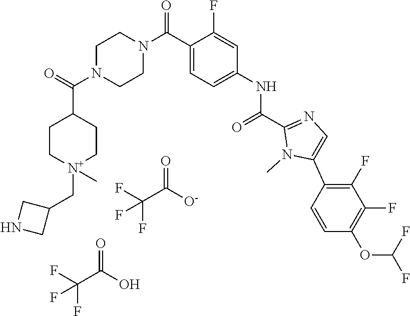 [SMILES: Cn1c(-c2ccc(OC(F)F)c(F)c2F)cnc1C(=O)Nc1ccc(C(=O)N2CCN(C(=O)C3CC[N+](C)(CC4CNC4)CC3)CC2)c(F)c1 ; O=C(O)C(F)(F)F ; O=C([O-])C(F)(F)F] | 704.2 | Intermediate E28 and tert-butyl 3- formylazetidine-1- carboxylate and methyl iodide |

### Example C22

### N-[3-chloro-4-[[1-[(1,1-dimethylazetidin-1-ium-3-yl)methyl]pyrazol-4-yl]methylcarbamoyl]phenyl]-5-(2,3-difluoro-4-methoxy-phenyl)-1-methyl-imidazole-2-carboxamide;formate

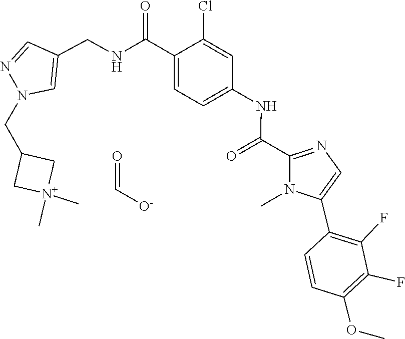

### Step 1: tert-butyl 4-[[[2-chloro-4-[[5-(2,3-difluoro-4-methoxy-phenyl)-1-methyl-imidazole-2-carbonyl]amino]benzoyl]amino]methyl]pyrazole-1-carboxylate

Intermediate G1 (400 mg, 948 μmol) was dissolved in N,N-Dimethylacetamide (4.74 mL). To this solution were added tert-butyl 4-(aminomethyl)-1H-pyrazole-1-carboxylate (514 mg, 1.04 mmol), HATU (397 mg, 1.04 mmol) and N-ethyl-N-isopropylpropan-2-amine (613 mg, 4.74 mmol) successively. The resulting solution was stirred at rt for 15 min, and then poured into 100 mL water. The aqueous phase was extracted with EtOAc (50 mL×3). The organic layers were combined, washed with 50 mL brine, dried over sodium sulfate and concentrated in vacuum. The residue was purified by silica gel flash chromatography. The product was a gum like solid, which was azeotroped with 20 mL toluene to become powder, 423 mg, 73%. ESI MS [M+H]⁺: 601.3.

### Step 2: N-[3-chloro-4-(1H-pyrazol-4-ylmethylcarbamoyl)phenyl]-5-(2,3-difluoro-4-methoxy-phenyl)-1-methyl-imidazole-2-carboxamide;hydrochloride

tert-butyl 4-((2-chloro-4-(5-(2,3-difluoro-4-methoxyphenyl)-1-methyl-1H-imidazole-2-carboxamido)benzamido)methyl)-1H-pyrazole-1-carboxylate (420 mg, 699 μmol) was dissolved in MeOH (3.49 mL). To this solution were added 1 mL 4 M HCl/MeOH solution. The resulting solution was stirred at rt for 2 h, and concentrated in vacuum. The residue was azeotroped with 20 mL toluene to become powder, 326 mg, 87%. ESI MS [M+H]⁺: 501.2.

### Step 3: tert-butyl 3-[[4-[[[2-chloro-4-[[5-(2,3-difluoro-4-methoxy-phenyl)-1-methyl-imidazole-2-carbonyl]amino]benzoyl]amino]methyl]pyrazol-1-yl]methyl]azetidine-1-carboxylate

N-(4-(((1H-pyrazol-4-yl)methyl)carbamoyl)-3-chlorophenyl)-5-(2,3-difluoro-4-methoxyphenyl)-1-methyl-1H-imidazole-2-carboxamide (200 mg, 399 μmol) was dissolved in THF (8.0 mL). To this solution were added tert-butyl 3-(iodomethyl)azetidine-1-carboxylate (178 mg, 599 μmol) and cesium carbonate (260 mg, 799 μmol). The resulting solution was stirred at 65° C. for 47 h, and then cooled to rt. Silica gel (100-200 mesh) was added to absorb the sample. The residue was purified by silica gel flash chromatography to give white powder, 162 mg, 60%. ESI MS [M+H]⁺: 670.3.

### Step 4: N-[3-chloro-4-[[1-[(1,1-dimethylazetidin-1-ium-3-yl)methyl]pyrazol-4-yl]methylcarbamoyl]phenyl]-5-(2,3-difluoro-4-methoxy-phenyl)-1-methyl-imidazole-2-carboxamide;formate

tert-butyl 3-[[4-[[[2-chloro-4-[[5-(2,3-difluoro-4-methoxy-phenyl)-1-methyl-imidazole-2-carbonyl]amino]benzoyl]amino]methyl]pyrazol-1-yl]methyl]azetidine-1-carboxylate (160 mg, 239 μmol) was dissolved in 5 mL 4M HCl/MeOH, the solution was stirred at rt for 1 h to remove the Boc protection. The solvent was removed in vacuum, and the residue was dissolved in MeCN (4.78 mL). To this solution were added N-ethyl-N-isopropylpropan-2-amine (309 mg, 2.39 mmol) and iodomethane (169 mg, 1.19 mmol). The resulting solution was stirred at rt for 1 h. The solvent was removed in vacuum and the residue was purified by preparative HPLC to give white powder, 33 mg, 24%. ESI MS [M+H]⁺: 598.0.

### Example C23

### [2-[[5-[[[2-chloro-4-[[5-(2,3-difluoro-4-methoxy-phenyl)-1-methyl-imidazole-2-carbonyl]amino]benzoyl]amino]methyl]-3-methyl-thiazol-3-ium-2-yl]amino]-2-oxo-ethyl]-trimethyl-ammonium;formate

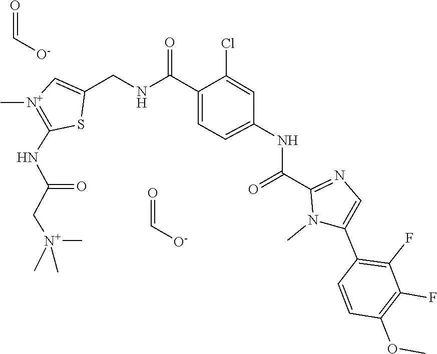

### Step 1: tert-butyl N-[5-[[[2-chloro-4-[[5-(2,3-difluoro-4-methoxy-phenyl)-1-methyl-imidazole-2-carbonyl]amino]benzoyl]amino]methyl]thiazol-2-yl]carbamate

At room temperature, a mixture of 2-chloro-4-[[5-(2,3-difluoro-4-methoxy-phenyl)-1-methyl-imidazole-2-carbonyl]amino]benzoic acid (425 mg, 1.01 mmol), tert-butyl N-[5-(aminomethyl)thiazol-2-yl]carbamate (347 mg, 1.51 mmol), HATU (575 mg, 1.51 mmol) and DIPEA (391 mg, 3.02 mmol) in DMF (5 mL) was stirred for 16 h. Then the mixture was poured into water. The solid was collected and dried to give the crude title compound tert-butyl N-[5-[[[2-chloro-4-[[5-(2,3-difluoro-4-methoxy-phenyl)-1-methyl-imidazole-2-carbonyl]amino]benzoyl]amino]methyl]thiazol-2-yl]carbamate 600 mg as a yellow solid. ESI MS [M+H]⁺: 633.2.

### Step 2: N-[4-[(2-aminothiazol-5-yl)methylcarbamoyl]-3-chloro-phenyl]-5-(2,3-difluoro-4-methoxy-phenyl)-1-methyl-imidazole-2-carboxamide

A solution of tert-butyl N-[5-[[[2-chloro-4-[[5-(2,3-difluoro-4-methoxy-phenyl)-1-methyl-imidazole-2-carbonyl]amino]benzoyl]amino]methyl]thiazol-2-yl]carbamate (600 mg, 948 μmol) in DCM (5 mL) and TFA (5 mL) was stirred at room temperature for 1 h. Then the mixture was concentrated. The residue was basified by NH₃·H₂O. The water layer was extracted with DCM, dried over anhydrous Na₂SO₄ and concentrated to give the crude product (400 mg) as a brown solid. The crude product (100 mg) was purified by Prep-HPLC to give the title compound N-[4-[(2-aminothiazol-5-yl)methylcarbamoyl]-3-chloro-phenyl]-5-(2,3-difluoro-4-methoxy-phenyl)-1-methyl-imidazole-2-carboxamide (20 mg) as a white solid. ESI MS [M+H]⁺: 533.0.

### Step 3: N-[3-chloro-4-[[2-[[2-(dimethylamino)acetyl]amino]thiazol-5-yl]methylcarbamoyl]phenyl]-5-(2,3-difluoro-4-methoxy-phenyl)-1-methyl-imidazole-2-carboxamide

At room temperature, a mixture of N-[4-[(2-aminothiazol-5-yl)methylcarbamoyl]-3-chloro-phenyl]-5-(2,3-difluoro-4-methoxy-phenyl)-1-methyl-imidazole-2-carboxamide (300 mg, 563 μmol), 2-(dimethylamino)acetic acid (116 mg, 1.13 mmol), HATU (428 mg, 1.13 mmol) and DIPEA (218 mg, 1.69 mmol) in DMF (5 mL) was stirred for 16 h. Then the mixture was poured into water. The water layer was extracted with DCM. The combined organic layers were washed with water, dried over anhydrous Na₂SO₄ and concentrated to give the crude title compound (300 mg) as a yellow solid. ESI MS [M+H]⁺: 618.3.

### Step 4: [2-[[5-[[[2-chloro-4-[[5-(2,3-difluoro-4-methoxy-phenyl)-1-methyl-imidazole-2-carbonyl]amino]benzoyl]amino]methyl]-3-methyl-thiazol-3-ium-2-yl]amino]-2-oxo-ethyl]-trimethyl-ammonium;formate

At room temperature, a mixture of N-[3-chloro-4-[[2-[[2-(dimethylamino)acetyl]amino]thiazol-5-yl]methylcarbamoyl]phenyl]-5-(2,3-difluoro-4-methoxy-phenyl)-1-methyl-imidazole-2-carboxamide (100 mg, 162 μmol), iodomethane (230 mg, 1.62 mmol) and DIPEA (209 mg, 1.62 mmol) in Acetonitrile (5 mL) was stirred for 16 h. Then the mixture was dissolved in DMF and was purified by Prep-HPLC to give the title compound [2-[[5-[[[2-chloro-4-[[5-(2,3-difluoro-4-methoxy-phenyl)-1-methyl-imidazole-2-carbonyl]amino]benzoyl]amino]methyl]-3-methyl-thiazol-3-ium-2-yl]amino]-2-oxo-ethyl]-trimethyl-ammonium;formate (30 mg) as a white solid. ESI MS [M+H]⁺: 646.3.

### Example C24

### N-[3-chloro-4-[4-[2-(1-methylpiperazin-1-ium-1-yl)acetyl]piperazine-1-carbonyl]phenyl]-5-[4-(difluoromethoxy)-2,3-difluoro-phenyl]-1-methyl-imidazole-2-carboxamide;2,2,2-trifluoroacetate;2,2,2-trifluoroacetic acid

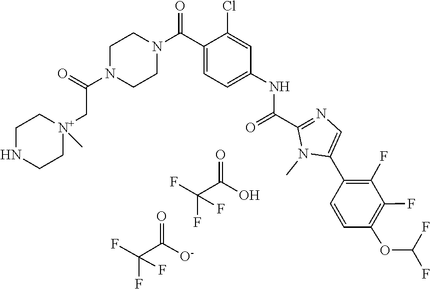

### Step 1: N-[3-chloro-4-[4-(2-chloroacetyl)piperazine-1-carbonyl]phenyl]-5-[4-(difluoromethoxy)-2,3-difluoro-phenyl]-1-methyl-imidazole-2-carboxamide

In a 100 mL round-bottomed flask, N-(3-chloro-4-(piperazine-1-carbonyl)phenyl)-5-(4-(difluoromethoxy)-2,3-difluorophenyl)-1-methyl-1H-imidazole-2-carboxamide (127 mg, 241 μmol) and DIEA (93.6 mg, 724 μmol) were combined with DCM (10 mL) to give a light brown solution. 2-chloroacetyl chloride (35.5 mg, 314 μmol) was added. The reaction was stirred at room temperature for 1 h. The crude reaction mixture was concentrated in vacuum. The crude material was purified by preparative HPLC to afford N-[3-chloro-4-[4-(2-chloroacetyl)piperazine-1-carbonyl]phenyl]-5-[4-(difluoromethoxy)-2,3-difluoro-phenyl]-1-methyl-imidazole-2-carboxamide (75 mg). ESI MS [M+H]⁺: 660.0.

### Step 2: tert-butyl 4-[2-[4-[2-chloro-4-[[5-[4-(difluoromethoxy)-2,3-difluoro-phenyl]-1-methyl-imidazole-2-carbonyl]amino]benzoyl]piperazin-1-yl]-2-oxo-ethyl]piperazine-1-carboxylate

To a 5 mL microwave vial was added N-(3-chloro-4-(4-(2-chloroacetyl)piperazine-1-carbonyl)phenyl)-5-(4-(difluoromethoxy)-2,3-difluorophenyl)-1-methyl-1H-imidazole-2-carboxamide (75 mg, 125 μmol), tert-butyl piperazine-1-carboxylate (46.4 mg, 249 μmol) and DIEA (32.2 mg, 249 μmol) in MeCN (3 mL). The vial was capped and heated in the microwave at 80° C. for 1 h. The reaction was directly used to the next step, to afford tert-butyl 4-[2-[4-[2-chloro-4-[[5-[4-(difluoromethoxy)-2,3-difluoro-phenyl]-1-methyl-imidazole-2-carbonyl]amino]benzoyl]piperazin-1-yl]-2-oxo-ethyl]piperazine-1-carboxylate (93.7 mg). ESI MS [M+H]⁺: 751.9.

### Step 3: tert-butyl 4-[2-[4-[2-chloro-4-[[5-[4-(difluoromethoxy)-2,3-difluoro-phenyl]-1-methyl-imidazole-2-carbonyl]amino]benzoyl]piperazin-1-yl]-2-oxo-ethyl]-4-methyl-piperazin-4-ium-1-carboxylate;iodide

In a 25 mL round-bottomed flask, tert-butyl 4-[2-[4-[2-chloro-4-[[5-[4-(difluoromethoxy)-2,3-difluoro-phenyl]-1-methyl-imidazole-2-carbonyl]amino]benzoyl]piperazin-1-yl]-2-oxo-ethyl]piperazine-1-carboxylate (93 mg, 124 μmol), Mel (351 mg, 2.47 mmol) and DIEA (160 mg, 1.24 mmol) were combined with MeCN (3 mL) to give a light brown solution. The reaction was stirred at room temperature for 15 h. The crude reaction mixture was concentrated in vacuum. The crude product was directly used to the next step, to afford 4-(tert-butoxycarbonyl)-1-(2-(4-(2-chloro-4-(5-(4-(difluoromethoxy)-2,3-difluorophenyl)-1-methyl-1H-imidazole-2-carboxamido)benzoyl)piperazin-1-yl)-2-oxoethyl)-1-methylpiperazin-1-ium (94.9 mg). ESI MS [M+H]: 766.8.

### Step 4: N-[3-chloro-4-[4-[2-(1-methylpiperazin-1-ium-1-yl)acetyl]piperazine-1-carbonyl]phenyl]-5-[4-(difluoromethoxy)-2,3-difluoro-phenyl]-1-methyl-imidazole-2-carboxamide;2,2,2-trifluoroacetate;2,2,2-trifluoroacetic acid

In a 50 mL round-bottomed flask, 4-(tert-butoxycarbonyl)-1-(2-(4-(2-chloro-4-(5-(4-(difluoromethoxy)-2,3-difluorophenyl)-1-methyl-1H-imidazole-2-carboxamido)benzoyl)piperazin-1-yl)-2-oxoethyl)-1-methylpiperazin-1-ium (93 mg, 121 μmol) was combined with THE (3 mL) to give a light brown solution. HCl (in water) (1.01 ml, 12.1 mmol) was added. The reaction was stirred at room temperature for 30 min. The crude reaction mixture was concentrated in vacuum. The crude material was purified by preparative HPLC to afford Product 1 (30 mg). ESI MS [M+H]⁺: 666.2.

The following Type III Examples were prepared in analogy to Example C24:

| | | | MS | |
|---|---|---|---|---|
| | | | ESI | |
| Int | Name | Structure | [M + H]⁺ | Starting Material |
| Example C25 | N-[3-chloro-4-[4- [2-(1-methyl- pyrrolidin-1-ium- 1-yl)acetyl]piper- azine-1-carbonyl]- phenyl]-5-[4- (difluorometh- oxy)-2,3-difluoro- phenyl]-1-methyl- imidazole-2- carboxamide; formate | 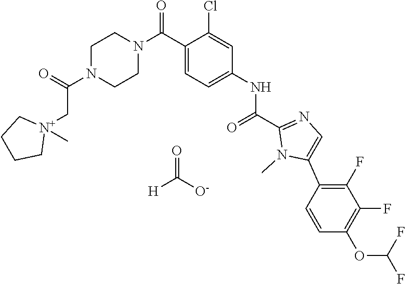 | 651.8 | Intermediate E13 and 2-chloroacetyl chloride; pyrroli- dine and methyl iodide |
| Example C26 | N-[4-[4-[2-(3- carbamoyl-1- methyl-pyrrolidin- 1-ium-1-yl)acet- yl]piperazine-1- carbonyl]-3- chloro-phenyl]-5- [4-(difluorometh- oxy)-2,3-difluoro- phenyl]-1-methyl- imidazole-2- carboxamide; formate | 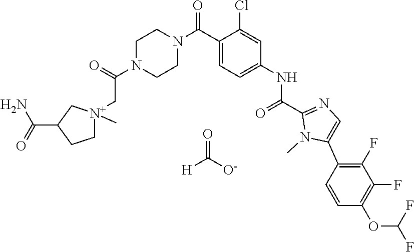 | 694.7 | Intermediate E13 and 2-chloroacetyl chloride; pyrrol- idine-3-carbox- amide and methyl iodide |
| Example C27 | N-[3-chloro-4-[4- [2-(4-methyl- morpholin-4-ium- 4-yl)acetyl]- piperazine-1- carbonyl]phenyl]- 5-(2,3-difluoro-4- methoxy-phenyl)- 1-methyl-imid- azole-2-carbox- amide; formate | 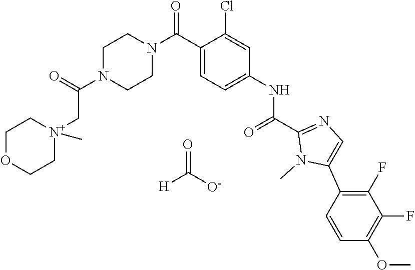 | 631.6 | Intermediate E6 and 2-chloroacet- yl chloride; pyr- rolidine-3-carbox- amide and methyl iodide |
| Example C28 | N-[3-chloro-4-[4- [2-(1-methylpyr- rolidin-1-ium-1- yl)acetyl]piper- azine-1-carbonyl]- phenyl]-5-(2,3- difluoro-4-meth- oxy-phenyl)-1- methyl-imidazole- 2-carboxamide; formate | 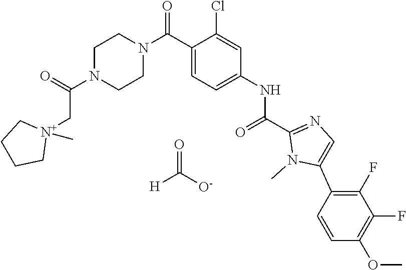 | 615.3 | Intermediate E6 and 2-chloroacet- yl chloride; pyr- rolidine and methyl iodide |
| Example C29 | N-[3-chloro-4-[4- [2-(3-hydroxy-1- methyl-pyrrolidin- 1-ium-1-yl)acet- yl]piperazine-1- carbonyl]phenyl]- 5-(2,3-difluoro-4- methoxy-phenyl)- 1-methyl-imid- azole-2-carbox- amide; formate | 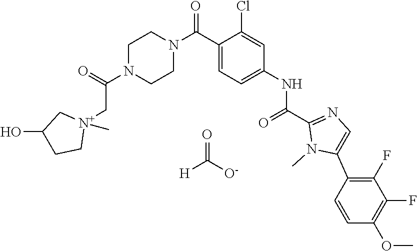 | 631.3 | Intermediate E6 and 2-chloroacet- yl chloride; pyr- rolidin-3-ol and methyl iodide |

### Example C35

### 3-aminopropyl-(carboxymethyl)-[4-[4-[2-chloro-4-[[5-(2,3-difluoro-4-methoxy-phenyl)-1-methyl-imidazole-2-carbonyl]amino]beuzoyl]piperazin-1-yl]-4-oxo-butyl]-methyl-ammonium formate

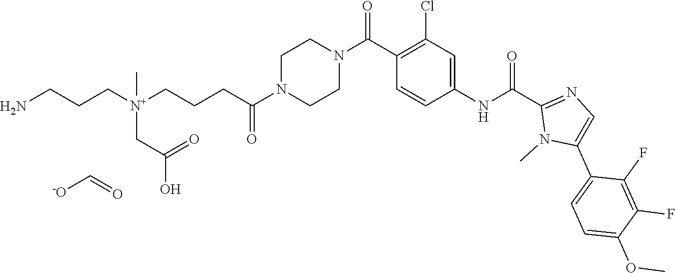

### Step 1: tert-butyl N-[4-[4-[2-chloro-4-[[5-(2,3-difluoro-4-methoxy-phenyl)-1-methyl-imidazole-2-carbonyl]amino]benzoyl]piperazin-1-yl]-4-oxo-butyl]carbamate

A mixture of N-[3-chloro-4-(piperazine-1-carbonyl)phenyl]-5-(2,3-difluoro-4-methoxy-phenyl)-1-methyl-imidazole-2-carboxamide 2,2,2-trifluoroacetate (intermediate ELN028612-068, 1.6, 2.65 mmol), BOC-gamma-abu-OH (0.54 g, 2.65 mmol, 1 eq), triethylamine (1.11 mL, 7.95 mmol) in THF (30 mL) was added propylphosphonic anhydride (1.89 mL, 3.18 mmol) at 25° C. and stirred for 12 h. The solution was concentrated in vacuum. The residue was purified by reversed phase-HPLC (FA) to afford the title compound (1.4 g, 2.07 mmol, 78.27% yield) as yellow solid. ESI MS [M+H]⁺: 675.5.

### Step 2: N-[4-[4-(4-aminobutanoyl)piperazine-1-carbonyl]-3-chloro-phenyl]-5-(2,3-difluoro-4-methoxy-phenyl)-1-methyl-imidazole-2-carboxamide 2,2,2-trifluoroacetate

A mixture of tert-butyl N-[4-[4-[2-chloro-4-[[5-(2,3-difluoro-4-methoxy-phenyl)-1-methyl-imidazole-2-carbonyl]amino]benzoyl]piperazin-1-yl]-4-oxo-butyl]carbamate (1.7 g, 2.52 mmol) in DCM (10 mL) was added trifluoroacetic acid (10.0 mL, 129.8 mmol), the mixture was stirred at 25° C. for 1 h. The solution was concentrated in vacuum. The residue was purified by reversed phase-HPLC (FA) to afford the title compound (1.1 g, 1.6 mmol, 63.4% yield) as white solid. ESI MS [M+H]⁺: 575.3.

### Step 3: tert-butyl N-[3-[[4-[4-[2-chloro-4-[[5-(2,3-difluoro-4-methoxy-phenyl)-1-methyl-imidazole-2-carbonyl]amino]benzoyl]piperazin-1-yl]-4-oxo-butyl]amino]propyl]carbamate

To a mixture of tert-butyl N-(3-oxopropyl)carbamate (0.41 g, 1.42 mmol) and N-[4-[4-(4-aminobutanoyl)piperazine-1-carbonyl]-3-chloro-phenyl]-5-(2,3-difluoro-4-methoxy-phenyl)-1-methyl-imidazole-2-carboxamide 2,2,2-trifluoroacetate (0.65 g, 0.940 mmol) in methanol (5 mL) was added sodium acetate (155 mg, 1.89 mmol) and sodium triacetoxyborohydride (600 mg, 2.83 mmol). The mixture was stirred at 25° C. for 16 h. The solution was concentrated in vacuum. The residue was purified by reversed phase HPLC (FA condition) to afford the title compound (300 mg, 0.410 mmol, 43.43% yield) as light yellow solid. ESI MS [M+H]⁺: 732.1.

### Step 4: tert-butyl 2-[3-(tert-butoxycarbonylamino)propyl-[4-[4-[2-chloro-4-[[5-(2,3-difluoro-4-methoxy-phenyl)-1-methyl-imidazole-2-carbonyl]amino]benzoyl]piperazin-1-yl]-4-oxo-butyl]amino]acetate

To a mixture of tert-butyl N-[3-[[4-[4-[2-chloro-4-[[5-(2,3-difluoro-4-methoxy-phenyl)-1-methyl-imidazole-2-carbonyl]amino]benzoyl]piperazin-1-yl]-4-oxo-butyl]amino]propyl]carbamate (250.0 mg, 0.340 mmol) in ACN (10 mL) was added triethylamine (0.14 mL, 1.02 mmol) and tert-butyl bromoacetate (0.17 mL, 1.02 mmol). The reaction mixture was stirred at 25° C. for 16 h. The mixture was concentrated in vacuum. The crude was purified by reversed phase-HPLC (FA condition) to afford the title compound (200 mg, 0.240 mmol, 69.21% yield) as yellow solid. ESI MS [M+H]⁺: 846.5.

### Step 5: 3-(tert-butoxycarbonylamino)propyl-(2-tert-butoxy-2-oxo-ethyl)-[4-[4-[2-chloro-4-[[5-(2,3-difluoro-4-methoxy-phenyl)-1-methyl-imidazole-2-carbonyl]amino]benzoyl]piperazin-1-yl]-4-oxo-butyl]-methyl-ammonium formate

A solution of tert-butyl 2-[3-(tert-butoxycarbonylamino)propyl-[4-[4-[2-chloro-4-[[5-(2,3-difluoro-4-methoxy-phenyl)-1-methyl-imidazole-2-carbonyl]amino]benzoyl]piperazin-1-yl]-4-oxo-butyl]amino]acetate (100.0 mg, 0.120 mmol) and triethylamine (0.05 mL, 0.350 mmol) in ACN (3 mL)/water (0.300 mL) was added iodomethane (0.11 mL, 1.18 mmol) and stirred at 20° C. for 16 h. The mixture was concentrated and purified by prep-HPLC (FA) to afford the title compound (50 mg, 0.060 mmol, 49.13% yield) as white solid. ESI MS [M+H]⁺: 860.5.

### Step 6: 3-aminopropyl-(carboxymethyl)-[4-[4-[2-chloro-4-[[5-(2,3-difluoro-4-methoxy-phenyl)-1-methyl-imidazole-2-carbonyl]amino]benzoyl]piperazin-1-yl]-4-oxo-butyl]-methyl-ammonium formate

A solution of 3-(tert-butoxycarbonylamino)propyl-(2-tert-butoxy-2-oxo-ethyl)-[4-[4-[2-chloro-4-[[5-(2,3-difluoro-4-methoxy-phenyl)-1-methyl-imidazole-2-carbonyl]amino]benzoyl]piperazin-1-yl]-4-oxo-butyl]-methyl-ammonium;formate (50.0 mg, 0.060 mmol) in DCM (2 mL) was added trifluoroacetic acid (1.9 mL, 24.67 mmol) and stirred at 20° C. for 16 h. The mixture was concentrated in vacuum. The residue was purified by prep-HPLC (FA condition) to afford the title compound (31.6 mg, 0.040 mmol, 75.37% yield) as white solid. ESI MS [M+H]⁺: 704.3.

### Example C36

### 2-[4-[4-[2-chloro-4-[[5-(2,3-difluoro-4-methoxy-phenyl)-1-methyl-imidazole-2-carbonyl]amino]benzoyl]piperazine-1-carbonyl]-1-methyl-piperazin-1-ium-1-yl]acetic acid formate

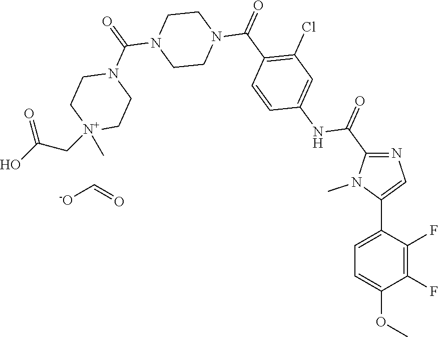

### Step 1: tert-butyl 2-[4-[4-[2-chloro-4-[[5-(2,3-difluoro-4-methoxy-phenyl)-1-methyl-imidazole-2-carbonyl]amino]benzoyl]piperazine-1-carbonyl]piperazin-1-yl]acetate

To a mixture of tert-butyl 2-piperazin-1-ylacetate (65.0 mg, 0.320 mmol, 1 eq) and bis(trichloromethyl)carbonate (35.6 mg, 0.120 mmol, 0.370 eq) in DCM (10 mL) was stirred at 20° C. for 1 h. Then, N,N-diisopropylethylamine (0.12 mL, 0.710 mmol, 2.2 eq) and N-[3-chloro-4-(piperazine-1-carbonyl)phenyl]-5-(2,3-difluoro-4-methoxy-phenyl)-1-methyl-imidazole-2-carboxamide hydrochloride (205 mg, 0.390 mmol, 1.2 eq) was added to the above solution. The reaction mixture was stirred at 20° C. for 2 h. The mixture was poured into water (30 mL) and extracted with DCM (20 ml×3), dried over sodium sulphate, filtered and concentrated in vacuum. The crude was purified by reversed phase-HPLC (FA) to afford the title compound (82 mg, 0.110 mmol, 35.28% yield) as white solid. ESI MS [M+H]⁺: 716.6.

### Step 2: 2-[4-[4-[2-chloro-4-[[5-(2,3-difluoro-4-methoxy-phenyl)-1-methyl-imidazole-2-carbonyl]amino]benzoyl]piperazine-1-carbonyl]piperazin-1-yl]acetic acid

To a mixture of tert-butyl 2-[4-[4-[2-chloro-4-[[5-(2,3-difluoro-4-methoxy-phenyl)-1-methyl-imidazole-2-carbonyl]amino]benzoyl]piperazine-1-carbonyl]piperazin-1-yl]acetate (82.0 mg, 0.110 mmol, 1 eq) in DCM (3 mL) was added trifluoroacetic acid (3.0 mL, 38.94 mmol, 340.09 eq) and stirred at 20° C. for 3 h. The mixture was concentrated in vacuum. The crude was purified by prep-HPLC (FA) to afford the title compound (49.5 mg, 0.070 mmol, 65.5% yield) as white solid. ESI MS [M+H]⁺: 660.3.

### Step 3: 2-[4-[4-[2-chloro-4-[[5-(2,3-difluoro-4-methoxy-phenyl)-1-methyl-imidazole-2-carbonyl]amino]benzoyl]piperazine-1-carbonyl]-1-methyl-piperazin-1-ium-1-yl]acetic acid formate

To a mixture of 2-[4-[4-[2-chloro-4-[[5-(2,3-difluoro-4-methoxy-phenyl)-1-methyl-imidazole-2-carbonyl]amino]benzoyl]piperazine-1-carbonyl]piperazin-1-yl]acetic acid (20.0 mg, 0.030 mmol, 1 eq) in ACN (2 mL) and water (0.200 mL) was added triethylamine (0.004 mL, 0.030 mmol, 1 eq) and iodomethane (0.008 mL, 0.120 mmol, 4 eq). The mixture was stirred at 10° C. for 12 h. The mixture was concentrated in vacuum. The crude was purified by prep-HPLC (FA) to afford the title compound (6.5 mg, 0.010 mmol, 29.4% yield) as white solid. ESI MS [M+H]⁺: 674.3.

### Example C37

### 2-[(3aS,6aR)-5-(3-aminopropyl)-2-[4-[2-chloro-4-[[5-(2,3-difluoro-4-methoxy-phenyl)-1-methyl-imidazole-2-carbonyl]amino]benzoyl]piperazine-1-carbonyl]-1,3,3a,4,6,6a-hexahydropyrrolo[3,4-c]pyrrol-5-ium-5-yl]acetic acid chloride hydrochloride

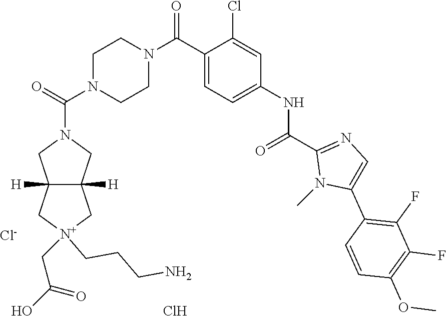 [SMILES: [Cl-] ; COc1ccc(-c2cnc(C(=O)Nc3ccc(C(=O)N4CCN(C(=O)N5C[C@@H]6C[N+](CCCN)(CC(=O)O)C[C@@H]6C5)CC4)c(Cl)c3)n2C)c(F)c1F ; Cl]

### Step 1: (3aR,6aS)-2-[4-[2-chloro-4-[[5-(2,3-difluoro-4-methoxy-phenyl)-1-methyl-imidazole-2-carbonyl]amino]benzoyl]piperazine-1-carbonyl]-1,3,3a,4,6,6a-hexahydropyrrolo[3,4-c]pyrrole-5-carboxylic acid tert-butyl ester

In a dried flask to a solution of triphosgene (8.22 mg, 0.028 mmol, 0.400 eq) in dichloromethane, extra dry (1.78 mL) was added N-[3-chloro-4-(piperazine-1-carbonyl)phenyl]-5-(2,3-difluoro-4-methoxy-phenyl)-1-methyl-imidazole-2-carboxamide hydrochloride (38.8 mg, 0.069 mmol, 1 eq) and DIEA (45 mg, 60.5 uL, 0.346 mmol, 5 eq). Residues were rinsed with dichloromethane, extra dry (0.691 mL). Then the reaction mixture was stirred at 0° C. for 25 minutes. Then (3aS,6aR)-2,3,3a,4,6,6a-hexahydro-1H-pyrrolo[3,4-c]pyrrole-5-carboxylic acid tert-butyl ester (17.7 mg, 0.083 mmol, 1.2 eq) in dichloromethane, extra dry (0.691 mL) was added. The reaction mixture was allowed to warm to room temperature and stirred for 4 hours. The reaction was quenched with sat. NaHCO₃ solution. The mixture was extracted with DCM and the organic layers were washed with brine. The combined organic layers were dried with magnesium sulfate, filtered and concentrated in vacuum. The residue was dissolved in DCM and the crude material was purified by silica chromatography using Hept/(EtOAc/EtOH 75:25) as eluent to afford the title compound (29.7 mg, 57.68%) as off-white solid. ESI MS [M−H]⁻: 726.60.

### Step 2: N-[4-[4-[(3aS,6aR)-2,3,3a,4,6,6a-hexahydro-1H-pyrrolo[3,4-c]pyrrole-5-carbonyl]piperazine-1-carbonyl]-3-chloro-phenyl]-5-(2,3-difluoro-4-methoxy-phenyl)-1-methyl-imidazole-2-carboxamide dihydrochloride

To a solution of (3aR,6aS)-2-[4-[2-chloro-4-[[5-(2,3-difluoro-4-methoxy-phenyl)-1-methyl-imidazole-2-carbonyl]amino]benzoyl]piperazine-1-carbonyl]-1,3,3a,4,6,6a-hexahydropyrrolo[3,4-c]pyrrole-5-carboxylic acid tert-butyl ester (29.7 mg, 0.040 mmol, 1 eq) in 1,4-dioxane, extra dry (0.200 mL) was added 4 M HCl in dioxane (200 uL, 0.799 mmol, 20 eq). The reaction mixture was stirred at room temperature for 1 hour. The suspension was sonicated in the ultrasonic bath and then stirring was continued overnight. The reaction mixture was directly lyophilized to afford the title compound (31 mg, 108.43%) as light yellow solid. ESI MS [M+H]⁺: 628.31.

### Step 3: N-[3-[(3aS,6aR)-5-[4-[2-chloro-4-[[5-(2,3-difluoro-4-methoxy-phenyl)-1-methyl-imidazole-2-carbonyl]amino]benzoyl]piperazine-1-carbonyl]-1,3,3a,4,6,6a-hexahydropyrrolo[3,4-c]pyrrol-2-yl]propyl]carbamic acid tert-butyl ester

To a solution of N-[4-[4-[(3aS,6aR)-2,3,3a,4,6,6a-hexahydro-1H-pyrrolo[3,4-c]pyrrole-5-carbonyl]piperazine-1-carbonyl]-3-chloro-phenyl]-5-(2,3-difluoro-4-methoxy-phenyl)-1-methyl-imidazole-2-carboxamide dihydrochloride (31 mg, 0.043 mmol, 1 eq) and triethylamine (13.2 mg, 18.1 uL, 0.130 mmol, 3 eq) in 1,2-dichloroethane (0.942 mL) was added molecular sieves. The mixture was stirred at RT for 20 minutes. Then the reaction was concentrated in vacuo. The residue was re-dissolved in 1,2-dichloroethane (0.942 mL) and triethylamine (13.2 mg, 18.1 uL, 0.130 mmol, 3 eq) was added. Then new molecular sieves were added and the mixture was stirred at RT for 20 minutes. Then N-(3-ketopropyl)carbamic acid tert-butyl ester (15.0 mg, 0.087 mmol, 2 eq) in 1,2-dichloroethane (0.3 mL) was added and the mixture was heated to 45° C. for 1 hour. Then sodium triacetoxyborohydride (27.6 mg, 0.130 mmol, 3 eq) was added and the mixture was stirred at RT for 3 hours. The reaction mixture was diluted with DCM and filtered. The filtrate was diluted with water and extracted with DCM. The combined organic layers were dried over magnesium sulfate, filtered and concentrated in vacuum. The material was purified by reverse phase prep HPLC using ACN/water+Et3N 0.01% as eluent. The obtained fractions were lyophilized to afford the title compound (11.6 mg, 31.36%) as white powder. ESI MS [M+H]⁺: 785.34.

### Step 4: 2-[(3aS,6aR)-5-[3-(tert-butoxycarbonylamino)propyl]-2-[4-[2-chloro-4-[[5-(2,3-difluoro-4-methoxy-phenyl)-1-methyl-imidazole-2-carbonyl]amino]benzoyl]piperazine-1-carbonyl]-1,3,3a,4,6,6a-hexahydropyrrolo[3,4-c]pyrrol-5-ium-5-yl]acetic acid tert-butyl ester bromide

To a solution of N-[3-[(3aS,6aR)-5-[4-[2-chloro-4-[[5-(2,3-difluoro-4-methoxy-phenyl)-1-methyl-imidazole-2-carbonyl]amino]benzoyl]piperazine-1-carbonyl]-1,3,3a,4,6,6a-hexahydropyrrolo[3,4-c]pyrrol-2-yl]propyl]carbamic acid tert-butyl ester (11.6 mg, 0.014 mmol, 1 eq) in acetonitrile (0.200 mL) was added DIEA (2.6 mg, 3.6 uL, 0.020 mmol, 1.5 eq) and tert-butyl bromoacetate (4 mg, 3.0 uL, 0.020 mmol, 1.5 eq). The reaction mixture was heated to 45° C. for 16 hours. Then the reaction mixture was concentrated in vacuum to afford the title compound (13.1 mg, 90.46%) as light grey solid which was used without further purification. ESI MS [M+H]⁺: 899.48.

Step 5: 2-[(3aS,6aR)-5-(3-aminopropyl)-2-[4-[2-chloro-4-[[5-(2,3-difluoro-4-methoxy-phenyl)-1-methyl-imidazole-2-carbonyl]amino]benzoyl]piperazine-1-carbonyl]-1,3,3a,4,6,6a-hexahydropyrrolo[3,4-c]pyrrol-5-ium-5-yl]acetic acid chloride trihydrochloride

To a solution of2-[(3aS,6aR)-5-[3-(tert-butoxycarbonylamino)propyl]-2-[4-[2-chloro-4-[[5-(2,3-difluoro-4-methoxy-phenyl)-1-methyl-imidazole-2-carbonyl]amino]benzoyl]piperazine-1-carbonyl]-1,3,3a,4,6,6a-hexahydropyrrolo[3,4-c]pyrrol-5-ium-5-yl]acetic acid tert-butyl ester bromide (13.1 mg, 0.012 mmol, 1 eq) in 1,4-dioxane (0.200 mL) was added 4 M HCl in dioxane (30.7 uL, 0.123 mmol, 10 eq). The reaction was stirred at room temperature for 7 hours. The reaction mixture was directly lyophilized to afford the title compound (12.3 mg, 99.03%) as light yellow solid. ESI MS [M+H]⁺: 743.38.

### Example C38

### 2-[1-(3-aminopropyl)-4-[4-[2-chloro-4-[[5-(2,3-difluoro-4-methoxy-phenyl)-1-methyl-imidazole-2-carbonyl]amino]benzoyl]piperazine-1-carbonyl]piperazin-1-ium-1-yl]acetic acid formate

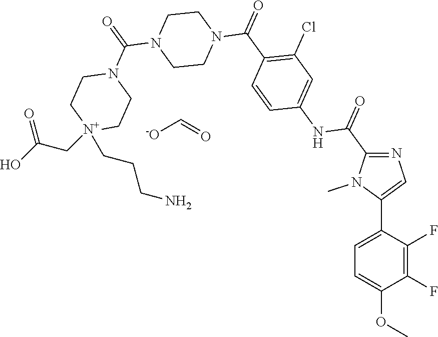

### Step 1: tert-butyl 2-[1-[3-(tert-butoxycarbonylamino)propyl]-4-[4-[2-chloro-4-[[5-(2,3-difluoro-4-methoxy-phenyl)-1-methyl-imidazole-2-carbonyl]amino]benzoyl]piperazine-1-carbonyl]piperazin-1-ium-1-yl]acetate formate

To a mixture of tert-butyl 2-[1-[3-(tert-butoxycarbonylamino)propyl]piperazin-1-ium-1-yl]acetate (Intermediate H22, 60.0 mg, 0.170 mmol, 1 eq) and bis(trichloromethyl)carbonate (18.4 mg, 0.060 mmol, 0.370 eq) in DCM (10 mL) was stirred at 20° C. for 1 h. Then, N,N-diisopropylethylamine (0.06 mL, 0.370 mmol, 2.2 eq) and N-[3-chloro-4-(piperazine-1-carbonyl)phenyl]-5-(2,3-difluoro-4-methoxy-phenyl)-1-methyl-imidazole-2-carboxamide hydrochloride (106 mg, 0.200 mmol, 1.2 eq) was added to the above solution. The reaction mixture was stirred at 20° C. for 2 h. The mixture was concentrated in vacuum. The crude was purified by reversed phase-HPLC (0.1% FA)-ACN to afford the title compound (25 mg, 0.030 mmol, 17.08% yield) as colorless oil. ESI MS [M+H]⁺: 873.3.

### Step 2: 2-[1-(3-aminopropyl)-4-[4-[2-chloro-4-[[5-(2,3-difluoro-4-methoxy-phenyl)-1-methyl-imidazole-2-carbonyl]amino]benzoyl]piperazine-1-carbonyl]piperazin-1-ium-1-yl]acetic acid formate

tert-butyl 2-[1-[3-(tert-butoxycarbonylamino)propyl]-4-[4-[2-chloro-4-[[5-(2,3-difluoro-4-methoxy-phenyl)-1-methyl-imidazole-2-carbonyl]amino]benzoyl]piperazine-1-carbonyl]piperazin-1-ium-1-yl]acetate formate (25.0 mg, 0.030 mmol, 1 eq) 20° C. was added into 4M HCl in dioxane (3.0 mL) and stirred at 20° C. for 16 h. The mixture was concentrated in vacuum. The crude was purified by reversed phase-HPLC (0.1% FA)-ACN to afford the title compound (12 mg, 0.020 mmol, 56.4% yield) as white solid. ESI MS [M+H]⁺: 717.3.

### Example C39

### 2-[4-[4-[2-chloro-4-[[5-(2,3-difluoro-4-methoxy-phenyl)-1-methyl-imidazole-2-carbonyl]amino]benzoyl]piperazine-1-carbonyl]-1-[2-(dimethylamino)ethyl]piperidin-1-ium-1-yl]acetic acid bromide

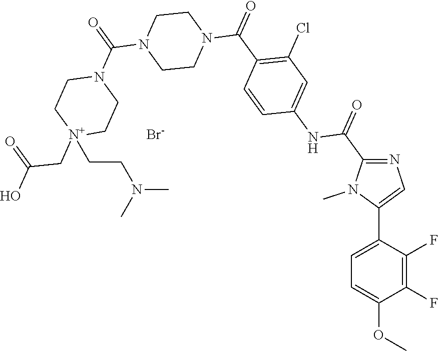 [SMILES: COc1ccc(-c2cnc(C(=O)Nc3ccc(C(=O)N4CCN(C(=O)N5CC[N+](CCN(C)C)(CC(=O)O)CC5)CC4)c(Cl)c3)n2C)c(F)c1F ; [Br-]]

### Step 1: O4-benzyl O1-tert-butyl piperidine-1,4-dicarboxylate

To a solution of N—BOC-isonipecotic acid (20.0 g, 87.23 mmol, 1 eq) and potassium carbonate (13.26 g, 95.96 mmol, 1.1 eq) in ACN (100 mL) was added benzyl bromide (11.41 mL, 95.96 mmol, 1.1 eq) at 50° C. and stirred for 3 h. Then the mixture was filtered and concentrated to afford the title compound (27.8 g, 87.04 mmol, 99.78% yield) as colorless oil, which was used without further purification. ESI MS [M+H]⁺: 264.2.

### Step 2: benzyl piperidine-4-carboxylate hydrochloride

A solution of 4M hydrochloric acid in MeOH (50.0 mL, 200 mmol, 2.3 eq) and in methanol (50 mL) was added 04-benzyl 01-tert-butyl piperidine-1,4-dicarboxylate (27.8 g, 87.04 mmol, 1 eq) at 50° C. and stirred for 16 h. The mixture concentrated in vacuum and the residue purified by trituration with MTBE (200 mL) to afford the title compound (16 g, 62.56 mmol, 71.88% yield) as white solid. ESI MS [M+H]⁺: 220.1.

### Step 3: benzyl 1-[2-(benzyloxycarbonylamino)ethyl]piperidine-4-carboxylate

A solution of benzyl piperidine-4-carboxylate hydrochloride (5.5 g, 21.51 mmol, 1 eq) and triethylamine (6.29 mL, 45.16 mmol, 2.1 eq) in ACN (30 mL) was added benzyl N-(2-bromoethyl)carbamate (5.55 g, 21.51 mmol, 1 eq) at 30° C. and stirred for 16 h. The mixture was filtered and concentrated in vacuo. The residue was purified by silica column DCM/EtOAc/MeOH=10:10:1 to afford the title compound (6.2 g, 15.64 mmol, 72.71% yield) as colorless oil. ESI MS [M+H]⁺: 397.2.

### Step 4: benzyl 1-[2-(benzyloxycarbonylamino)ethyl]-1-(2-tert-butoxy-2-oxo-ethyl)piperidin-1-ium-4-carboxylate trifluoroacetate

A solution of benzyl 1-[2-(benzyloxycarbonylamino)ethyl]piperidine-4-carboxylate (3.0 g, 7.57 mmol, 1 eq) and triethylamine (0.53 mL, 3.78 mmol, 0.500 eq) in ACN (30 mL) was added tert-butyl bromoacetate (1.97 mL, 12.11 mmol, 1.6 eq) at 70° C. and stirred for 16 h. The mixture was concentrated and the obtained residue purified by reversed phase-HPLC (FA) and Prep-HPLC (TFA) to afford the title compound (1.1 g, 2.15 mmol, 28.41% yield) as colorless oil. ESI MS [M+H]⁺: 511.4.

### Step 5: 1-(2-tert-butoxy-2-oxo-ethyl)-1-[2-(dimethylamino)ethyl]piperidin-1-ium-4-carboxylic acid trifluoroacetate

A solution of benzyl 1-[2-(benzyloxycarbonylamino)ethyl]-1-(2-tert-butoxy-2-oxo-ethyl)piperidin-1-ium-4-carboxylate (1.0 g, 1.95 mmol, 1 eq) and formaldehyde (793 mg, 9.77 mmol, 5 eq) in methanol (30 mL) was added 10% palladium/C (208 mg) under N₂, the mixture was stirred at 10° C. and for 16 h under a hydrogen atmosphere. The mixture was filtered and concentrated to afford the title compound (615 mg, 1.56 mmol, 89.78% yield) as white solid. ESI MS [M+H]⁺: 315.0.

### Step 6: tert-butyl 2-[4-[4-[2-chloro-4-[[5-(2,3-difluoro-4-methoxy-phenyl)-1-methyl-imidazole-2-carbonyl]amino]benzoyl]piperazine-1-carbonyl]-1-[2-(dimethylamino)ethyl]piperidin-1-ium-1-yl]acetate

To a solution of 1-(2-tert-butoxy-2-oxo-ethyl)-1-[2-(dimethylamino)ethyl]piperidin-1-ium-4-carboxylic acid (93.0 mg, 0.290 mmol, 1 eq) in DMF (0.500 mL) was added N-[3-chloro-4-(piperazine-1-carbonyl)phenyl]-5-(2,3-difluoro-4-methoxy-phenyl)-1-methyl-imidazole-2-carboxamide hydrochloride (intermediate ELN028612-068, 40.0 mg, 0.080 mmol, 0.260 eq), O-(7-azabenzotriazol-1-yl)-N,N,N′,N′-tetramethyluronium hexafluorophosphate (112 mg, 0.290 mmol, 1 eq) and triethylamine (0.5 mL, 3.59 mmol, 12.17 eq). The resulting mixture was stirred at 25° C. for 16 h. The mixture was purified by prep-HPLC (0.1% FA)-ACN to the title compound (230 mg, 0.290 mmol, 99.08% yield) as brown oil. ESI MS [M+H]⁺: 786.4.

### Step 7: 2-[4-[4-[2-chloro-4-[[5-(2,3-difluoro-4-methoxy-phenyl)-1-methyl-imidazole-2-carbonyl]amino]benzoyl]piperazine-1-carbonyl]-1-[2-(dimethylamino)ethyl]piperidin-1-ium-1-yl]acetic acid bromide

The title compound was prepared in analogy to Example C11, step 5 and was obtained (13.7 mg, 0.020 mmol, 5.71% yield) as white solid. ESI MS [M+H]⁺: 730.3.

### Example C41

### 2-[1-(azetidin-3-ylmethyl)-4-[4-[2-chloro-4-[[5-[2-chloro-3-fluoro-4-(fluoromethoxy)phenyl]-1-methyl-imidazole-2-carbonyl]amino]benzoyl]piperazine-1-carbonyl]piperidin-1-ium-1-yl]acetic acid formate

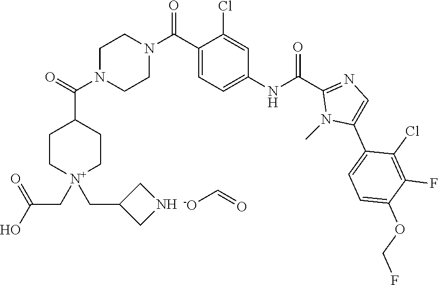

### Step 1: 5-(2-chloro-3-fluoro-4-hydroxy-phenyl)-N-[3-chloro-4-(piperazine-1-carbonyl)phenyl]-1-methyl-imidazole-2-carboxamide

To a stirred solution of tert-butyl 4-[2-chloro-4-[[5-(2-chloro-3-fluoro-4-methoxy-phenyl)-1-methyl-imidazole-2-carbonyl]amino]benzoyl]piperazine-1-carboxylate (1.0 g, 1.65 mmol, 1 eq) in DCM (10 mL) was added dropwise boron tribromide (0.62 mL, 6.6 mmol, 4 eq) at 0° C. Then the mixture was stirred at 20° C. for 16 h. The mixture was concentrated in vacuum. The residue was purified by reversed phase HPLC (FA condition) to afford 5-(2-chloro-3-fluoro-4-hydroxy-phenyl)-N-[3-chloro-4-(piperazine-1-carbonyl)phenyl]-1-methyl-imidazole-2-carboxamide (690 mg, 1.4 mmol, 85% yield) as white solid. ESI MS [M+H]⁺: 492.4.

### Step 2: tert-butyl 3-[[1-(2-tert-butoxy-2-oxo-ethyl)-4-[4-[2-chloro-4-[[5-(2-chloro-3-fluoro-4-hydroxy-phenyl)-1-methyl-imidazole-2-carbonyl]amino]benzoyl]piperazine-1-carbonyl]piperidin-1-ium-1-yl]methyl]azetidine-1-carboxylate formate

A mixture of 1-[(1-tert-butoxycarbonylazetidin-3-yl)methyl]-1-(2-tert-butoxy-2-oxo-ethyl)piperidin-1-ium-4-carboxylate (605 mg, 1.46 mmol, 0.900 eq), N,N-diisopropylethylamine (0.57 mL, 3.25 mmol, 2 eq) and O-(7-azabenzotriazol-1-yl)-N,N,N′,N′-tetramethyluronium hexafluorophosphate (927 mg, 2.44 mmol, 1.5 eq) in DMF (10 mL) was stirred at 15° C. for 30 min. Then 5-(2-chloro-3-fluoro-4-hydroxy-phenyl)-N-[3-chloro-4-(piperazine-1-carbonyl)phenyl]-1-methyl-imidazole-2-carboxamide (800.0 mg, 1.62 mmol, 1 eq) was added and stirring continued at 15° C. for 12 h. The mixture was concentrated in vacuum, the residue was purified by reversed phase HPLC (FA condition) to afford the title compound (500 mg, 0.560 mmol, 34.66% yield) as light yellow solid. ESI MS [M+H]⁺: 886.2.

### Step 3: tert-butyl 3-[[1-(2-tert-butoxy-2-oxo-ethyl)-4-[4-[2-chloro-4-[[5-[2-chloro-3-fluoro-4-(fluoromethoxy)phenyl]-1-methyl-imidazole-2-carbonyl]amino]benzoyl]piperazine-1-carbonyl]piperidin-1-ium-1-yl]methyl]azetidine-1-carboxylate formate

To a stirred mixture of tert-butyl 3-[[1-(2-tert-butoxy-2-oxo-ethyl)-4-[4-[2-chloro-4-[[5-(2-chloro-3-fluoro-4-hydroxy-phenyl)-1-methyl-imidazole-2-carbonyl]amino]benzoyl]piperazine-1-carbonyl]piperidin-1-ium-1-yl]methyl]azetidine-1-carboxylate (100.0 mg, 0.110 mmol, 1 eq) and cesium carbonate (0.04 g, 0.140 mmol, 1.2 eq) in DMF (4 mL) was added dropwise bromo(fluoro)methane (0.04 mL, 0.560 mmol, 5 eq) at 10° C., then the mixture was stirred at 20° C. for 16 h. The mixture was concentrated in vacuum. The residue was purified by reversed phase HPLC (FA condition) to afford the title compound (30 mg, 0.030 mmol, 28.96% yield) as white solid. ESI MS [M+H]⁺: 918.3.

### Step 4: 2-[1-(azetidin-3-ylmethyl)-4-[4-[2-chloro-4-[[5-[2-chloro-3-fluoro-4-(fluoromethoxy)phenyl]-1-methyl-imidazole-2-carbonyl]amino]benzoyl]piperazine-1-carbonyl]piperidin-1-ium-1-yl]acetic acid formate

To a stirred mixture of tert-butyl 3-[[1-(2-tert-butoxy-2-oxo-ethyl)-4-[4-[2-chloro-4-[[5-[2-chloro-3-fluoro-4-(fluoromethoxy)phenyl]-1-methyl-imidazole-2-carbonyl]amino]benzoyl]piperazine-1-carbonyl]piperidin-1-ium-1-yl]methyl]azetidine-1-carboxylate formate (30.0 mg, 0.030 mmol, 1 eq) in DCM (2 mL) was added trifluoroacetic acid (2.0 mL, 25.96 mmol, 795.98 eq), then the mixture was stirred at 10° C. for 16 h. The mixture was concentrated in vacuum. The residue was purified by prep-HPLC (FA condition) to afford the title compound (13.2 mg, 0.020 mmol, 53% yield) as light yellow solid. ESI MS [M+H]⁺: 762.1.

### Example C43

### N-[3-chloro-4-[4-[[(2S,4R)-4-hydroxy-1,1-dimethyl-pyrrolidin-1-ium-2-carbonyl]amino]piperidine-1-carbonyl]phenyl]-5-(2,3-difluoro-4-methoxy-phenyl)-1-methyl-imidazole-2-carboxamide;formate

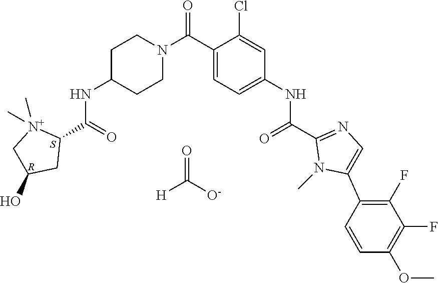

### Step 1: tert-butyl (2S,4R)-2-[[1-[2-chloro-4-[[5-(2,3-difluoro-4-methoxy-phenyl)-1-methyl-imidazole-2-carbonyl]amino]benzoyl]-4-piperidyl]carbamoyl]-4-hydroxy-pyrrolidine-1-carboxylate

At room temperature, a mixture of N-[4-(4-aminopiperidine-1-carbonyl)-3-chloro-phenyl]-5-(2,3-difluoro-4-methoxy-phenyl)-1-methyl-imidazole-2-carboxamide (2.2 g, 4.37 mmol), (2S,4R)-1-tert-butoxycarbonyl-4-hydroxy-pyrrolidine-2-carboxylic acid (1.21 g, 5.24 mmol), HATU (2.49 g, 6.55 mmol) and DIPEA (1.69 g, 13.1 mmol) in DMF (15 mL) was stirred for 1 h. Then the mixture was poured into water. The water layer was extracted with DCM. The organic layer was concentrated and the residue was purified by flash column to give the title compound (1.8 g) as a brown solid. ESI MS [M+H]⁺: 717.3.

### Step 2: N-[3-chloro-4-[4-[[(2S,4R)-4-hydroxypyrrolidine-2-carbonyl]amino]piperidine-1-carbonyl]phenyl]-5-(2,3-difluoro-4-methoxy-phenyl)-1-methyl-imidazole-2-carboxamide

At room temperature, a solution of tert-butyl (2S,4R)-2-[[1-[2-chloro-4-[[5-(2,3-difluoro-4-methoxy-phenyl)-1-methyl-imidazole-2-carbonyl]amino]benzoyl]-4-piperidyl]carbamoyl]-4-hydroxy-pyrrolidine-1-carboxylate (1.8 g, 2.51 mmol) in DCM (15 mL) and TFA (10 mL) was stirred for 1 h. Then the mixture was concentrated and dissolved in DCM. Then the solution was basified by NH₃·H₂O to PH 8-9. The water layer was extracted with DCM. The organic layer was dried and concentrated to give the crude product (1.3 g) as a brown solid. ESI MS [M+H]⁺: 617.2.

### Step 3: N-[3-chloro-4-[4-[[(2S,4R)-4-hydroxy-1,1-dimethyl-pyrrolidin-1-ium-2-carbonyl]amino]piperidine-1-carbonyl]phenyl]-5-(2,3-difluoro-4-methoxy-phenyl)-1-methyl-imidazole-2-carboxamide;formate

At room temperature, a mixture of N-[3-chloro-4-[4-[[(2S,4R)-4-hydroxypyrrolidine-2-carbonyl]amino]piperidine-1-carbonyl]phenyl]-5-(2,3-difluoro-4-methoxy-phenyl)-1-methyl-imidazole-2-carboxamide (0.8 g, 1.3 mmol), iodomethane (1.84 g, 13 mmol) and DIPEA (2.51 g, 19.4 mmol) in acetonitrile (20 mL) was stirred for 16 h. Then the mixture was concentrated and dissolved in water. A part of the solution was purified by Prep-HPLC to give the title compound (30 mg) as a white powder. ESI MS [M+H]⁺: 645.4.

### Assay Procedures

### Antimicrobial Susceptibility Testing:

### 90% Growth Inhibitory Concentration (IC90) Determination

The in vitro antimicrobial activity of the compounds was determined according to the following procedure:

The assay used a 10-points Iso-Sensitest broth medium to measure quantitatively the in vitro activity of the compounds against Acinetobacter baumannii ATCC17961.

Stock compounds in DMSO were serially twofold diluted (e.g. range from 50 to 0.097 μM final concentration) in 384 wells microtiter plates and inoculated with 49 μl the bacterial suspension in Iso-Sensitest medium to have a final cell concentration of ˜5×10⁽⁵⁾ CFU/ml in a final volume/well of 50 ul/well. Microtiter plates were incubated at 35±2° C.

Bacterial cell growth was determined with the measurement of optical density at λ=600 nm each 20 minutes over a time course of 16 h. Growth inhibition was calculated during the logarithmic growth of the bacterial cells with determination of the concentration inhibiting 50% (IC50) and 90% (IC90) of the growth.

Table 1 provides the 9000 growth inhibitory concentrations (IC90) in micromoles per liter of the compounds of present invention obtained against the strain Acinetobacter baumannii ATCC17961.

Particular compounds ofthe present invention exhibit an IC90 (Acinetobacter baumannii ATCC17961)≤25 μmol/l.

More particular compounds of the present invention exhibit an 2C90 (Acinetobacter baumanni ATCC17961)≤5 μmol/l.

Most particular compounds of the present invention exhibit an 2C90 (Acinetobacter baumanni ATCC17961)≤1 μmol/l.

| | ATCC 17961 IC90 |
|---|---|
| Example | [μM] |
| Example A1 | 0.079 |
| Example A2 | 0.34 |
| Example A3 | 0.16 |
| Example A4 | 0.21 |
| Example A5 | 0.46 |
| Example A6 | 1.5 |
| Example A7 | 0.093 |
| Example A8 | 0.31 |
| Example A9 | 0.13 |
| Example A10 | 0.084 |
| Example A11 | 0.57 |
| Example A12 | 0.35 |
| Example A13 | 0.64 |
| Example A14 | 0.6 |
| Example A15 | 0.77 |
| Example A16 | 0.088 |
| Example A17 | 0.34 |
| Example A18 | 0.24 |
| Example A19 | 1.2 |
| Example A20 | 0.32 |
| Example A21 | 0.33 |
| Example A22 | 0.39 |
| Example A23 | 0.4 |
| Example A24 | 0.35 |
| Example A25 | 0.41 |
| Example A26 | 0.41 |
| Example A27 | 0.056 |
| Example A28 | 0.088 |
| Example A29 | 0.17 |
| Example A30 | 0.14 |
| Example A31 | 0.17 |
| Example B1 | 0.28 |
| Example B2 | 0.23 |
| Example B3 | 0.19 |
| Example B4 | 0.11 |
| Example B5 | 0.12 |
| Example B6 | 0.29 |
| Example B7 | 0.099 |
| Example B8 | 0.18 |
| Example B9 | 0.056 |
| Example B10 | 0.075 |
| Example B11 | 0.15 |
| Example B12 | 0.051 |
| Example B13 | 0.068 |
| Example B14 | 0.076 |
| Example B15 | 0.14 |
| Example B16 | 0.23 |
| Example B17 | 0.25 |
| Example B18 | 0.3 |
| Example B19 | 0.31 |
| Example B20 | 0.076 |
| Example B21 | 0.28 |
| Example B22 | 0.17 |
| Example B23 | 0.41 |
| Example B24 | 0.12 |
| Example B25 | 0.12 |
| Example B26 | 0.54 |
| Example B27 | 0.17 |
| Example B28 | 0.22 |
| Example B29 | 0.25 |
| Example B30 | 0.11 |
| Example B31 | 0.25 |
| Example B32 | 0.2 |
| Example B33 | 0.13 |
| Example B34 | 0.14 |
| Example B35 | 0.13 |
| Example B36 | 0.22 |
| Example B37 | 0.19 |
| Example B38 | 0.18 |
| Example B39 | 0.11 |
| Example B40 | 0.26 |
| Example B41 | 0.076 |
| Example B42 | 0.14 |
| Example B43 | 0.12 |
| Example B44 | 0.42 |
| Example B45 | 0.093 |
| Example B46 | 0.12 |
| Example B47 | 0.039 |
| Example B48 | 0.076 |
| Example B49 | 0.12 |
| Example B50 | 0.25 |
| Example B51 | 0.54 |
| Example B52 | 0.52 |
| Example B53 | 0.091 |
| Example B54 | 0.35 |
| Example B55 | 0.15 |
| Example B56 | 0.061 |
| Example C1 | 0.023 |
| Example C2 | 0.034 |
| Example C3 | 0.057 |
| Example C4 | 0.031 |
| Example C5 | 0.073 |
| Example C6 | 0.16 |
| Example C7 | 0.25 |
| Example C8 | 0.64 |
| Example C9 | 0.12 |
| Example C10 | 0.1 |
| Example C11 | 0.091 |
| Example C12 | 0.043 |
| Example C13 | 0.073 |
| Example C14 | 0.049 |
| Example C15 | 0.073 |
| Example C16 | 0.1 |
| Example C17 | 0.22 |
| Example C18 | 0.22 |
| Example C19 | 0.27 |
| Example C20 | 0.08 |
| Example C21 | 0.22 |
| Example C22 | 0.23 |
| Example C23 | 0.076 |
| Example C24 | 0.063 |
| Example C25 | 0.043 |
| Example C26 | 0.069 |
| Example C27 | 0.12 |
| Example C28 | 0.13 |
| Example C29 | 0.1 |
| Example C30 | 0.54 |
| Example C31 | 0.41 |
| Example C32 | 0.81 |
| Example C33 | 0.49 |
| Example C34 | 0.57 |
| Example C35 | 0.51 |
| Example C36 | 1.1 |
| Example C37 | 0.34 |
| Example C38 | 0.73 |
| Example C39 | 1.3 |
| Example C40 | 0.54 |
| Example C41 | 0.48 |
| Example C42 | 0.1 |
| Example C43 | 0.29 |

### Example 1

A compound of formula (I) can be used in a manner known per se as the active ingredient for the production of tablets of the following composition:

| | Per tablet | |
|---|---|---|
| Active ingredient | 200 | mg |
| Microcrystalline cellulose | 155 | mg |
| Corn starch | 25 | mg |
| Talc | 25 | mg |
| Hydroxypropylmethylcellulose | 20 | mg |
| | 425 | mg |

### Example 2

A compound of formula (I) can be used in a manner known per se as the active ingredient for the production of capsules of the following composition:

| | Per capsule | |
|---|---|---|
| Active ingredient | 100.0 | mg |
| Corn starch | 20.0 | mg |
| Lactose | 95.0 | mg |
| Talc | 4.5 | mg |
| Magnesium stearate | 0.5 | mg |
| | 220.0 | mg |

### Example 3

A compound of formula (I) can be used in a manner known per se as the active ingredient for the production of an infusion solution of the following composition:

| | |
|---|---|
| Active ingredient | 100 mg |
| Lactic acid 90% | 100 mg |

NaOH q.s. or HCl q.s. for adjustment to pH 4.0
Sodium chloride q.s. or glucose q.s. for adjustment of the osmolality to 290 mOsm/kg

| | |
|---|---|
| Water for injection (WFI) | ad 100 ml |

### Example 4

A compound of formula (I) can be used in a manner known per se as the active ingredient for the production of an infusion solution of the following composition:

| | | |
|---|---|---|
| Active ingredient | 100 | mg |
| Hydroxypropyl-beta-cyclodextrin | 10 | g |

NaOH q.s. or HCl q.s. for adjustment to pH 7.4
Sodium chloride q.s. or glucose q.s. for adjustment of the osmolality to 290 mOsm/kg

| | |
|---|---|
| Water for injection (WFI) | ad 100 ml |

1. A compound of formula (I): 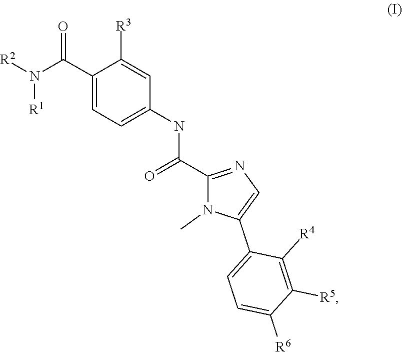
or a pharmaceutically acceptable salt thereof, wherein:
R¹ is selected from (C₁-C₆-alkyl)₃N⁺—C₁-C₆-alkyl-O—C₁-C₆-alkyl-, (C₁-C₆-alkyl)₃N⁺—C₁-C₆-alkyl-, (C₁-C₆-alkyl)₃N⁺—C₁-C₆-alkyl-C(O)—NH—C₁-C₆-alkyl-, carbamoyl-C₁-C₆-alkyl-(C₁-C₆-alkyl)₂N⁺—C₁-C₆-alkyl-O—C₁-C₆-alkyl-, carbamoyl-C₁-C₆-alkyl-(C₁-C₆-alkyl)₂N⁺—C₁-C₆-alkyl-, a group 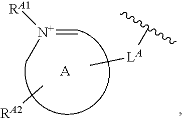
and a group 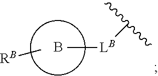
and R² is hydrogen; or
R¹ and R², taken together with the nitrogen atom to which they are attached, form a group 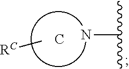
R³ is halogen or C₁-C₆-alkyl;
R⁴, R⁵ and R⁶ are each independently selected from hydrogen, halogen, cyano, halo-C₁-C₆-alkyl, C₁-C₆-alkoxy, halo-C₆-alkoxy, and cyano-C₁-C₆-alkoxy;
R^{A1} is C₁-C₆-alkyl or cyano-C₁-C₆-alkyl;
R^{A2} is selected from C₁-C₆-alkyl, hydroxy-C₁-C₆-alkyl, hydroxy-C₁-C₆-alkyl-O—C₁-C₆-alkyl-, carbamoyl-C₁-C₆-alkyl, C₁-C₆-alkoxy-C₁-C₆-alkyl-, C₁-C₆-alkyl-S(O)₂—C₁-C₆-alkyl-, and (C₁-C₆-alkyl)₃N⁺—C₁-C₆-alkyl-C(O)—NH—;
R^{B} is selected from (C₁-C₆-alkyl)₃N⁺—C₁-C₆-alkyl-NH—, (C₁-C₆-alkyl)₃N⁺—C₁-C₆-alkyl-C(O)—NH—, carbamoyl-C₁-C₆-alkyl-(C₁-C₆-alkyl)₂N⁺—C₁-C₆-alkyl-C(O)—, and a group 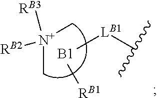
R^{B1} is hydrogen or hydroxy;
R^{B2} and R^{B3} are each independently C₁-C₆-alkyl or carbamoyl-C₁-C₆-alkyl;
R^{C} is selected from (R^{C8})₃N⁺—C₁-C₆-alkyl-L^{C2}-, a group 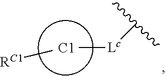
a group 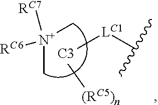
and a group 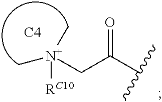
R^{C1} is (C₁-C₆-alkyl)₃N⁺—, (R^{C9})₃N⁺—C₁-C₆-alkyl-C(O)—, or a group 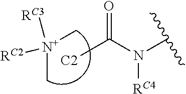
R^{C2}, R^{C3}, R^{C6}, R^{C7}, and R^{C10} are independently selected from C₁-C₆-alkyl, carboxy-C₁-C₆-alkyl-, amino-C₁-C₆-alkyl, carbamoyl-C₁-C₆-alkyl, (C₁-C₆-alkyl)₂N—C₁-C₆-alkyl-, (3- to 14-membered heterocyclyl)-C₁-C₆-alkyl-;
R^{C4} is hydrogen or C₁-C₆-alkyl;
each R^{C5} is independently selected from hydrogen, hydroxy, carbamoyl, C₁-C₆-alkyl, hydroxy-C₁-C₆-alkyl, and (3- to 14-membered heterocyclyl)-C₁-C₆-alkyl-;
each R^{C8} and each R^{C9} is independently selected from amino-C₁-C₆-alkyl-, carboxy-C₁-C₆-alkyl-, carbamoyl-C₁-C₆-alkyl-, and C₁-C₆-alkyl-;
A is 3- to 14-membered heteroaryl;
B is selected from 3- to 14-membered heteroaryl, 3- to 14-membered heterocyclyl, and C₃-C₁₀-cycloalkyl;
B1, C, C2, C3, and C4 are all 3- to 14-membered heterocyclyl;
C1 is 3- to 14-membered heterocyclyl or 3- to 14-membered heteroaryl;
L^{A} is C₁-C₆-alkyldiyl;
L^{B} is a covalent bond or C₁-C₆-alkyldiyl;
L^{B1} is selected from —C₁-C₆-alkyldiyl-, —C(O)—C₁-C₆-alkyldiyl-, carbonyl, —NH—C(O)—, and —C(O)—NH—;
L^{C} is carbonyl or —C₁-C₆-alkyldiyl-NH—C(O)—;
L^{C1} is selected from carbonyl, —CH₂C(O)—, —NH—C(O)—, and —C(O)—NH—;
L^{C2} is selected from carbonyl, a covalent bond, —O—C₁-C₆-alkyl-NH—C(O)—, —C(O)—NH—, and —NH—C(O)—; and
n is 1 or 2.

2. The compound of formula (I) according to claim 1, or a pharmaceutically acceptable salt thereof, wherein
R¹ is selected from (C₁-C₆-alkyl)₃N⁺—C₁-C₆-alkyl-O—C₁-C₆-alkyl-, (C₁-C₆-alkyl)₃N⁺—C₁-C₆-alkyl-, (C₁-C₆-alkyl)₃N⁺—C₁-C₆-alkyl-C(O)—NH—C₁-C₆-alkyl-, carbamoyl-C₁-C₆-alkyl-(C₁-C₆-alkyl)₂N⁺—C₁-C₆-alkyl-O—C₁-C₆-alkyl-, carbamoyl-C₁-C₆-alkyl-(C₁-C₆-alkyl)₂N⁺—C₁-C₆-alkyl-, a group 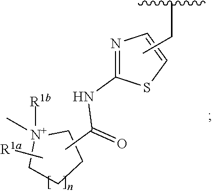
a group 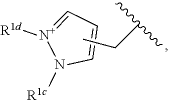
a group 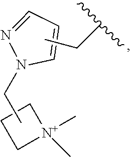
a group 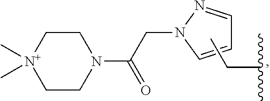
a group 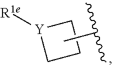
a group 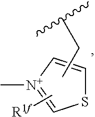
and a group 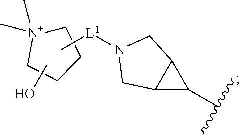
and R² is hydrogen; or
R¹ and R², taken together with the nitrogen atom to which they are attached, form a group 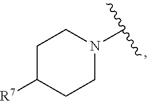
a group 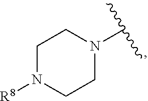
a group 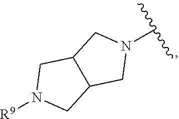
a group 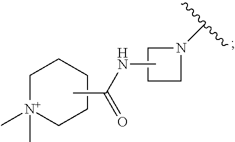
R^{1a} is hydrogen or hydroxy;
R^{1b} is C₁-C₆-alkyl or carbamoyl-C₁-C₆-alkyl-;
R^{1c} is selected from C₁-C₆-alkyl, hydroxy-C₁-C₆-alkyl, hydroxy-C₁-C₆-alkyl-O—C₁-C₆-alkyl-, carbamoyl-C₁-C₆-alkyl, C₁-C₆-alkoxy-C₁-C₆-alkyl-, and C1-C₆-alkyl-S(O)₂—C₁-C₆-alkyl-;
R^{1d} is C₁-C₆-alkyl or cyano-C₁-C₆-alkyl;
R^{1e} is selected from (C₁-C₆-alkyl)₃N⁺—C₁-C₆-alkyl-NH—, (C₁-C₆-alkyl)₃N⁺—C₁-C₆-alkyl-C(O)—NH—, and carbamoyl-C₁-C₆-alkyl-(C₁-C₆-alkyl)₂N⁺—C₁-C₆-alkyl-C(O)—;
R^{1f} is (C₁-C₆-alkyl)₃N⁺—C₁-C₆-alkyl-C(O)—NH—;
R⁷ is selected from (C₁-C₆-alkyl)₃N⁺—C₁-C₆-alkyl-, (C₁-C₆-alkyl)₃N⁺—C₁-C₆-alkyl-O—C₁-C₆-alkyl-NH—C(O)—, a group 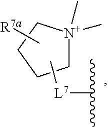
a group 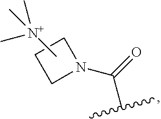
and a group 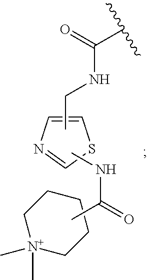
R^{7a} is hydrogen or hydroxy;
R⁸ is selected from (R^{8a})₃N⁺—C₁-C₆-alkyl-L^{8a}-, a group 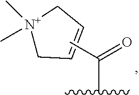
a group 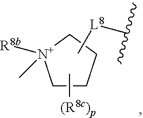
a group 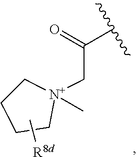
a group 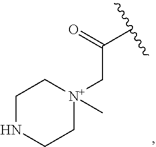 [SMILES: *C(=O)C[N+]1(C)CCNCC1]
a group 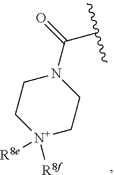
a group 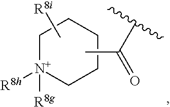
a group 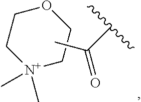
a group 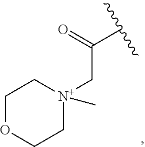 [SMILES: *C(=O)C[N+]1(C)CCOCC1]
a group 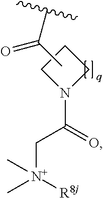
a group 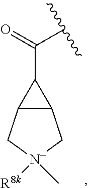
and a group 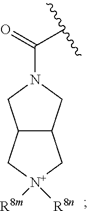
each R^{8a} is independently selected from amino-C₁-C₆-alkyl-, carboxy-C₁-C₆-alkyl-, carbamoyl-C₁-C₆-alkyl-, and C₁-C₆-alkyl-;
R^{8b} is C₁-C₆-alkyl or a group 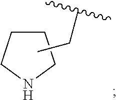
each R^{8c} is independently selected from hydrogen, hydroxy, C₁-C₆-alkyl, hydroxy-C₁-C₆-alkyl, and a group 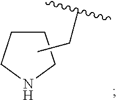
R^{8d} is selected from hydrogen, hydroxy, and carbamoyl;
R^{8e} is carboxy-C₁-C₆-alkyl-;
R^{8f} is C₁-C₆-alkyl or carboxy-C₁-C₆-alkyl-;
R^{8g} is selected from C₁-C₆-alkyl, carboxy-C₁-C₆-alkyl, and carbamoyl-C₁-C₆-alkyl;
R^{8h} is selected from C₁-C₆-alkyl, amino-C₁-C₆-alkyl, carbamoyl-C₁-C₆-alkyl, carboxy-C₁-C₆-alkyl, (C₁-C₆-alkyl)₂N—C₁-C₆-alkyl-, and a group 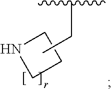
R⁸ⁱ is hydrogen or hydroxy;
R^{8j} is C₁-C₆-alkyl or carbamoyl-C₁-C₆-alkyl;
R^{8k} is carbamoyl-C₁-C₆-alkyl- or carboxy-C₁-C₆-alkyl-;
R^{8m} is carboxy-C₁-C₆-alkyl-;
R⁸ⁿ is amino-C₁-C₆-alkyl-;
R⁹ is (C₁-C₆-alkyl)₃N⁺—C₁-C₆-alkyl-C(O)— or a group 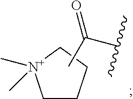
L¹ is carbonyl, —NH—C(O)—;
L⁷ is —NH—C(O)— or —C(O)—NH—;
L⁸ is carbonyl, —CH₂C(O)—, —NH—C(O)—;
L^{8a} is selected from carbonyl, a covalent bond, and —C(O)—NH—;
Y is N or CH;
n and p are each independently 1 or 2;
q is 1 or 3; and
r is 1, 2 or 3.

3. The compound of formula (I) according to claim 2, or a pharmaceutically acceptable salt thereof, wherein
R¹ is selected from a group 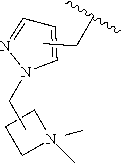
and a group 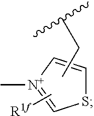
and R² is hydrogen; or
R¹ and R², taken together with the nitrogen atom to which they are attached, form a group 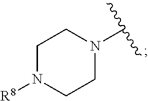
R^{1f} is (C₁-C₆-alkyl)₃N⁺—C₁-C₆-alkyl-C(O)—NH—;
R⁸ is selected from (R^{8a})₃N⁺—C₁-C₆-alkyl-L^{8a}-, a group 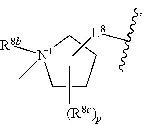
a group 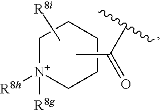
a group 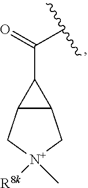
and a group 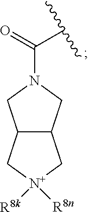
each R^{8a} is independently selected from carbamoyl-C₁-C₆-alkyl- and C₁-C₆-alkyl-;
R^{8b} is C₁-C₆-alkyl;
each R^{8c} is independently selected from hydroxy and C₁-C₆-alkyl;
R^{8g} is selected from C₁-C₆-alkyl, carboxy-C₁-C₆-alkyl, and carbamoyl-C₁-C₆-alkyl;
R^{8h} C₁-C₆-alkyl or a group 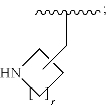
R⁸ⁱ is hydrogen or hydroxy;
R^{8k} is carbamoyl-C₁-C₆-alkyl-;
R^{8m} is carboxy-C₁-C₆-alkyl-;
R⁸ⁿ is amino-C₁-C₆-alkyl-;
L⁸ and L⁸a are both carbonyl;
p is 1 or 2; and
r is 1.

4. The compound of formula (I) according to claim 3, or a pharmaceutically acceptable salt thereof, wherein
R¹ is selected from a group 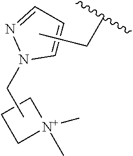
and a group 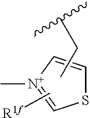
and R² is hydrogen; or
R¹ and R², taken together with the nitrogen atom to which they are attached, form a group 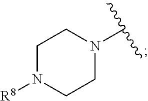
R^{1f} is (CH₃)₃N⁺—CH₂—C(O)—NH—;
R⁸ is selected from (R^{8a})₃N⁺—CH₂-L^{8a}-, a group 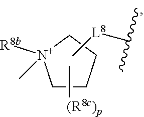
a group 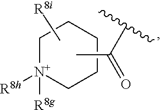
a group 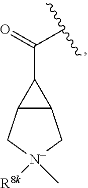
and a group 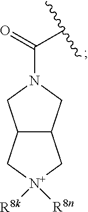
each R^{8a} is independently selected from 2-amino-2-oxo-ethyl and methyl;
R^{8b} is methyl;
each R^{8c} is independently selected from hydroxy and methyl;
R^{8g} is selected from methyl, carboxymethyl, and 2-amino-2-oxo-ethyl;
R^{8h} is methyl or a group 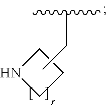
R⁸ⁱ is hydrogen or hydroxy;
R^{8k} is 2-amino-2-oxo-ethyl;
R^{8m} is carboxymethyl;
R⁸ⁿ is 3-aminopropyl;
L⁸ and L^{8a} are both carbonyl;
p is 1 or 2; and
r is 1.

5. The compound of formula (I) according to claim 1, or a pharmaceutically acceptable salt thereof, wherein R³ is halogen or C₁-C₆-alkyl.

6. The compound of formula (I) according to claim 5, or a pharmaceutically acceptable salt thereof, wherein R³ is halogen.

7. The compound of formula (I) according to claim 6, or a pharmaceutically acceptable salt thereof, wherein R³ is chloro.

8. The compound of formula (I) according to claim 1, or a pharmaceutically acceptable salt thereof, wherein:
R⁴ is selected from hydrogen, halogen, and halo-C₁-C₆-alkyl;
R⁵ is selected from hydrogen, halogen, cyano, and halo-C₁-C₆-alkyl; and
R⁶ is selected from C₁-C₆-alkoxy, halo-C₁-C₆-alkoxy, and cyano-C₁-C₆-alkoxy.

9. The compound of formula (I) according to claim 8, or a pharmaceutically acceptable salt thereof, wherein:
R⁴ and R⁵ are each halogen; and
R⁶ is C₁-C₆-alkoxy or halo-C₁-C₆-alkoxy.

10. The compound of formula (I) according to claim 9, or a pharmaceutically acceptable salt thereof, wherein:
R⁴ and R⁵ are each independently fluoro or chloro; and
R⁶ is selected from methoxy, fluoromethoxy, and difluoromethoxy.

11. The compound of formula (I) according to claim 1, or a pharmaceutically acceptable salt thereof, wherein:
R¹ is selected from (C₁-C₆-alkyl)₃N⁺—C₁-C₆-alkyl-O—C₁-C₆-alkyl-, (C₁-C₆-alkyl)₃N⁺—C₁-C₆-alkyl-, (C₁-C₆-alkyl)₃N⁺—C₁-C₆-alkyl-C(O)—NH—C₁-C₆-alkyl-, carbamoyl-C₁-C₆-alkyl-(C₁-C₆-alkyl)₂N⁺—C₁-C₆-alkyl-O—C₁-C₆-alkyl-, carbamoyl-C₁-C₆-alkyl-(C₁-C₆-alkyl)₂N⁺—C₁-C₆-alkyl-, a group 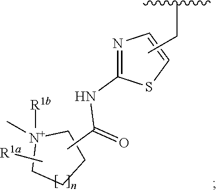
a group 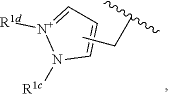
a group 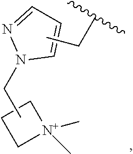
a group 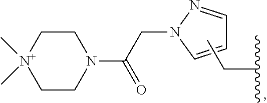
a group 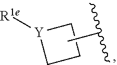
a group 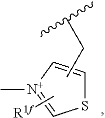
and a group 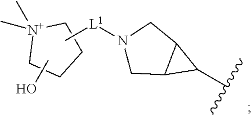
and R² is hydrogen; or
R¹ and R², taken together with the nitrogen atom to which they are attached, form a group 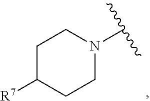
a group 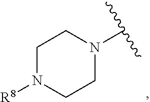
a group 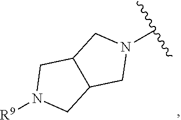
or a group 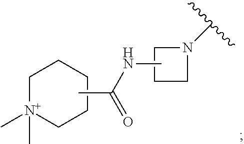
R^{1a} is hydrogen or hydroxy;
R^{1b} is C₁-C₆-alkyl or carbamoyl-C₁-C₆-alkyl-;
R^{1c} is selected from C₁-C₆-alkyl, hydroxy-C₁-C₆-alkyl, hydroxy-C₁-C₆-alkyl-O—C₁-C₆-alkyl-, carbamoyl-C₁-C₆-alkyl, C₁-C₆-alkoxy-C₁-C₆-alkyl-, and C1-C₆-alkyl-S(O)₂—C₁-C₆-alkyl-;
R^{1d} is C₁-C₆-alkyl or cyano-C₁-C₆-alkyl;
R^{1e} is selected from (C₁-C₆-alkyl)₃N⁺—C₁-C₆-alkyl-NH—, (C₁-C₆-alkyl)₃N⁺—C₁-C₆-alkyl-C(O)—NH—, and carbamoyl-C₁-C₆-alkyl-(C₁-C₆-alkyl)₂N⁺—C₁-C₆-alkyl-C(O)—;
R^{1f} is (C₁-C₆-alkyl)₃N⁺—C₁-C₆-alkyl-C(O)—NH—;
R³ is halogen or C₁-C₆-alkyl;
R⁴ is selected from hydrogen, halogen, and halo-C₁-C₆-alkyl;
R⁵ is selected from hydrogen, halogen, cyano, and halo-C₁-C₆-alkyl;
R⁶ is selected from C₁-C₆-alkoxy, halo-C₁-C₆-alkoxy, and cyano-C₁-C₆-alkoxy;
R⁷ is selected from (C₁-C₆-alkyl)₃N⁺—C₁-C₆-alkyl-, (C₁-C₆-alkyl)₃N⁺—C₁-C₆-alkyl-O—C₁-C₆-alkyl-NH—C(O)—, a group 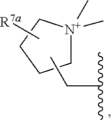
a group 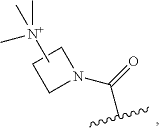
and a group 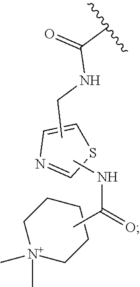
R^{7a} is hydrogen or hydroxy;
R⁸ is selected from (R^{8a})₃N⁺—C₁-C₆-alkyl-L^{8a}- a group 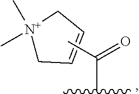
a group 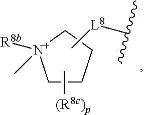
a group 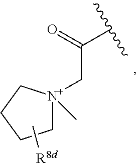
a group 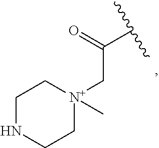 [SMILES: *C(=O)C[N+]1(C)CCNCC1]
a group 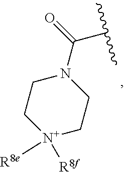
a group 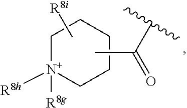
a group 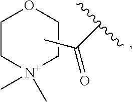
a group 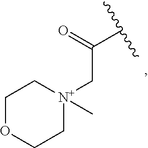 [SMILES: *C(=O)C[N+]1(C)CCOCC1]
a group 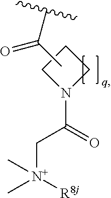
a group 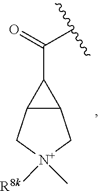
and a group 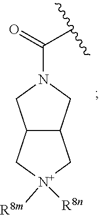
each R^{8a} is independently selected from amino-C₁-C₆-alkyl-, carboxy-C₁-C₆-alkyl-, carbamoyl-C₁-C₆-alkyl-, and C₁-C₆-alkyl-;
R^{8b} is C₁-C₆-alkyl or a group 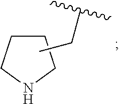
each R^{8c} is independently selected from hydrogen, hydroxy, C₁-C₆-alkyl, hydroxy-C₁-C₆-alkyl, and a group 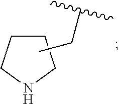
R^{8d} is selected from hydrogen, hydroxy, and carbamoyl;
R^{8e} is carboxy-C₁-C₆-alkyl-;
R^{8f} is C₁-C₆-alkyl or carboxy-C₁-C₆-alkyl-;
R^{8g} is selected from C₁-C₆-alkyl, carboxy-C₁-C₆-alkyl, and carbamoyl-C₁-C₆-alkyl;
R^{8h} is selected from C₁-C₆-alkyl, amino-C₁-C₆-alkyl, carbamoyl-C₁-C₆-alkyl, carboxy-C₁-C₆-alkyl, (C₁-C₆-alkyl)₂N—C₁-C₆-alkyl-, and a group 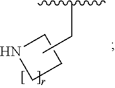
R⁸ⁱ is hydrogen or hydroxy;
R^{8j} is C₁-C₆-alkyl or carbamoyl-C₁-C₆-alkyl;
R^{8k} is carbamoyl-C₁-C₆-alkyl- or carboxy-C₁-C₆-alkyl-;
R^{8m} is carboxy-C₁-C₆-alkyl-;
R⁸ⁿ is amino-C₁-C₆-alkyl-;
R⁹ is (C₁-C₆-alkyl)₃N⁺—C₁-C₆-alkyl-C(O)— or a group 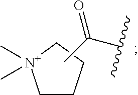
L¹ is carbonyl, —NH—C(O)—;
L⁷ is —NH—C(O)— or —C(O)—NH—;
L⁸ is carbonyl, —CH₂C(O)—, —NH—C(O)—;
L^{8a} is selected from carbonyl, a covalent bond, and —C(O)—NH—;
Y is N or CH;
n and p are each independently 1 or 2;
q is 1 or 3; and
r is 1, 2 or 3.

12. The compound of formula (I) according to claim 11, or a pharmaceutically acceptable salt thereof, wherein:
R¹ is selected from a group 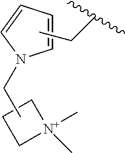
and a group 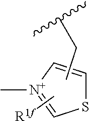
and R² is hydrogen; or
R¹ and R², taken together with the nitrogen atom to which they are attached, form a group 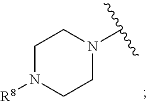
R^{1f} is (C₁-C₆-alkyl)₃N⁺—C₁-C₆-alkyl-C(O)—NH—;
R³, R⁴ and R are each halogen;
R⁶ is C₁-C₆-alkoxy or halo-C₁-C₆-alkoxy;
R⁸ is selected from (R^{8a})₃N⁺—C₁-C₆-alkyl-L^{8a}-, a group 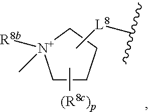
a group 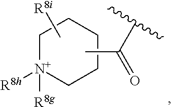
a group 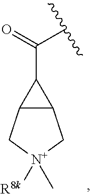
and a group 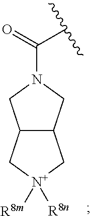
each R^{8a} is independently selected from carbamoyl-C₁-C₆-alkyl- and C₁-C₆-alkyl-;
R^{8b} is C₁-C₆-alkyl;
each R^{8c} is independently selected from hydroxy and C₁-C₆-alkyl;
R^{8g} is selected from C₁-C₆-alkyl, carboxy-C₁-C₆-alkyl, and carbamoyl-C₁-C₆-alkyl;
R^{8h} is C₁-C₆-alkyl or a group 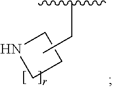
R⁸ⁱ is hydrogen or hydroxy;
R^{8k} is carbamoyl-C₁-C₆-alkyl-;
R^{8m} is carboxy-C₁-C₆-alkyl-;
R⁸ⁿ is amino-C₁-C₆-alkyl-;
L⁸ and L⁸a are both carbonyl;
p is 1 or 2; and,
r is 1.

13. The compound of formula (I) according to claim 11, or a pharmaceutically acceptable salt thereof, wherein:
R¹ is selected from a group 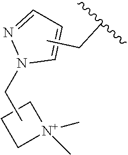
and a group 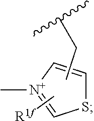
and R² is hydrogen; or
R¹ and R², taken together with the nitrogen atom to which they are attached, form a group 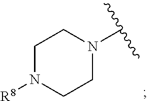
R^{1f} is (CH₃)₃N⁺—CH₂—C(O)—NH—;
R³ is chloro;
R⁴ and R⁵ are each independently fluoro or chloro;
R⁶ is selected from methoxy, fluoromethoxy, and difluoromethoxy;
R⁸ is selected from (R^{8a})₃N⁺—CH₂-L^{8a}-, a group 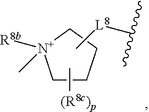
a group 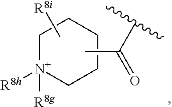
a group 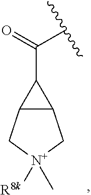
and a group 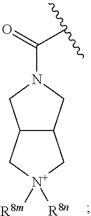
each R^{8a} is independently selected from 2-amino-2-oxo-ethyl and methyl;
R^{8b} is methyl;
each R^{8c} is independently selected from hydroxy and methyl;
R^{8g} is selected from methyl, carboxymethyl, and 2-amino-2-oxo-ethyl;
R^{8h} is methyl or a group 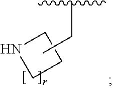
R⁸ⁱ is hydrogen or hydroxy;
R^{8k} is 2-amino-2-oxo-ethyl;
R^{8m} is carboxymethyl;
R⁸ⁿ is 3-aminopropyl;
L⁸ and L^{8a} are both carbonyl;
p is 1 or 2; and
r is 1.

14. The compound of formula (I) according to claim 1, or a pharmaceutically acceptable salt thereof, wherein said compound of formula (I) is selected from:
2-[1-[2-Chloro-4-[[5-(2,3-difluoro-4-methoxy-phenyl)-1-methyl-imidazole-2-carbonyl]amino]benzoyl]-4-piperidyl]ethyl-trimethyl-ammonium;formate;
5-[[2-Chloro-4-[[5-(2,3-difluoro-4-methoxy-phenyl)-1-methyl-imidazole-2-carbonyl]amino]benzoyl]amino]pentyl-trimethyl-ammonium;formate;
(2-amino-2-oxo-ethyl)-[5-[[2-chloro-4-[[5-(2,3-difluoro-4-methoxy-phenyl)-1-methyl-imidazole-2-carbonyl]amino]benzoyl]amino]pentyl]-dimethyl-ammonium;formate;
(2-amino-2-oxo-ethyl)-[2-[4-[2-chloro-4-[[5-(2,3-difluoro-4-methoxy-phenyl)-1-methyl-imidazole-2-carbonyl]amino]benzoyl]piperazin-1-yl]ethyl]-dimethyl-ammonium;formate;
2-(2-(4-(5-(2,3-difluoro-4-methoxyphenyl)-1-methyl-1H-imidazole-2-carboxamido)-2-ethylbenzamido)ethoxy)-N,N,N-trimethylethan-1-aminium iodide;
3-(4-(4-(5-(2,3-difluoro-4-methoxyphenyl)-1-methyl-1H-imidazole-2-carboxamido)-2-ethylbenzoyl)piperazin-1-yl)-N,N,N-trimethylpropan-1-aminium iodide;
[2-[4-[2-chloro-4-[[5-[4-(cyanomethoxy)-2,3-difluoro-phenyl]-1-methyl-imidazole-2-carbonyl]amino]benzoyl]piperazin-1-yl]-2-oxo-ethyl]-trimethyl-ammonium;formate;iodide;
[2-[4-[4-[[5-[4-(cyanomethoxy)-2,3-difluoro-phenyl]-1-methyl-imidazole-2-carbonyl]amino]-2-methyl-benzoyl]piperazin-1-yl]-2-oxo-ethyl]-trimethyl-ammonium;iodide;
2-[4-[2-chloro-4-[[5-(2,3-difluoro-4-methoxy-phenyl)-1-methyl-imidazole-2-carbonyl]amino]benzoyl]piperazin-1-yl]ethyl-trimethyl-ammonium;iodide;
[2-[[3-[[2-Chloro-4-[[5-(2,3-difluoro-4-methoxy-phenyl)-1-methyl-imidazole-2-carbonyl]amino]benzoyl]amino]cyclobutyl]amino]-2-oxo-ethyl]-trimethyl-ammonium formate;
2-[[3-[[2-chloro-4-[[5-(2,3-difluoro-4-methoxy-phenyl)-1-methyl-imidazole-2-carbonyl]amino]benzoyl]amino]cyclobutyl]amino]ethyl-trimethyl-ammonium; formic acid;
formate;
[2-[3-[[2-chloro-4-[[5-[4-(cyanomethoxy)-2,3-difluoro-phenyl]-1-methyl-imidazole-2-carbonyl]amino]benzoyl]amino]propylamino]-2-oxo-ethyl]-trimethyl-ammonium formate;
(2-amino-2-oxo-ethyl)-[2-[2-[[2-chloro-4-[[5-[4-(cyanomethoxy)-2,3-difluoro-phenyl]-1-methyl-imidazole-2-carbonyl]amino]benzoyl]amino]ethoxy]ethyl]-dimethyl-ammonium formate;
[2-[2-[[2-chloro-4-[[5-[4-(cyanomethoxy)-2,3-difluoro-phenyl]-1-methyl-imidazole-2-carbonyl]amino]benzoyl]amino]ethylamino]-2-oxo-ethyl]-trimethyl-ammonium formate;
2-[2-[[2-chloro-4-[[5-[4-(cyanomethoxy)-2,3-difluoro-phenyl]-1-methyl-imidazole-2-carbonyl]amino]benzoyl]amino]ethoxy]ethyl-trimethyl-ammonium formate;
[2-[[3-[[2-chloro-4-[[5-[4-(cyanomethoxy)-2,3-difluoro-phenyl]-1-methyl-imidazole-2-carbonyl]amino]benzoyl]amino]cyclobutyl]amino]-2-oxo-ethyl]-trimethyl-ammonium formate;
2-[[3-[[2-chloro-4-[[5-[4-(cyanomethoxy)-2,3-difluoro-phenyl]-1-methyl-imidazole-2-carbonyl]amino]benzoyl]amino]cyclobutyl]amino]ethyl-trimethyl-ammonium formate;
[2-[[3-[[4-[[5-[4-(cyanomethoxy)-2,3-difluoro-phenyl]-1-methyl-imidazole-2-carbonyl]amino]-2-methyl-benzoyl]amino]cyclobutyl]amino]-2-oxo-ethyl]-trimethyl-ammonium formate;
2-[[3-[[4-[[5-[4-(cyanomethoxy)-2,3-difluoro-phenyl]-1-methyl-imidazole-2-carbonyl]amino]-2-methyl-benzoyl]amino]cyclobutyl]amino]ethyl-trimethyl-ammonium formate;
N-[3-chloro-4-[[1-(2-hydroxyethyl)-2-methyl-pyrazol-2-ium-4-yl]methylcarbamoyl]phenyl]-5-(2,3-difluoro-4-methoxy-phenyl)-1-methyl-imidazole-2-carboxamide;formate;
N-[3-chloro-4-[[1-(3-hydroxypropyl)-2-methyl-pyrazol-2-ium-4-yl]methylcarbamoyl]phenyl]-5-(2,3-difluoro-4-methoxy-phenyl)-1-methyl-imidazole-2-carboxamide;formate;
N-[3-chloro-4-[[1-(2-cyanoethyl)-2-methyl-pyrazol-2-ium-4-yl]methylcarbamoyl]phenyl]-5-(2,3-difluoro-4-methoxy-phenyl)-1-methyl-imidazole-2-carboxamide;formate;
N-[4-[[1-(2-amino-2-oxo-ethyl)-2-methyl-pyrazol-2-ium-4-yl]methylcarbamoyl]-3-chloro-phenyl]-5-(2,3-difluoro-4-methoxy-phenyl)-1-methyl-imidazole-2-carboxamide;formate;
N-[3-chloro-4-[[1-methyl-2-(2-methylsulfonylethyl)pyrazol-1-ium-4-yl]methylcarbamoyl]phenyl]-5-(2,3-difluoro-4-methoxy-phenyl)-1-methyl-imidazole-2-carboxamide;formate;
N-[3-chloro-4-[[1-(3-methoxypropyl)-2-methyl-pyrazol-2-ium-4-yl]methylcarbamoyl]phenyl]-5-(2,3-difluoro-4-methoxy-phenyl)-1-methyl-imidazole-2-carboxamide;formate;
N-[3-chloro-4-[[1-[2-(2-hydroxyethoxy)ethyl]-2-methyl-pyrazol-2-ium-4-yl]methylcarbamoyl]phenyl]-5-(2,3-difluoro-4-methoxy-phenyl)-1-methyl-imidazole-2-carboxamide;formate;
N-[3-chloro-4-[[2-[[(2S,4R)-4-hydroxy-1,1-dimethyl-pyrrolidin-1-ium-2-carbonyl]amino]thiazol-5-yl]methylcarbamoyl]phenyl]-5-(2,3-difluoro-4-methoxy-phenyl)-1-methyl-imidazole-2-carboxamide;formate;
N-[3-chloro-4-[[2-[(1,1-dimethylpyrrolidin-1-ium-2-carbonyl)amino]thiazol-5-yl]methylcarbamoyl]phenyl]-5-(2,3-difluoro-4-methoxy-phenyl)-1-methyl-imidazole-2-carboxamide;formate;
N-[5-[[[2-chloro-4-[[5-(2,3-difluoro-4-methoxy-phenyl)-1-methyl-imidazole-2-carbonyl]amino]benzoyl]amino]methyl]thiazol-2-yl]-1,1-dimethyl-piperidin-1-ium-4-carboxamide;formate;
1-(2-amino-2-oxo-ethyl)-N-[5-[[[2-chloro-4-[[5-(2,3-difluoro-4-methoxy-phenyl)-1-methyl-imidazole-2-carbonyl]amino]benzoyl]amino]methyl]thiazol-2-yl]-1-methyl-piperidin-1-ium-4-carboxamide;formate;
N-[5-[[[1-[2-chloro-4-[[5-(2,3-difluoro-4-methoxy-phenyl)-1-methyl-imidazole-2-carbonyl]amino]benzoyl]piperidine-4-carbonyl]amino]methyl]thiazol-2-yl]-1,1-dimethyl-piperidin-1-ium-4-carboxamide;formate;
(2-Amino-2-oxo-ethyl)-[2-[4-[4-[[5-(2,3-difluoro-4-methoxy-phenyl)-1-methyl-imidazole-2-carbonyl]amino]-2-methyl-benzoyl]piperazin-1-yl]-2-oxo-ethyl]-dimethyl-ammonium;formate;
(2-amino-2-oxo-ethyl)-[2-[4-[4-[[5-[4-(cyanomethoxy)-2,3-difluoro-phenyl]-1-methyl-imidazole-2-carbonyl]amino]-2-methyl-benzoyl]piperazin-1-yl]-2-oxo-ethyl]-dimethyl-ammonium;formate;
(2-amino-2-oxo-ethyl)-[3-[4-[4-[[5-(2,3-difluoro-4-methoxy-phenyl)-1-methyl-imidazole-2-carbonyl]amino]-2-methyl-benzoyl]piperazin-1-yl]-3-oxo-propyl]-dimethyl-ammonium;2,2,2-trifluoroacetate;
(2-amino-2-oxo-ethyl)-[2-[4-[2-chloro-4-[[5-[4-(cyanomethoxy)-2,3-difluoro-phenyl]-1-methyl-imidazole-2-carbonyl]amino]benzoyl]piperazin-1-yl]-2-oxo-ethyl]-dimethyl-ammonium;formate;
(2-amino-2-oxo-ethyl)-[2-[[1-[2-chloro-4-[[5-(2,3-difluoro-4-methoxy-phenyl)-1-methyl-imidazole-2-carbonyl]amino]benzoyl]-4-piperidyl]amino]-2-oxo-ethyl]-dimethyl-ammonium;formate;
(2-amino-2-oxo-ethyl)-[2-[3-[[2-chloro-4-[[5-(2,3-difluoro-4-methoxy-phenyl)-1-methyl-imidazole-2-carbonyl]amino]benzoyl]amino]azetidin-1-yl]-2-oxo-ethyl]-dimethyl-ammonium;formate;
(2-amino-2-oxo-ethyl)-[2-[4-[2-chloro-4-[[5-(2,3-difluoro-4-methoxy-phenyl)-1-methyl-imidazole-2-carbonyl]amino]benzoyl]piperazin-1-yl]-2-oxo-ethyl]-dimethyl-ammonium;formate;
[3-[4-[4-[[5-(2,3-Difluoro-4-methoxy-phenyl)-1-methyl-imidazole-2-carbonyl]amino]-2-methyl-benzoyl]piperazin-1-yl]-3-oxo-propyl]-trimethyl-ammonium;2,2,2-trifluoroacetate;
[3-[4-[2-chloro-4-[[5-[4-(cyanomethoxy)-2,3-difluoro-phenyl]-1-methyl-imidazole-2-carbonyl]amino]benzoyl]piperazin-1-yl]-3-oxo-propyl]-trimethyl-ammonium;iodide;
N-[3-chloro-4-[4-(1,1-dimethylpiperidin-1-ium-4-carbonyl)piperazine-1-carbonyl]phenyl]-5-(2,3-difluoro-4-methoxy-phenyl)-1-methyl-imidazole-2-carboxamide;formate;
[2-[4-[2-chloro-4-[[5-[4-(difluoromethoxy)-2-fluoro-phenyl]-1-methyl-imidazole-2-carbonyl]amino]benzoyl]piperazin-1-yl]-2-oxo-ethyl]-trimethyl-ammonium;formate;
[2-[4-[2-chloro-4-[[5-[4-(difluoromethoxy)-2,3-difluoro-phenyl]-1-methyl-imidazole-2-carbonyl]amino]benzoyl]piperazin-1-yl]-2-oxo-ethyl]-trimethyl-ammonium;formate;
N-[3-chloro-4-[4-[2-(1,1-dimethylpyrrolidin-1-ium-3-yl)acetyl]piperazine-1-carbonyl]phenyl]-5-(2,3-difluoro-4-methoxy-phenyl)-1-methyl-imidazole-2-carboxamide;formate;
[2-[4-[4-[2-chloro-4-[[5-[4-(difluoromethoxy)-2,3-difluoro-phenyl]-1-methyl-imidazole-2-carbonyl]amino]benzoyl]piperazine-1-carbonyl]-1-piperidyl]-2-oxo-ethyl]-trimethyl-ammonium;formate;
[2-[4-[2-chloro-4-[[5-[4-(difluoromethoxy)-3-fluoro-phenyl]-1-methyl-imidazole-2-carbonyl]amino]benzoyl]piperazin-1-yl]-2-oxo-ethyl]-trimethyl-ammonium;formate;
[2-[3-[4-[2-chloro-4-[[5-(2,3-difluoro-4-methoxy-phenyl)-1-methyl-imidazole-2-carbonyl]amino]benzoyl]piperazine-1-carbonyl]azetidin-1-yl]-2-oxo-ethyl]-trimethyl-ammonium;formate;
N-[1-[2-chloro-4-[[5-(2,3-difluoro-4-methoxy-phenyl)-1-methyl-imidazole-2-carbonyl]amino]benzoyl]azetidin-3-yl]-1,1-dimethyl-piperidin-1-ium-4-carboxamide;formate;
N-[4-[(3aS,6aR)-5-[(3R)-1,1-dimethylpyrrolidin-1-ium-3-carbonyl]-1,3,3a,4,6,6a-hexahydropyrrolo[3,4-c]pyrrole-2-carbonyl]-3-chloro-phenyl]-5-(2,3-difluoro-4-methoxy-phenyl)-1-methyl-imidazole-2-carboxamide;formate;
[2-[(3aS,6aR)-5-[2-chloro-4-[[5-(2,3-difluoro-4-methoxy-phenyl)-1-methyl-imidazole-2-carbonyl]amino]benzoyl]-1,3,3a,4,6,6a-hexahydropyrrolo[3,4-c]pyrrol-2-yl]-2-oxo-ethyl]-trimethyl-ammonium;formate;
[2-[4-[2-chloro-4-[[5-(2,3-difluoro-4-methoxy-phenyl)-1-methyl-imidazole-2-carbonyl]amino]benzoyl]piperazin-1-yl]-2-oxo-ethyl]-trimethyl-ammonium;formate;
[2-[4-[4-[[5-(2,3-difluoro-4-methoxy-phenyl)-1-methyl-imidazole-2-carbonyl]amino]-2-methyl-benzoyl]piperazin-1-yl]-2-oxo-ethyl]-trimethyl-ammonium;formate;
[2-[4-[2-chloro-4-[[5-[2-fluoro-4-(fluoromethoxy)phenyl]-1-methyl-imidazole-2-carbonyl]amino]benzoyl]piperazin-1-yl]-2-oxo-ethyl]-trimethyl-ammonium;formate;
2-(2-(1-(4-(5-(2,3-difluoro-4-methoxyphenyl)-1-methyl-1H-imidazole-2-carboxamido)-2-ethylbenzoyl)piperidine-4-carboxamido)ethoxy)-N,N,N-trimethylethan-1-aminium iodide;
(S)-3-(1-(2-chloro-4-(5-(4-(cyanomethoxy)-2,3-difluorophenyl)-1-methyl-1H-imidazole-2-carboxamido)benzoyl)piperidine-4-carboxamido)-1,1-dimethylpyrrolidin-1-ium formate;
(2S,4R)-2-(4-(2-chloro-4-(5-(2,3-difluoro-4-methoxyphenyl)-1-methyl-1H-imidazole-2-carboxamido)benzoyl)piperazine-1-carbonyl)-4-hydroxy-1,1-dimethylpyrrolidin-1-ium formate;
4-(4-(4-(5-(4-(cyanomethoxy)-2,3-difluorophenyl)-1-methyl-1H-imidazole-2-carboxamido)-2-methylbenzoyl)piperazine-1-carbonyl)-4-hydroxy-1,1-dimethylpiperidin-1-ium formate;
(2S,4S)-2-(4-(2-chloro-4-(5-(4-(cyanomethoxy)-2,3-difluorophenyl)-1-methyl-1H-imidazole-2-carboxamido)benzoyl)piperazine-1-carbonyl)-4-hydroxy-1,1,4-trimethylpyrrolidin-1-ium formate;
(3R,4R)-3-(4-(4-(5-(4-(cyanomethoxy)-2,3-difluorophenyl)-1-methyl-1H-imidazole-2-carboxamido)-2-methylbenzoyl)piperazine-1-carboxamido)-4-hydroxy-1,1-dimethylpyrrolidin-1-ium formate;
(3S,4S)-3-(4-(4-(5-(4-(cyanomethoxy)-2,3-difluorophenyl)-1-methyl-1H-imidazole-2-carboxamido)-2-methylbenzoyl)piperazine-1-carboxamido)-4-hydroxy-1,1-dimethylpyrrolidin-1-ium formate;
(2S,4R)-2-(4-(2-chloro-4-(5-(2,3-difluoro-4-methoxyphenyl)-1-methyl-1H-imidazole-2-carboxamido)benzoyl)piperazine-1-carbonyl)-4-hydroxy-1,1,4-trimethylpyrrolidin-1-ium formate;
[1-[1-[2-chloro-4-[[5-[4-(cyanomethoxy)-2,3-difluoro-phenyl]-1-methyl-imidazole-2-carbonyl]amino]benzoyl]piperidine-4-carbonyl]azetidin-3-yl]-trimethyl-ammonium;formate;
N-[3-chloro-4-[[1-[2-(4,4-dimethylpiperazin-4-ium-1-yl)-2-oxo-ethyl]pyrazol-4-yl]methylcarbamoyl]phenyl]-5-(2,3-difluoro-4-methoxy-phenyl)-1-methyl-imidazole-2-carboxamide;formate;
N-[3-chloro-4-[4-(3-hydroxy-1,1-dimethyl-piperidin-1-ium-4-carbonyl)piperazine-1-carbonyl]phenyl]-5-(2,3-difluoro-4-methoxy-phenyl)-1-methyl-imidazole-2-carboxamide;formate;
N-[3-chloro-4-[4-[(2S,4S)-4-hydroxy-1,1-dimethyl-pyrrolidin-1-ium-2-carbonyl]piperazine-1-carbonyl]phenyl]-5-(2,3-difluoro-4-methoxy-phenyl)-1-methyl-imidazole-2-carboxamide;formate;
N-[3-chloro-4-[4-[(3S)-4,4-dimethylmorpholin-4-ium-3-carbonyl]piperazine-1-carbonyl]phenyl]-5-(2,3-difluoro-4-methoxy-phenyl)-1-methyl-imidazole-2-carboxamide;iodide;
N-[3-chloro-4-[4-[(2S,3S)-3-hydroxy-1,1-dimethyl-pyrrolidin-1-ium-2-carbonyl]piperazine-1-carbonyl]phenyl]-5-(2,3-difluoro-4-methoxy-phenyl)-1-methyl-imidazole-2-carboxamide;formate;
N-[3-chloro-4-[4-[(2R,4S)-4-hydroxy-1,1-dimethyl-pyrrolidin-1-ium-2-carbonyl]piperazine-1-carbonyl]phenyl]-5-(2,3-difluoro-4-methoxy-phenyl)-1-methyl-imidazole-2-carboxamide;formate;
N-[3-chloro-4-[4-[(2S,4R)-4-hydroxy-1,1-dimethyl-piperidin-1-ium-2-carbonyl]piperazine-1-carbonyl]phenyl]-5-(2,3-difluoro-4-methoxy-phenyl)-1-methyl-imidazole-2-carboxamide;formate;
4-[2-chloro-4-[[5-(2,3-difluoro-4-methoxy-phenyl)-1-methyl-imidazole-2-carbonyl]amino]benzoyl]-N-(4-hydroxy-1,1-dimethyl-pyrrolidin-1-ium-3-yl)piperazine-1-carboxamide;formate;
N-[3-chloro-4-[4-[(2S,4R)-4-hydroxy-1,1-dimethyl-pyrrolidin-1-ium-2-carbonyl]piperazine-1-carbonyl]phenyl]-5-[2-(difluoromethyl)-3-fluoro-4-methoxy-phenyl]-1-methyl-imidazole-2-carboxamide;formate;
N-[3-chloro-4-[4-[(2S,4R)-4-hydroxy-1,1-dimethyl-pyrrolidin-1-ium-2-carbonyl]piperazine-1-carbonyl]phenyl]-5-[4-(difluoromethoxy)-2,3-difluoro-phenyl]-1-methyl-imidazole-2-carboxamide;formate;
N-[3-chloro-4-[4-[(2S,4R)-4-(hydroxymethyl)-1,1-dimethyl-pyrrolidin-1-ium-2-carbonyl]piperazine-1-carbonyl]phenyl]-5-(2,3-difluoro-4-methoxy-phenyl)-1-methyl-imidazole-2-carboxamide;formate;
5-(2-chloro-3-fluoro-4-methoxy-phenyl)-N-[3-chloro-4-[4-[(2S,4R)-4-hydroxy-1,1-dimethyl-pyrrolidin-1-ium-2-carbonyl]piperazine-1-carbonyl]phenyl]-1-methyl-imidazole-2-carboxamide;formate;
N-[3-chloro-4-[4-[(2S,3R)-3-hydroxy-1,1-dimethyl-pyrrolidin-1-ium-2-carbonyl]piperazine-1-carbonyl]phenyl]-5-(2,3-difluoro-4-methoxy-phenyl)-1-methyl-imidazole-2-carboxamide;formate;
N-[3-chloro-4-[4-[(2S,4R)-4-hydroxy-1,1-dimethyl-pyrrolidin-1-ium-2-carbonyl]piperazine-1-carbonyl]phenyl]-5-[2,3-difluoro-4-(fluoromethoxy)phenyl]-1-methyl-imidazole-2-carboxamide;formate;
N-[3-chloro-4-[4-[(2S,5S)-5-hydroxy-1,1-dimethyl-piperidin-1-ium-2-carbonyl]piperazine-1-carbonyl]phenyl]-5-(2,3-difluoro-4-methoxy-phenyl)-1-methyl-imidazole-2-carboxamide;formate;
5-[3-chloro-4-(difluoromethoxy)-2-fluoro-phenyl]-N-[3-chloro-4-[4-[(2S,4R)-4-hydroxy-1,1-dimethyl-pyrrolidin-1-ium-2-carbonyl]piperazine-1-carbonyl]phenyl]-1-methyl-imidazole-2-carboxamide;iodide;
5-(3-chloro-2-fluoro-4-methoxy-phenyl)-N-[3-chloro-4-[4-[(2S,4R)-4-hydroxy-1,1-dimethyl-pyrrolidin-1-ium-2-carbonyl]piperazine-1-carbonyl]phenyl]-1-methyl-imidazole-2-carboxamide;formate;
N-[3-chloro-4-[4-[(2S)-1,1-dimethyl-2,5-dihydropyrrol-1-ium-2-carbonyl]piperazine-1-carbonyl]phenyl]-5-(2,3-difluoro-4-methoxy-phenyl)-1-methyl-imidazole-2-carboxamide;formate;
N-[3-chloro-4-[4-[(2S,3R,4S)-3,4-dihydroxy-1,1-dimethyl-pyrrolidin-1-ium-2-carbonyl]piperazine-1-carbonyl]phenyl]-5-(2,3-difluoro-4-methoxy-phenyl)-1-methyl-imidazole-2-carboxamide;formate;
N-[3-chloro-4-[4-[(2S,4R)-4-hydroxy-1,1-dimethyl-pyrrolidin-1-ium-2-carbonyl]piperazine-1-carbonyl]phenyl]-5-(3-cyano-2-fluoro-4-methoxy-phenyl)-1-methyl-imidazole-2-carboxamide;formate;
N-[3-chloro-4-[4-[(2S,4R)-4-hydroxy-1,1-dimethyl-pyrrolidin-1-ium-2-carbonyl]piperazine-1-carbonyl]phenyl]-5-[2-fluoro-4-methoxy-3-(trifluoromethyl)phenyl]-1-methyl-imidazole-2-carboxamide;formate;
5-[2-chloro-4-(difluoromethoxy)-3-fluoro-phenyl]-N-[3-chloro-4-[4-[(2S,4R)-4-hydroxy-1,1-dimethyl-pyrrolidin-1-ium-2-carbonyl]piperazine-1-carbonyl]phenyl]-1-methyl-imidazole-2-carboxamide;formate;
N-[3-chloro-4-[[(1R,5S)-3-[(2S,4R)-4-hydroxy-1,1-dimethyl-pyrrolidin-1-ium-2-carbonyl]-3-azabicyclo[3.1.0]hexan-6-yl]carbamoyl]phenyl]-5-(2,3-difluoro-4-methoxy-phenyl)-1-methyl-imidazole-2-carboxamide;formate;
(1S,5R)-6-[[2-chloro-4-[[5-(2,3-difluoro-4-methoxy-phenyl)-1-methyl-imidazole-2-carbonyl]amino]benzoyl]amino]-N-[(3R,4R)-4-hydroxy-1,1-dimethyl-pyrrolidin-1-ium-3-yl]-3-azabicyclo[3.1.0]hexane-3-carboxamide;formate;
[2-[4-[2-chloro-4-[[5-[2,3-difluoro-4-(fluoromethoxy)phenyl]-1-methyl-imidazole-2-carbonyl]amino]benzoyl]piperazin-1-yl]-2-oxo-ethyl]-trimethyl-ammonium;formate;
N-[4-[4-[1-(2-Amino-2-oxo-ethyl)-1-methyl-piperidin-1-ium-4-carbonyl]piperazine-1-carbonyl]-3-chloro-phenyl]-5-[2-chloro-4-(difluoromethoxy)-3-fluoro-phenyl]-1-methyl-imidazole-2-carboxamide;formate;
N-[4-[4-[1-(2-amino-2-oxo-ethyl)-1-methyl-piperidin-1-ium-4-carbonyl]piperazine-1-carbonyl]-3-chloro-phenyl]-5-[4-(difluoromethoxy)-2,3-difluoro-phenyl]-1-methyl-imidazole-2-carboxamide;formate;
N-[4-[4-[1-(2-amino-2-oxo-ethyl)-1-methyl-piperidin-1-ium-4-carbonyl]piperazine-1-carbonyl]-3-chloro-phenyl]-5-(2-chloro-3-fluoro-4-methoxy-phenyl)-1-methyl-imidazole-2-carboxamide;formate;
N-[4-[4-[(1R,5S)-3-(2-amino-2-oxo-ethyl)-3-methyl-3-azoniabicyclo[3.1.0]hexane-6-carbonyl]piperazine-1-carbonyl]-3-chloro-phenyl]-5-[4-(difluoromethoxy)-2,3-difluoro-phenyl]-1-methyl-imidazole-2-carboxamide;formate;
N-[4-[4-[1-(2-amino-2-oxo-ethyl)-1-methyl-piperidin-1-ium-4-carbonyl]piperazine-1-carbonyl]-3-chloro-phenyl]-5-(2,3-difluoro-4-methoxy-phenyl)-1-methyl-imidazole-2-carboxamide;formate;
[2-[4-[4-[2-chloro-4-[[5-(2,3-difluoro-4-methoxy-phenyl)-1-methyl-imidazole-2-carbonyl]amino]benzoyl]piperazine-1-carbonyl]-1-piperidyl]-2-oxo-ethyl]-trimethyl-ammonium;formate;
(2-amino-2-oxo-ethyl)-[2-[4-[4-[2-chloro-4-[[5-(2,3-difluoro-4-methoxy-phenyl)-1-methyl-imidazole-2-carbonyl]amino]benzoyl]piperazine-1-carbonyl]-1-piperidyl]-2-oxo-ethyl]-dimethyl-ammonium;formate;
2-[4-[4-[2-chloro-4-[[5-(2,3-difluoro-4-methoxy-phenyl)-1-methyl-imidazole-2-carbonyl]amino]benzoyl]piperazine-1-carbonyl]-1-methyl-piperidin-1-ium-1-yl]acetic acid;formate;
N-[4-[4-[1,1-bis(2-amino-2-oxo-ethyl)piperidin-1-ium-4-carbonyl]piperazine-1-carbonyl]-3-chloro-phenyl]-5-[2-chloro-4-(difluoromethoxy)-3-fluoro-phenyl]-1-methyl-imidazole-2-carboxamide;formate;
N-[4-[4-[1-(2-amino-2-oxo-ethyl)-1-(azetidin-3-ylmethyl)piperidin-1-ium-4-carbonyl]piperazine-1-carbonyl]-3-chloro-phenyl]-5-[2-chloro-4-(difluoromethoxy)-3-fluoro-phenyl]-1-methyl-imidazole-2-carboxamide;2,2,2-trifluoroacetate;2,2,2-trifluoroacetic acid;
N-[4-[4-[1-(2-Aminoethyl)-1-methyl-piperidin-1-ium-4-carbonyl]piperazine-1-carbonyl]-3-chloro-phenyl]-5-(2,3-difluoro-4-methoxy-phenyl)-1-methyl-imidazole-2-carboxamide;formate;
N-[4-[4-[1-(2-aminoethyl)-1-methyl-piperidin-1-ium-4-carbonyl]piperazine-1-carbonyl]-3-chloro-phenyl]-5-[2-chloro-4-(difluoromethoxy)-3-fluoro-phenyl]-1-methyl-imidazole-2-carboxamide;formic acid;formate;
N-[4-[4-[1-(azetidin-3-ylmethyl)-1-methyl-piperidin-1-ium-4-carbonyl]piperazine-1-carbonyl]-3-chloro-phenyl]-5-[4-(difluoromethoxy)-2,3-difluoro-phenyl]-1-methyl-imidazole-2-carboxamide;2,2,2-trifluoroacetate;2,2,2-trifluoroacetic acid;
N-[3-chloro-4-[4-[1-methyl-1-(pyrrolidin-3-ylmethyl)piperidin-1-ium-4-carbonyl]piperazine-1-carbonyl]phenyl]-5-[4-(difluoromethoxy)-2,3-difluoro-phenyl]-1-methyl-imidazole-2-carboxamide;formic acid;formate;
N-[4-[4-[1-(azetidin-3-ylmethyl)-1-methyl-piperidin-1-ium-4-carbonyl]piperazine-1-carbonyl]-3-chloro-phenyl]-5-[2-chloro-4-(difluoromethoxy)-3-fluoro-phenyl]-1-methyl-imidazole-2-carboxamide;formic acid;formate;
N-[4-[4-[1-(azetidin-3-ylmethyl)-1-methyl-piperidin-1-ium-4-carbonyl]piperazine-1-carbonyl]-3-chloro-phenyl]-5-(2,3-difluoro-4-methoxy-phenyl)-1-methyl-imidazole-2-carboxamide;formic acid;formate;
5-[4-(difluoromethoxy)-2,3-difluoro-phenyl]-N-[3-ethyl-4-[4-[1-methyl-1-(pyrrolidin-3-ylmethyl)piperidin-1-ium-4-carbonyl]piperazine-1-carbonyl]phenyl]-1-methyl-imidazole-2-carboxamide;2,2,2-trifluoroacetate;2,2,2-trifluoroacetic acid;
N-[3-chloro-4-[4-[2-[1-methyl-1-(pyrrolidin-3-ylmethyl)pyrrolidin-1-ium-3-yl]acetyl]piperazine-1-carbonyl]phenyl]-5-[4-(difluoromethoxy)-2,3-difluoro-phenyl]-1-methyl-imidazole-2-carboxamide;2,2,2-trifluoroacetate;2,2,2-trifluoroacetic acid;
N-[4-[4-[1-(azetidin-3-ylmethyl)-1-methyl-piperidin-1-ium-4-carbonyl]piperazine-1-carbonyl]-3-ethyl-phenyl]-5-[4-(difluoromethoxy)-2,3-difluoro-phenyl]-1-methyl-imidazole-2-carboxamide;2,2,2-trifluoroacetate;2,2,2-trifluoroacetic acid;
5-[4-(difluoromethoxy)-2,3-difluoro-phenyl]-N-[3-ethyl-4-[4-[1-methyl-1-(4-piperidylmethyl)piperidin-1-ium-4-carbonyl]piperazine-1-carbonyl]phenyl]-1-methyl-imidazole-2-carboxamide;2,2,2-trifluoroacetate;2,2,2-trifluoroacetic acid;
N-[4-[4-[1-(2-amino-2-oxo-ethyl)-1-(azetidin-3-ylmethyl)piperidin-1-ium-4-carbonyl]piperazine-1-carbonyl]-3-ethyl-phenyl]-5-[4-(difluoromethoxy)-2,3-difluoro-phenyl]-1-methyl-imidazole-2-carboxamide;2,2,2-trifluoroacetate;2,2,2-trifluoroacetic acid;
N-[3-chloro-4-[[1-[(1,1-dimethylazetidin-1-ium-3-yl)methyl]pyrazol-4-yl]methylcarbamoyl]phenyl]-5-(2,3-difluoro-4-methoxy-phenyl)-1-methyl-imidazole-2-carboxamide;formate;
[2-[[5-[[[2-chloro-4-[[5-(2,3-difluoro-4-methoxy-phenyl)-1-methyl-imidazole-2-carbonyl]amino]benzoyl]amino]methyl]-3-methyl-thiazol-3-ium-2-yl]amino]-2-oxo-ethyl]-trimethyl-ammonium;formate;
N-[3-chloro-4-[4-[2-(1-methylpiperazin-1-ium-1-yl)acetyl]piperazine-1-carbonyl]phenyl]-5-[4-(difluoromethoxy)-2,3-difluoro-phenyl]-1-methyl-imidazole-2-carboxamide;2,2,2-trifluoroacetate;2,2,2-trifluoroacetic acid;
N-[3-chloro-4-[4-[2-(1-methylpyrrolidin-1-ium-1-yl)acetyl]piperazine-1-carbonyl]phenyl]-5-[4-(difluoromethoxy)-2,3-difluoro-phenyl]-1-methyl-imidazole-2-carboxamide;formate;
N-[4-[4-[2-(3-carbamoyl-1-methyl-pyrrolidin-1-ium-1-yl)acetyl]piperazine-1-carbonyl]-3-chloro-phenyl]-5-[4-(difluoromethoxy)-2,3-difluoro-phenyl]-1-methyl-imidazole-2-carboxamide;formate;
N-[3-chloro-4-[4-[2-(4-methylmorpholin-4-ium-4-yl)acetyl]piperazine-1-carbonyl]phenyl]-5-(2,3-difluoro-4-methoxy-phenyl)-1-methyl-imidazole-2-carboxamide;formate;
N-[3-chloro-4-[4-[2-(1-methylpyrrolidin-1-ium-1-yl)acetyl]piperazine-1-carbonyl]phenyl]-5-(2,3-difluoro-4-methoxy-phenyl)-1-methyl-imidazole-2-carboxamide;formate;
N-[3-chloro-4-[4-[2-(3-hydroxy-1-methyl-pyrrolidin-1-ium-1-yl)acetyl]piperazine-1-carbonyl]phenyl]-5-(2,3-difluoro-4-methoxy-phenyl)-1-methyl-imidazole-2-carboxamide;formate;
2-[(1R,5S)-6-[4-[2-chloro-4-[[5-[2,3-difluoro-4-(fluoromethoxy)phenyl]-1-methyl-imidazole-2-carbonyl]amino]benzoyl]piperazine-1-carbonyl]-3-methyl-3-azoniabicyclo[3.1.0]hexan-3-yl]acetic acid;2,2,2-trifluoroacetate;
2-[4-[4-[2-chloro-4-[[5-[4-(difluoromethoxy)-2,3-difluoro-phenyl]-1-methyl-imidazole-2-carbonyl]amino]benzoyl]piperazine-1-carbonyl]-1-methyl-piperidin-1-ium-1-yl]acetic acid;2,2,2-trifluoroacetate;
2-[1-(3-aminopropyl)-4-[4-[2-chloro-4-[[5-(2,3-difluoro-4-methoxy-phenyl)-1-methyl-imidazole-2-carbonyl]amino]benzoyl]piperazine-1-carbonyl]piperidin-1-ium-1-yl]acetic acid;formate;
2-[1-(azetidin-3-ylmethyl)-4-[4-[2-chloro-4-[[5-(2,3-difluoro-4-methoxy-phenyl)-1-methyl-imidazole-2-carbonyl]amino]benzoyl]piperazine-1-carbonyl]piperidin-1-ium-1-yl]acetic acid;formate;
bis(3-aminopropyl)-(carboxymethyl)-[4-[4-[2-chloro-4-[[5-(2,3-difluoro-4-methoxy-phenyl)-1-methyl-imidazole-2-carbonyl]amino]benzoyl]piperazin-1-yl]-4-oxo-butyl]ammonium;formate;
3-aminopropyl-(carboxymethyl)-[4-[4-[2-chloro-4-[[5-(2,3-difluoro-4-methoxy-phenyl)-1-methyl-imidazole-2-carbonyl]amino]benzoyl]piperazin-1-yl]-4-oxo-butyl]-methyl-ammonium;formate;
2-[4-[4-[2-chloro-4-[[5-(2,3-difluoro-4-methoxy-phenyl)-1-methyl-imidazole-2-carbonyl]amino]benzoyl]piperazine-1-carbonyl]-1-methyl-piperazin-1-ium-1-yl]acetic acid formate;
2-[(3aS,6aR)-5-(3-aminopropyl)-2-[4-[2-chloro-4-[[5-(2,3-difluoro-4-methoxy-phenyl)-1-methyl-imidazole-2-carbonyl]amino]benzoyl]piperazine-1-carbonyl]-1,3,3a,4,6,6a-hexahydropyrrolo[3,4-c]pyrrol-5-ium-5-yl]acetic acid chloride hydrochloride;
2-[1-(3-aminopropyl)-4-[4-[2-chloro-4-[[5-(2,3-difluoro-4-methoxy-phenyl)-1-methyl-imidazole-2-carbonyl]amino]benzoyl]piperazine-1-carbonyl]piperazin-1-ium-1-yl]acetic acid formate;
2-[4-[4-[2-chloro-4-[[5-(2,3-difluoro-4-methoxy-phenyl)-1-methyl-imidazole-2-carbonyl]amino]benzoyl]piperazine-1-carbonyl]-1-[2-(dimethylamino)ethyl]piperidin-1-ium-1-yl]acetic acid bromide;
2-[1-(azetidin-3-ylmethyl)-4-[4-[2-chloro-4-[[5-(2-chloro-3-fluoro-4-methoxy-phenyl)-1-methyl-imidazole-2-carbonyl]amino]benzoyl]piperazine-1-carbonyl]piperidin-1-ium-1-yl]acetic acid;formate;
2-[1-(azetidin-3-ylmethyl)-4-[4-[2-chloro-4-[[5-[2-chloro-3-fluoro-4-(fluoromethoxy)phenyl]-1-methyl-imidazole-2-carbonyl]amino]benzoyl]piperazine-1-carbonyl]piperidin-1-ium-1-yl]acetic acid;formate;
N-[4-[4-[1-(azetidin-3-ylmethyl)-1-methyl-piperidin-1-ium-4-carbonyl]piperazine-1-carbonyl]-3-fluoro-phenyl]-5-[4-(difluoromethoxy)-2,3-difluoro-phenyl]-1-methyl-imidazole-2-carboxamide;2,2,2-trifluoroacetate;2,2,2-trifluoroacetic acid; and
N-[3-chloro-4-[4-[[(2S,4R)-4-hydroxy-1,1-dimethyl-pyrrolidin-1-ium-2-carbonyl]amino]piperidine-1-carbonyl]phenyl]-5-(2,3-difluoro-4-methoxy-phenyl)-1-methyl-imidazole-2-carboxamide;formate.

15. (canceled)

16. A pharmaceutical composition comprising a compound of formula (I) according to claim 1, or a pharmaceutically acceptable salt thereof, and a therapeutically inert carrier.

17. A compound of formula (I) according to claim 1, or a pharmaceutically acceptable salt thereof, for use as antibiotic.

18. A compound of formula (I) according to claim 1, or a pharmaceutically acceptable salt thereof, for use in the treatment or prevention of nosocomial infections and resulting diseases.

19. A compound of formula (I) according to claim 1, or a pharmaceutically acceptable salt thereof, for use in the treatment or prevention of infections and resulting diseases caused by Gram-negative bacteria.

20. The compound for use according to claim 19, wherein said Gram-negative bacteria are selected from Klebsiella pneumoniae, Acinetobacter baumannii, Pseudomonas aeruginosa, Enterobacter species and E. coli.

21. The compound for use according to claim 20, wherein said Gram-negative bacteria are Acinetobacter baumannii.

22. (canceled)

23. A method for the treatment of infections and resulting diseases caused by Enterococcus faecium, Staphylococcus aureus, Klebsiella pneumoniae, Acinetobacter baumannii, Pseudomonas aeruginosa, Enterobacter species or E. coli, or a combination therof, which method comprises administering a compound of formula (I) according to claim 1, or a pharmaceutically acceptable salt thereof, to a mammal.

24. (canceled)

25. (canceled)

26. (canceled)

27. (canceled)
